SEQUENCE LISTING (20 sequences)

SEQ ID NO: 1 - DNA, 86267 bp, Homo sapiens
  source 1..86267: mol_type = other DNA
tttgatatgacgattagagcataacccgagtgacacgttgaattcgccataatcaaggaaaccttttccgggtggggatctctgaaattactcaggtatgatccatgtgttttgcttttttctatttcaactctgtgaaactgtgtattgtggtatcagtgggaatgcagtgaaaacagcttttaccctcttctgttatcgagcctaattcaaagaattatctagcttgtttttagactaatgaaacaaccagaaaacaaaagaatatattggaattattatataatctctaaatgagatagctgccaggatttaataaatttctgtagacttaccacggagggttcaagagtggagccaatgttgtgagaagtttatttagctctaaccagacagcagggagagttgtggttgagtgcctgtcccaatcctaggaccctgagaaacaaaaatataattaacaaaagaaatagtattaacatcaccagcagattttgagttctgtgtgtgggtcataattcctatcatactttagacttcatatattcaattatctccagagctactaccaccataaaaaagaaagtcttggtacaactcttgcgattccgcctgccattatcttgctaaccaaaggaatgtttaaactattcacttttgattctgaccaaaaaaatattatggaaaatgactggttttcagtgtgttaaatctgaaccttggaatggctaattattatattgtttccacaatttgggcaactgtgctgaggtggatatattttcacatattttttttgtgtactccaaaaacattataatggtattttagctaaagaaagaaatcaatctggacatttaccaaaaatatcccatttttaccagaaaccccctaaactctctgactttttgtcatgattacctatgtaaattccaagaagaaaggattccaaactaaacaattttatttactataggacttttcagatatgtattgtatatctccaagaagatgaagaacacagcattatttgcacattgtgcaggattagtgttttatagatcgtattttggcaaattcagctctagttatagtttaggagcatcaaaaaatgtgttggtctgctctgaaattttctttataacgtgtgctatggagcatgcaatgggagcacaggtctggaaatgacagtgatatcacttatttgtcaaagcagtaaggttttgccctgctaaatacagagaaagagacaaatgacttataaggttcatactttctggaataacattgactctgcccatttcagacaactcttaacattttactgttctattttactatttatcgatttcaagggaaaaaagaagacagctggcaaggaaaagaaatattgttgtgggcaaacattataaaatgacataatggatattaaaacactataaatactttgttcttattttctactgcactaatgccaaggtggttttatgtcatttcaaatatcaacatttcctacactatattcagtggatgttaataaatgtggctcaaaagaaaagcaactccattgtcaagtaagttgaggaaacagtgttagaaaagttatagcaggttttgttaccacatgagtgttttgagtaataatgataacaagcatctgtgaagcactatattatcgggtctacttctaagttctctccatgcattatctcatgttattcttggaacaactctatggtgtgatactattataacagtttataaatgggaaaatggaggcacagacaaattacctaacaggctcagctactcagttagcaagtggcagagccaggctcctttgtctggtagtctgatccagagcccaagcacgtaaccattatatcatacgctcgagcactggggactcatgacacatgcaaatattgtcatttatcacaactatctaagagagtaggcaacgttttgtttcctgggcttaatttacaacagaacatcttctttcaggaacatctatcaatatctctcaaaattggcatttctcagaatacagaacaagaaagaccaaatcgcttttatagcccatgtccagcattcagaacaatcagaatccattgaaataatcacagttgaaaaatctaaaggactttagagtctagtcttccaccaaataggactatgcctatctgagaaacttttaaaattaattacggaaagaacactaggctttgtgcctcgacacagctaagtggaaacgcctattgtaaaactccctaggtttgtgaccttgggcatattgtttgactccactgaatctcagattcctcaaatgtgaaatgggggtaagatctgacccatagtattttgtgaagtttaaattagatgataaagcaaaggagagaaatgtggtccccggtacttaagaattcagcaaaaaccccacttcccttttcattttcctatttgtaacaacctccagaaaaattttctaaaattgcccaagtatacctattaggccttgtttttcaaaacaatactaataaagttgaaatcacaaactgtacataaaaaattttgattaatcacaaatagtagcactattccatttattcagagaaagacagtctgtaaatttttttagtattaaagcacacacacagagtatatttttattttagagattattgctgaagaaaactcttagttataaaaccccaacaccttttcacccaaacccatgggacaccctgacacagtgcatgctcatttcatcatggaggcagttgatgttaaaaatctgtttcagtaactgtcttttaaaaattgtcatcccttagaaagctgagtagtactgtgggtactattgcacaatggttttcattaccttatgtgtctctaaaatgactcaataagttcaaacaagacagtattgatcgacagaggataaacaccattttatcttattcacagaaacagtaaccaggctcctgcaaagtgcagctctcacaatccaactgcccccaggaaaagcatctggaaaaggagcagcactcttgcggtgacattctgtattagaaatctgtatcccatctgctccttggcctgatcctgtaaaatctggagagttattaacaaatgtttgctgctgattcaaccaggaaagctagttcatcatttcatttcatcaaatctactccaactaggttatcagttttttgttgttgatgatggtttgtttgtatgagatggagtatcgttctgttgcctaggctggagtggtggtgcaatctcagctcatcacaacctccacctcctggattcaagtgattcttgtgcctcagcctcccgagtagctgggattacaggcacacgccaccatgccaggctaacttttgtatttttagtagagacggggtttcattatgttggctaggctggtctcgaactcctgaactcatgatctgcctgcctcagcctcccaaagtgctgggattacaggcatgagccaacatgcccggtctaggttatcagttttaatgaccacttagaatttttaatttaataaactaaaattttgtattgaatatttttgattgatgcaatccaaaaattaaaacatgcccttttgtaaaaaatcatcgtcctgtataagatatgctggtatcttgagagctctaatttgatttcttttaaacatatgtaagctaagatttttatcctatatagatttatatgtggagttttatgcatatatgtattaacaatcacatggagaacattttccaagtttgtttattcaataggaattggtccctggtaatattaagagagaaatactaataaggatgtaagataaatccttctgatttgcacagttaagtgcaatgctctgctccagatgtaatatgactaaagagaattaacacatgacaatttaggcagacagaatgtctaagtgagcagattggtttataattctataaaatcccacctgcagtagtgatgtagtgatgccttccctccctccagcaattaaaaactattcattgttttaggatgcccaattgatttaaaaatcactaggtttaagtagtagaactaactacaatatcttatttccataggatattctagtttataaagcttttcatatacaagaacttataaagatttttaatggcaaatttcttatagttgatgtcaattcagtatgcctagtttcttcaaggtaactgtgtcaatattcaccattccaattgtacaaatctgattaactgaaagatgctcccacctatcgtttataagcccaactctgtttatgtaacacaatgatgtccaacttcactggacattaactaatgtacctatcagttaagaggctagatgaggccgggcgcagtggctcacgcctgtaattcgaacactatgggaggccaaggcaggcagatcacctgaggtcaggagttcaagaccagtctggctaacatgacgaaaccccacctccagaaaaatacaaaaattagccaggcatggtggtgggtacctgtaatcccagctacccaggaggctgaggcgggaaaatcacttgaacccaggaggcgaaggttgcagtgagccgagatcgcaccattgcactccagcctaggcgacagagtgagactccacctcaaaaaaaaaaaaagagtctaaatgaaaacagtgaagagaggacataaaggtccatgtggaaggtgagagaagaaccacccccatatgggacctagaagaaaacagccctaaaaagatgtaattataactcagtccagatgctgtgactaacacagtgaaagatataaaggaacagaggtttataaaatctcctacctgcctggcaggggactggcaaaacattgtgcacaaatgaaattttttatttctattgattttagtattctatccagctcttctactgggattctgcttagtgcttatctttccatctggggccttcagcatgaactaaaatgtactttaaattattagatgtaattaaactctaatgcctctgattatttgaaatggaagtcagaaatcaaaatcaggttccatggggggaaaactgtgttggcacatatagatgttcatctcaggaatcctctttctaaccttcttttccgaagatccctaagagttcctcgccattcatgtacttactaccaactaattataattggcaccctacgatgatgataacaatgatgttaataaaatctcttacatcctcttacatcctgccacatgccaggcacataggttaactcatttaatcctccatcactctatggaatgggtactattgctacgccatttttccaataaagcagctacagcttagggacacagagaaatttgccccaggtcacaccactaataagtggtgcaggagtgagtccgggcagtctggcacaggccaccctgtgaacaaaggacaaatggaacactgggaagcatcaaggcttagaactgaagaaaacttttgagatcctttatttgacagagaaaagcaagttgggttccccagcttgggtttttgttagaatgagaacccaagagcagaacctgaaccctctgtggatacttttccattataccacttcccactgtaaagcatttctaattcagcctgattctctgtgaaaattctgagagacgaattaatagaatccctatagttaaacaggaatactcccttcactccaagtctcaccttccttgctgtgcccagactcatggactgacctagccagagactcagttaaaggagacccattttgtgacttggagagactgacaggcccacatcacagaggaaatacatgcagacacagacatggatatggtttagctttttgtccccacccaaatctcatcttgaattgtaatccttaggcctgaaggaaagaatctggtgagaagtgataggattatgggggtggtttcccccatgctgttttcatgataatgagtgaattctcatgagacctggtggttttataagtgtttgacttttccttctgtacacactacttctctcccctgctgtcacataagaaggcctaagcttgcttcccctttgccttccaccatgattgtgagtttcctgaggcctccccagccatgcagaactgtgagtcaattaaacctctttctttataaattacccagtctcaggtatttctttgtagcagtatgagaacagactaatacaggcaccatgaacatagtgacaggataaaccatggaggcattgataaaaataaaaagtaattgtgatgaatgacttggttgcaattacactcaaattctgttagtgtctgaagaggacatagcacctcctacctggaggacatacccttttccccatggaaaaaataattgggctgctaggtctgtgtctgctaagggagtcccatatttaaactgacctaattcagaactttaaattgagactcagatgctcccaaatagcagctccaccaacctgaatagagttactcatttagaataagagtttgttcccacaagttttgttaaaatacaaattagttgggatgcggggtgtgagtgatagggactaaaatcgcttaacagactagcctggcaaagccctgtgggaagcaagttttgatgatagggaagagaagatgtcaagagcagatggtccagggaggaagtgtcacaagtaacttgggggtgatataagggacttgagtagcaggtatttgtctgatctttaaggaattaacttattaaagaattttgttagtaaagtcatttgagtatcacacttgcaacatactaattcatccatgtctagttttctcattaaatcacagatatttaacaggtgcattaacttgaattgacatcatgcagcaaatgccattatatggacccttaagatattttaagagaaaaacctataatgcctggaaactcttagttttctctgacatttaaaaacaagccgtaaaatcaaatcctttctataataatttagtaactcagaattgcaaaggatttcctaaagcccaatgatccaaagctctgacctgtgatgttggcatagcagagtgggagacaaatcatatgttaaagcagaaggctctggatctggaatgcttggatacaaattctggctctaccacttgctacaatttaactctaggcaaattactgaactctgtgactatttcctcttctgtaaagtcagggttgtttcaataattagatcagaaattgtaaagtgcttagaataataccaagcaagtgtgcataaactttagccatttagctataactctcatcatatttctgtgttgattacaaaatgctttactagaggaaaatagatgagaaagaggccatgaggtaaaaatgtacagtgctttttaaagtagaatgaggtggcttagaaattaattctatccccatcactggcttgtagtagaaatttgaaacagtctctccctgattctctggcagtttttctctttttttcttttttttctttttgagatggagtctcactctgtcacccaggctggagtgtagtagcgtgatctcagctcactgcaacctccgcttcccgggttcaagcgattctcctgcctcagcctcctgagtagctgggactacaagcatgtgccaccacaactggctaattttttgtatttttggtagagacagggtttcaccttgttagccaggatggtctcgatctcctgacctcgtgatcctcctgcctcagcctcccaaagtggggggattacaggcgtgagccacggcgcccggccctctttcagttttttctaagacagagacactccctatcacagacagggtcttctaaattagtagagaatgatttgaaggaaactgggtaattatcagaggaagtaaaagagaaatacagatcctcacaccagaatgcaaatctctaagactttattattttcatcactatgaaagaaaagatcagtactcaaagccacttatgatgatgcatgtaaattagcataaaatcagaacaaatataatatggcttttaaaatagaaacataaggctatctttccttacaagtgtcagattgcttctgtgccactgtaacacagagctttagatggattttttaaagcttcactcagagatcaaatgtactccattagtcagtgattggcagacttggtgaaaattgagttttcaacttaaatttctccaaattggtctgaggaaaccagatgcagcaactaagttcagaaggtcagagtctcaataaggttaaaaagagagggagaaacataacactagaagtatgaatgaatgaaatagaccacacacagctcatcaggtctctataatataccacgcaaataattggtaccaggactattgaataatcaactgcttttaagctgaagaatatctacattgtataccatgattaccactttaccaattcaaaagacaacttaaaaattaaacatatatacaaagacattctgcttttttcattaggccatgtcattctataatttctaaattctgtcaactctcttccccaggaatcttctagaagtcaggcattttgggaatagttgagtatcataattgaataaagatagcacaacagtcacctgtagtagtctgatcttacaggtactagaacaataacagcacaaaaacagagttacgtgtatgtattattcagtgttctctagaagaaaacatagtatatgtatatataggcaatacatttataaatgcataaaacaaatataaatttgtgtaagtatgcatataaatatgtaagtctacacatataaatacaaataaagagatttattctatgggattggctcatgcaattatagaagcaggagtcccaagatttgcagtaagcaagctggagattcaggggagcccatgcatagttccagcccaagtccaaaggcttgagacccagaaaagcaatagtgtaagttccagtccagaagccatcaggttagagacgcaagaagagccaatatttcaagtccgaagatcaggaaaggccaatgtccctgctctgtcatgcaggcccagctccctcttactcagcttttatgttttattcaggttttcaattgattggatgaggcccactcacattgcatctgctgttaaaattttccaagatctttttggggttaaaaacctatcattcatctctcaatcttcagtgttgacaaggacagagcacttatgagtattcctgtttctgagtttctgataagcttgtccaagtggagcacaggctggacaatttataagacagtattgaagtcagagcaattacaaaaggaaaggggtaggtttcttgtgtgatgtgctgcaaacactgacctctcagaatttattcggccctgtgtgtatatacgtattcatgtctgcacatatgtgctagttcacttatacatctatacatacacacaatcacatatacccaagatctgatggaatagacatgaatttggcttagccagggaaattatagaaagaataatgggaatcctaggcatgtgggaaatgggaaacaaggacaaaggcgcctaggtaaaataaaaagcaactggtgtgcctaggagacctgatggacttgagcatggaatagctccagacttgtattgaaagaaaatcaggaaagtcaagttacgaccaggtcatggcaggcactgaatgtcaggttaaggggctgaactgtttctttctagttagtgatgggtccagtgaaggatttggggcaggagaatactagaaggaatattttagggagattgatcttccagtggtgtgcagaacagatatgccatgcctagagggtagtaagcagtgacatttctttcactctacactttagctgagctcagggtaagtccagtaatggaagatggggtgggaaatagagctaggagtgtagagagggcaacaggtaaggatacatatgtaccaaaagcagaaaacaagttaagatccaggcttcatgaggtgcctatattgaagattgctctggataaagagactgttaggacttcagagttagaattcatagcaattcacagaactgctcaggctcttcattattcacctgactcaactctcctacatctgctcctttctcctaagagatttccttgctctctgtcctttctctaccccacagcttttgcttcctttccgtacatagaaccacacaacaagtactagttttatgcatgcagtggtcatataaatatcttattggcgagtcctcatcaaaactgagttgaaacaaaccatttgagccagaatttagtaagagtcagattttttaaggtacttcaaggattttatttataaatacaactaggaaaacatttactcattagaaatgatgaaaccatagacaaaacaattattagagaaaattttgaattattcttttctcctgctgtctaatttctacctttctcatgtagtctttttctttttaattagattttttatgtgagtatatggtccatgtatttggctggtggaataaattcaattattctcaattgttttgtttgacctgtcacgtgtgtaaattagagcttcagtcagctgttctagatgcaactgcatattgttatcacctgagaagctttaaaatctgtcatttcctgggccccattccaaaccagctgaatcagaatccttccgggagggtaagatgatgcctaagcacaggtgattctaccatgagtccaggcagggagtctggtttttgtgcacagcttctgtatgtcatggctattggcacctggctctgctacggcggttcttaagtgatcttttaatgccgattgggagtatgttttaatagtggcacaatattttaagaataaggctttaaaggaagttattgcaagtaacagttaagggatttactcaaactggacacaacctctgttggagcagattacagtgaagttttgggggcatgggataaaaaagaggaaggagagatttaatggagcgaatattatttgctaaaatttaagaacgtttgcagttaaatctcgtactattgcaacaagatgtgcacaggtgcataggtgaatgcttcctcttctggtatttccttgtgtcatttatagttcaagttaaatcctagggatttaaagtgcttctcattgatatttaaagggcatttttaacaaatggagaaatgtctttcgaaacagataaaatgtacctttataaaatccactttatgagaatatgctaaaaaggaagatgtgtttctattggatcataaattatacaattttaaacagatttttatatgaaagtaaattgttccaactatttgaaattactttttcctcaaacaaaacaaaaaagatttctagatgccaggattgttttgcaaaaatgatgatctgatgatctgtgggccagatgaatatgcttaactttcctaagagttaaggtaacatttttcctttaatgagcctgtatctgatttctgatttgatgtagttttttaaaaattgagaaaatatgcctcctctaccccttgcatgttttaatgccctgatttaatgccggtgtgtgtacatatgtgcatatacacagatatatacatatatacacaaacacatattaaaattataataaaataatcatttgttcattttatacctgcttgttctttttctctgataaactctaagacagtggttcctatggtattttttctctgatatagtttttcttttctctatagatattttcattacagtgcaatttttccagtgtcttaggttttgctattttaatttactctacttaggacttagaatgattccatataggatggtatatagattttaaacttcaacaaacagtaatcaaacatcagctagccctacgcatccttgcaaaaataacaatttatttagaaagttgtatcagcattactcctctacaacatttaatatcttgccttaaaagttcaatatacatgtcagatggattcagaaacttctttagccatgggtgtcaccaaaagagcaggaaagggaacaaaaaattaaagaataaaacagaaaaagagaaaaaggaaaaatgaattatatggtcatttgaggatggagagtgagaagaccttactcattccctgtgtgtagctgtgtgtaacacaactacacatagttgtgttagctgtcttgagaagcacttggggctttgattacctccattgcaacctcatgagatatttggtctatagataatttcttcttctcttgctatctacaatccagaagaactaaagatcagtggaaagaagaatcaacactttaaagaatttaaatggctgggcgaggtggctcacacctataatcccagcattttgggaggccaaggtgagtgaatcactggaggtcaggagttcgagaccagcctggccaaaatggcaaaaccctgtctctactaaaaatacaaaaattagccaggcatagtggcacacatctgtaatcccagctacttgggaggctgaggcaggagaatttcttgaacccaggaggagaaagttgcagtgagctgagatcaccccaccacactccagcctgggtgatagagcgagattctgtctcaaaaaaaaaaaaaaaaaaaaaaaaaaaggaacttaaatataagaataaaagtaagcatgaaagctgagccagatcttgccaaatgatacatcatttggtaaaccatgaaggcgccacacttgccaaggagtaacatttcaatgagatatgactctgcatagcagacagctgggtccatttggaccatatccacaggggatcaatccttgatgtgtccgagtgaagcatcagacaatccacccacaccactcctcgaaggagctattacaatagctcactttcattccggcaaagaggctaagttaccatagtgcttcagatgctccagaccatgtgtccctatggaagagaatggggaccacagcatccccctcattggttcattatggtgtcctgtgttcttcacagcaacactttaaggcaggcagggtagatataatttccacaccccctgcctatttttttttaagatgaggaaactggaaagaagtaaaattagtaattaggaacacaatctcagcctccaggctacctaaattcaaatcatctgtctgccactcattaccagtgtaaccttgggcactttagcctctctctaccttgatttattacccatatcataggaataataatagctacatcactgagcttttatgaggattaaatgaattatgtttaaaattttcagaacatagcatatgtgttcagtaaatgttagctatgtttattatccttatttaaaatatttttacatattgccacataataggcaaaaaaatgttatcttggttttttgttttaatttgccttttcctgatcactaaagattaccatttttaaatcatgtttataagctattttgatttctttaaattgacagttcatttcctttgctcatttgtccactgatatgtgtctattatttaaaagtaatctgtatattacagataatatttctctatctattatataaataacaattattctcttcctgtctgtagactctttttaacttcatttattttgccattacagaagtttgtaatatttttgtagtcaaatctgctagtcttttcatttatggttcttaatatgcttggtttagaagaccttctgtaccatgagattataaaaacactctactatatttacttctggaagttacacaatgtttttaaaggttatttctttattccatttagaatgctggcatgttatgaggtgatatgcccactttattttttttctcaaattgtttatgatccccctatgaatttgaaatagcacctattggtgaattccagtataaacaaaactatgggtccagagtttttattttactccattaatctggttctctatttctatgcttgttttaatctaaactgttaattagggctgttccaaatcattatacactttcaatatatgttgtggcatttattgcacattgttcctgcccggtgaactatgaaatcaatttgtcaaatttcagaataatttcattggactttaaattagaattgcattaaagtaataatttgggaaaaactgatatttttacatcagcaaaacttccatttactaaggcctttagatctttcagtcaaattttatcatttttttatataggttttaaacttttctcgttaagtttattcaacttttatgtttttattctattgtttagaccttattatacttcacttgttatttctattaaatacagaacatttattttataattttccttgtagtaatctttcattagtttcaatcatattatatagttgatttacttgtttttaatgagaataaatgattgtggtgtttcattactaagtataatgtttgtttcatgtttctgagagatgctctttatcaatttaaggatgttttgttctattgttcacttatgaagattttttttaaaccaatactgggtattaactttcattaagtgccatttttagatttactgtgatgatcacgtgttttctttttaacagtgaaatgaattaaacttcgataatccctaatgtttaataaactttatatttttgtagtgatcccatcttgttcattgtatattattattttggatgctttttcatatgtagttcatatctatactcctatgatactggtctagtgttctactttgtagtatatttttctctatcagatatgatgagtttgtagataatttcagaagttttctattattttctactcttcaaaaggctttacatggcaaaggaataatttgcttttaaagagatttataaaacttgcctaataaaaccatctggatctggtgttatttgggaggaaaacattgaatgttttcaatatagattatgattactagtttattcaaatttttaacttatccttgagattaaaaattcacacttttctaaggttttgtccactacatgtatatttttcaaattattggtaaaagtattctcttttttaaaagtctatgaatctttggttatatccaatttttctcttgttttgaaaatttattttctcctttttaccttgtcaggattgccagctgaatgctaatttactactacactcagctgttacatacatttatcaagtctacaaatttgttttattaattctacttttatctttgattccttccaatttctttttgtccatttcattcttttcctagcttctttagttgaatgcttattttcagtgtttattgtcttgtaattaataataaacagactatcgtttacaaaaatctagtgtacatttcaaaatagctagagaaaatgattttaatgtttttagcataaagaagagacaaatgtttaaagtgatagatatcccaattatacttctttaagctttacaaattatataaatggattaaattatcacatgtaccctcaaaatatgttaaatctattatgtattaattaaaaaagaaaaacaagaatagaaacttgaccacatcttcatgaaaagtatgatattttcattaacatccatttattaacataattttaacttttatattttatattatgagcaagagctatatatcataggaagctaaaaatagtcaaataagcaagaatgaagaaaacaaaaataatttgttaatccactcacccagagataacaaattagccttgtgtaatcaaagcatatttgaatataaaaattaatacaaatgaaatcagaccatacattctgttttgtaacctgccttttctactttattatatttcacaagtatctgtctaaattattatatattcttctagaacattgatttgattagctgtactgtatcactgcttattcaaatatttctttatcattagatatttcagttgtgcctacctttcataacaatactaatatattctatgagaggtgacaaacatgaacagtcttaggaccttaacaacttttctactaggtagcaaaaacaaagataaatgaaataactggatccattagtaaggcaatataaaagagctcaagataatgcattcgaaattgaatttttctctgcattggagaaacaggaggagaataaccaatgagaaaatcacaggtcatggctaagatgagttaaacaaagcagcaaataaagaataagaatagaatatttctacagcaccttctatatgccaggtactatgttaggcgctctcacattcattaccttatttattccatgcttaaaagaaccttcaagttaggtatcagtattcttatccttttttagactagaaaactagacctcagaaaagttaaaatcatggtggcagaactaaattttttttaacttttttttttaagttcagggatacaagtgcagatttattacacaggtgaacttgtgtcatgggggtttgttgtgcagattatttcatcacccaggtgttaagcctagtacccactagttatttttcctgttcctctccttcctcccaccctgcatcctctgaaaggtgccagtgtgtgttgtccccctctatgtgtccatgtgttcccatcatttagctcccacttatatgtgagaacatatggtatttggttttctgttatcctcctctgcagcaggttccacaattttaaagatgcttacatttacatttcagttctgtccctgtgtaagtttgctacagttaattcaagaacagaactgaaatttcagtgtaagcttgttcttctttaaaaatgtggaaacctactacagaggaaaatttttgtcaatacttacaagtaaaattaacatgtccactatgaacaacttttacatttcttttctttctttaatttaagccttgtcttggcttaaattgacaagttccaaattaatatatgattttttgctaatgcctgaaaaacatagaggaggttctggttagttttttttctagtattgttttaaaaaactaagatgttgcacattgggactttttgtgatgtcttcatggctatattttgaaattttatgtatacatattctctattaattttgctatggctcatgatccatgaccaaagtgcatgctaccacaagaattaatttccaaaatagacttaagatcaattgtaaatatttttccaaggtggatattagagctttgtatcaagcaatgcaaagcaatatttacttagcatcatcaggcaaatatatagacttcagtcataaatccttcaccaaccatttcagtactatctagaatttctttcacgtaaaattcccaggagatactcaagggctgaaattcgtgctaaaaccacttgtacaacttgcatgttacataaaagttatgtccagcctacatcatgtaatgtcaaggcacaactaaacattgaggaaagactaggttcagtttggagaggtcccagaggcaagtgaagccatcatggatttgaaatgcacgtggagcacctcactgtacgggccccagtggcttctcattcaattatgaaagaacacttctctgcttaccaagtcagaaatagtctttgtccccattcagtttccttatccaatttcttccgaaaaatcaaatcaagatagtaaacatactattaacagaatgccttcatttcctaactctaaattgtctaccacgttgcagatattaaattaatatttgctgatccttttgttaaagtcgaggataattactttaatttttaaccaagaaatcttctaactttaattctgtctttatcttttgtctcctttctgttcctctctgcctgtcattcacaatgaccatttcttttgtttgtataaacttatagggtgcaagtgtaattttgttatatgcctagattgcagagtggtcaagacttttagggtatccatcatccaaataatgtacattgtaaccattaaggaatccctcatcatccaccaccttttcacttcattacccttccaaatctccattgtctatcatttcactctctacctccatgtgtacacattatgtagctcccatttatgaatgagaacagatttgtttttctgtgtctgacttgtttcacttaagaaaatagcctccagttccaaccaggttgctgcaaaatataaaataaaataaaagaatttcattctttttcatggctgaataaaattctactatacatatataatattatataaaattttctttatccaattgttgatggatacttaggttgattccatatctttgccattatgaatattgctgctataaacatatgagtgcaggtatgtttttgatatattgatttcttttcctttggatagatacccagtagtgcccaactacgatttctagttctgtttttgccaagctatgctttcatgactcgttaaatgtgtttttctagtatgtactcctcttacctttgtcttgtatcttccccaatttaccatttgaaagacattaagaaaaaaaaaaaaacaggccaggcatgttggatcacgcctgtaatcccagcagtctaggaggccgagatgggaagactgcttgagcccaggagttcgagaccaacctgggcaacatagctagccccatctctattttcttttaaaaacttatatacatatattactcattttccctctttgtctgtttccctagctgccaacttatggtctgctagggcagtaagaccttctaccattctcataacttcaggcaaatctagataacaaatctcacttaaaatcctcgcttagttttctttccatgacttggttcttcatgtgggatcagcttttaggtacaagataaatgtgtttcctctttctagctcagggttataatcaaagagcaggacatactatccagctaacaggtctagatttagaaaagtcagaatgtactacactgcctgtgtctctcttccaacttccttctcatctgtttccctgccaacttccctgaaactggacccactggcacctgaggccaacagggtcgggaactgcagctctaactttgcccaatagtgaactatggcaatttccattgagcagctgcagccaagtgacgaggtgagctattcattccaatccctctctgcctctcaagaaggcagaagtcttgaacccccaagaatttctagatctttgttttcttggtcaaataggcactgatgttgcaaatataaaaccagaaagagatagaaaagtgtatacaaatatcaactttatattttaatcactctgcactctgaatttttttacttttcttgtctctctcacaagtatgatgacatattcataaatgcagagaaacaacaaaaatcaatttcttatataaaacaaattcattccaggatggaaatttcaggaaggaactggaaaaagagaaataataagtattaggactttcaattcagattaaatttaattctgattgactgcaagtctctgtaatttataaaattgctgatccaaagttttgcatttcttctaaaagtatcaattaagcacttagcataatgtcaagctgagccttctgggcaaacaaagatgaggtacaagatagtccctgtgctcagaaagctcacagtttagtgaaaaagttagacatgtaagccaataaatacagtcatgtgcgaaacaatacaaggatgtacaggtttttttgagaggactgaggagctgacacttgaaggttacctacagaggttgtcaaagaaagaaagcatcacagagcaaagaagatgttaatcaagtctagaaggatcagtaaggccaatgtggcaaagttaggaggcatgggaaggaaaagagattccaagcagagaaactggaagtgtaagaaagcatgggaaggccaagtggcagggcatgtgaatgacactggaaggggagtctagggtctagtcctgaagggacaatgtgtcatgctaagaacctgggactttatccttcacacagttggttttgaaattctgcctcttggagttagagattgataattataataattaaaatgatataatagctaatatttattggatggttattaaattccagaaaccattttaagtattttaaaggcatattcattatttagctcccaatattaatattgaatcctcaaagaaccctatgatggaattatcatcatcctaattgtatttattttagtagtagtagtagtagtagtaatagcagcagcagcagcagtagtagtaggagtacctttgagacagggtctcattctgtcacccaggctggagtgcagtggcctaatcgcagctcactgtaagctcaacctcccaggctcaggtgatccacctcccaggtagcatgactacagatgcatgacactacacctggctaaatttttgtattttttgttgaaatgagcttttgccatgttacccaggctggtctggaactcctgggcctcccaaattgctggaattacagggatgagccactgcacccggccccatcgtaattttatatgtaaagaaacaggctcagatagcttcctctagggtgaaagtggaagaagaaaagaggagacctgccgggaggattggccaggcacggtggctcactcctgtaatcccagcactttcggaggccaaggctggcggatcacctgaggccaggaggtcgagaccagcctgaccaacatggagaaaccctatctctactaaaaatacaaaattagccaggcgtagtggtgcctgcctgtaatcccagcttctcgggaggctgaggcaggagaatcgcttgaacctgggatgcggaggttgcggtgagctgagatcatgccattgcactccagcctgggcaacaagagcaaaactccatctcaaaaaaaaagtaaagaaaagaggggaccatttttacatagcaggtttggtggtgaggatacatttaaaccaagtgttccattgctaataaaaacacagtgtctgagaggacaagaagactctgatcgccagttaatggaagctgtaagaactagaggaagccctggctttcaatactttgtgtcctttgctcatcccattgttagaagggatataaaggagaagggaagggaaaggggaggaaattatacaaggaggagttgacatttcatcaggccctttctggagtccttttgctgttcctctactctgcttctctcctccaatatgagatattagcaaacctagggcaatgcttcacaggtgttttctcatttcatcttcacaacaatatgttgcacagcctgctcatcaccccaattcacaaatgaggaaattgatgcttagagacagtatgtgaatatatgattctttcctgtaatactaaccgtagccttcccctccatccaatctccatcttttttgaccctttctgctctcacccatcctcaactagataacagtgctgtgccaaaaacctgtggattttctctacaaaaattatgtaagtatactatattttacttactgtgtattggcatatattagttgtacatattctgagggtatatgtgatattgtgatacctctatacaatgtgtaatgatcaaatcagagtaattggaatatcacatcaaacatttattttctctttatgtttggaacattataaatcttctagttattttaaaatatataattattgttaactataatttccctactatactatcaaatagtagaacttctgtctcactgtattttgtaccccttaaccaacttctattcatctccttctccccactacctttctcagcctctagtaaccacctttctacttttaacctccatgagatccactttttgtattctacttattgatcttatttattacctctctcatcccctgtaaaactccatgatattttgggggatgagaggtttggttttattttttctcatctacatccctatgtcttgaaaattcttagaagacagtagatactcaataaataatttctgaattaataggtgataaatactcatgaaagcttttctctatctttccaaagtcttaatgcctgctctatttgttttaaattctattgcttcaataagatactttaagaactattctttcataattctgatctgaagaacttacaaggtgctaaattagaagatgtattgcaaaggctttatttcaatttggccaagctttttaataaaagtttgacttgcttaaaagaaatatttatctgggattcgagttcactccttagactaggtcaacactttgagagggggttgtttttgcaaaataaaaattaaagattaagtgtttctcatgacaaattttgaattttatttccatgccctgattcccaatgacttgacactataattgagatattatacagattttctcaataacttcagagtgcatttatatgtgatcaaaagacctttctgatttcaagatacctttaataacctttactaattattaaccaaaactaaaggcagattttgcaaatataaacttgtcatttcagcatgactgttatctttttgatggaaaagatgaagttcagcgaagaacaaatctacaacttggccagaagactgtagactttgccttcaaagaggtgtaacacatacgtaaataatcccatcagaccccatccacagcctccctgctcttgcaatttaattaagcacagtgagagggtttgagagtatcctcacgaaaagacaattttggtagcatttgggaaaaaagcaagagttaatttttatctgaaagactagtttcctttgtaagtcttcaaaatgttttgccttttgcaaagtacagacactgaagctggcatgttaacttactaagttagcagattccccctaaaatgcaatgccctaaagctggacagagggtagaaatagcaaacagagagcattaactatatgcttcttcaaacaatcagtgaatgcaaactgaattgccctgagtcagcagcaggcaagaaccatcagaaaggtgaaggcaagaatatccatgtttatataaagaggatgacattgttctgtccctaaaaacagctaactcttgataatctcattttttcctttgatgttctaaattataatgaaaatatacttccttttttttttgaaacagagtctcactctgttgcccaggctgcagtgcaatggcatgatctcagcttactgcaaccttcacctcccaggttcaagcaattctcatgcttcagcttcccaagtagctgggattacaggcatgcaccaccactcctggctaatttttgcatttttaatagagacagggtttcaccatgttgggcaggctggtctcgaactcctgacctcaggtgatctgccttccttggcctcccaaagtgctgggattacaggccagagccactgcacctggccaaaatatcactttctaaatgatgcttatctattagcttggggacatttatttaattctactacttcagtagtagaatacccattgctattactaatagtagtactattagtattactatagtattactattattacgattagtattgctaatcataatatatatagtattagcatactatatgcactactatagtaatactatactagtatactaatagtagtattataaatactattaatattactattgctactatcatagtattattataattactagtaataattactattactattattatgattagtattactataatcataatagtagtaatagtaataattaccagtaattataacaatatattactattgctactactattactatattggtattactactattagtaatactagcactgtcattattaatagtgctacaattagtaatactagtgttattagtaatattggtactaatattggtgctattagtaatagtagtactattaatactaatattagtaggtagtcctaataatattgttattgttactaatagtatatcttattactattctgttattcaagtaatagtaattttagtaatagaatacctctactttagtaatagaatacctattactatattgttatcattactactattagtgatactagcactgtcattactactagtgctgcaattagtaatactagtgttagtaatattggtactaatattagtgctatcagtaatagtagtactattaatactaatattagtaggtagtcttagtaatattgctattgttagtaatattctattactattctgttattcaagtaatagtaattttagtaatagaataccttaaagctgcatttttcagaaacgatcacttcagcttcacttttttctttcacccaaattttcacccagtttcttatattattaatggtgtcttcattctgagtctttaaaggacaatgtgaagcagtacaggaattctcaacgcatatgacagaaaattttcaaaagtacctatattcaaatacattctagttgcatttgattcagatcattgtaaaacatattcctctgtcctggttggcagagcacattaagcagggtgaaaacccagcaaagaaccttccctgagccactaataactagaacccagtgtggcagccttcacctctgtgtatgtgggaacttccagggcctcacactcctacagcctagaaccttcaaaatggacattatcttgttctgggaacttgtggatgtgagcaaccatcatatttggaatgtgattcaaatcatgttcccaaaatagaaaatgtagacaatgaaatgctgaaattatacatttatatctgtacacacaaacgtgaacatatgtaaactttcatcattttttaaaatttacaaaagttttcatgtactctactgaacatgtctgtggtatgtgatctgggtgttagcaaacccttcccacgtcaagtcaaaagcatccattgttcttgttttgttaaaattaatgtcacatttttattacatagagtactttgcatgatgacttctgtgtatgtgatactggcatattttgctctaaaaatttatctttggagaataagtagataactatcttagctgacacacagactaaaactcaatctctgagaattctgatttcttatcttagtggttagaatgagcgacagtgtcctcttctcacgtctgtaaggaacattcttcatattcttttaatagatgtgagctgatctgtttttgagtatttggtgacatcctgagataagtggattcagaattactaatctgcattataattggagaatgggtattgttcatttttttttgtttctaaatttcaaccaatattgtgcatttttaaaagtttttttttaagagcacatttacacagccttgtttgtatgtaactcttagttgctctaactttaatgacgcatttacagagaatatttcctaaagatgtccttcatgaaaaggagaagtttggacttgacaacagtaatttacttaatttatcttaaatttggtttcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggtaggaacttgtgaaatctgctgagactcaccactgaatttgcacagtggtgggaatattggaggtcctaagatgcaaaatgggaatggtcagttacatagctctttagagaaatgtccctttctaaaagagaaggaaattgcaacatttctgttttgtagatgacactgaatgtaaccttctttctactctaggttatcaattcaaccttaacttgtcacagaatcagtgtccacagttatagagactatcccaaaaaggcagctagaatttgccacccaaggctatgaagtagaaccacattggtgtttcaggaatcagcagaaaataaactacagcaggaaccaggcttagtttccagagagatcactgcacacctaagacttctagatgtgggatccaaggaatgaactttttgtgtctagctgctgcagtttgttctctcagaacacttgctcatccagtctaggatatgtaatactaacttgtctccatgcttagcattttgtaaaaggaaaaaagaaaacaccacaatcttaaaactgtctatttatagacactgccttttagaaattatctgtatattattattatctttatgatatcttcttttagagaacttatgctgcatttaaaaaaatgtagtgttagatacatatattatcttatttaaagaaaaaaatgttgctgtttttacagtaggacaaagtattttaatttctcagctattttctttcactcacatactttcccaacttaaccaaatcttgaattcaaactactatgaattcaaatgtcatgtgtgattagcaataaaatatgcaagagtgtccttccattagatgctcctttgtcagttgcaaaatatacatttattttttaattaattttataaatcatggcatgctttgttagacatagtaatagttattttcttcatcctaatgaagagcgttagtcttttttaagatttcaaaaagtttctattatttcatagttgactttttgaagtttcacttaagtccatggagatactgacatattagtttttttaatacttaaaattcaaattattttagactcattaaacttttgaaaagagggttttctttctttttgcaatcaacacttccatagatgttatacaatcaagaatgtttttgaatctttttaaaattttgaacttcttgatattagaaacatttgtgcaaagacagtagaaatagaaagcatttcaaccctttcaaacagcaatctgttaatatttatatatgagtgttccttttctggaactgtatttctaatgacccataatataagatagaaatatgtataaatctgtcaagggtgacattagctgtaatgcatatcacaaacttaaatgtataaatacaagaaaattattaagtaaattatgaatgtataattattatacccaacaagcagaatttatgaaaaataatcctaaaacaatataaattatcaattacatttcattcaattctggatactggttcttccttttccccccacatgaaattcaggtaaagtctcataacttttctggtaacgttcaggtaataaaaatccttcatttaaaaaaattgatatttttttcatattgatgttattccaaactagattgtttggaataatagtaaagacataaaatcattcaaagttgcagttatttccctttctcaagtatgtcagggtggaggataatttcttttctgtttttctgcactatgttttcctctttcaacaccagtttggtaactacaatttctgactcctgattgaagcagaagggagaaataaaagcaaagaaaatggtaaaggaaagtttttactgagctaccttcattgggctctcaggcctgacaggtatcgaagcttgtgagattctttttacgatcttcagacacccagcctcctgctgggttctctttgatatttgaagcagccctcatggagaggatggaagataatcattaatttctacacgtggccaaactctgatccccggaaacacacattccctgacccaacaaaatctggaaattgaggtgacacaaagcaaccacttctccttgccaacaacaaagctgcaggccaacccattgttcacagtgagagatttcaggagtgagttagatccaccagcttagtgtgttccccaaacacaaaggcagggaatacacatcaagctgtctaagtggtttctcctgctttaggcaaaatgacaggcgcctcccaattcctcccacttagggggaactaagagtcacaacacaacagcagtcatctccaaatatctttgtgcaaattccaaaaacttctctcaaactctccaatctcttttctaagcttagggtgttgggtgacagttaacaatggaaaaatagtttcctgctaccttctcatcctgcttggtgctctagtcacttggtttcatacctaaatcagaggctagagttaatgataatttattacatatttcaaaacagctagaagagaagatttgaaatgttcccaacacaaataaatgtttgaggtgagggatatcctaattaccctgatttgatcattacatattgtacgcgtgtattaaaatatcatacatggactccttaaatgtgtacaattattatgcaccaattttaaaagtccaattccgtcttaaaatcctgtcacagatgaaatagtattcagtaactaacagtaatcatataaaaaaatctgaaggacaattgtaacacattattgttaaagctatgaagaaacctttcaaataatttttttgaaaagagacccaaataacaatggagataatcttactccatttaaaaattttgctccattgtaaggtcaaatgttatgatttaaggacatttatctgatagagaaaataatgatatgtataattgaaaatgttaacatataaaatactcctaaaatatatggccaatctttattttactttgatcttatctattaaatgatattcagaaaatgcttataataaggacctagaccacaagttaattgtaataataataataacaaaaaatattctatctgctttgtgtatattaactcatctagtccttactatacactgtgtgatagctactatgtttatccccaatttcctgatgaagaaaacaaggcacagacagtttagataacttgtgagattaagattctgttgttattggccaaaatttttggttagactcagaaaactgagagttgttataatgtgacttttaagattgcaattttagagatcagcatcctagagcatgtgacaaacatttttttggaacctaaactttgtaacatgattcactttaagtaatattgactagcagcgtgactgttgaatcagaagtgcttcaataatgtagaagcttaatgttccaaaagctagttatctatacctaaggcctctaatcaaaatgtaaatacctatataactcaatcattttgaatgaattaaagaatccctcctgattttggctacagagtgtggaaaattatccaagactttaaatcagaaggcctgtaatctaatctagtccctatgacatatgagttgtatgatctttaaaaagtcccttaacatctccaggccataatctcctggaagatcaggtacgcaatacccaactatttgtccatgatttttgtgattctagtatcttctgaatctagtcactggttagcacactggactatttggtcgaaagggtagcctaaatatgtagaaacacaacttcaaactctttgtaacttagggaaaatcctttagtcttactaatacttcagccttcctactctggattatacaaaagatcaatatgattatccaaaaagaaagaaagaaaggcaacattaagaacatcaagtttactttttaaaaaagtttttgtacaaaaattaagataaagaaacatcctaatacaatccaagcagcatacccataaaatgatagtttagaattttatagattagggtttgaaggatttacttttgctgtatctttgcagtctctgatattgaatgttgaatatttacagaacaagaattcaaaaaacagagggtgatgaatttgtgctctcttatcactagcaggacaaatacagctatcaaaaatagaattttgtgagttacttaataacgtgacccaggcttttatgtagcaactgatgcaataatgcctgcttctaagggtttggaaatatgctttgtgatctttttaaaataatcaaacatctttggatattgatgactgaagtttttatcaaagtactgtagagaaaaaagagatataatgtagtaccccacatatagtatgtatttaatacatactcttgttgaaaattgtataatattactaagttaaactttaagcttcataattgtattatgccttataaaaatattttgtaaggaagtgtgctgcagtattaacatgtgaacagtctttgtctctgtgtaatggaattatgtgtggggattcttttctctacttttttattcttaactgtacttcctgatttcgaaagctagcatatacttctttgatagctaaagacaaaaaaaattacattatgatgagtctttcctcaagaatgcattaatgatgtgggtcataaactatgatatgtgatgctcctctcacctgaaattttctccttagaactagaaaaatatattctcatatgtgagcatattcagaaaaatatacacttccacccaacgtggtgtctgttgacatcaagtttaattctattgcaactaaacctaaatatatatttaataaaaatcaccctcaggtagtcttaattttttaaaaatagactcattatatctaatatatataaaatttaaaatgtggtgtgtattagctttgtttatctagttatcctattggtagtctgttttctaaaaatcattacagcacaatcaatttttctgaggtgtgatagtccccttgctttcgtaaaccaatgatcatcctaaataaacatcaatattttttgttttgttttgttttgttcttctgttttgcttccttagtccctattattgagaaaaatgtattagtgctgggtctcgggattatagagtataaaaatgcagtgcctgacctaaagttcatataaagtgggtgaaatgaaaaagaatgtatctaaaatttacaacaacataaggcagcctcaaatgagcaatacagctgtgggtaccaaaaagatcagcattttctatatagagccttaaaattgacaatgctctttgacctgaggaatgaggacaaatatttagacataatgatgttattatagtgaaatgtataattttttagaaatttaaacttaaacatatcctaaatatccaatgtaatattaatggtcaggcaaattatgatcattcttatattttcaaattatacagtaatataaaattatgatttcaaagaattttcaaagacatggagaagtattcaattttaacatttagttaaaaatctgaaggctacatataaatttgtgaagaatattttaaaaaattattcacatacagtaaagaatattgtaaaataacaaagtataatctctaagtttttcttgcctgatgctcttcttcattttctaaattttctataataaatgcagatcgtttttatcacaaggaaaaaatctcataaggtagaaaagatttctaagggtcagtcaggagttaaaaaaaagaaaagaaaaaaatcatggaaaaaaactttattattaaatttgggtgataggtccctaggttcactagactattctttctaattctacatatacatgaaatttgtttaatattgatatagtccaatccttttgttctgcagttagaaaataagccccagaattacacaattattaatcaggggtcaacaaactatgccccatgaaccaaatgtggctacagcttgtttatgtagtaattgcaagatatgattttttttttacattttaagcattttttttaaaaaaaagcctaaaatatcaactatttggccttttacaaaaaaaaaaaaaaattgctgactccaactcgattccttagcctactgctctatattagaagcgaatctagtttttaaagaaaattctttaatttagtatttaatctcccttaaaagaagtaggtgcatcccttcttgtgctgcccattcacaccacctcaagcactgggccctcactcgttcatgtattaattcatttcttgattccaccacaaagagagacatgtttctacctttatgaagattagtctcatagaaaagatagaaaattaacagataaagaaacaagcaaagtaattacagatcatggtaagtgtcaagaatgtgataaaccaggctgtgtaaaagtcaaagccaggtgaccttctttaggaaaaattacattgaagctaagacttaccagatgagagcaaacaaactctgcaaggagccaagtatccctgggagatggagcacacaggcagtactccttaaacaaaatatttatacagtgcagaacacttttgggtagtgaagacattaatgtgtcttactttcaaagatctagtgagattacagcagataagtcggtggatttcatctttcccttcctctgttaatcagccatgaacctggtgtcctgggaacaatctcctcaaactaggaacaaaatacagtattcaattcccacccctcacactttcctttatatgcattctctcttgtatggaatttcaatgaaatcacatgttttgattctaatttgttcagttcttcagcaaatacaagccatcaggtacaaagaatggagctagatcaagtctaattagaataccgttcctcttctaagagtgccccaaattaagctaactctccatctttccggtttgagataatgtacttaggtaattttaattgaatgttcctcaaatggcattttttcctataaataatacaataaaaataacctgagggtctcaggtttactaattggcttcatctagactggctccgagaggagcagatggaaaaacactgtggcattgacattctttttcaaccactgcacaaaaaatcctagggacagaaataggaatttgacttcatggcaaaatatagctcctgggtcctactttttcctctattgcctgttttcaccacctgctcacatccctgctatccaaggacttaggtcattcaccaagtgcacggataccaattctttaaataggtagcaataagcattagaacaggatgggaccagtagattatcacaggctcatgcttccctaagcttatcaccccagccacccacaggccaactttattttaaagctcagaagcagtcttacttctactagccagagatgccatagcgtggtgtgaaagactccaaatgaaggagcagttctgtgaatccctttgtggagggggtgtgtgcccaacatttgtgggaacttcattatcccaaggaacacagatatgttccagaagctattatttccaaatagcaatgaatttctcctcgaatagcctcatagggtgttgactatatatgaagtgtttgatataagattcaaaatcttcataaacgacacaaggtagtgctttcccttaagctagaagcacagactttttctattttatcagcttaaaaattggtgtgacctcatgacccaaatatgcagaagcatgagcaggtcttgtggactttggactgcgcacgctgacaaattcacactttttcttaaagtgctattcatttgtgagtactgattggcaactaaaattaagtgactcttcatcttcagaacctgaaagcttagcattatgagatgcaaaagacagctttcaaacaagctaggcaaagaagaaattattgagatcacagttccttgcaatcatgaatttgtataggatttcagcagctgtgatcctttgtaacattgttaaagaagtgctgagcagggaaccaagatgatgttaaatggcacattgtccacatcattaaataacatccaatcccatatagagagaattattccactttcagttctctctcaggcttccaggtgaggaaagttagttgttggaggtatgatttaaacacctcttcaggccctgctaacctttttaagtcccgagagcagaactagagtgcagagcttttagtagacagtgtctatgtagaagttgaccatgatttcccttgtttgcaattgtttttataatcaccgaccatttatgggaaatgatactgatattccacttataagaacaatatattttctttttgatataagcttatttaagtaacaaagtgaatcaatgcataaagtcattaggtaattaacagaatagatgtcaattggacatggaattgaagatacactgatttttaaaattacaatatcctcttaaggctccaattgactaactcacaaaacatcctatgaacattttcctccattaaacatgatatcaaagtcagccttccttttacaagcaccctcacacccctacaggactactgcaatcacctagtcaaccctcctactccttcatagatgaaataattaaagtcagaatagatttagtgccttgccaagtccctcagcttgtcagaaattaaaattcacaagagtgttgtaaggagaagaatgattatacaaaattccttacatgcctcaaacccggtgcattggatgggattatgtttggcatatgttgaactaggaaaagatagaagagcaattacccaaaccaaatgttctttattatgtctttatctgcacatgggaagttttgcataacttaatcagatctagcggccataataatagttaattcaaaaaggcaaattgtaagtaaacaaaaaagttagatgggcctattgaataaattacacattctttaaggataaaagctccaaacggattaaggaatgactaaagctgtcacctaattacttcttgaaaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggtaagcagagggtcttagaagtgagttggcattttactcgttctcttttacagaaacacacacacacacacaaatgctttttgttttctaattttgaaaaacactcgaataagttttctgttttgtaattttgaaaaacactgacaaaatgttttctgttttctaattttgtctattttcaaattcaatattcttatgaatgaccaaaatcttacattataaaaagctttgcaaaacacatcatttaaaaaagcctatttgcttttgaatgcttcaaacgttgatactgaataatccttaaacataaagggaaattattaaaagatattattaaatgcatcaacttgaaaacataaattacttctgcttccataagatagttaacaacttcaagtgaaattgggattaatttccttggaaatagactttcaatggtcaatcttccaaagtgaaacaggaaggtgactcataatttactcatagttgtttccttagaagaaccatatcatacattttttaaagataaaatgtcagggctttgtaaaaagtgaaaccaataaatgaaagtcaagagccaagagccatttgcatctttcttcatgatttttgccatagatacatgccaccaatattatttatttagaattcattaaatctcctggctaacacagtgaaactccgtctctactaaaaagtacaaaaaattagccaggcatggttgcaggcgcctgtagtctcagctactggggaggcagaggcaggagaatagcgtgaagccaggaggcggagcttgcagtgagctgagattgcgccactgcactccagcctgggcgacagagcgagactccgtctcaaaaaaaaaaaaaaaaaaaaaaaaagaattcattaaatcaactcactatttttagtcgtttatgttaaaagcaaactttttctcacatatgtaagaaacccacaggttttgtgtgctaattatactttttctaatgtacattaaaataaatgcataattattaaaataagaaatactgacctataaaccatctaatatcatcctcacatattttgagaaacactaggctatatgatgataaagctaaattaatatttgataatgttcatatcgtagcttttcctgtctttgtttctaatctacatttccacaaaatttcaacaactcatttagtctttatacacaatttttattattcttcctactttaggaacattatatctggcattcaagttgcatacatcttgtaaaaattgtagaaaaatagccaatagaaataatgccaacttctacagtatattaagccaaataaattctgctttctaaattccagtctaaaaacagattttaccaacatgaagtcactgattttaacaagatgtgaaggtgaacaaactatacaaaatagataattattttatatacaatttagaaatgaatattccctgcagcactgtgtgttgttctcaggcaacaaaaactttagaagagaccaaaggtaaaatcacattgaagaggatcctgttggtaccttttatggccccatagactttccttaaactatctatcacataatgaaggatctagtatataggtggaatacaatagaaataatgtctaataaatacttttattcaatttcacaaatcaacaactaggaaaatacagtatagactcatgaaacacaagtgacacagtcaagagtgatgtcctccatgccttgttgtcaactttaagaaccatgcacaggagtctgatcacagaaccactgttctgggtatggtcctggtgactggcaagctggtaaaatcttgaaatgctactaagcaagggtagatttggctaggacccaagggaacaggagcaggaataaatactaacctggtcaattggaaaatgtacaggaaaacatctgaaacaagtttagttatgaaaaatacacagtttcttccctcttagagagaggggagaaaaagaaacaaagaatcaaagagaaccacaaagacgattacaaacttgaaatatgaactttccaaagagccattaaagaaactgaaattactactcaggaaaaaacaggctgaatgagggttcattaattaatgtccttccggttatgaatgggctcatttatgggaaacaataaccatctggtgtccatgattggtgaagttgagcaggaaaataaatgtaacacacaacatgaggatttatgaggctgataagcctgagaagaataatcatcacaatttttgagtactatatatgccataaagttcaccaaattgagaatgataggacctttattctagtcttttctacaataaattaaaccagtgtgatattagacaagttatgtatctctggacttagtttcctcatctataaagttagtatattgtgttaatgtaaaaatgaaggaaagataatggaactacaagcgtcttcatattttttctctgctgtttactgcttgtatgaatttggacatattttatgccttggattccttgcctataaaatggaagtaatagtagtagcatcctcatagatttgttgagaagattaaataaatgtaaagcactcagaaaaatacctaattcaattggagatagttgttgttttttgctgtcattactgtcatgtgcaagtaacagaagctaactctgactggctaagtataaaagagtttattaaaagggtactggttcacagaaactttagatgtggagaatccaattcaagggtaagcttttaagaacaaaacccaaaaccacatcacataattgtcatgataaggaaatgttatctgccatcacagaatgctcaacattaaagtttgcaccatttgtggcttttgcatcagaactcaattttagtaagggaaaccatgattgtccatggtttagtaaaggaaaccatgattttccaataaccatgatggccccagctgcaaggaaggctagaaaaatcaagtatctgtcatttttaagttctataggagaaggcaatttttgcccagattccccaggtatataacaagttcaaacgctcagtggccaaaattaactcagttcattcaatcttcatgctaagacttactgcttagaatgatttattgaggaaggttttgtggcttttatccaaggtcaagaagatggtagacaaaatctcttgaagactcaatggcttcatcatgaaggacttgaaatatctgtgataggaggaaatttccaagtactcttgacatataaactgagtaactgggtaggctcagcatccaatatgtaaattaaagccactggatttatagttagaattgtgtcctcaattgttaatttacataggtgaagactttccacctatttgtatatgactctagagagacaacggtttgtaagtctttggaaaaataaaagtttccagagtttaataaacattctctcatgaaaaacaaaaatctatattcttttagtctgaactattaatacatttgtgctgctggggagaatgcttcccttgaggaagacatcagctaggagagaaaagagcagttataactgggctttatctattttaaactcctcaactatgaaaatagtacctgacatattagcaacccagagcccaactgtcctttctgagtaatggacagctattctaaccccatgactaatgcccattgcctgtcttaaattgatctgaacttccattactggtacattccaagaccagcgggtggctcacagagcaaatctcccctactgagtgaactacagggcatccctgaactggccagactctttacagtctcaaaaaatattctttggattttttttcaagtgtttataaaaaacaaagaaagacaattaatatgctttcttgcccagttatttcagattgtaagcaggtaaatcttcctatatgcatcatatgcataaatagttggatggcatttttagcaattatagcaatcaagaaagttgaagatatatttaaacaggtcaatctgctggttgcctatttattttcaaactatcaaagctattcaaagcagaatttctggaattaccattttactattatgagattaaagtttggtttcctcaacttgaagggtaaaaataccacccaatgtatagtaaacttataatctgttgaatgttacagacatcaaagatacataaaaatgggctcagaagcatcattatcaccgaaatggaaagagtttgcccagggagaaggatggtaatttggcaattcaggttgagtatcttttatccaaaatgcttaggaccagaagtattttggatttcagacgtttttaattttagaatttttgcatatacattataaagatatcttggagttgggacccaaagtataaacacagaatacacttataattcatatataccttatacacatagcttgaaggtaattttatacaatgcttttaacgatttcgtatacccatcacatgaggtcaggtgtggaattttctacttgtggcatcatgttggcactgaaaagttttgaattttggattttggggttagaaatatgagatatgttcttccaaatattttatgtaataaaatgatcggaaattgaaaggattttaaattgcagtagagaatttgctatttaatggcatgatacattttttcataaataaggagttttttttttttttttgagacagagtcttgctctatcacccaggtgggagtgcagtgacaacatctcagttcactgcaacctctacttcctgggttcaagtgattctcatgcctcagactcccaagtagctggaattacaggcatgtgccaccatgtccagctaatttttgtatttttagtagagatggggtttcaccatgttggccaggctggtctcgaactcctagactcaagccatccacccacctcagcctcccaaagtgctgggattacaggcataagccaccatgccagaccaataaggaatattttaatgtatgtatctgttataagtggcttccacgtatcaaaggagcaaaatgcaaaaattaagctgatgaactttgatgcctgactgcctgggttcaaattctggcttctccacttcccaaccatgtgaccttggcaaattaatttatctgggctttgataccttatgtaaatgggataataactgtccttaacccatgacattaagtaatgacagagcagtatctgacatatagtaaatgctatataggtatttgtttgaaaagaagtaaataaataaaactcattaatgtctctgaaaatgtcaattgcacatttttaaggtaattcttggttgagatataaaccactaaattaccaaggttttggggtttttttctataagcataagtatttcaatttctcagatttctcagtgatatcaaagttttactaatggacaagaatattagggtaagaaaaaagtacaggaactctctaggcaaaaatttaaatattcaattagattttaaaaatactcagttgtttgaaacaaaaaaagtaatccaggcaaagtagaagactcatatgatatctttttaagttattatttttaaaaaacgtggtatgaattattagaaataatgtgtgatcatgacccacagacaccacagatatgaattaaatactgtgatccaaagagcctttgtgctaacacttacaaaatataattgagaaagactcaagttggtgtatatgaagatttaacagggacactacaagctgctttacttcaattttaaacaacagtcatacaaagcagtgtatttaaacaactaacatatgtcttttaaaaatatacatagaaaaatatttgaaaaggcctatgaaatttttgacagtaactacttctaaggatggtacagtgatacattggagaatttttctttttactttacatgtcactgtgttttttccacaacttgtatcattttatagcatgaaacttcttaaaatattagttgtagaagttagtagtgaactcttgggctcaagggatccttctgcctcagtcagagtaagttctattgttctatagcactgcaggatgactacgtttaacaataatatattattaatatgtagttgtaaatagctagaagaaggctattccttgtgttcccaacacaaagaaatgataaatgtttgagatgatggatatactaattaccctgatctatttactctttaatgtggtaagaagatagatctcttaagtgttcttactcaatgaaattaaaaaagaaaaaaaaaatatttgcctagcagtttacaagttttcacaatgtttatctcaagcaagctgctgaagcacagagtgtggtactataatccacataaaaagctttaatttttctcctttaaccagtccactattattacttgtcaatgataaatacattcattaatgtgaaattattttgaaaaatactcagggaaattgaattaaagtcttgatcattttctcctctccttcacacctgccctggatttgcaggaaaatggacaaagaaaatatggtggccctccacctggttgggatgctgcaccccctgaaaggggctgtgaaatttttattggaaaacttccccgagacctttttgaggatgagcttataccattatgtgaaaaagtgagtccaaatttaacatttaatttttcttatcgttcaagggaaaaatactgttctcttcactttgctttcattctcagccttcttggtcagtggctgtttccattccaattaccattcctcaattatagacaaagtttctagtgattatctctttataatcactgccttgacctagctcttgtcctgttactgacagcttgaattgctacaatttctaatgactgtcaaatctatatcttgttccccaatctttccactgtcatgccatctgactacaaactatccatcaacaataaccctacaataacctgtctaaaattagatattgctccaacttgaaattttaatacagttttataaagtacaacattttcattttaaaagaatgtgtatgtttgacaaggctcaacatgtatctataatccagaatgaaatctccaggtctgaattttttgtctacctaaatgcatcaagaagattaagaacatggcttttcagttaaaggaagaatacaaaattaaattttatttttaataatttaccaagtagtttttagcaacaagcacattcctgaaaaatttagtaaatataagaatttagctctctgcctgaatgccaaaatcaattttgtgtgtgtgtgttaacagcctcagattcagaactgcacctaattttacacatacaatccctcttggcatgaggacaaatgatgaggacttaattctgtaagaatcttatatactgaatctgaaaatgagcatcaagaaacacctgaaacatagagatgtaacatgttcaaactatttcctaatgagcccagaagaaataggtgttcattactgaatacgtgagggtgggctagggtaaggctgagctgagaagtgaccaaccacaaatggcatgtgcttaatggcacttctctccagacatgcgccatcatatttatttgggcaacccttcttaaagcagccagtgatagtaaattgctcagtctagattgctgcaggacagttgtggagtagaagggagtattacatgggcaagtagagtcactatagccaggggaatagagtgttctcctgctctggaggccctagccctgagttagtagtatttaggctagcatttacattcagccagcagacagagaaaatgacaagcctcccagatcatcccctgaaacatatctgtgttcattttgatgtcatggctgaggaaaaagactgctaagaagttagtttggcgaatgagcattcttggattttattttaaacatcaatgatagaataggaccaactggaaattaaactatggagatttcagaggacaatttagaggaaatggacatatacatcaaaaaattgcactttttttgagacagtgtcttgctctgttttccaggctggagtgcagtggcacaattatggctcattacagcctctattgcctgggctcaagcaattctcccacctccgccttccaagtagctgggactacaagcacgtgccaccacgctcagctaatttttgtattttttgtagactcagggtgtcaccatcttgcccaaactggtttcaaattcccagggttaagcaatccacctgccttggcctcccaaaatgctggaattacaggtgtaagaaactgtacttatgataaaatgtgttttcatgtctctaagatgttccagataattatgtgataaaaggtcatttgccctaccaaaaacacattctgttaactcaggctgaaatgtcacttgtacacattttatttaacttaacttctctcatgaatcagataactgatctgaacccacatgaaaatatgcaaaccattccacatgggatgcaaatgccaaatagtactggcaggaggcaaacttcagaaagcattgtgtgtgtgtgtgtttgtgtgtgtatcagcacatgcacacatacattcagaagtacaaacgtataaatataaatgttctatttctattacttgcagatcggtaaaatttatgaaatgagaatgatgatggattttaatggcaacaatagaggatatgcatttgtaacattttcaaataaagtggaagccaagaatgcaatcaagcaacttaataattatgaaattaggtaagctccgcctcttctagattaaaagtgaaacaaacaatgcacctgtgtctgtttttctatcaagtttatctaatatggtgtatgccaaaaaaaaagccaagccagaaatgaaatagattagtatttttccttggcaaaaataaatttgattaaatatttattaatatcaatactatatgaataccaaactaagttctatgagtgttcaaagaaacaaaacttaataatttgcctgtttcataaatatatattgggcactgttcaaaatattgggaatatatcaaggtataatttcctacttgcagagcaaatatataagatcactcaaaatagtatgaaaaaatcaaaccagggatgggataaagttgtgtgagaaggtggctactctaatttggtgtatttaagaaaatactctctggcagagtgaaacttaatctgagacttgaatgaaaagagccagtcatgacagtcagaccttgggaaggttatttcaggaaaggagagaacatcaagtataaagttcccgaagtgaaaacaagcgtgatatgtttgaggggcagaaagcaagcccatgtggccggagagggtaaagagaggaggactgaaagcaatacagttggagagctgtgcactgtgtgaggaagtgtagatgaagtacagaagtctctgtggtgatgaatttgaattttcttctaatagaagggaggaaagagttttgattacatttatagttttttaaacattcctctgctgtttggttgagaatgtactatagcaggtgaggtttgaaagaggacaccagttagaaggctgctgcagttgtctagatttggacaagagatggtggtagttggatgagggggtccatggtggacaagataagaaagggtgagatctgggatatacacaaatctggctagagattggatttagactgagagagaattcagaattcagggaggaaacctaggattttggtccgagtatctgtgaacttgttagtgccattgcaatggaaaagactggaagaacagcagaggaaggaccttttagggcactttacatttgagatgcacattgaacatcccaggggaggtgttgagaaggtgactgtctgcattgtggtggagtcaggagacaggtcacagttggaatataaatttgggggtcagctaaatatagatagcaattaagaccatgggactaggggagagtcccatgtaattgactttgtgcaaccaattgcccgttttgataaactggaaataaaactgacatcatgaaattgtctgatttctaaaaccttcacttggagagagaaccgtcataaattctttcttaaaggaagccctgagccacgtgagaggaagattagtgcagcagttaaagagcaatagttttggagtcagccatgcttcatttgagtgccaccaagtaacttcaccattctaagcctcaatttcctcatctataaaatggggataccatctatttcataggtttattgctataacaactttacagtgcttaacccagtgtttggaacacagtgataagccgacaaatgataaaaaaaaaaaaattgtattttagcacagttcatcaattctagatgcagtttttttttttacatcttatccccttctgaaagagaactgcaccttaaaatgggtagatcttaaatttgatgaatgcagaaagagcagatttaccacaattatttttctctcactttctataggaccaaaagaagtcatacattatccttacagtctgtaaatcaattaactactggcttattttattaaaaattcctgggccggacacagtgactcacgcctgtaatcctagcactttgggaggccaaggcgggcggatcacctgaggttgggagtttgagaccagcctgaccaacctggagaaacctcatctctactaaaaatacaaaattagcagggcgtggtggcgcatgcctgtagtcccagctactcgggaggcaggagaattgcttgaacctggcagggtggagattgcagtgagccgagatcacaccactgctctccagcctgggcaagaagagcgaaactctgtctcaaaaaacaaacaaacaaacaacgccttagccacaggaagatcagttggattcggcaactacttgtagtctaaccattcaattccaaggctggattctagaaaagagtcttggagaaaacataaaggaggcttccctcccacaggtgcctccatggacccaaaagagctttgtgtagtatgtggaggattgtttgcagtaggaatcaggagatctggggtccagcccaggttcctttggacgaatgctttaacctctttgagcatcacttttatcattggcacctaaacacagagatgttctgagcattcaccatatgaaagaatatatactaaagtgctttgaaagggctacaaaacgctacattacagttttatgatttccagaattggaagtttcccccattccttggtctttcatggaaccttgacatcgtaacagtaagggttattcccaaaacggtatttgagttgagatagatatggaattggctatgtagggaatcaagtggatttactgtatctacttaacacattgttacccaatagggcctgtaaatacttttggacagcagataggatatgaataatgaataaaatcaatccactacggccactgataattaacagtatcctctgtgaattttagtgtctttgtctgtgaaatagagataaaaatatacctacctcatgaggtgttttgaaaaataaatgagataatatacatacatctcagtgctgacacatactaggtgctctatgaatgtttgcagaaggttatggtggtgatgatggaggaggagaagggaaaagaaaaggaaaaatatgacagaaattaggaagagaaagttactggccaaaattccagtcaaagcacaggttggatgccagaagcaaggtaaaggcagagtgttaccttaaaaattagggggtagaataaacggaataagacaagccagaataaacaccccatagacaacctcacctacttgcactctgccttgggtcagtcctgccagggaaagaacttttgttgcagagaagaaaccttcaatttgcttttatccaccatttctcacccacctatcattatatcagcttctacttcatcagatattcactgatcgtcatctaaatgtctggcttctagagacattaaagttgtggcctcagcttttgaaaggagatggctctttgtctccaaataattccgggaaaggtgcctgacaaaccacaaccatttaaaaataaaatatggaattctaggctcctcctgctggtaactgatatatgaccagccccctaactatcaaaaataatataatttctaaatacattaatactgtggaaaacaaggttttggtgttttcttttttaagtaaagtttaaaaaaaaacttggaagattattttttcaacccttccctcaccttgagaaccacactcctggcacaattcttactagactgagatggacctgtcacagatctgacacacggtccttggttcttcagcaaagcgtaggagacagctaggacagtggcctaaccctggtcagaaaacctgccacacaaccaagagccattcgctccatggatatcagaccaatcgcccactcacctggctgagagcttttattcatggcaaatgggtcgtttctgctaagcatacttcacccatttttaataacttagtgttattacaaaagtcatatagcctaatatgtaacacatggaaaacatcaaaaagcataaagtaataaaagttaaaaccatttctccgcaactccattgccccaaaataaccactatgttgatattttattgtctttccagatttttctaaacatttttaatcaaaatggccttcacatatacttattttatagccttttaaagcctagcagccaattgtgaactgtttttttaaaccaatatatattcttctacgttactctttttaacgactgcagagcgtgcaatggtatgaatgcctgtcttttgtttaccagtgattctcaatcaggagcagtactgtccacccaagggggcatttagaaaggtggggaggcacttttgaatgatcagattggtcactattggcatgtagtacgcaagaactagagatggtaaacgtactacagtatgcaatgagaaaatgtccaatccaaagccagtaacatctcagttgagagacattaaaccagttctttaccgtgaggtattttgctcatttccaatttgggggagattttaaagagcagtaataaatatccttaaacataaattctagggctcacttttattttccagggacatattcccagaagatactttatttggtcaaaggttgtgcacacttaagacttttgatacattttgccaaatgttctccagaatagtttaatccatgtgtactctgccagtgaaggtgtacatacctcaacttgctaagaagcaactctgtacagtggaagaaacaggaactgagatcccacagctaggtttcattccttaaaatctataacattttaagcaagtctttttatttcttggagtttcaagttcatctgtaaatggaaataacttctactctgccgatctcacagtctttttgtgtggggcagataaggtcatatgtgaaaatgtgttcaagtataaaagactgtttttaacagagatcacatcagaacagcttttctctttggctacaaaagaaaaggaatgtcaggtgttgcaaactcaagcgactgcagggccaggcaggtggcttcagtaacctgggttaggtgtagggacctcagtgttggggatgcttcagggagcggtgaggattgacaactcttgaatccacatctagcctaaagaaagcagatgctatgcagccatgaaaaaggatgagttcatgtcctttgtagggacatggatgaaactggaaaccatcattctcagcaaactatcgcaaggacaaaaaaccaaacactgcatgttctcactcataggtgggaattgaacaatgagaacacttggacacaggaaggggaacattacacaccagggcctgttgtggggtggggggaggggggagggatagcattaggagatatacctaatgcaaatgacgagttaatgggtgcagcacaccaacatggcacatgtatacatatgtaacaaacctgcatgttgtgcacatgtaccctagaacttaaagtaaaataaaataaaaaaagaaagcagatgctactcagcttcagataacttttttgtttgaaaatgtagagcacatgttgacacatctaataatcttgcaaaagaagccacaactctccaaaaatggtttcaacactatgaacccaaacatcccacatcttcaggcaacatttggcccacagccatacattttcaacctgaatcctacaaggattctcatcaatcaaaatccctgctctttttgcagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggttagcaatcccctcctcaaagatgagacttctctgcctatcctgcctgcctccgcaaagaaaataagcatttttaattttttttggctatctttcattacgctgtattttaaattttttatccttcttcactgtctcacagttgattccattccctgaagttttcagactcagttttgaaaaatattcattctgtgcattgaggctaatttacatgcactaaagaaacccagaactttaagtacagcaatcctttgatgaataaagagtcttcttaaaattaagtatatcaatcacagttacttttgtgtatagattttgttaccaggcttattagaattagggtatattctccattacagtgtagaaataaaaacctcaagtgataccttattccaatgacaactaggtgacatactttcattatgactccctgttgtctaataatttctgtctaatttgagagaagagagataaaagctctccttagaaagccacctttatttgttagttattacaaccacaggatagatcccatttggtgaataactatcaaattctctatccaccccaacactgcctcggtcagatcaacttaggaaaattttttacagttcttggctcattctaaaacattttctacaacttacacaggtttttaaatgaatactaaaataagaggatttcaaaaatgacagactggacacactggaatgaacttttctcatgaaaattatgggagagccaccagaattcaaaacaaaacaaccacaacaaagatgtctacatttatgttgcctttgtaaacactcttcaaaacaaaccaatactagatcagccccctagatatcttcaaaactaggagacagtttctggcttggggaagatctttcaataatatcttcatgaaggcaacttaaagggaaaaaaaatgagacagtcccaaaggttgctgaatcttttagagaagtagatatttttaatgttgaatctttcattcacaacttcttccctcttcactggccagcatttggtcaagtcatctaaatcacaggtccatagatagactcactaagactcctcatgggggcataccttttggtgacctggacgtaatattttgctatctgcttcaattcttctttcttcttcaacatcgacaactgtaccattgaaaagtcttttgtatccatcctgttcttaggccaccactgaagtctaaacacacatattctaaaccttatgtaatttttttcaagatctaattattctcccagctctgctttctcttccatgtgccccaccctccgacttctccatcctgaacattaccaccagattaatgtttccaaaatatcctattatcctttcaacaccttcttgcaaatagtaggaattcaatctctatctataggctgaatgatgtctgtcactaactggccagttcaaaaatattcataactccttgctgtgtgtaagaactaaatatgtgatttcattattcaataccttttattcatttcctggtcttttatgaccatatttgctaccttctatttcccaataaaaactcttccttctatcaggacagacacctcaatgacctagacacttccatgcttatgtctgtctcgtttattcctaatgctatatcctctactaggaatacctgcccctccgtcacttgtgacattcaccccagcccagccagcaccttcttgagaaacctacctccagtattccagcccgaattagtcactttcctccctcaatgcaggaagccattcattatctgtaaaaacagatatgtgaaataatttggggtgtgggggagtaggacagtgaaagaaggtccaaaacaagtgaaagacagtttccagggtcaagacaatccagtaatcccagagaagctccacctcctgaaagcttccctatctcttcagggagaatggcacattggcccaccgaaggaactcctttttctaagtcaagcagactagctagccaggtcctatcagataagcttcagaaattttcaagcacttcattaagcttccacccacagtcctgacttttccttgtggattctctgtccaggatatgccatagggaggctgcctcccctccccgtccacaactcatattcactatgatccaatgcaacccccttacctatggccctttgagccagtctcaggtggatttcactcagctggattttagtcagcaactaactcatcagcaaactaacaagatgaggcacagtggtgagtaagccagccagagcaaacttcttcacctctaatgagatctcagaacaaaacctatcacccatttggatcactctacctgttaccccattgcagactcacccacaacaaaggaattctttaattgccaaccctttgagaacctagtttaaaagaacaaggagactgttgagaaaaatgataagtgaagacaacaagaaacttttctttcttaattgacccagacttggagacagagagacaatagttgtctaaagggatgtaactgctcctcagtcacttacctcctctctaaacagtggcagttcccccttgagaaatctcacctttattgagctctttacagaacaaagttgctgcatcccactcagaaactagaaaggtgttctaagaatgctctgaagaaaagtcctggcagtgattctcaatgtcaggctgcatcagcatcacctggagggcttgttatagcacagattgctagatcccaccgccagagttcttgattcagtaggtctggtgtcaggccaggtaattcgcatttacaacaagctcccaggatgctactactcccaggactgcacatttgaaaacccatgatttgaatcaatgattttccatcctaggctgcacattggaattacctgatgttttcaaacaatgtgagtacccatgtcagcctctaatcaattgaatctgcatcactggaatataaagttagggaactggtgttttaagttttccagatgattctattcttcagtcaaattttgtgattcacagctaaatctatcagctaatgatactgggtaatcatcttcatggcatgtaaataggaatacctgggaactgctcagtcagtaaatatttattaagcatctagaacacatattgctcccttctgaatgacatgagagattctaaaaataaaaaaaaaaacatagtacctaaccattgcactcaaagagtgcatattctgagaaaactatacatacacaaaaacaggaaaaattattaagaggagagtgtattgttaaatcataatttaaaagttattaagtcttctctgcatcatcttttcctgacctcacaacaatagtaggttgtagataaggaaactgatgttagaaaggatatgttttctagccaggatgattgttctgacatttgaacttgtcttacttctagctcatgctctcaaatcactctgtaacactcactttcaccagaggccttcacaaggatgttggtggaaatcactgcttcccatatttactctcatttctgttcccttagccttatgcagctcttcgtggtcctaggctgagaagactaaagctagagcagtagtactcaactgtggctgcattttagaattaactaagctacttttaaaactcctgagatccagcccacttatatcagaattgggaagggtaggtagtccagaggtcagtaacgagctggtaaatctttaacagccagctcccagaggaaaaaacaaaagccctgatttgtagcatttgccagtttccatggtattaacactcccactgtggctgatttcaggctaccaatgcgactttaactggctcacaaaattctgatactttctttatctatttatctgtcaatgaacatttaggttgattccataccatggtaattgtgaataatgcttcaataaacatgggaatgcaggaatctctttaacataatgatttcatttggggtgaatacatacccagtatggtgattgcgggatcatatggtagctctatttttatttttttgaggaacctgcatactgttttccataatggctctaccaacttacattcccatcaacagtatgcaagtattctcatttctccaaatccttgccaacatcatctttgatctttttgataatggccattctaccaggtgtgaggtgataactcattttagttttgatttatatttgcctgatgattagagatgttaagcattttttcatatacctgttggcgatttatatttcttcttttgagatatgtctattcaaatccattgccctgtttttgttttttatttttgagacggagtttcactcttattgcccaggctagagtgcaatggcatgatcttggctcactgcaacctctgcctcccgggttcaagtgattctcctgcctcagcctcctgagtagctgggattacaggcatgtgccaccacgcctggctaactttgtatttttagtagagatggggtttctccatgttggtcaggctggtctcgaactactgacctcagatgatctgccagccttggcctcccaaagtgctgggattacaggtgtgagccaccacgcccggccttttgcccatttttaattgggttatctgttttcctactattgattgtttgagttctctatatatttgggatattaactccttatcaaatgtatgctttgtaaatatttttttcccattccttaggttatctcttcactccattgattgtttcctttgctgtgcggaaagtttagtttgatgcaatcccattcatctatttttgcttttgttgtctgtgctttgggggttatagccaaaaaataactgcccagaccaatgtcaagaaactttcccttatgtttctttctagtagttttactgtttccagtcttacatttaagtctttattccattttaacttggcttatgtgtatattgtaagataagggtcccatctcattcttttgcatatggatatccaattttccttacaccatttgttgaagaggctgttccttctccattgtgtgttcttggcacctttgtcaaagatcaattggccataaatgtgtggatttacttctggactctattttgtttcattggcctaaatgtctgtttttatgccagtatcatactattttgattactatagctttgcagtatattttgagatcaagtagtgtgatgaccccagctttgttcttttggttcagatttctttggctagggtcttttgtggccccatacaaattttagaattgtattttctatttctgtgaaaaacatcactggaatttcgatagagactgcattgaatctgtagattgctctgagtagcgtggactttataacaataataatgcctccaatccatgaacataggatatttttccatttatttgtttcttctttaattttttatcaatattttacagttttaagtatacagatctttcattttgttggttaaatttatttctaagtatttttggggggatgatactataaatgagattgttttcttgatttctgtttcagttttcttgatttcttgttgttagtgtatagaaaggctacttatttatgtatgttagttttgtattttacagctatactgagcttgtttattagttacaatggaatattagtcaatctttacaaagaaggaaattctgtcatttgcaacaacatggatgaaactggaggacattatattaagtgaaataagccagacacagaaagacaaatattgcataatctcacttatatgtggaatctaaaaagttgaattcatataagcagagtgtagaatggtggttaccagggctgggggcagaggtatggagagatattggtcaaacgatacaaagtttccattaaaatgaataatttctgcaaatctattgtataatatgactatagttaatgtatttgtatacttgaaaattggtaagagagtagatcttaaatgttatcaccaccaaaaaaaagtattgaggtgatgcatatgttaattagcttcatttaatcattttacatgtatacatgtatcaaaacatcatgttgtataccacaaatagatgcaattttgattgatcaattatacctttaaaaagatgggaaggaaaaatgcaaaaatttaatcatctatttttgcaagtagataagaactggctctagtacatcactgggtggaacccaggtttcattattttttaaacctcacccaatatggagccaaggctgagaaccactgagctaccaaagccagaattatgtgggagccttggggatcttggaataatacaaacaaaaaaagtctatgtaaagcatacaaggggaaaggaagagagtaatgaagaaaactatcccctgaaatgcaacacagaacaagatggtcacagctagggtaacaagagccagcttctccagggtcagttatctcttaagacatgcctattcttcatttgggttttagaatgccagggccacggatgcttttactgactgcttttcaaggaatataaagatgcattcttattgaaaataattgcttcctatgtgtagtaattaaacaaataaacccttaccaatactgagagttcctgattttgttctctcaagctttgtattgcttacgggataaagtttattcccaaataacgaataaaaaacaaactagatttccaatcaaatttagaacttgggttgactctcactctcctctcatcacttcaacaagatctgcactgcgtaggggtataggagcagagtcaccaaaaaacaaaacaaaacaaaacaacaacaaaaaagggggaggtggttaagtagaacaggcctgggaaagttatcctctctaaagctcatttcccattctgtaaaattgggagtaattcacctccccacagggttactgtgagaagtaactgagatggcacatgggaaacagatggtacagtacaagacactcaataaaatgtgttcatagtaatacaaaaatatatgtagccatggagaacaactaccctttacagaagaattaaaattctcattatagactcttccattgcttgtcatctgtcttcttaattaggactttttgaaaatgtctccagattcccaatttattcatgttgtgaaagaggattaagagggaacacagctgatctaagccttgaccaagtgtgagggacagagaacaaattaaggatgcctgagtcccacccacctttgcccaaacaccattttttgaacatctcaattgtggtacctagcctaattaacaacacagattccagcagcagtaaatgctcttcctagtaaccttcttaatgttctgtggagtcagattctatatctgggccctaaatccataattttcctatctcccttgtgctatcttaccttaagtgactaccttcacccaatgagtgttgttctaagagccaaatgtggcctctgctgcctcatttctgttcaacctcaaaccacgtcctggttccctttataatcgatagttataacaagccaccggtcacacagaactcccttctaatcagctactccacttggaatgtctcttaagatcaacactcaccagttttaatggtcttcttgctcattcaagacctcaagggtcaggtcaatttaaaagaccattggtataaattgagatgatgttgtcctgattcctacataattaatcataatggtgatcattttcaacaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtaggacatatatacaagattagtttttcaaaacattctttgtcctggtttagtcttgggagggtgtatgtgtcgaggaatttgtccatttcttctagatttctagtttatttgcatagaggtgtttatagtattctctgacggtagtttgtatttctgtgggatcggtggcgatatcccctttatcattttttattgtgcttatttgattcttctctcttttcttctttattagtcttgctagtggtctatcaattttgttgatcttttaaaaaaaacagctcctggattcactgatttttttgaaggatttttgtgcctctatctcattcagttctgctctgatcttagttatttcttgccttctgctagcttttgaatgtgtttgctcttgcttctctagttcttttaattgtgatgttagggtgtcaattttagatctttcctgctttctcttgtgggcatttagtgctataaatttccctctacacattgctttaaatgtgttccagagattctggtatgttatgtatgtctttgttctcatcggtttcaaagaacatctttatttccaccttcatttcgttgtgtactcagtagtcattcaggagcaggctgttcagtttccttgtagttgagtggtttggggtgagtttcttaatcctgagttctagtttgattgcactgtggtctgagagacagtttgttataatttctgttcttttacatttgctgaggagtgctttacttccaactatgtggtcaattttggaataagtgtgatgtggagctgagaagaatgtatattctgttgatttggggtggagaattctgtagatgtctattaggtccgcttggtgcagagctgagttcaattcctgaatatccttgttaactttctgtctcgttgatctgtctaatgttgacagtggggtgttaaagtctcccattattattgtgtgggagtctaagtctctttgtaggtctctaaggacttgctttatgactctgggtactcctgtattgggtgcatatatatttaggatagttagctcgtcttgttgaattgattcctttaccattatgtaatggccttctttgtctcttttgatctttgttggtttaaagtctcttttatcagagactaggattgcaacccctgcctttttttgttttccatttgcttggtagatcttcctccatccatttactttgagcctatgtgtgtctctgcatgtgagatgggtttcctgaatacagcgcactgatgagtcttgactctttatccaatttgccagtctgtgtcttttaattggagcatttagcccacttaccttttaaggttaatattgttatgtgtaaatttgatcctgtcattatgatgttagctggttattttgctcattagttgatgcagtttcttcctagcctggatggtctttacaatttggcatgtttttgcagtcgctggtactggttattcctttccatgtttagcgcttccttcaggagttcttgtagggcaggcctggtagtgacaaaatctctcagaatttgcttgtctgtaaagtattttatttctcctttgcttatgaagcttagtttggctggatatgaaattctgggttgaaaattattttctttaagaatgttgaatattggcccccactctctcctggcttgtcgagtttctgccgagagatcaaaacattctttaaaagcacctatgcttgagatgacttcccataactttaaatagaaatgcttttatttagttcatctcataattccatgcagacctctcaaactatgttcctaactttggattagaaaatcaagccatgtggtcctacaacatttatcaatattataattgcatacttatctatgtgattatttaatgaatatctattttcttcactaaactataaatttcatgagtttttcccctcttattgtttcctccatcttcagacatttaatctctgggcctggggtacaatgattgctcaataaatatttactgtaatgaagaatgcatgccataaccataggtctgatgcagggtttctcagcctcaccactattgatattttggaacagatcattcttggctgtgggggtctgtccagtggattgtaggatgtttagcagcatcccaggcctgtctctgttagataccagtagcaacctccaagatgtgacaacgaaaaatgccttcagacattgcaaaatatactctacgggtgggcaaaattgcccccagttgagaaccactggcctaatggaatacacttgtgacataagtaaaaaggtgacaatcctaggaaactgatttgagtgaagaggaagggaaaggaatagagggaaggtggaaggggatagttagttgctactcatcttgtcaatacttttcagtatcaattctaagccatcagattgactccccatctctttcttctccatataggagctacaacactgagagaggtagagcatttcaggaaacaatatccaaattaccatttcccaaaaacagttcagaacattaattctacaatcttttggtggatgttgctgataaaagggctccatggtcaaataagcttgtaaacaccagattctgcaaaattaaatgaatttcttcattatggaacttatcaaagtcttttgccttctaacatgctttgaaattctccaagaagcgatgcagaaatcttcatttttcaagtttctgaaacatgactgctcttttttgccatatcattttgtgggattactgtgtctttaaacacactctgggcagtagtggcttagggttatcatagtagtaatatcctttcataactgagaatttcccagtctcctggcaaattatctaggttgttaataagggtgggccatggtgctgtcatgttaaagctgcctaagaatattcacttgcacatactcacatcgtctgtttccaactctgggtgctacttcagatagaacagaatgataagctgatgatttgctctgaaggacattcaggaaggatgtcactgctccattttgaaagattcaccaaagatctttctccagcctcagggactctgccttccctgaaatattagtgtgagggtgtggctccacaccaacagctgtgaatccatgactaagctagaatctgcaaatgatggaccaccttactgtgtgccaagcattgcagtgggcactttcacagcatgtgtcatttattcatcacaaccctataatacaggttgctttattcattccattactttctagataagaaaaacaggacccagagtagttaagtagtttgcccaggttcacacagctagcaggtcgcagatcctggatttgaactcaggtctggtaaagggcatgttcttaaaaactttaatactctcatgccgcctccttattccattcactcactcattcattcaactgtctttccttcagtagctcctacatgccaggcactgtggaaggaacaaacaagtatgatgtgtgcatggaaaaagtttaagaaattccagaaaaatacagctttgacagactactcttggagtgcagagaagacttgtgttttgaagcgacatctaagctgagatctgaatgttgatgcagttgtaggcataggaacattatgtgtgtataaatattgcccactattctcccctcattccatgttgactttctaccttcccagagtgaataatacttcttatttttatctaattaaaacagcacctttttttgccaagggctagaatgagaaactactaaaaggctgcttcctttcctagtccctgattatcagctttcccagtctgcgtattggcatatattaaagagacatgtgtgcccggatcctactaattatcatgaaaataatgaacatcacatatctctctcttgctcactctctcactttctctttcttcttgggactaaagtgtgtccagcgatttcagggcacagtctgcagctggtggcacaactctcagggctatataatgatgtatggcttgttcttcgtggccctctgcaaaaaaaaatatgaatttcagaacgtgaacaactagcagacatctgctgcattaagagggaaaagtcagagctaactagtttatcttggaaagtatgtactgtgccaaggctatacttatctagaaaaatttctggaaacaaacagaaaaatgactctaaaaattccatttaaagtttaggtccctcagaggaacctacatattacctcaaaatatttgaccacgtatcagtttaaccatgatgaacttgaataatattatattgccactgagagacacaaaagataattagaacaaaaattgagacctgaagatggtttgttcttccccactgtagcctaagggtatctgctgaatttccaattatagtcattctacaaaatctgatcttcctacaagagtttagcagtattagaatcacctgaagggcatgttaaatcactgattgcttggcccatcatgggtgggttcaagcatttccatttctattaagtttccaggcaaagtggattctgctggtcagggcaccacacttcgggaaccatgaccctctgataaccttccctttggcatgcaggtgtgtttctcctcaggtttatgtgttgctgacaagttgataagaaacacagcaataattttccaattccttgcatcatgatttcagaggatctttttcttctttgctaatggatgattgagtaataatgatcactcatattctatggactcaccctctgccaaatgaggatcaattattaaacaaactcctatgcactaaagaagaaacataataaaataagccatgtggtgaatactattgtagagaaaaccaacttttatgacacagactctaactttttaacttgtctgttttatgaggctaagtttttaaatatcaaattttccaaaaccagatgcatctggaagttatcgtccaacagtgtaagataatttcagatatctgcaaacagaatttcagaaaatctaaattcattatcatctcaaatgccactaactggtgtaaatgacagaagttgggatgtgaattaactgataatattcccttattccatgtgtatttatagagtagctcctgtgtccaaggcagtaggcaggaaccaggtgaagatgataccaagaagtctctgaagagggtcctgttctcagggagataacagaccagtgagcctaggtggagaatgacattgaaatatgaaccaagcaaagactggaaaggtgaaggtcatcattggccaacgtcttccttctcagttactaaaacaagtcatgggattaagtcatgatcatcaccccatgtgacactgaaaggaatgaagagactatattggatattcagttaattatatgtctcatgcttcatggtctcagagagctttatttatccttagcctggccagataattttgctctgtataaccattgccataagaagtcaagggtcgactgttgaagggagtgaaaagagaagtatcacaatgtctttaaattgtccttatgttacaggccgactatcattccaaaggaagataattcttagtcattgaaccacgtgggagagtaaaaactctaattacctttgttaccttgaatctaagaagataatgaaactttgataaggacagaaaggacttccccctcttcttttccctggcataatgtctcatcctatgttgtctcccggcactctcaaggtacacccaagtgagtgtagggattaaagaagataattggcatgctaaactaatagtggttaaacaaatttccaccttaccttcactctaagcaaaaggtctatttttaaaattgagtaaggaaaaacattttaaaaatataacatccaactatctcggtacaactatctttttcatttttatatgttgccatcatgttcagcagttcccttacataaaaatagatagttggtttgtttgggttttctacacaaataatataaataaactttgcaaatatttgtaaaataccaaatatatcaattaaaatatgaaaacctccttcattcttcccaacttttctccccattcctgaagtaacctctgttaagagtttggtctctatcttttcagttcactttctgtatgagtgtatttatatttgtgtgcacatatatatggttttggattattaaaaacacaaatgctgtaatattgtacttactattttataactttttaaaaattaacaataggtcttgggccaggcacggtggctcatgcctgtaatctcaacattttggcaggccgaggtaggcagatcacctgaggtcgggagttcgagaccagcctgaccaacatggtgaaactccttctctactaaaaatacaaaattagccaggcacatggtgatgcatgcctgtaatcccagctactcgggaggctgaggcagaagaatcgcttgaaccagggaggggtgggttgcggtgagctgagatcacgccattgcactctagcctgggcaacaagagcaaaactccgtctgaaaaaaaaaaataggtctctaatctttccatgtaacaacatacaggtctattttctttatagactccttcgtgttggtataaatgcagcggatttaatcactcatgaaaatagacattaaattgttttatttttgctctttcaacagtagtgtaattgatatccttatgcatgatccttgtacatgtgtgtgagtgtttctgtaagagacattactcaaagtagaatagtagatatttacattgtttccaatctttagctatcatgacagcaaggcaggcaacttatctttgttcccagatgttctttcggaaacaactttctttttcttctttttgttttttatttaaagattattggataaggatatagagaatccagttgtagttcctctctgtgtgcactctaaatctcagttcccttgtcagaactgtagttacctaatatttaaatttttaaaagtaatttaaagttaattacataagacaaaagagataaagacattttgctggggtgaaaagcaaaagtaatcacctaaatatatgtttgtaaaatgatcaataattgagttcaactcaattcaacaagtattttaccatgcaaaatgtactatcttttacattacataaaatgcaaaagaagatggaattcccacccttagacaccttgtaatctaggaagtggaagacacaaatccataacaaattataataagtcagaatataaattttattttaaaaactgcaaaaaaaatcttctgttgaatgttcttgatgagatccttacttaaatatgaggtatccataaaaaaaatcaaatactctttgcagctagatgctaaaatatatcactttcaaggctctcttaattttgttaatggggcaaccttcactctattttacaggtgctgtggagagggtgaagaaaattcgagactatgcttttgtgcacttcagtaaccgagaagatgcagttgaggctatgaaagctttaaatggcaaggtaaggtagaagaaagaaaaaaatatgcagattacatccaagtttggtgtggatattacatccaaaagcacagtcctgcaagcctgtctcactccagccttctctgacatggctgcctcagtccatttaagatgtactaaagagaaatgatggaaaatacaatggaagttggagatgtacctaatatatccaactttacctctatttgttttttataatttggacaagtcactggccctcaatttccatggaaagaggagatttaagtagattatttcaagatgtttttcacaattccagttttctctcattacttcaaagagattatcatcattttagtgcagtattttagaagctgatgaattaaaaacatgtcaagatagctgttattctgccttcatagtttgatcttccacttggaaagcttagagtctctcaccttgccaagagtaggggctggggaagcttctatatcctcactactgagttgtctacagaaagcaatataatatttatatatcaagccaataaacatttaaaaccttctgtagaccacttatcatgctaggatacaaagagaatcaaagatgtttgacaaagagttaaaagttggttggaggaataagacaactacacaaactcgtgcaaagtataggcaagtaaatatcataaagagagataaacgtggtgagcaagttcaaaggaagaagtgggctataaagaataaaacatcttcatggatatgagaagataatatttaagagaagccacgaaaattgggtataatttcaaaaatcaaaatatggaaagacaggaagtcttagaggcacagaatttgagaagagcaagacaagattggagaacaggaaaatgtggagtaacttggctggaacataatgcacgtgggactgtgttacctatcggatctggcatcggtcagggtcccaaaagaaaacaaatagcaaaggaattaattacaaaagtgcgagtggggactagggaaatcaaaaaagagtgtcagaatttggggctcacagcattctggagtcaaaggtacaaggggctggagaattaatagaaatcctgaaaggggagagaattgtgtagagcagatctctttgcaagaagttgctgggtgcagtggctcgcacctgtaatctcagcactttgggaggctgaggtagaaggattgcttgagcccaggagtgcaagaccagcctggacaagatggcaagacctcatctctacaaaaaattagccgagcatggtagcatggactactacagtagtcccagctactccgggggctgaggtgggaggatctcttgatcccaggaggctgaggctgcagtgatctatgattgtgcctttgcactccagcctgggcaacagagtgagaccccaccattaaggaaagaaagaaaagagaaaagaaagaagcagtgattttggtcaaaggacacagtcagcccaaggtgacctctcaaagacgggtccagcagaataaatgccccgaattcactgttgtctcttctccaacctcctaccagtgttccccattggctaaatgcaatcacaatctagaaggctaaagaggaccccatgtagaacagcttcccagagcagagagcaggctggagctggtaggtggtgaattccataggcagtggatccagatggcaaagggaggatctcttagcctaggcccttcagacactgtggagcctcttgcatattagctcctcaggggcccactcaaactcaaaaagtttgaatactggagggtggggtggagaaagtattcactctgaaatacaaaaaagaaaactgcaaagaaaaactaatgcataaatacatgtctataaagaaaagcattatttcatcttcattgatgatcacatttagtatcataaaagtgttattacttggaaaacaatttgtaagttagattttctctatatggaaacattcctgagtatacatgatgattgctgggaaggaacagccatcacaaattaaatgccaaaaattacgttggaataattacaattgaaacatattcaaattcccaatttttgcagctagatatttttagagtaattatttacacatattatttagttaaatatatatttaatattatatttgactataatgtggtatggaaaaatcaaaagtaagttttcctaggaactatgaaggtttttgagcatatatagagatatttatatttaatttgttagatgaagtgagatcttgaaattttttctacgataattatcacatttacaatttaggaaaattagcctagtaagaactgactttttaatacaggtgagagaaacatgtcatagaaactagttataaagctattatagtcaaccaaatgggacaaaataaagtcttgaacttaggtaggaatggaatgtaggagacagccatatgcagaacagtaagggataaattcaaaggacgcaggaactgattggagatggggtgaaaaagaaggacaagtcattataagcttccaatatggtaggttgttatgaaagtcaggaagggaagcctattcagagagaaaaatgatgaattgagcttcagaatgattattcatttattctttttgtaagtctatgaagtgaaagaaattttactaagcttgggctatacagattaataagatattgtcctagctctggaaatatctaaaccaaataactaaaatacaatattgaatttaaaatgcttttggatgggacaaaggaaggcagaacaaaatttgttacaagatatctgaatttgccgcaaagaatcattccatgcaaaacatctcttgatatggaagtgcctataagcagagacctatttgggtctctcatcttcctcaagactggactgaagatataaagaatcttatacccaagttcctcccctattttcagtatgccaggtctactttttctaattacatcaacatcaatataagattgttgaatatcagctggattctgtctgtctctcagagtattccatctaacttatttgacactggctcctgctactaaaaagtaaggtaaatacataatactggggacaaattgaccaagaggctgaacaaaatggtcaggatgaccatgaagcacaagctttgtccagcagacttcaataatccaggaaccactgagtcactgttgaaatcagagggtaagggtgcacactcagctgctgtgaatgggaatttctgtagaaacactttacataaaagtgttctttatataaaacagaggatcttcacattcttattcaaaaacaaagccacaaatgctttcagacaatactttgttcctttgataataaatacatcaatctatcaagaatttcttgagagccatcattgtttattgtgtaggcaagcaatacatagaagttagtagcacacgtagatctacatacatccattatctttgacatctggagctgtttgcagtctcagaatttgtgtcagaattcttaaggacaggttttaagcagtgcatgatctgagaacctcaatactactctcactcccaactactcctgacccctgtcatccctgtttattagctcccaactgttcagcagatcaaaaggtggcttcacaaggaagaacctgtggcaggcaatgcaacagcctcccagattcctcttcctttggtagagccacagtatgtctctcttctccagtgaaaacttacttgtgtggctgctctaaggagcaagacagttagcgggctatcagctggaagggcctgatttggctgcccatgctaaatgctaataattgataagatttctttattggtttcctttcaccttcatcttattgtgcaaggtaatggagattttataccatccataacaaacctaaattgtcttgtagttatataattatataaatatgtaatttaaaacacatatatgacctaataaaaatattacataactttctgtctcagtgactgtaaccagattgtaaatttataatgaatatgtcaatggacactggccaatatgtcactgtcaaacctgttggcatcttatgcaacttaccaacaaaatggctcagaaaaagttcaactggcatacaaaacaaacctataaaatgacaagatacaatcaataaacctacagcaaatagtctaacacttagaaggagtcatgttattagggctcagccacacttggttatcttcatatcatctcgcgttgatatttctttgctctctacaacaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggtaacaccagtcagatcttgctttagagtaatcccaagatttcaaatgactcaaattgcagtagccttagatacattggatgggtccacttggagaaaagcactgaaaaattgtttattaaaaatcaactattatatcaatagttttcctttaataggaatctttttcacattacaactggttattgaaaaaacgtcatcagaacattgtgcaaatgagcctagtgcacagattcaaactttgcatgggcccataaaatttgcacaaactaaaacctagttctataattgttagcttcttctatatcacttgtccaccactgtgtagtcatctgtggctcattacaacacttctggaatagactagtatagtccagtggaaaccaattacctcaaatagcaaaaccctttcaaggcatgtgtgtatttgatggtgactcagtggcattgtcttaatatgtaaatcacaaattattataaatataatctaacagacatatgagatgttctcatctgacctcctccaggaaatatgttatactttcttagaagccaaataattaaggacattactatatttctttaatacatggagaactgaacaacaaatagaaaaaaaaccctcaatcagactgctaataaaacaataaatatttcacatttgaatcctgtaagaattctaaatgtttgagcagttttccaagctccatttcttcaagaaagctagaagataaagaaagcctttattctatttccaagaaactagcttgtccctgtctttttggctccttcagatgagtttttctaggatgtggctaatgccaactgaatcccagttctatcctccaaatacgacatgaatctgggcaattcgataaactgtgcatttaagtctattcttgggtttgacttagaagcaaaaaggcacatctagattactgtggaaacaattatcttactatgttttactacttttcagtataactaaattgatcatattaggagctctagtcttcaacagccttaaaagtttcatactcataattacttgccccaaaattgtaatgcaaatgaaataatcaagatttcctttaaggtaacttgtctatgtgataataaattatatcattgcaacattatgcatttagaatataaaatcacctcattaaccaatccatacattaaatacaaattgtcaaccagttatgatgtcgaaaaaaaacaaactccactataagggaaaaaatgcctatcttataacaagaaaggaatcaatgaatgatattcatggatgaagggtaatcaatttaattttttatctctatgccaccaatataaaatcaaagtagtaacatggcaaaacaaaaatcaaaaggaatctatgcatgaaactcaatggaaatttaagcctcacattgaccgaacatcagagagcatggtgaacctaaaagcatgctttctaaatgtttgttttacaataagtcattggaagtgggataattagggaagccagaatttatttctgatatactgatcagtgacagtctctatgcaagactgatttcaaaataagtggtcgcattatgttggaatgttctaataaatacattcccagtctttattagtctgtataaaatatcaaggttacctaaagacaaaaattgcaaattttctatcccaaaatttttaagttgtttgtgtacttatcaatagtattgtatttatcaatggcattgttttcttctgaaggtagtttttcaaaaaagagagacacctatgttaaaccttaccttatgttgagtttcattacattagaatcctagagtctcccacccatcttcagggcaataatcaacgtgaattgggctaacttttccattacagtaagattcagggtgattttctggtggtttgattcagtttaatgccttcttacgttcctagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccaggtaggtaatgttattccagaaatagtttctttaagttgtgccaatgagcatcattcacttttatcaaatgatttgacttgatccctactcaagctatgggtttttttcttttaatatataaagcatccaatgaatgtcacagtctttgaaggaatgctgggcatctggtgccattattctattataggtcctttctcttttttaaaaaatcataaagtctaacaccttgaacaatattccactgaaatgcaaagaatctgagagctattaagaattatcaacccatacccgaaatgaggcaaagaccccatctcagaataaatgcctattgtatgtgagccaggttggctggcaaatgaagagtattatggcagtgccctgaatagaccatttaggacatcttttcttggaaatactgtatctaagagaaaactccaccatgatatctgggtgaattattaactatgagatgtcatcagtcataccaaattaactcttcctccatagttatattttggcagtggcattacagagagtttttactgtgttcatttaacagttctaagtttccacacttcaacattaacctgtggtttttattattggaaaattaaaagctataaaagtttcaatatggaaactgactcccggggttaaaaataaaacaaaacagagtctatgctaaccctgagactgttccctgtgggagtgtgataggtgctgatctctctgaaatacattttggctgaataatcagagttacagtttgtgtatcttgaaggaaacttccctattcccatgacaaaggtgggcttatagcctcacatttatggaagtagcccttataatttcaatccagaagaaaactggttttctaaaagctcatcttgggtaattttttaatgggatttctggctttcttttatgcacaaattagcttatctaaggtttctgctagccccacccagggaattttaaaaaaaatgcctatcagactgctaaacagcaatcatttgttcacataagatttagtaaacttctcttatgcaccatgtagtgcgctaggtgctggagatgtagaagtgaatataacaaaatccttatcatgaaaaattagatggcaatccagtgggaaaaaactgacacgtaaacatataatgaaatctggaagtgatatgatgaagtttgttcagaaaatataaaagccactcatccagtttctcttaggttaaaacccaaaacaaaaaacctgattaaagtactggaatacttagaaatattttaagaaaataaaaaataatagtcgcaaattctgtataataatagttccgagagctgcaatagtatgaaaagagatctcttcattttacattaaactcaagttagaaattcatcatgtcttttattagtatgtgagtaacaatctctatcaattaataaaagttctttaatttgtgtctgaccttctacaactatggcttagaaatgaagaccatgccaccaccaaccaacacagatggggcctaaacaatcccaaagtagagttgtgaacttccagccagaagcagagagacaagccctttcctgcaaagtcagcagtgagagctggtgttccttccgcccaggtttgctcacctggcataaacaagcaggcataatatctaccagcaaaagctcctttattattgcccaagaactgtagagaatgctctgaggattacaagagatcctacacaacaagagtattaaaaagaaaatatcaatagaaaaaacagtaacaatctacaaattaataaactggaaaatatcatgctattgcagctgggttagttagcataaatgaggtaagaaacctcagagacacatcctgagacattgttctgtcttacagagttattttctcaggcttatgccctgctttccacagtctactgaatctgctgactaacgagagcatcaggacctctggacaaccaaaaatggcttcagaataaagaaactatgaatctttagaaaatattcagtttggagtaattacagaagaaaaaacatgcaggaggatgctaaaaagcatattgtcattaagcaaaaaaacaaacaaacaaacaaaaaaggcagaaatagtgtcttaccaaacaaataaaaacattgcaactgtcaacacccatctaggaacactcgataaataccaagcattaaactttttaattctatttttatgaaattgtgagattaaaaccctatgacaaatgtcacgaaataaaaatatattcttaggctatcatctctaaaccattataaacacagagattgagagtaataatgagtaaatctatacaaatctatacacacagtgttggagcaacaatttttacaaaaacaaatattccaaacaaatcataaaattaagcttttggtaaaaagttacagctaatagactaaactaaaaacatattacagtaaatggtgtgaaagtgattttatgaactaatagacctaacattttaaatggagttcaaattgagttcatcttaaattttatgagtcattaaatcatgtggcattttatgtcttcagatactagcttgaactctgaaaattagagtttaaaaacctgaaaacttgggaaataatgtgaatgtgtcatttgagggggaaataactttttttattcttgtagatattagaagagatttgtcagaaaaataactggggacagccagtgtaccagctgcactctgctattggacaagaccaaagacagctattcttgtacaaaataactattcctgctctagccagccagaatcctgcaatgtgcgtaacttctccaaattccttaccattttagagcaggatacacttataaattaaatgcatttagtcaccatctaaacaagatactgaggtctactacctactcatttgtcattcaatgcaaatattgagatgtgtagttgggatgtgccagccatgaaaccagcatataaaggcccccaaatttcatatacacaatgaaggttgtgtagtatcaccagccagtggataacagtcagtagtgtaaaatgtaatagaaataaaaactatagcccattcttggctggatcacttgaggtcgggagtttgagaccaacctggccaacatggtgaaaccccgtctctactaaaaatacaaaaattagccggatgtagtagccggtacaggcaggcgcctgtagtcatagttactcagcaggcgcctgtagtcatagttactcagcaggctgaggcaggagaatcacttgaacctgagaggtggagtttgcttagattgtaccactgcactccaacctgggcaatagagtgagactccatttcaaacaaacaaagctatagcccattctttcatctgtgcagtgtttaggttgaagcatatgaaactaccattttatacataataagacaatggcccatcatttaattttggaccaaagatatgacctctctgttagtggatatcacctcctgaattttaccttgagcctgcatttctatatattttaggaacaatttgtttcacttgtgctttagaaaagccatgtagtgaattacttgtatgtcccaggaatgtcatcaaccactgatgttctagatcaatttgtaaatatttctacatgcaacctatatttagaagctttaatagcaaacagaatgaaaaacacccactcaaaataccaccatccagtcatcttacaaagctgatagtgccttaagttttcacctgactcagttgtaccagtttaaaaatgcctaccctgactgacttgctttccagccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggtatgggaaaattgtgccagaaatggcgccctgcagagagtatttgggaaacccttttttggtaccattgaatcacagaaatgtttgtgtaccctacttacagcaatctgacttgcacaaccttgggaccaaaaagagtttcccaaatgagtgactcattaaaacacgaggcaagaaaatcaagctgggcagcagagtgggttctggtctcggctcagctttaaactgctgtgtgatcttgaatgagtcagtctctctgagtctggtttgttttggtttttacttttagtacgagagtttgaaactacctatagatatatgatgtttattggagatcttactgtgtgctaggcactcacttaaatggtcaggaagcgttataatatcttagtaatactcatgataccaccaagagttaggttccatcagaaagtctggtatacaaatgaagagcccatatacagaaagattaagtaatttggccactggcaattagcttttcagcaatcttttctccctctccagtcttcaatctacttttattaatttgatttcaaaaaagattttttaaagtcaaaaattttgtgagagtgagagacagacagagatcctttcaatatgtgaataatcaggactcaactcaatttatttgttctataattttggtctggggcatatgctgattgcttaaagcaaagcctatccgtggatcaacaatgccttcagaggtcacattttaaaagatttgtccccatttgttatgtacccacaacaaatggtaataattaaaactgaatcctttagagattgaaggggaccaggcaagaaagttgtttaggaaattttttctcagctgaaaattttttatcctttttttttttagtaaaagattatttttattaaagaaatctgtcattttttacttctcattgtgtcagcgtgtgccacataagtccaaattagagctctaagtagacctagaaacatgatattcttattgattaattaattcatatattggccatgcgtaggtgtgaaaaattttatattcctcacacccactgcacctggggctccttctcagtagtgttttttggttttggggttttttttctcaaaagttttgaatatatcctccattctggccagtggggctaacattaatgatgtttcacatctgaaagtgacctcaattctggctttgtttctctgtaagaacatggagatgctctgtggtgagtccctcggaggttaccaaggtaacagctgacattgcagagggcaaggtccttcctgggcagtgtggggccagcagccagtgcttgctttccacataacagggtgtttacatgtccatttactcaggatgcctgccagggcttctcagttcctagagaaagttgagcccctgtcttacagaaagctgcaccccaaagacacctaacaaatgtactattaaactaatgagccactgtcagaattttaaagtttgctttcacattggaaaggatttgcttttacccaagaatgttttatcttattctcacttccactggcttctgtgagaagttaaaaatatttagggcatgagtgttgtcttgggaatgcctaagaatctgagagcctctcttataactttgctgtggagtcaagcaaagaataataggctccaagggaagaatcagtttgaaacccaaacaccctggcacttaacaaattgaccaagttgaaaaagaaaattcaaaatccctaaaggtaaaatctgtgccacaaacacatgcatgcatgtgtgcacacactcacacagttacattactcacttagtttagaaatactgtttccaaaaccatgaaagtaactgttaacacatgtctcctatccttggaaaatggaccaaacttcactagtgcattaatcagcattactgaccaaaatgaatgcaccaagagtggattcaatttttgttcaaaataatccaaccagcttcctgccaccactttctgagaagccactagaaaaaaatcaatgtgtctggggttgggcaggcttctactgcacataaactaaatggggtttaaaacaacaatgaaaaataggtgcccatttgtacttaagtatacttctctctttttcttgctatcttttacaatttctggataatctcagaaatactcaggttttaaactaaaacaatacagagaaataatgctttagatagtagctaatatgctctcttcttcaaaagtgaatgctcatccactagacacaatgctaacatgaaagcgcatagattatttttttaattctcagatactttggttctctgaggcggagaaccagcatcaaggcctctgaatacaacaagcacaggcttgatgaggggcttgggaggtaagaaggtgcttttgccattcatagttacatgtaaatatggacaggttaggcatttgcttttaaggtattatggaaaagcacacttaacctttacagtatttctcctactgatctcccagtctttacacatcttcttgggtaaaggcaaatgttttaaatgtttcatttaggcaggtgtggggttacaggagaaccagtcccacagatccccacctccccacataccatgcccagccagcccctcagtcaccttttctcatttataaatttggagaccctcaaaacgctaaaaatcacaagctttaatcaactcagtatcctgttaagtttctctttcttggaccctatcccaagcatctttaattctcttaagtataaagtgattctgacacaggtggttgaccccactttagggaacattgtcctaggactgtttaaatatcattgaaaatcactgagtccacatattgtgtccttaaggacactttttaaaacatatatattaacttttagctttaagcaatatgttaaatgttcgtatttctaaaagaagacaaataagccaaaagggagcaaatgtgaattcctttcatcaagttttctctgcctcttcctccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 2 - DNA, 77253 bp, Macaca fascicularis
  source 1..77253: mol_type = other DNA
gctgtagtgacccaattatctggtcaaagggctgcatggatgcaagagacagggtgagttaaagtttaactcccaggaattgtaaaccaaacctaaagccactgaacctttggagtttgatatgacgattagagcataacccgagtaacacgttgaattcgccataatcaaggaaaccttttccgggtggggatctctgaaattactcaggtatgatccatgtgtttcgcttttttctatttcaactctgtggaactgtgcattgtggtatcagtgggaatgcagtaaaaacagctttcaccttcttctattatcgagcctaattctaggaattatctagcttgtttttagacaaatgaaacaagcagaaaacaaaagaatagattggaattattatataatctcaaaatgagacagctgccaggatttaataaatttctgtagacttaccacggagggttcaagagtggagccaatgttgtgagaagtttatttagctctaatcagacagcagggagagttgtggttgagtgcctgtccagatcctaggaccctgagaaacagaaatgtaattaacaaaagaaacagttttaatatcaccggcagattttcagttctgtgtgtgggtcgtaattcctatcatactttagacttcgtgtattcgattatctcctgagctactaccaccataaaaaagaaagtcttggtaaactcttgtgatttcccctgccattatcttgctaaccaaaggaatgtttaaaatattcacttctgattccgtcccaaaaaatattatggaaaatgactggttttcagtgtgttatatctcaatcttggaatggctaattattatattgtttccacaatttgggcaactgtgctgaggtggatgtattttcacatttttttctatactccaaaaacattattcacaatcagaatataatggtatttaaagaaagaaatcaatctggacatttaccaaaaatatcccgtttctaccagaagccccctaaactctctgactttttgtcatgattacctatgtaagttccaagaagactggattccaaactaaacgagtttatttactataggaattttcagatatgtattgtaattctccaagacgacgacgaacatagcattatttgcacattgtgcaggattagtgttttgtagagcatattttggtaaatgcagctctagttataatttaggatcatcaaaaaatgtgttggtctgctctgaaactttctttataatgtgtgctatggagcatgcaatgggagtataggtctggaaatgacagtgatatcactgatttgtcaaagcagtaaggttttgccctgctaaatacagagaaagagacaaatgacttataaggttcatactttctggaataacattgactctgcccattttagacaactcttaacattttactgttccgttttactatttatcgattttaagggaaaaaagaagacagctggcaaggaaaagaaatattgttgtgggcaaacattataaaatgacataatggatattaaaacgctataaatacttagttcttattttctactgcactaataccaagttggttttatgtcatttcaaatatcaacatttctgacactatattcagtggatgttaatgaatgtggctcaaaagaaaagtgactccattgtcaagtaagttgaggaaatggtgttagaaaagttatagcaggtcttgttaccacatgagtgttttgagtaataatgatagcaagtatctatgaagtactatattatcaggtctacttcttttttttgttttgtttttgtttgttttttgtttttgtttttgttttttctttttgtttttgttgttgttgttttgttattatactttaagttctagggtacatgtgcacaacgtgcaggtttgttacatatgtatacatgtgccatgttggtgtgctgcacccattaacttgtcatttacattaggtatatctcctaatgctatccctccccgctcccacctccccacagtaggatctggtgtgtgatgttccccttcctgtgtccaagtgggtctacttctaagttctctccatgcattatctcatgttattcttagaacaactctatggtgtagatactattataacagtttataaatgggaaaatggaggcacagacaagttacctaacaggttcagctactcagttagcaagtggcagagccaggctcctttgtctggcggtctcatccagagcccaagcatgtaaccattatatcacaccctcaagcactggggactcatgacacatgcaaatattgtcatttatcacaattatctaagagagtaggcaatgttttgcttcctgggcttaatttacaacagaacatcttctttcaggaatatctatcaatatctctcaaaattagtatttctcagaatacaggagaagaaagaacaaattgcttttatagcccatgtccagcattcataacaatgagaatcaattgaaatcatcacaattgaaacatctaaaggactttagagtctagtcttcttccaccaaataggactacgcctatctgagaaacttttaaaattaattacagaaagaacactaggctttttgcctcgacacagctaagtggaaacgcctattgcaaaactccctaggtttgtgaccttgggcatattgtttgactcctctgaatctcagattcctcaaatgtgaaatgggggtaagatctgacccacagtattttgtgaaggttaaattagatgataaagcgaaggagagaaatgtggtcccgggtgcttaagaattcagcaaaaatcccacttcccttttcactttcctatttgtaataacctccagaaaaattttctaaaattgcccaagtatacctattaggccttgtttgtcaaaacaatactaataaagttgaaatcataaactgtacataaaaaatttttgatgaatcacaaatagtagcagtattccatttattcagagaaagtcagtcggtaatttttttcagtatcaaagcacacacacagagtatatttttattttagagattattgctgaagaaaactcttagttataaaaccccaacaccttttcacccaaacccgtgggacaccctgacacggggcatgcttatttcatcatggaggcagttgctgttaaaaatctgtttcagtaactgtcttttaaaatattgtcatcccttagaaagctgagtagtactgtgggtactattgcacaatggttttcattgccttatgtgtctctaaaatgactcaataagttcaaacaagacagtattgatcgacagaggataaacactattttatcttattcacagaaacagtaaccaggctcctgcaaagtgcagctctcacaatccaactgcccccaggaaaagcatctggaaaaggagcagcacccttgcggtgacattccatattagaaatctgtatcccatctgctccttggcctgatcctgtaaaatccagagagttattaacaaatgtttgctgctgattcaaccaggaaagctacttcattgttttatttcatcaaatctactctgatgaaataaaacctagtttatccgttttttgttgttgttgtttgtttgagatggaatatcattctgttgcctaggctggagtgcagtggtgcaatctcagctcatcagaacctccgcctcctgggttcaagtgattctcctgcctcagcctcccgagtagctgggattacaggtgcacaccaccatgcccaactaatttttgtatttttagtagagacgaggtttcattatgttggccagactggtctcgaactcctgacctcgtgatctacttgcctcggcctcccaaagtgctgggattacagatgtgagccaccacgcgcagcctaggttatcagttttaataaccacttagaatttttaatttaataaactaaaattttgtattgaatattttttattgatgcaatccaaaaattaaaacatgtccttttgtaaaaaaattattgtcttgactaaaacatgctggtatcttgagagctctaatttgatttcttttaaacatatgtaagctaagatatttttcctatatagatttatatggggagttttatgcatatatgtattaatcacatgaagaacattttccaagtttgtttattcaataagaattggtccctggtaatattaagagggaaatattaataaggatgtaagataaatccctctgatttgcacagttaggtgcaatgctctgctccagacataatatgactgaagagaattagctcatgacaatttaggcagacagaatgtctaagtgagcagatcggtttgtaattctataaaatctcacttgcaatgatgtagtgatgccttcccttcctccagcaattaaaaaataatcattgctttaggatgcccaattgatttaaaaatcactaggttgaagtaatagaactaactacaacaataccttatttctataggatattctagtttataaagcttttcatatagtacttatatagatttttaacgacaaatttcttcatagttgatgtcaattcagtatgcctagtttcttcaaggtaactgtgtcaatgttcaccattccaattgtacaaatctgattaactgaaagatgctcccacctaacgtttataagcccgactctgtttatgtagcacaatgatgattaggtagtgggcctttaacatcttcactggatattaatgtgcctatcaattaagaggttagatgaggacaggcgcagtggctcatgcctgtaattccagcactttgggaggccgaggtcggcagatcacctgaggtcaggagttcaagaccagcctggctaatatggtgaaaccctgtctctagaaaaatacaaaaattagccaggcctggtggtgggtgcctataatcccagctacctgggaggctaaggcaggagaatcgcttgaacccggtaggcagaggttgcagtgaacctagatcatgccattgcactccagcctgggcaacagagtgagactcaatctcaaacaaaaaaaaaaaagaaaagaaagaaagaaaaagaggtaaatgaaaacagtgaagagcggacatatgggttcatgtggaaggtgagagaagaaccactcccatataggacctagaagaaaatagccctaaaaagatgtaattataactcagtccagatgctgtaactaacacagtgaaagatataaaggaacagagatttataaaatctcctacctgcctggcaggggactggcaaagcattgtgtacaaatgaaattttttatttctattgattttagtattcttctatccacctcttctattgggattctgcttagtgcttatctttccagctggggccttcagcatgaactaaatagtactttaaattattaggtgtaattaaaccctaatgcctctgactttttgcaatggaagtcagaaatcaaaatctggttccatagggggaaaattgtgttgatacatatagatgttcatctcaggaatcctctttctaaccttcttttctgaagatccctaacaactccttgccattcatgtacttattaccaactaattataattggcactctatgatgatgataacgatgatgttaataaaagcccttacatcctgttacatcctgccacatgccaggcacataggttaactcatttaatcctccatctctctatggaatgggtactattgctacgccatttttcccataaagcaactacggcttagggacacagagaaacttgccccaggtcacaccactagtaagtggtgcaggagggggtccaggcagtctggcacaggccaccctgtgaacaaaggacaaatggaacactgggaagcatcgaggcttagaactagaagaaaacttttgagatcttttatttgacagagaaaagcaagttggcttccccaagttaggtttttgttagaatgaaaacccaagggcagaacccaaaccctcaggggggcttttccattgtaccacttcccactgtatagcatgtctaattcaagccgattctctatgaaaattctgagagatgaattactagaacccctgtgattacagaggaatactccattcactccaagtctcaccttcttttctgtgcccagactcatggactgacgtagccagagactcagttaaaggagacccattctgtgacttggagagactgacaggcccacatcacagaggaaatacatgcagacataggcatggatatggtttagcttttcgtccccacccaaatctcatcttgaactgtaatccttagacctgtaggaaagaacctggtgggaagtgataggattatggggtggtttccccagctgttttcatgataatgagtgaattctcatgagatctggtggttttgtaagtgtttgacttccctcctgcacatgctacttctttctcctgctgtcatgtaagaaggcccaagcttgctacccctttgccttccgccataattgtgagtttcctgaggcctccccagccatgcagaattgtgagtcaattaaacctcttttcttgataaattacccagtctcaggtattctttatagtgatgtgagaacaggctaatacaggcaccatgaccatagtgacaggataaaccatggagacattactgaaaatacaaagtaattgtcgtaaatgacttggttgccttacactcaaattctgttagtgtctgaagaggacatagcacctcctacctggaggacataccgttttccccatggaaaaaataattgggctgccaggtctgtatctgctaagggagtcccatatttaaactgacctaatttagaactttaaattgagactcagatgcccccaaatagcagctccaccaacctgaatagagttattcatttagaataagagtttgtttccacaagttttgttaaaatacaaattagttgggatgtggggtgtgagcgatagagactaaaatcgcttaacagattaactagcctggcaaagccttgtgggaagcaagttttgatgatggggaagagaagatgtcaagagcagatggtccagggaggaagtgtcaccagtaacttaggggtgatataagggacatgagtagcagctatttgtccgatctttaagaaataattaacttattagagaattttgtcagtaaagtcatttgagtatcacacttgcaagatactaattcatccatgtcaagttttctcattatttcacagatattaaacaggtgcattgacttgaattgacatcatgcaggaaatgccattatatgaacccttaagatattttaagagaaaaacctataatgcctggaaactcttagttttctctgacatttaaaagcagctgtaaagttaaatcctttctataataatttagtaaatcagaattgcaaagcatttcctaaagcccaaggatccaaagctctgacctgtggtgttggcatagcagagtgagagacaaattacatgttaaagcagaaggctctggacctggaatgcttggatacaaattctggctccaccacttgctaccgtttaactctaggcaaattactgaactctgtgactatttcctcttctgtaaagtcagggttgttttaataattagatcagaaattataaagtgcttagagtaataccaagcaagtgtgcataaactttagccatttagctataactctcatcatatttctgtgttgattacaaaatgctttactagaggaaaatagatgagaaagaggccatgaggtaaaaatgtagagtgctttttagagtagaatgagcttagaaattaactctatccccatcactggcttgcagtggaaatttgaaacagtctctccataattttcttgtagtgtttttttttcttttttttttttttaagaatctcagtccatctcccaggctggagtgcagtggtgcaatctcggctcactgcaacctccgcttccccagttcaagcaattctcctgcctcagcctcctgagtagttgggactacaagcgtgtgccatcacgcctggctaattttttgtatttttagtagagacagagtttcactgtgttagccagaactcctgacctcatgatcctcctgcctgagcctcccaaagtgctgggattacaggcatgagccactgcacctggccctctttcagatttttttctaagacagagacactccctatcacagacagggtcttctaaattagtacagaatgatttgaaggaacctgggtaattatcagaggaagcaaaagagaaatacagatcctcccatcagaaagcaaatctctaagactttatcattttcatcgttatgaaagaaaagatcagtactcaaagccacttatgatgatgcacgtaaattagtataaaatcagaacaaatacaatatggctttaaaatagaaacataaggctatctttccttacaagtgtcagattgcttctgtgccactgtaacacagagctttagatggatttttttaagcttcaatcagagatcaaatatattccattagtcagtgattgacagacttggtgaaaattgagttttcaacttaaacttctccaaattgtctgagaaaatcagatgcagcaactaagttcagaaggtcagagtctcaggctaaaaagagagggagaaacctaacactagaagaagaatgaatgaaatagagcacacacagctcattaggtctctataatataccacgcaaatcattggtaccaggactatcgaataatcaaacgcttttaagctgaagaatatctacattatatgccatgattactgctttaccgattcataagacaacttaaaaattaaacatgtatacaaagacattctacttttctcattagtccatgccattatataatttctaaattctgccaactctctttcctaggaatcttctagaagtcaggcattttgggaatagctgagtatcataactgaataaagacaccacaacagtcacctgtagtagtcagatcttacaggtactagaacaataacagcacaaagaccgagttatgtgtatgtattagtcagcattctctggaaaaaatgtaatatatgtatatataggcaataaatttataaatgcataaaacatataaaaatataaatttatgtaaataggcatataaatatgtaatactacacatataaatacaaataaagagatttattctatgggattggctcatgcagttatagaagcagaagtcccaagatttgcagtaagcaagctggagattcaggggagcccatgcatagttccagcccaagtccaaaggtttgaaacccagaaaagtcaatgatataagttccagtccagaagccatcaggttacagatgcaagaagagccaatatttcaagtccgaagatcaggaaaggccaatgtcactgctctgtcatgcaggccgagctccctcttactcagcttttatgttttattcaggtcttcggttgactggatgaggcccactaacattgcatctgctgttaaaatttttcaagatctttttggggtgaaaaaaacctatcattcatctttcaatcttcagtgttgacaaggacaggacatttatgagtattcctgtttctgagtttctgataagcttgtccaagttgagcacaggctggaaaatttataagagtattggaggcagagcagttacaaaaggaaaggggcatatacaaaggtctcttgtgtgatgttgtgctgcaaacactgacctttcagagtttattcacccctgtgtatatatacatattcatgtctgcacatgtgtgctagtacctatatacatctatacacacacacaatcacatatacctgagacctaatggaatagacatgaatttggcttagccagggaaattatagaaagaataatgggaaacaaggacaaaggcacctagataaaataaaaagcaactggtgtgcctaggagacctgatggacttgagcgtggaatagctccagacttgcattgaaagaaaatcaggaaagtcaagttaggaccaggtcatggcaggcactgaatgtcaggttaaggggctgaactgtttctttctagttagtgatgggtccagtgaaggatttagggcaggagaatactagagggaatattttagggagattgatcttccagtggtgtgcagaacagatatgccatgcctacagggtagtaagcagtgacgtttctttcactctacactttagccgagctctggttaagtccaataatggaagatggggtgggaaatagagctaggagtgtagagggggcaacaggtaaggatacgtatgtaccgaaagcagaaaacaagttaagatccaggcttcatgagatgcctatattgaaaattgctctggataaagagactgttaggacttcagagttagaattcatagcaattcacagaactgctcaggctcttcattattcacttgactcaacgttcctacatcggctcctttctcataagagctttccttgctctctgtcctttttctaccccacagcttttgcttcctttccatacatagaaccacacaacaaatacttgttgtatgcatgcagtggtcatataaatatcttattgtctagtcctcatcaaaactaagttgaaacaaatcatttgagccagaatctactaagagtcagattttttaaggtagttcaaggtttttatttataaatactaacaactaggagaacatttactcattcgaaattatgaaaacatagacaaaacaattattagagaaaattttgaattattcttttctcctgctatctaatttctacctttctcatgtagtctttttctttttaattagattttttatgtgagtataaggcccatttatttggcaggtggaataaattcaattactctcaattgttttgtttgacctgtaacatgtataagttagagcttaagtcagctgttctagatgcaactgcatattgttatcacctgagaagctttaaaatctatcatttcctgggccccattccaaaccagctgaatcagaatccttccggggtggggaagatgatgcctaagcacaggtgattctaccatgagtccaggcagggagcctggtttttgtgcacagctcccgtatgtcatagctatcggcacctggctctgctctagctgttcttaagtaatcttttaatgccaattgggagtatgttttaatagtggtacaatattttaagaataagggtttaaagcaagttattgcaagtaacagttaagggatttactcaaactggacacaacctctgttggagcagattacagtgaagttttgagggcacgggataaaaaagaggaaggagagatttaatggaatgaacattatttgctaaaatttaagaacgcttgcagttaaatctcgtgctattttgacaagatgtgcacaggtgcataggtgaatgcttcctcttctggtatttccttgtgtaatttatagttcaaactaaatcctagggatttaaagtgcttctcattgatatttaaagggcattttaaaccagtgggaaaatgtctttcgaaacagataaaatgtacctttataaaatccacttaatgagaatatgctaaaaagcaagatgtgtttctattggatcataaattatataattttaaacgagtttttaaatgaaagtaaattgttccaactatttgaaattactttttcctcaaacaaaacaaaaaaaatttctagatgtcaggattgttttacaaaaatgatgatctgatgatctgttggccagattaatatgcttaactttcctaagagttaaggtaacattattcctttaatgagcttgtatattatttctgatttgatgtagttttttaaaaattgggaaaacatgccttctctaccccttgcatgttttaatgccctgatttaataccggtgtgtgtgtgtgtatgtgcatatacacagatatacacatatatacacaaacacatattaaaattataataaaataatcatttgttcattatatatttgcttgttctttttctctgataaactctaagacagtggttcctatggtattttttttctctgacatagtttttcttttctctatagatattttcactacagtgcaatttttccagtatcttaggttttgctatcttaagttactctacttaggacttagaatgattccaaatagggtggtatatagattttaaaccttaacaaacagtaatcaaacatcagctagccctatacatccttgcaaaaataacaatttatttagaaattgtatcagcattactccttcacaacatttaatatcttgccttgcaagttcattatacgtgtcagatggattcagaaacttctttagccatagatatcaccaaaagagcaggaaagataacaaaaaaattaaagaataaaacagaaaaagagatagaagaaggaaaaatgaattacatggacatttgagaatggagagagggaagaccctactcattccctgcgtgtagctgtatgtaacacaactacacaaagtagttgtgttgggtgtcttgagaagcacttggggctgtgattacctccactacaacctcatgagatatttggtctatagataatttcttcctctcttgctatctacaatcctgaagaactaaaaatcagtggaaagaagaatcaacatcttaaataatttaaatggctgggcacagtggctcacacctgtaatcccagcattttgggaggctgaggcgggtggatcactagaggtcaggagttcaagaccagcctggtcaacacggcaaaaccccgtctctactaaaaatacaaaaattagccaagcatagtggcatgcatctgtaatcccagctaccccggaggctgaggcaggagaatttcttgaacccaggaggaggagtttgcagtgagctgagatcaccccaccacactccagcctgggtgatagagcgagattctgtctttaaaaaaagcaaaagaatttaaatataagaataaaagtaagcatgaaagctgagccagatcttgccaaatgatacatcacttggtaaaccacgaaggtgccacacttgccaaggagtaatatttcaatgagatgtgactcagtgcatagcagacagctgagtccatttggaccatatccacagggggtcaatccttgatgtgtccgagtgaagcatcagacaaaccacccacaacactcctcaaaggagctattaccatagctcactttcattccggcaaagaggctaagttaccatagtgcttcagatgctccagaccgtgtgtccctatggaagagaatgtggaccacagcatcaccctcatcagctcattacggtgtcctgtgttcttcacagcaacactttaaggaaggcagggtagctataatctccacacctcctgtctaactttttttaacatgaggaaactggaaagaagtaaaattagtaattaggaacacaatcttagcctccaggctacttaaattcagatcatctgtctgccactcattaccagtgtaaacttaagcactttaagctctctattccttgatttattacccgtatcataggaataataatagctacaccactgagcttttatgaggattaaatgaattatgtttaaaattttcagaacatagcatttgtgttcagtaaatgttagctatgtttattattcttacttaaaatatttttaaatattgccacataataggcaaaaaaatgttatcttggttttttgttttaatttgccttttcctgatcactaaagattaccatttttaaatcataagtttataagctattctgatttcttttctttaaattgactgttcatttcctttgctcatttgtccattgatttgtgtctattatttatttaaaagtaatctgtatattacagataatatttctctatctactatataaataacaattattctcttccagtctgtgggcttttaaattttcatttattttgccattacagaagtttgtaatatttttgtaatcaaatctgctagtcttttcatttatggttcttaatatgcttggttcagaagaccttctgtaccatgagattataaaaacacgcctacattttcttctggaggttacacaatgtttttaaaggttatttctttattctatttagaatgctggcatgtaatgaggtgatatgcccgctttattttttttctcaaattgtttatgatccccctatgaatttgaaatagcacctattggtgaattccagtacaaacaaaactatgggtccagagtttttattttactccattcatctggttgtctacttctatgcttgttgtaatcttaactgttaattagggtggttccaaatcattataaactgtcaatatatgttgtggcatttattgcacattgttcctgccaggtgaactttgaaatcaatttgtcaaatttcagaataatttcattggactttaaattagaattgcattaaagtaataatttgggaaaaactgatctttttacatcaataaggcttccatttattaaggcctttagatctttcagtcaaattttataatttttatatagggtttaaacatttcttattaagtttattcatattttatgtttttattctattgtttagaccttattatacttcacttgctatttctgttaaatacagaacatttattttataagtttctttgtagtagtctttcattagtttcaattgtattatatagttgatttacttgtttttaatgagaataaatgattgtggtgtttcattactaagtctaatgtttgtttcatgtttctgagagacgctttttatcaatttaaagatgttttgttctactgctaacttatgaagatgttttcttaaaccaatagtgggtattaactttcattaagtgccacttgtagatttactgtgatgatcgcgtgttttctctttatcaatgaaatgaattcaaacttccagaatccccaatgttcaataaactttatatttttgtactgctctcatcttgctcattgtatattattattttgtatgctttttcatatatagttcatatctatactcctatatttgtctagtgttctactttgtagtaaatttttctctatcatatatgatgagcttgtagataatttcagaagttttctattagtttctactctccaaaaggctttatatagcaaaggaataatttgcttctaaagtgatttataaaacttgcctaataaaacaatctggacctggtgttatttgggaggaaaacattgaatgttttcaatgtagattatgattactagtttattcaaattttaaacttatccttgagataattttaaaaattcacacttttctaaggttttgtccactgtatgtatatttttcaaattattggtaaaagtattctcttttgtaaaagtctatgactctttggttatatccaatttttctcttgttttgaaaatttatttactcctttttaccttgtcaggattgctagctggatgctaatttactactacaatcagctgttacatacatttatcaagtatacagatttgttttattaattctacttttatctttgattccttctaatttctttttgtccatttcactcttatcctagcttctttagttgaatgcttattttcagcgtttattgtcttgtaattaataataaacagactatagtttacaaaaatctactgtacatttcaaaatagctagagagaatgatgtgaatgttttcagcataaagaagagacaaatgtttaaagtgatagatatcccaattatactgttttaagctttacaaacaatataaaaggattaaattatcacatgtaccctcaaaatatgtacacctattaggtattaatttaaaaagaaaagcaagaatagaaacttgaccacatcttaatgaaaagcatgacattttcattaacattcatttattaacataattttaacttttatattttgtattatgagcaagagctatatatcatagaaagctaaaaatagtcaaataagcaagaatgaagaaaacaaaaataatttgttaattcactcacccagagataaccaattagccttgtgtaatcaaagtatttttgaatataaaaattaatacaaatgaaatcagaccatacattcggttttgtaacctgccttttccactttactatatttcacaaatatctctctaaattattatatattcttctagaacattgatttgattagctatactgtatcactgcttatccaaatatttctttatcattagatatttcagttgtgcctacctttcataacaatactaatatattctatgagaggtgacaagcatgaacaatcttaggaccttaacgacttttctactaggcagcaaaaacaaagataaatgaaataactggatccattattaaggcaacataaaagagctcaagataatgcatttgaagttgaatttttctctgcattggagaaacaggaggagaataaccaaagagaaaatcacagatcatggctaagatgagttaaacaaagcagcaaataaagaataagaatggaatatttctatagcaccttctacatgccaggtactatgttaggcactctcacattcattactttatttatttcatgcttaaaaaaatcttcaagttaggtatcagtattcttatccttttttagactagaaaaccagacctcagaaaagttaaaatcatggtggcagaactaatttttttttaacttttttttaagttcaggggtacaagtgcagatttattacataggtaaacttgtgtcatgggggtttgttgtgcagattatttcatcacccaggtgttaagcctagtacccattagttatttttcctgttcctctcctttctcccaccctccatcctccaaaaggcaccagtgtgtgttgtccccctctatgtgtccatgtgttcccatcacttagctcccacttataagtgagaacatatggtatttggttttctgttaccctcctctgcagcaggttccacaagtttaaagatgcttacatttacatttcagttctgtgactgtgtaagtttgctacagataatgcaggaacagaactgaaatttcagtgtaagcttgtccttctttaaaaatgtggaaacctactacagaagaaaatttttgtcaatacttataagtaaaattaacatgtccactatgaacaacttttacatttcttttctttctttaatttaagccttgtcttggcccaaattgacaagttccaaattaatatatgatttattactaatgccttaataacatagaggaggttctggttaatatttttttctagtattgttttaaaaaactaagatgttgcatattgggactttgtgtgatgtcttcatggctacatttggaaatcttatgtatacgtattctctattaattttgctacagctcatgatgcatgaccaaaatgcatgctaccacatgaattaatttccaaaatagacttaagatcaattgtaaatatttttccaatgtggatattagagctttatatcaagcaatgcaaagtaatatttacttagcatcatcaggcaaattagacttcagtcataaatccttcaccaaccatttcagtactatctagaatttctttcatgtaaaattcccaggagatactcaagggctgaaattcatgctaaaaccacttgtacaactcgcatgttacagaaaagttatgtcaaggcacaattaaacgttgaggaaagactggattcagtttggagaggtcccagtggcaagtgaagccatcatggatttgaaatgcacatggagcacatcactgtacaggccccagtggcttctcactcaattacaaaagaacacttctctgcttaccaagtcagaaatagtctttgtccccattcagttttcttatccaatttcttctgaaaaatcaaatcaagatggtaaacatactattaagagaatgcctctatttcctaactctaaattgtttaccacggtgcagatattaaattaatatttgctgaccattttgttaaagtcaaggataattactttaatttttaaccaagaaaccatctaactttaattctgtatttatcttttgtctcctttctgttcttctctgcctgtcattcacactgaccatttcttttgtttgtataaacttttggggtgcaagtgtaattttgttatatgcctagattgcagagtggttaagacttttagggtatccatcacccaaataatctacattgtaaccatgaaggaatccctcatcatccaccaccttctcacctcatgacctttccagatctccattgtctatcatttcactctctacctccatgtgtacacattatgtaactcccatttatgaatgagaacagatttgtttttctgtgcctgacttgtttcacttaagaaaatagcctccagttccacctaggttgctgcaaaagataaaataaaatgaaataatttcgtgattttttatggctgaataaaattctactatatattatatatataatacattaatacataatatatatactatatatatataattttctttatccaattgttgatggatacttatgttgattccatatctttgccattatgaatattactgctataaacatatgagtgcaggtatgtttctgatatattgatttcttttcctttggatagatacccagtagtgcccaactaccatttctagttctgtttttgccaagctatgctttcatgactctttaaatgtgtttttctagtatgtactcctcttacctttgtcttgtatctttcccactttaccatttgaaagactttaagaaaaaaaaaaaacaggccaggcatgttggatcacgcctgtaatcccagcactctaggaggccgagatgggaagactgcttgagcccaggagttcgagaccaacctgggcaacatagatagccccgtctgtattttcttttaaaaaaatttatacatatataataaaatactcattttccctctttctctgtttccctagctgccaacttaatggtacactagggcagtaagaccttctaccattctcataacttcaggcaaatctagataacaaatctcacccaaaatcctcgcttagttttctttccatgacttggttcttcatgtgggatcagttttttaggtacaagataaaagtgtttcctctttctagctcagggttataatcaaagagcaggacatactatccagctaacaggtttagatttagaaaagtcagaatgtactaccctgcctgtgtctctcttccaacttccttctcatctgtttccctgccaacttccctgaaactggacccactggcacctgaggccaacagggtcaggaactgcagctccaactttgcccaatttccattgagtagttgcagccaagtgacaaggtgagctattcattccaacccctctctgcctttcaggaaggcagaagtcttgagcccccaataatttctggatctttgttttcttggtcaaatagtcactgatgttgcaaatataaaaccagaaagagatagaaaagtgtgtacaaatatcaattttatattttaatcactctgcactctgaatttttttacttttcttgtctctctcacaagtatgatgacatattcataaacgcggagaaaccacaaagatcaatttcttatataaaacaaatccattccaggacagaactttcaggaagtaactggaaaaagagaaataataagtatcaggactttcaattcagattaattttacttctgattgactgcaagtcactgtaatttataaaattgctgatccaaagttttgcatttcttctataagtatctattaagcacttagcataatgtcaagctgagccttctgggcaaacaaagatgaagtacaagatagtccctgtgctcagaaagctcacagtttagtgagaatgttagacatggaagccgacaaatacagtcatgtgtgaaacaataaaaggacatacaggttttctttgagagcactgaggagctgacacttaaaggttacctacagaggttgtcaaagaaagagagcatcacagagcaaagaagatgttaatcaagtctagaagaatcagtaaggccaatgtggtgaagttaggaggcatgggaaggaaaaggcattccaagcagggaaactggaggtgtaagaaagcatgggaaggcccagtggcagggcatgtgaatgacaccgaaaggggagtctaggatctagtcctgaagggacaacgtgtcatgctaagaacctgggactttatccttctcacagttggttttgaaattctgcctcttagatttagagattgataattataataattaaaatgatataatagctactttttattggatatttattaaatgccagaaaccattttaagtgttttaaatgtatattcattatttagctcccaatattaatattgactcctcaaagaacattatgatggatgtattattatcctaattgtattaatttagtattaggagtagtattttgagacaggatctcattctgtcacccaggctgaagtgcagtgcagctcattgtaggctcaacctcccaggctcaagtgattcacagcccaggtagcatgactacaggtgcatgccactacacctggctaattttttgtattttttgtagaggtgagcttttgccaggttacccgggctggtctggaactcctggacctcccaaaatgctgggattacaggcatgagccaccacatccggccccctcctaattttatgtgtaaagaaacaggctcagatagcttcctctaggacgaaagtggaagaagaaaagatgggacccgccgggaggattggccaggctcagtggctcacgcccataatcccagcactttcggaggctgaggcgggcagatcacctgagggcaggagttcgagaccagcctgaccaacatggagaaaccccatctctactacaaatacaacattagccaggcgtggtggcgcatgcctgtaatcccagcttctcaggaagctggggcaggagaattgcttgaacccgggaggcggaggttgcggtgagccaaaatcatgccattgctcttcagtctgggcaacaagagcaaaactccatctccaaaaaaaaaaaagaaagaaagaaagaaagaaaagaggtgaccatttttacatagcaggtttggtggtgaggatacatttgaaccaagtgttccattgctaataaaaacatactgtctgagaggacaaggagacactgatcgccagttaatggaagctgtaagaactagaggaagccctggctttcaatactttgtgtcttttccttatcccattgttagaagggatataaagaagaagggaaaggggaggaaattatacaagtaggagttgacatttcatcaggccctttctggagttcttttgctgttcctctactctgcttctcctccaacatgagctattagcaaacctagggcaattcttcacaggtgttttctcatttcatcttcacaacaatatgatgcacagcctgctcatcactccaattcacaaatgaggaaattgatgcttagagacagtatgtgaatatatgattctttcctgtaatattgactgtagccttcccctccatccaatctccatcttttttgaccctttctgctctcagccatcctcaactagataacagtgctgtgccacaaacctgcggattttctccacagaaattatataagtacactatattttacttagtctgtattgatatatattggttgtacatattttgagggtacatgtgatattatgatacctctatacaatgtataatgatcaaatcagggtaattagaatatcacatcaaacatttattttctctttatgtttgcaacattatgaatcttctagttattttaaaatatataatgaattattgttaactataatttccctactatactatcaaatatagaacttctgtcttgctgtaccttgtaccccttaaccaacttctattcatctccttctccccattacctttctcagcctctggtaaccacctttatacttttaacctccatgagatccactttttgtattctagttattgatcttatttattatctctctcaccccctataaaactccatgatattttgggggatgagaggtttggttttttttctcatctccatctctatgtcttgaaaattcttagaagacagtagatattcaataaataatttcagaattaataggtgataaatactcatgaatgcttttctctatctttccaaaatcttaatgcctgccctgtttgttttaaattctattgcttcaataagatagtttgagaactatcctttcataattctgatctgaagaacttacaaggtgctaaattagaagatgtattgcaaagcctttatttcaaattggccaatatgtttaataaaagtttgacttgcttaaaagaaatatttatctgggattcaagttcactccttagaataggtcaacactttgaggggggttgtttttgcaaaataaaaattaaaggttaagtgtttcttgtggcaaattttgaattttatttccatgccctgattcccaatgacatgacactatagttgagatattataccgattttctcaataacttcagaatgaatttatatgtgatcaaaagacctttctgatttcaagatacctttaataacctttactaattattaaccaaaactaaaggcagattttgcaaatacaaacttgtcatttccccatgactgttatctttttgatggaaaagatgaagttcagcaaagaacaaatctacaacttggccagaagacggtagactttgccttcaaagagctgtaatacgtacataaataatctcatcagaccccatccttgctcttgcaggttaattaagcacagtgggagggtttgagagtatcctcatgaaaagataattttagtaacatttgggaaaagagcaagggttaaattttatctgaaagactagtttcctttgtaagtcttcaaaatgttttgccttttgcaaagtacagacgctgaagctggcatattaacttactaagttagcagattccccctaaaacacaatgccctaaagccaggcagagggtagaactggcaaacagagagcattaactgtatgcttcttgaaacaatcagtgaatgcaaactgaattgccctgggtcagcagcaagcaagaacaatcagaaaggtgaaggcaagaatgtccatgtttatataaagaggatgacattgttctgccctaaaaagagctaactctgtataatctcatgttttcctttgatattctaaattatactgaaaatatacttcctttttttttgagacagagtctcactctgttgcccaggctgcagtgcaatggcatgatctcagcttactgcaaccttcacctcccaggttcaagcaattctcatgcttcagcttcccaagtacttgggattacaggcatgcaccaccactcctggctaatttttacatttttgatagagatggggtttcaccatgtaggccaggctgatctcagactcctgacttcaggtgatctacctgccttggcctcccaaagtgctgagattacagggatgagccaccgcgcctggcaaaaatattactttctaaatgatacttatctgttagcttggggacatttatttaattatactacttcagtagtagaatacctatttctattactaatagtagtactatagtaatactaatcataatgatagtagtaatagtaataactagtaattataatactctaatgatagtagcaatagtaatattaatagtattagtaatactactattagtattgctattgctactaatattgctattgctactactattactatattggtattattactactattagtaatactagcactgtcattactaatagtgctacaattagcaatactagtgctactagtaatattagcactaatattagtgttattagtaatggttgtactattaatactaatattagtaggtagtcctaataatattgttattgttactaatagtaatactctattactattctgtcattcaagtaatagcaattttagtaatagaataccttaaagctgcatttttcagaaatgatcacttcagcttcacttttttctttcacccaatttttcacccagtttcttatattataaatgatgtcttcattctgagcctttaaaggacaatgtgaagcagtacatgcatatgacagaaaattttcaaaagtacctatattcaaatacattcaggttgcatttgattcagatcattgtaaaacatatttcctctgtcctggttggcagagcacattcagcagggtgaaaacccagcatagaaccttccccgagccactgataactgcaacccagtgtggcagccttcacctctgtgtatgtgggaaactccagggcctcacactcccacagcttagaaccttcaaaatggacattatcttgttctgggaacctgtggatatgagcaaccatcacatttggaatgtgattcaaatcttgttcccaaaatagaaaatatagacaaggaaatgctgaaattatatatttatatctgtatacacaaacatgaacatatgtaaactttcaacaccttttagaatttacagaagtttcattgtactctactgaacacgtctgtggtatgtgatctggatgttagcaaacccttccacgtcaactcaaaagcatccattgttcttgctttgttaaaattaatgtcacatttttattacatagagtactttgcatgatgacttctgtgtatatgatactggcacattttgttctaaacgtttgtctttggagaataagtagataactatattagtggacatatagactaaagatcaatctctgagaattctgatgccttatcttagtcgttagaattagcgacagtgccctcttcctgcatctgtaaggaacatccttcatattattttaatagatgtgagttgatctgttttggagtatttggtgacatactgagataagtggattcagaattgctaatctgcattataattggagaatgggtattgttcattttttttttgtttctaaatttcaactaatattgtgcatttttaaaagttttttttagagtacatttacagagccttgtttgtatgctactcttagttgctctaactttaatgactcatttgcagagaatatttcctaaagatgtccttcataaaaaggagaagtttggacttgacaacagtaatttacttaatttatcttaaatttggtttcagtgagcaaccctaattaacctgattttttgctgataatcactctcaatggaatcaaatcacaaatccggggatggattgagcggcacccagaaggaagcagccctccgcgcactggtccagcgcacaggatatagcttggtccaggtaggaacttcttgtgaaatctgctgagactcaccactgaatgtgcacagtggtgggaatattagtggtcctaagatgcaaaatgggaatggtcagttacatagctctttaaagaaatgtccctttctaaaagagaaggaaattgcaacatttctgttttgtagatgaccctgaattcataggatgtaaccttctgaatgtaaccttctttctactctaagttatcagttcaaccttaacttgtcgcagaatcagtgtccacagttatagagactatcacaaaaaggcagctagaatttgccacccaacactgtgaagtagcaccacattggtgtttcaggaatcagcagaaaataaactacagcaggaaccaggcttggcttccagagagatcactgcacacataagacttctacatgtgggatccaaggaatgaacgctttgtgtctagctgctgaagtttattttctcagaacacttgctcatccagtctaggatatgtaatactaacttctctccatgttcagcattttgtaaaaggaaaaaataaaacaccacaatcttaaaactgtctatttgtagacactgtcttttagaaattatctgtgtgtatatatatatatctttatgatatcttcttttagagaactaaaatgcattaaaaaatgtagtgttagatacatatattatcttatttaaagagaaaaatggtgcttttctacagtaggacaaaatattttaatttctcagctattttctttcactcacatattttcccaacttaaccaaatctagatactactatgaattcaaatgtcatgtgtgattagcaataaaatatacaatagtgtcctttcattagatgctcctttgtcaggtgcaaattttacatttattttatttatttacttatttatttatttatttttgagatggagtctcgctctgtcacccaggttggagtgcagtggcacaatcttggctcactgcaagctccgcctcccaggttcacaccattctcctgcctcagcctctggagtaactgggactacaggcgcccgccaccacgtctgactaatttttgtgtgtgtgtgtatatgtgtatatatatatatatatatatatattttttttttagtagagacggggtttcactgtgttagccaggatggtctcgatctcctgacctcgtgatccatccaccttagcttcccaaagtgctgggattataggcgtgagccaccgctcccagccaaaatgtacatttatttttaattaatttttataaatcatagcatgctttgttagacataatataataataaagttattttcttcatcctaattaagagtgttagtcttttttaagatttcaaaaagtttctattatttcataggtgactttttgaagtttcatttaagtcaatggagatactgacatattagtttttttaatacttaaaagtcaaattacttcaggctcattaataaacttttgaaaagagcgttttctttctttttgcaatcaacactttcataaatgttataaaaacaagaatgtttttgaatctttttaaaattttgaacttcttgatattagaaacatttgtgcaaagacaatggaaatagaaaacatttcaaccctttcaaacagcaatctgttaatatttatatataagagtgttccctttctggaactgtatttctaatgacccataatataagatagaaatatgtataaatctgtctagggtgagattagctgtaatgcatatcacaaacttaaatgtataaatataagaaaattattaagtgaactatgaatgtgtaattattatactcaacaagcagaatttatgaaaaataatcctaaaacaatatacattgtcaattacattaaattcaattctggatactggttcttccttttcccccagatgaaattcaggttaagtctcataacttttctggtaacgtccaggtaataaaagatccttcattaaaaaaattgatattttttcatattgatgttattccaaactagatcgtttggaataatagtaaagacataaaatcattcaaagttacagttatttccctttcccaagtatgtcagagtggaggataatttcttttctgtttttctgcactgtgttttcctctttcaacaccagtttggtaactacaatttctgactcctgatggaagcagaagggagaaataaaagcaaaggaaatgatgaagggaagcttttactgagctaccttcattgggttcttgggcctaacaggttatcgaagcttgtgagattctttgtaggatcctcagacacctagccttctgctgggttctctttgatatttgaagcagccctcatggagaggatggaagataatcattaattcctacatgtggccaaactctggtcccctggaaacacgcattccctggaatgtggaaacacctagcaaaatctggaaacacccctggaaacacccagcaaaatctggaagttgaggtgacacaaagcaaccacctctccttgccaacaacaaagctgcaggccaacccattgttcacagtgagagatttcagaactgagttagacccaccagcttagtgtgttccccaaacacaaaggcagggaatacacatcaagctgtctaagtggtttctcctgctttaggcaaaatgataggcgcctctcagtccctcccacttagggggaactaagactcacaacacaacagcagttgtctccaaatatctttctgtaaattctaaaactcctctcaaactctccaatctcttttctaagcttagggtgtaaggtgacagttaacaatagaaaaatagtttcctgctaccttctcatcctgcttggcgctctagtcacttggtttcatacctaaaccagaggccagagttaatgataatttattacatatttcaaaatagctagaagagaagatttgaaatgttcctaacacaaataaatgtttgaggtgaaggatatcctataccctgatttgatccttacctgatttgatcattacatattgtatgcatatattaaaatatcacacatggactccttaaatgtgtacaatcattatgcatccattttaaaagtccaattccatcttaaaatcttgttacagatgaaatagtattcagttactaacagtaatcatataaaaaattctgaagggcaattgtaacacattactgttacagctatgaagaaagcttgcaaataatttttttgaaaagagacccaaataacaatggagataatcttactccattaaaaaattttactccattgtaaggtcaaatgttatgatttaaggacatttatctgatacagaaaataatgatatgtataatttaaaatatttacatataaaatactcctaaaatatatggccaatctttattttacgttgatcttatctattcaataatattcagaaaatgtttataataacgacttagaccacaagttattgtaataataataataataacaaaaaatattctaactgctttgtgtatactaactcatctagtccttacaatacactgtatggtagctactatgtttatccccaatttcctgatgaagaaaacaaggcacaaacaatttaggtaacttgtgagatgaagattctgttgttattggccaaaatttttggttagactcagaaaactaagagttgttataatgttgttgcaatgtgacttttaagattgcaattttagagatcagcatgctacagcatatgaaaaacatttttttggaacctaaactttgtaacatgattcactttaagtaatattcattagcagcgtgactgttgaatcagaagtgcttcaataatgtagaagcctaatgttccaaaagctagttatctatacctaagtcctctaatcaaaaatgtaaatacctatataattcaatcattttgaatgaattgaagaatgcctcctgattttggctacagagtgtggaaaattatccaagactttaaatcagaaggcctataatctaatctagtccctaccacatatgagttgtacgatcttgaataaatcccttaacatctccaggccataatctcctggaagatcagctaggcaatacccaactatttgtccatgatttttgtgattctagtatcttctgaatctactcactggttagcacactggactattcggtcaaaagggtagcctaaatatgtagaaacacaacttcaaactctttgtaacttaaggaaaatcctttagtcttactaatacttcagccttcctactctggattatacaaaagattaatacgattatccaaaaagaaagaaagaaaggcaacattaagaacatcaagtttacttttttaaaaaagtttttgtacaaaaattaagataaagaaacatcctaatacaatccaagaagcatacccataaaatgatagtttagaattatataaattagggtttgaaggatttacttttgctgtatctttgcagtctctgatattgaatgttgaatatttgcagaacaagaattcaaaaaacagagggtgatgaatttgtgctctcttatcactagcaggacaaatacagctatcaaaaatagaattttgtgagttacttaataacgtgacccaggcttttatgtagcaactgatgcaataatgcctgcttctaagggtttggaaatatgctttgtgatctctttttaaaataattaaatatctttggatattgatggctgaagtttttatcaaagtactttagagaataaaagagacgtaatgcagtaccccgcatatagtaagtattcaatacatactcttgatgaaaactgtgtaatgttactaagttaaactttaagcttcataattgtattatgccttgtaaaaatatttttaaagaaagtatgctacagtattaacatgtgaatagtctttgtctctgggtaatggaattatgtatgggaattcttttctctacttttttattcttaactgtacctacaaatttcaaaagctagtatatacttctctgatagctaaagacaaaaaaaaaaaatcacattatgatgagcctttcctcaagaatgcgttaatgatgtgggtcataaactatgatatgtgatgctcctcccacatgaaattttctccttagaactagaaaaatacattctcataggtgagcatattcagaaacatatacacttccacccaatgtggtgtctgttgacatcaagtttaattctactgcaactaaacctaaatgtatatttaataaaaatcaccctcaagtagtctgaatatttttaaaaatagactcattatatctaatatatataaaatttaaaatgtggtatgtattagctttgtttacccagctatcctattggtaatctattttctaaaaatcgttacagcacaatttttatgaggtatgatagtccccttgctttcgtaaaccagtgatcatcctacataaacatcaagattttttgttttgttttgttcttctcttttgcttccttagtccctattattgagaaaaatgtattagtgccaggtcttgggattatagaatataaaaatgcagtgcctgacctaaagttcatataaagtgggtgaaatgaaaaagaatgcatccaaaatttacaacaacaacataaggcagcctcaaatgagcagtccagctgtgagtaccaaaaagatcagcattttctgtatagagccttaaaagtgacaatgctctttgacctgaggaatgaggacaaacatttaggcataacaatgttcatcatgctgaaatttataattttttagaaatttaaacttaaacatatcctaaatatccaatataatattactggtcaaataaactatgatcattcttatattttcaaattatatagtaatataaaattatgatttcaaataattttcaaagacatggagaagtattcaaattttaacatttagttaaaaatctgaaagctacatataaatttgtgaagaatattttaagaaattattcacatataataaagaatattataaaataaagtataatctcttagtttttcttgcctgatgctcttcttcattttctaaattttctataataaatgcagatcgtttttattacaaggaaaaaatctcatgaggcagaaaatatttctaagggtcatttggtcaggagtaaaaagaaaaaaaagaaaaaaatcatggaaaaaaacttcatgattaaatttgggtgataagtccctaggttcactagactattctttctaattctacatatacatgaaatttgtttaatattgatatagtccaatccttttgttttgcagttagaaaataagccccagaggtacacaattataaattaggggccaacaaactatgccccatgaaccaaatgtggctacagcttgtttatgtagtaattgcaggctatgaattttgtttttttacattttaaggatttttttaaaacagcctaaaatatacaactatttggccttttacaaaaaaaaaaaaaaaaaaattgctgactccaacctgattccttaacgtagcactctatattagaagtgagtctaggttttaaagaaaattatttaatttagtatttaatcttctgtaaaagaagtaggtgcatcccttcttgtgctgcctattcacaccaactcaagcactgggccctcactcattcatttattaattcatttgttgattccaccacaaagagagacacgtttctacctttatgaagattagtctcatagaaaagatagaaaataaacagataaagaaacaagcaaagtaattacagatcatggtaagtgccaagaatgtgataaaccaggttgtataaaacagaaagccaggtgaccttctttaggaaaaatgacagtggaagctaagacttataagatgagagcaaatgtactttgcaaggagccaagtatccctggaagatggaacacacaagcagtactccttaaagaaaatatttataaaatacagaacacttttgggtagtgaagacattaatgtgtcttaatctcaaagatctagtgggattacagcagataagtaggtggctttcatctttcccttcctctattaatcagccatggacctggtgtcctgggaacaatctcctcaacctaggaacaaagtacaattttcaattcccacccctcacactttcctttctatgtatactctcttgtatggaaattcaatgaaatcacttgttttgattctaatttgttcaattcttcagcaaatacaagccatcaggtacaaaggatggcgctagatcaagtctaactagaataccgttcctcttctaagagtgccccaaattaagttaactctccatctttccggtttgagataatgtatttaggtaattttaattgaatgtttctcaaatggcatttttttcctataaataatacaataaaaataacctgagggcctcgggtttactaattggcttcatctagaatggctccaagaggagcagatggaaaaacactgtggcattgacattctttttcaaccactgcacaaaaaatcctagggacagaaataggaatttgacttcatggcaaaatatagctcctgggtcctactttttcctctattgcctgttctcgccacctgctcacatccctgctatccaaggacttaggtcattcaccaagtgcatggataccaactctttaaataggtagcaataagcatcagaacaggatgggaccagtagatcatcacagggtcatgcttccctaagcttatcaccccagccacccagaggccaactttattttaaagctcagaagcaatctttcttctactagccagagatgccatagcacggtgcaaaagactccaaatgaaggagcagttctgcgaatccccttgtggagggggtgtgtgcccaacatttgtggaaagttcattaccccaaggaacacagatatgttccagaagctattatttccaaatagcaatgaatttctcctcaaatagcctcatagggtattggctatacatgaagtctttgatataagattcaaaatcttcataaactacacaaggtagtgttttcccttaagctagaagcacagagtttttctattttatcggcttaaaaattggtgtgaacctcatgacccaaacatgcagaagcatgaccaggtcttgtgaactttggactgtgcgtgctgacaaattcacaattttttaaaagtgctattcatttgttagtactgattggcaaataaaattaagtgactcttcaacgtcagaacctgaaagcttaccattatgagatgcaaagacagctttcaaacaagctaggcaaagaagaaattattgagatcacagccttgcagtcatgaatttgtataggatttcagcagctgtgatcctttataacattgttaaagaggtgccgagcagggaaccaagatgacattaaatggtacattgtccacatcattaaataacatccaatcccatatagagagaattattccactttcagttctctttcaggctttcaggtgaggaaagttagttgttggaggtatgatttaaacacctctccaggccctactaacgtttttaagtcccgagagcaggactagagtacagagcttttagtagacgacagtgtctaggtagaagttgatcatgatttcccttgtttgcaattgtttttataattaccgtccatttgtgggaaatgatactgatattccacttataagaacaatatatattttctttttggtataagtttatttaagtaacaaagtgaatccacacataaagtcattaggtaattaacagaataggtgtcaattggacatggaattggagatacactgatttttaaaattacaacaacatcctcttaagactccaattgactaactcacaaaacatcctatgaacattttcctccattaaacatgatatcaaattcagccttcctttcacaagcaccctcacactcccacaggactactgcaatcacctagtcaaccctcctactccttcataaatgaaataaagtcagaaaagatttagtgccttgccgagtccctcagcttgtcagaaattaaaattcacaagggtgttgtaaggagaagaatgattgtataaaattccttacatgcctcaaacccggtgcattagatgggattatgcttggcatatgttgaactaggaaaagacagaagagcaattaaccaaaccaaatgttctttattatgtctttatctgcacatgggaagtttcgcataacttaatcagatctagagacaataataataattaattccgaaaggcaaattgtatctgtaagtaaacaaaatgttattgggcctattgaataaattacacattctgtaaggataaaagccccaaacggattaaggaatgactaaagttgtcatctaattacttcttggaaggaatttagaggttcctatagatggaagcagtgtgtctgggcacgtgcccagagccagaagtgagcatgagcacagccatttctgacctcaaaaaaaaggaaataatgctttgcagtccatcgttatgcaaactttgctggaaaaggtaagcagagggtcgtagaagtgacttggcattttacagattctcttttatagaaaaacacacacacaaatgctttttgttttctaattttgaaaaacactcaaaaaaattttctgttttgtaattttgaaagaaaaagtgttttgttttctaattttgtctattttcaaattcaatattcttatgaatgaccaaaatcttacattataagaagttttgcaaaacacaacattttaaaaagcctatttgcttttgaatgcttcaaatgttgatactgaataatcctcaaacataaagggaaattattaaaatacgttattaaatgcatcaatttgataacataaattgcttctgcttccataagatagttaacaacttcaagtgaaattaggattaatttccttggaaatagacttttaatagtcaaccttccaaagtgaaacaggaaggtgactcataatttactaatagttgtttcctcagaagaaccatatcatacattttttaaagataaaatgtcagggctttgtaaaaagtgaaaccaataaatgaattttcaagagccaagagccatttgcatctttcttcatgatttttgccatagatacatgccaccaatattatttatttagaattcattcaatcaactcactatttttagtcatttattttaaaagtaaactttttttcacttatgtaagaaacccacgggttttgtgtgctaattatgctttttctaatgtacattaaaataaatgcataattattaaaataagaaatattgacctataaactacctaatatcatcctcatatattttggaaactctaggctgtatgatgataaagctaaattaatatttgaatattatgttcatatcctagcttttcctgtcattgtttctaattctacatctctacaaaatttccacaactcatttagtctttatatcacaatttttattactcttcatactttaggaatattatatctggcattcatgttgcatataccttgtaaaaattgtagaaaataactaatagaaataatgccaacttctacagtatattaagccaaataaattctgctttctaaattccagtctaaaagcagattttaccaacatgaaatcactgattttaacaagatgtgaaggtgaacaaactatacaaaatagataattattttatgtacaatttaaaaatgaatattccctacatcactgtttgttgtcctcaggcaataaaaactttggaagagactaaaggcaaaatcacattgaagagggtcctgttggtacattttacggccccctagactttccttaaactacctatcacataatgaaggatctagtatataggtggaatacaatagaaataatgtctaataaatacttttattaaatttcacaaatcaataactaggaaaatgcagtatagactcatgaaacacaagtgacatagttaagagagatgacctccatgccttgttgtcaactttaagaaccatacacaggagtttggtcacagaaccactgttctgggcatggtcctggtgactggcaagctggtaaaggcttgaagtgctactaagcaagcgtagattggccaggacccaagggaacaggagcaagaataaatacaaacctggtcaattggaaaatgcataggaaaacatctgaaacaagtttagttatgaaaaatacacagtttcttccctcttacggagaggggaaaaaagaaacaaagaaccaaagagaaccacaaagatgattacaaacttgaaatatgaactttccaaagagccattaaagagactgaaattactactcaggaaaaaacaggctgaatgagggttcattaattaatgtccttccagttatgaatgagcttgtttatgggaaacagtaaacatctggtgtccatgattggtgaagttgagcaggaaaataaatgtaacacacaacatgaggatttaagaggctggtaagcctgagaagaataatcatcacaatttttgaatactatataggccataaagttcaccaaattgagaactgcaaggaagcctggaaaaatcaatgataggacctttattctagtgttttctacaataaattaaaccagtgtgatattagacaagttatgtacctctggccttagtttcctcctctataaaattagtatattgtgttaatgtaaaaatgaaagagagataatgtaactacaagtgtcttcatattttgtctcttctgtttactgcttgtatgaatttggacatattttatgccttggattcctggcctataaaatgaaaataatagtagtagcatcctcatagatttgttgagaagattaaatgaatgtaaaacactcagcaaaatacctaattcaattggagatagttgtttttgctgccattactatcatgtgcaactaacagaagctaactctaactggctaagtaggaaagagtttattgaaaggatactggttcacagaaactttagatgtggagaatccgattcaagggtaagcttttaacaacaaaacccaaaaccacatcacatatttgtcacgataagaaaatgttatctaccatcacagaatgctcaaaattaaagtttgcatcatttgtgacttttgcatcagaactcaattttagtaaaggaaaccatgattgtccatggtttagtaaaggaaaccatggttttccaataaccatgatggccccagctgcaaggaaggctggaaaaatcaagtgtctgtcatttttaagttctataggagaaggcaatttttgcccagattccccaggtatataacaagttcaaatgctcagtggccaaaattaactcagttcattcaacctccatgctaagacttatggcttagaatgatttattgaggaaggttttgtgggttttatccaaggtcaagaagatgatagacataatctcttgaagactcaatggctccattatgaaggacttgaaatatctgtgataggaggaaatttccaagtaatcttgacatataaactgagtaactgggtaggggctcagcatccaatatttaaattaaagccactagatttatagttagaaattgtgtcctcaattgttaatttacataggtgaagactttccacctatttgtatatgactctagagagacaaaggtttctaagtctttgattggaaaaataaaagttttcagagtttaataaacattctctcatgaaaagcaaaaatctatattcttttagtctgaactattaatacatttgtgctgctggggagaatgcttcccttgaggaagacatcagctaggagggaaaagaccagttataactgggctttatctattttaaactcctcaactgagaaaatagtgcctgacatattagcaacccagaacccaactgtcctttctgagtaatggacagctattctaaccccatgactaatgcccagtgcctgtcttaaattgatctgaacttccattactggtacattccaagaccagtgggtggctcacagagcaaatctcccccactgagtgaactgcagggcattcctgaactggtcagactctttacagtctcaaaaaatattctttagatttttttcatgtgtttatgaaaaacaaagacaattaatattctttcttgcccagttatttcagattataagcaggtaaatcctgctctacacgtcttacgtataaatagttggcattttcagcaattatagcaatcaagcaagttgaagatacattttaaaaggtcaatctgagggttgtctatttattttcaaactaccaaagcttttcaaagcataatttctggaattaccactttactactatggaatgaaagttgggtttcctcaacttgaagggtaaaaataccaccgaatgtatagtaaatttattcctataatctgttgaatgttatacacatcaaagatatacaaaaattggctcagaggaatcattgtcacccaaatggagtttgtccagggaggaggatggtaatttggcaattcaggttgagtatcttttagccaaaatgcttaggaccagaagtattttggatttcagattttttttaattttagaatttttgcatatgcataataaagatatcttggggatgggacctaaagtataaacacaaaattcatttatgtttcatatgcaccctacacacatagcttgaaggtgattttatacaatgcttttaataattttgttacccctcacatgaggccaagtgtggaattttctacttgtggcatgatgttgacactgaaaaattttgaattttggattttggggttagaaatatgagatacatttttctaaatattttatgtaataaaatgattggaaattaaaatgatttgaaattgtagtagggaatttactatttaatggcatgatatattttttcataaataaggagttttgtttgtttgttttgagacagagtctcgccctgtcgcccaggctggagtgcagtggcgcgatcttggctcactgcaagctccgcctcccaggttcagaacattctcctgcctcagcctcctgagtagctgggaccacaggtgcccgccactacgcccggctaattttgtgtatttttagtagagacggcgttttaccgtgttagccaggatggtctcgatctcctgacctagtgatccgccgacctcggcctcccaacatgctgggattacaggcatgagccactgtgcccagccctttctttctttctttcttttcttttttctttttctctttttttttttttttttttttttttgagacagagtcttgctctgtcacccaggctggagtgcagtgacaccatctcagctcactacaacgtctacttcccgggttcaagtgattctcatgcctcagactcccaagtaactgggattacaggcatgtgccaccatttccaactaatgtttgtatttttagtagagacagggtttcaccatgttggccaggatggtctcaaactcctaggctcaagccgtccacccaactcggcctcccaaagtcctgggattataggcgtgagccaccatgccacaccaatagggaatattttaatgtatgtatctgttgtaagtggcttccatacatcaaaggagcaaaacggagaaattaagctgatgcctgactgcctgggttcaaattctggcttctccacttccccaccatgtgactttgacaaattacttaatttatctgggctttggtaccttatgtaaatgggataataacagtccttaatccatgacattaagtaatgatagagcaatatctgacatatattaagtgttacataggtatttgtttgaaaataagtaaataaatgtgtctgaaaatatcgattgcacatatttaaggtaattattggttgagatataaaccactaaattaccaaggtttggggttttttctataagcataaagacttccatttctcaggtttctctgtgatatcaaagttttactactggacaagaatattagggtaagaaaaaaatacaagaactctctaggcaaaaatttaaatattcagttagattaaaaacatactcagttgtttaaaacaaacaaaaaaaaaggaatccaggtaaagtagaagactcatatgacattttttaaattactatttttaaaaacgttgtaggaattattataaataaaaaagtgatcatgaccacagacaccacagacatcaattaaataatgtgatccaaagaacctttgtgctaacacttacaaaataattgagaaagactcaagttagtgtatatgaagatttaacagggacaccacaagctgctttacttcaattttaaacaacaaacaactgtcatacaaaacagtgtctttaaacaactaacgtatgtcttttaaaaatctacatagaaaaatatttgaaaaggcctatgaaattttaaacagtaactacttctaaggatgagtagagtcatacattggataatttttctttttactttacatgtcaccatgttttttccacaacatgtattatgttatagcataaaacttcttaaaatattagttgtagaagttagtaatgaactcctgggctcaaagcatccttccgcctcagccagagtaagttctagtgttctatagcactgcaggatgactacagttaacaataatatattctcaatatgtagttgtaaatagctagaagaaggctattccttgcattcccaacacaaagaaatgataaatgtttgagaggatggatatactaattaccctgatctgttcactctttgatgtggtaagaagatagatctcttaagtattcttacttaatgaaattaaaaaagaaaaaaaaaaattggcctagcagtttacaagttttcacaatgtttatctcaagcaagctgctgaagcacagagtgtggtattataatccacataaaaagctttattttttctcctttaaccagtccactattattatttgtcaatgataaatacattccttattgtgaaatgattttgaaaaatactcaaggaaactgaattaaagacttgatcattttcttctgtccttcacacctgccctggatttgcaggaaaatggacaaagaaaatatggtggccctccacctggctgggatgctgcaccccctgaaagaggctgtgaaatttttattggaaaacttccccgagacctttttgaggatgagcttataccattatgtgaaaaagtgagtccaagtttaacatttaatttttcttatctttcaagggaaaaatactattctcttcactttgctttcattctcagcctttttggtcaatgattgtttctgttcaaattaccattcgtcaattatggacaaagtttctagtgattgtccctttataatcactgccttgacctagctcttgtcctgttactgacagcttgaattgctactatttctaatgactttcaaatctatatcttgttccccaatctttccactgtcatgccatctaactacaaactatccatctacaataatcctacaacaacctgtctacaattagatattgctccaacttgacattttaatatgattttataaagtacattttcattttaaaagaatgcatacgtttgacatggctcaatatgtacctataatccagaatgaaatcttctccaagtctgaatttttcatctacctaaatgcatcaagaagattaagaatatggcttttcaattaaaggaagaatatgaaattcaatttcatttttaataatttaccgagcagtttttagcaacaagcaattcctgaaaaatttagtaaatatgataattcagctctctgtctaaatgccaaaatcaaattgtgtgtgtgtgtatgttaacagcctcatattcagaactgcacctaattttacacatacaatccctgttggcacaaggacaaatgatgaggacttaattctgtaagaatcttatacattgaatctgaaaatgaacatcgagaaacacctgaaacatagagatgaaacgtgctcaaactctttcctaatgagcccagaagaaataggtgttcattactgaatacatgggggtggggtagggtaaggctgagctgagaagtgaccaaccacaaaggtaatggcatgtgcttaatggcacttctctccagacatgccccaacatatttatctgggcaacccttcctacagcagccagcaatagtaaattgctcagtctagactgctgcaggacagttgtgggggtagaagggagtattatatgggcaagtagagtcactatagccaggggaatagagtgctctcctgctctggaggccctggccctgagttagtagcatttaggctagcatttatattcagccagcagactaagaaaatgacaagcctcccagatcataccctgaaacacacctgtgttcattttgatgtcatggctgaggaaaaagacagctaagaagttagtttggcgaatgagcattcttgtattttgttttaaagatcaatgatagaataggaccaactggaaataaaactatggagatttcagaggacaatttagagggaatggacagatacatcaaaaaagtgctttttttttttttttttttttttttgagacagtgtcttgctctgttttccaggctggagtgcagtggcacaattatggctcattacagcctcgatcgcctgggctcaagcaattctcccacttctgccttccaagtagctgggactacaagcacgtgccaccatgctcagctaatttttgtgttttttgtagactcagggtgtcaccatcttgcccaaactggtttcaaattcccagggtcaagcaatccacctgccctggcctcccaaaatgctggaattacaggtgtaagaaactatacttatgataaaatgtgttttcatgtctctaagatgttccagataattatgtgatgaaaggtaatttgctctactaaaaacacattctgttaactcaggctgaaatgtcacttggacacattttatttaacttaagttctctcacgaatcagataactgatctgaacccacatgaaaatatgcaaaccattccacatgggatgcaaatgccaaatagtactggcaggaggcaaacttcagaaaacattgtgtgtgtgtgtgtgtgtgtgtgtgtgtgtgtgtgtgtgtgtatcagcacatgcacacatacattcagaagtacaaacatataaattttctatttctattacttgcagatcggtaaaatttatgaaatgagaatgatgatggattttaatggcaacaatagaggatatgcatttgtaacattctcaaataaactggaagccaagaatgcaatcaagcaacttaataattatgaaattaggtaagctctgcctcttctagattaaaagtgaaacagacaatgcacctgtatctgtttttctatcaagtttatctaatatggtatatgccaaaaaaaagccaagccagaaatgaaacagattaatagttttccttggcaaaaataaatttgattaaatatttgtttaatatcaatactatatgaagtaccaaactaagttctatgagtgttcaaagaaacaaaacttaataatttgcctgtttcataaatatattttgggcagtgttcaaaatattgggaatatatcaaggtataatttcctactcgcacagcaaatatataagatcactcaaaatagtatgaaaaaaatcaaacagggatgggataaagttgtgtgagaaggtggctactctaattttgtgtatttaagaaaatactgtctggcagagtgaaacttaatctgagatttgaatgaaaagagccagtcatgacagtcagaccttgggaaggttatttcaggaaaggagagaacatcaaatgtaaagttcccaaagtgaaaacaagcgtgatatgtttgaggggcggaaagcaagcccatgtggccagagagggtagagaggagaactgaaagcaatacagttggagagctgtgtactcagtggggaagtatagatgaagtatagaagtctctgtggtaaagaatttgaattttcttctaatagaagggaggaaagagttttgattacatttacagttttttaaacgttcctctgctattgggttgagaatgtgctatggcaagtgagatttgaaagaggacaccagttagaaggctgctgcagttgtccagatctggacaagagatggtggtagttggatgagggggtcagtggtggacaagataagaaagggtgagatctggaatatattttagatcaagtacacaaaactggctagatgttggatttagactgtgagagaattcagaattttgggaggaaacctaggattttcgtccaagtatctgtgaacttgctagtgccattgcaatggaaaagactggaaggatagcacaggaaggactctttggggccctttacatttgagatgcacattgaacatcccaggggaggtgttgagaaggtgactgtctgcattgtagtggagtcaggagacaggtcacagttggaatataaatttgggggtcagctaaatatagatagcaattaagaccatgggactaggggagagtcccatgtaactgactttgtgcaaccaataaccagttttgataaactggaaataaaactgatatcatgaaattgtctgatttctaaaaccttcacttggagagggaactgtcataaattatttcttaaatgaagccctgagccatgtgagaggaagattagtacagcagttacagatcaatagttttggagtcagccatgcttcgtttgagtgccaccaagtaacttcaccattctaagcctcaatttcctcatctataaaatggggatactatctatttcataggtttattgcagtgatattataacaactttacagtgcttaacccagtgtttggaacacagtaataggttgacaaatgatttttaaaaattatattttcacacagttcatcaattctagatgcagttttttaaatatctaatccctttgtgaaacagagctgcaccttaaaatgggtagatcttaaatctgatgaatgcagaaagaacagatttaccacaattatttttctctcactttctataagatgaaaagaagtcatacattatcctcacagtctgtaaatcaattaactactggctcattttattaaaaactcctgggctggacacagtggctcacgcctgtaatcctagcactttgggaggctgaagcgggtggatctcctgaggttgggagtttgagaccagtctgaccaacatggagaaacctcatctctactaaaaatacaaaattagcagggcgtggtggtgcgtgcctatagtcccagctactcgggaggctgaggcaggagaatcgcttgaacctggcggggcggaggttgcagtgagccgagatcacaccactgcactccagcctgggctacaagagcgaaactctgtctcaaaaaacaagcaaacaactccttagccacaggaagatcagctagattcagcaactacttgtagtctaaccattcagttccaaggttggattctagaagagagtattggagaaaacataaaggaggttcccctcccacaggtgcctccatagacccgaaagagctttgtgtagtatgtggaggattgtttgcagtaggaatcaggagacctggggtccagcccaggttcctttggacaaatgctttaacctctttgagcatcacttttattattggcacctgaacacagagatgttctgagcattcactatatgaaagaatatatactaaagtgctttgaaacagctacaaaatgctacattatagttttacgatttccagaattggaaggttcccccattccttggtctttcatggaacctggacatcgtaacagcaatacccaaaatggtatctgagttgagatggatatggaattggctctgtagggaatcaagtggatttgctgtatctacttaacacattgttacccaatagggcctgtaaatattcataggatatgaataatgaatacaatcaatccaccatggccactgataattaacagtatcctctgtgaattttagtgcctttgtctgtaaaatagagataaaaatatacctacctcatgaggtgttttgaaaaataaatgaaataatgtacatacatctcagtgctgacacatactaggtgctctgtgaatgtttgcagaaggttatggtggtgatgatggaggaggagaagggaaaaggaaaggaaaaatatgacagaaattaggaagggaaagttactggccaggattccagtcaaagaacgggttggatgccagaagcaaggtaaaggcagagtgttgtcttaaaaattagggggtagaataaatggaataagacaaaccagaataaacaccccatggacaacctcacctacttccactctgccttgggtcagtcctgccagggagagaacttttgatgcagagaagaaaacttcaatttgcttttatccaccatttctcacccacatatcattatatgagcttctacttcatcaaatattcactgatcatcatctaaatgtctggcttctagagacattaaagttgtggcctcagcttttgagaggagatggctctttgtctccaaataattccggcaaaggctcccgacaaaccacaacagtttaaaaataaaatatggaattctaggctccccctgctgatagctgatatatgaccggctccctaactatcaaaaataatataatttctaaatacattaatactgtggaaaaggttttggtgttttcttttttaaatgaagttaaaaaaaaaaacttggaagattattttatcaacctttccctgcccttgagaaccacgctcctggcacaattcttactagactgagatggagctgtcacagatctgacacacagtccttgcttcttcagcaaagcataggaggcagctgggatagtggcctaaccctggtcagaaaacctgccacacaaccaagagccattcgctccatggatagcagacaaatcgcccgcccacgcagctggctgagagcttgtgttcactgcaaatgggtcatttctgctaagcatacctcacccatttttaataacttagtgttattacaaaagtcatacagcctaatatgtcacacatggaaaacataaaaaaagcataaagtaataaaagttaaaaccatttctctgtaactccattgccccaaaataaccactatgttgacattttattgtctttccagatttttctatacatttttaatcaaaatggtcttcacatatacttattttatagctttttaaagccaacagcctattgtgaactggtttttttaaaccaataaatattcttctacgttactctttttcatgactgcagagcatgcaatggtatgaatgcctgtcttttgtttatcagtgatcctcaaccaggagaatactgtccacccaagggggcatttagaaaggtggggaggcatttttgaatgatctgattggtcactattggcatgtagtaggcaagaactagagatgataaacatactatggtatgcaatgagaaaatctccagttcaaatccagtaacatctcagttgagagacattaaaccagttctttaccgtgaggtactttgctcatttccaatttgggggagattttaaagagcagtaataaatatccttaaacataaattctagtgctgatttttattttccagggacatattcccagaagatactttatttggtcaaaggttgtgcacacttaagacttttgataaattttgccaaatgttctccataatagtttaatcaatgtatactctgccagtgcaggtgtacatacctcaacttgctaagaagcaactctgaacagtggaagaaacaggaactaagatcccagagctaggtttcattccttatattctataacattttaagcaagtcttttaatttcttggcgtttcaagttcatctgtaaatggaaataacatctactctgcccatcccacagtctttttgtgtgggccagataagatcatatgtgaaaatgtgttcaagtataagagactgtttttaacagaggtcacatcagaacagcttttctctttggctgcaaaaacaaaggaatgtcaggtgttgcaaactcaagtgactgcagggccaggcaggtggcttcagtaacctgggttaggtgtaggggcctcagtgttagagttgcttcagggagtggcgaggattcacaactcttgaatccacatctagcccaaagaaagcagatgctactcagcttcaaatagcttttttctttgaaaatgtagagcacatattgacacatctaataatcttgcaaaagaagccacaactctacaaaaatggtttcaacactatgaatccaaacatcccacatcttcaggcaacatctggcccagggccatacattttcaacctgaatcctacaaggattctcatcaatcaaaatccctgctctttttgcagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgcctttgtggagtatgagagtcatcgagcggctgccatggcaaggaggaaactgctaccaggttagcaataccctcctcaaagatgagatttctctgctctatcctgcctgcctccacaaagaaaataagcatttttaatttttttggctctctatctttcattatgttgcgttttaaattttttatccttcttcactatctcacagttgattccattccctgaagttttcacagactcagttttgaaaaatattcgttctgtgcattgagggtaatttacatgcactaaagaaacccagaactttaagtgacagcaatcctacgatgaataaagagtcttcttaaaattgagtatatcaatcacagttacttttgtgtatagattttgttatcaggcttattagaattagggtatattctccattacaatgtagaaataaaaacctcaagtgatacattattccaatgacaactaggtgacatactttcattatgactccctgttgtctaataatttctgtctaatttgagagaagagagataaaaactctctttagaaagtcacctttatttgttagttattacaaccacaggatagatcccatttgttgaataactagcaaattctctatccaccccagcactgcctcggtcagatcaacttaggaaagttgtttagggttcttgactcattctacaacattcactacaacttacacaggtttttaaatgaatactaaaataagaagatttcaagaatgacagactggacatattggaatgaacttttctcatgaaaattatgagagagccaccagaattcaaaacaaaacaaccacaacaaagacgtctacatttatgttgcctttgtacacactcttcaaaacaaaccaatactagatcagccccctagataccttcaaaactaggagacagcttctagcttggggaagatctttcaataataccttcatgaaggcaacttaaagtgaaaaacaaaagaagacagttccaaagattgctgaatcttttacaggagtaggtatttttaatgttgaatctttcattcacaacttcttccctcttcaccggccagcagtcaagtcatcaaaatcacaggtccatagatagactgactaagactcctcatgggggcatactttttggtgacctggacatagtattttgctatctgcttcaattcttctttcttcttcaacatcgacaactctaccattgaaaagtcttttgtatccatcctgttctcaggccaccactgaagtataaacacacatattctaaatcttatgtaatttttttcaagatctaattattctcccagctctgctttctcttccatgtgccccaccctccaacttctccatcctgaacattaccaccagattaatgtttccaaaatatcctattatcctttcaataccttcttgcaaacagtaggaattcaatctcaggaattcaatctctatctataggctgaatgatgtctgtcactaactggccagttcaaaaatattcataactccttgttgtgtataagaactaagtatgtgatttcattatttaagaccttttattcatttcctggtcttttatgaccatatttgctaccttctatttcccaataaaaactcttccttctatcaggacagacaacgcaatgacctagacacttccatgcttatgtctgtctcctttatttctaatgctatatcctctactaggaatacctgcccctccttcacttgtgacagtcaccccagttcagccagtaccttcttgagaaacctgcctccagtattccagcccgaattagtcactttcctccctcaatgtaggaagccattcattatctgtaaaaacagatatgtgaaataattttgggtgtaggggagtaggagagtgaaagagggtccaaaacaggtgaaagacagtttccagggtcaagacaatccagtaatcccggagaagctcaacctcctgaaagcttccccatctcttcaaagaaaatggcacattggcccactaaggaacttctttatgtaagtcaagctgactaggctggccagggcctgtcggataagcttagaaattcttaagcacagtcctgacttttccttgtggattctctgtccagaatacaccatagggaggctacctcccctccccatccactactcatattcaccatgatccaatgcaacccccttacctatggccctttgagccagtctcaggtggatttcattcagctggattttagtcagcaactaactcatcagcaaactaacaagatgaggcacagtggtgagtaagccagccagagcaaactccttcgcctctaatgagatctcagaacaaaacctatcacccatttggatcattctaccagttaccccattgcagactcacccacgataaaggaattctgtaattgccaaccctttgagaagctagtttaaaagaacaaggagactgttgagaaaaaatgataagtgaagacaacaagaaactttttcttaattgacccagacttggagacagagacaatggttgtctaaagggatgtaactgctcctcagccagttacctcctctctaaacagtggcagttcccccttgacaaatctcacctttactgagctctttagaggacgaagttgctgcatcccactcagaaactagaaaggcattctaagaacgctctgaagaaaagtcctggcagtgattctcaacgtcaggctgcatcagcatcacctgggaggcttgttatagcacagattgctagatcccaccaccagagttcttgattcagtaggtctggtgtcaggccaggtaatttgcatttacaacaagctcccaggatgctactgctcccaggactgcacatttgaaaaaccattatttgaatcaatgattttccatcctaggctgcacattggaattacctgatgttttcaaacaatattagtacccatgtcagcctctaatcaattgaatctgcatcactggaatataaagttagggaactggtgttttaagttttccagatgattctattcttcagtcaaattgtgtgattcacagctaaatctattagctaatgatactgggtattcatcttcatggcatgtaaataggaatacctgggaatcgctcagtcagtaaatatttattaagcatctagaacatatattgctcccttctgaatgacatgagagattctaaaaaaaaaaaaaaaaaaaggagtacctaaccattgcactcgaagagtgcgtattctgagaaaactatacacatacaaaaacaggaaaaattattaaaaggagagtgtattgttgaatcatcatttaaaagttattaagtcttatctgcatcatcttttcttgacctcacaacaatagtaggttgtagataaggaaactgatgttagataggatatgttttctagccaggatcattgttctgacatttgaacttgtcttacttcaagttcatgttctcaaatcactctgtaacactcgctttcaccagaggccttcaaaaggatattggtggaaatcattgcttcctatatttactctcatgtctgttctcttagccttatgcagcagttcatggtcctaggctgagaagactaaagctagagcagtagtactcaactgtagctgcattttagaattaactaagctacttgtaaaactcctgagatccagcccacttatatcagaattgggaagggcagtccggaggtcagtaatgagctggtaaatctttaacagccagctcctagagaaaaaaacaaaagccctgatttgtagcatttgccaatttccatggtattaacactcccactgtggctgatttcaagttaccaacgtggctttaactggctcacaaaattctgatactttctttgtctatttatctgttgatgaacatttaggttgattccataccatggtaattgtgaataatgcttccataaacatgggaatgcaggtatctctttaacataatgatttcatttggggagaatatatacccagtatggtgactgcgggatcatatggtagctctatttttattttttttgaggaacctgcatattattttccataatggctgtaccaacttacattcccatcaacagtatgcaagtattctcatttctccaaatccttgaaaacattatctttgatctttttcataatggccattctaccaggtgtgaggtgataactcattttagttttgatttatattttcctgattagggatgttgagcattttttcatatacctgttggtcatttatatttcttcttttgagatatgtctattcaaatccattgacctttttttttatttttgagacggagtttcactcttattgcccaggctagagtgcaatggcatgatcttggctcactgcaacctctgcctcccaggttcaagtgattttcctgcctcagcctcctgagtagctgggattacaggcatgtgccaccacaactggctaattttgtgtttttagtagagatggggtttctccatgttggccaggctggtgtcaaactcctcacctcagaagatccactggccttggcttcccaaattggtgggattacaggcatgagccagcacacctggccttttgctcatttttaattgggttatttgttttcctactattgattgtttgagttatttatatatttgggatattaactccttatcgaatgtatgctttgtaaatattttttcccattccttaggttatctctttcctccattgattgtttcctttgctgtgcagaaactttagtttgatgcaatcccattcatctatttttgcttttgttgcctgtgctttgggggttatagccgagaaacaattttccagaccaatgtcaagaaattttcccttattgtttctttctagtagttttactgtttcaggtcttacatttaaatcttattccattttaactaggcttatgtgtatgttgtaagataagggtcccatttcattcttctgcatatggatatccaattttccttacatcatttgttgaagaggctgttccttccccattgtgtgttcttggcatctttgcaaagatcagttgaccttaagtgtgtggatttatttctggactctattttgtttcactggcttaaatgtctgtttttatgccagtatcatactgtttcgattactatagctttgtagtatattttgaaatcaagtagtgtgatgaccccagctttgttcttttggttcagatttctttggctaaggtcttttgtggccccatacaaattttagaattgtattttctatttctgtgaaaaacatcactggattttcgatagagattgcgtggaatctgtagattgctctgggtagcgtggaccttttaacaataataatgcctccaatccatgaacataggctatttttccatttatttgtttcttctttaattttttatcaatattttatagttttaagtatacagatctttcatcttgttggttaaatttatttctaagtatttttgtgggggggatggtattataaatgagattgttttcttgatttctttttcagttttcttgatttcttgttgttagtgtatagaaaggctacttacttatgtatgttagttttgtattttacagctgtactgagcttattagttacaatggaatattattcagtctttacaaagaaggaaattctgtcatttgcaacaacatgggtgaacctggagcacattatattaagtgaaataagccaggcacagagagacaaatattgcataatctcacttatatgtggaatctaaaaagttgaattcatataagcacagtgttgaacggtggttaccagggcaggaggcagaggtatggagagatatttgtcaaatgatacaaagtttccattaaaatgaataatttctggaaatctattgtataatatgactatagttaatgtatttgtatactcgaaaattgctaagggagtagatcttaaatgttatcaccacaaaaaaaagtatgtgaggtgatggatatgttaattaacttcatttaatcattttacaatgtatacatttatcaaaacatcatgttgtataccataaatagatgcaattttgactaatcgtaactttaaaaagagtggaaggagaaatgcaaaaataatctatttttgcaagtagagaagaactggctctagtacatcattgggtggaatccaggtttcattattttttaaacctcacgcaatatggggccaaggatgagcttggccaaaaccagagctaccaaaaccagagttacatgggagccttggggatcttggggatcttgggataatacaaacaaaaaaagtctaatgtaaagcataaaaggggaagggaagagagtaatgaagaaaactagcccctggaatgcaatacagaacaagacggtcacagctagggtaacaagagccagcttctctaaggtcggttatctcttaagacatgcctattcttcatttgggttttagaatgccagtgtcgcggacacttttactgactgcttttcaaggaatataaagatacattcttattgaaaataactgcttcccatgtgtagtaattaaacaaataaacccttaccaatactgagtattcctgactttgttctcttaagctttgtattgcttatgggataaagtttgttcccaaacaatgaataaaaaacaaactagatttccaatgaaatttagaacttgggttgactctcactcccctctcatcacttcaacaagatctgcactgtgtaggggtacaggagcagagtcaccaaaaaaccaaaccaaaacaacaacaaaaaagggggaggcggttaagtagaacaggtctgggaaagttatcctctctaaagctcatttcccattctgtaaaattgggagtcattcacttccccacagggttactgtgagaaggaactgagatagcacatgaaaaacagatggtacagtacaagacacccaataaaatgtgtttatagtaatacaaaaatatatgtagccatggagaacaactacccttttcagaagagttaaatttttcatcatagactcttccattgcttgtcatctgtcttcttaattaggatttttaaaaaaatgtctccagattccagatttattcatgttgtgaaagaggattaagagggaacacagctggtctaagccttgaccaagtgtgagggacagaaaacaagctaaggatgcctgagccccacccacctttgccgaaacaccttttgaacatctcaattgtggtacttagcctaattaacaccacagattcaaacagcagtaaatactcttcctagtaaccttcttaatgttctgtggagtcagactctatatctggaccctaaatccatacttttcctttctcccttttgctatcttaccttaagtgactaccttcatccaatgagtgttgttgtaagagccaaatgtgccctctgctgcctcatttctgttcaacctcaaaccgcatcctggttccccttataatcaatagttataacaagtcactggtcacacagaactcccttctagtcagttactccattcatttggaatgtctcttaagatcaatactcaccaattttaatggtcttcttgctcattcaagacctcaagggtcaggccaatttaaaagaccatcggtataaactgagatgatgttgtcctgatttgtacataattaatcataattgtgatcattttcaacaggaagaattcagttatggggacatcctattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtaggacatatatacaagattagtttttcaaaacattctttaaaagcacctatgcttgagatgacttcccataactttaagtagaaatgcttttatttagctcatctcataattgcatgcaaacctctcaaactatgttcctaactttggattagaaaatcaagccatatggtcctataacatttatcaatattataattttatatttatctatgtgattatttaatgaatacctattttcttcactaaactataaatttcatgagtttttttcccctcttattgtttcctccttcctcggacatttaagctctggggtacaatgattgctcaataaatatttactgtaataaacaatgcatgccataactatatgtctgatgcagggtttctcagcctcaccactattgatattttggaacagatcattcttggctgtaggggtctgtccagtggattgtaagatgtttagcagcatcccaggcccgtccctgttagataccagtagcaacctccaagatgtaataatcaaaaatgccttcagacattgcaaaatatactctatgggtgggcaaaattgccccagtttagaaccactggcctaacggaatacacttgtgacataagtaaaaggtgacaatcctaggaacctcatttaaatgaagaggaagggaaaggaataaagggaaggtggaagaggatagttagttgctactcatcttgccaatatttttcagtatcaattctaagccaccagattgacttcccatctctttcttcttcatataggagctacaacactgagagaggtagagcatttcaagaaacaatgtgcaaattaccatttcccaaaaacagttcagaacattaattctataatcttttggtagatgttactgataaaagtgctccatggtcaaataaacttgtaaacaccagattctgcaaaattaaattaatttcttcattatggaacttatcagtctttagccttctgacatgctttgaaattctccgagatacagaaagcttcatttttcaaatttctgaagcacgactgctctttgttggcatatcatcttgtgggattactgtgtctttaaacacactctgggcagcagtggcttagggttatcatagtagtaatatcctttcataactgagaatgtcccagcctcctggcaaattctctaggttgttaataaacataggccatggtactgtcatgttaaagctgcctgagaatattcacttgcacatactcacattgtctgtttccaactctgggtgctatttcagatagaacagaatgataagctgatgatttgctctgaaggatattcagtaaggatgtcactgctccattttgaaagactcaccaaggatctttctccagcctcaagaactctgccttccctgaaatattagtgtcatggtgtggctccacaccaacagctgtgaatccatgactaagctagaaactgcaaacgatggactaccttactgtgtgccaagcattgcagtgggccctttcacagcatgtgtcatttattcatcacaactctataatacaggttgctttattcattccattactttatagataagaaaaccaggacccagagtagttaagtggtttgcccagggtcacacagctagcaggtggcagatcctggatttgaactcgaatctggtaaaggacatgttcttaaaaactttaatactctcatgcccaatctttattgcattcactcactcattcattcaactgtctttccttcagtagctcctatgtgcgaggcactggggaaggaaccaacaagtatgatgtgtgcgtggaaaaagtttaagaaattccagaaaaaatacatcttaggcagactactcttggagtgaagagaagatttgtgtttcgaagtgacatctaagctgagatctgaatgttgatgcagttgtaggcataggaaccataatgtgtgtgtataaatattgcccactattctcccctcattgcatgttgactttctaccttcccagagtgaataatacttcttatttttatctaattaaaacagcaccctttttttgccaagggttagaatgagaaactactaaaaggctgcttcctttcctagtccctggtcattagctttcccagtctgtgtattggcatatattgaagagacatgtgtgccagaatcctactaattatcgtgaaaataatgaacgtcacatatctctctgttgcttgctctctcactttctctttcttcttgggactaaagtgtgtccagagatttcaggtcatagtctgcagctggtggcacaactctcagggctatataatgatgtgtggcttgttcttcctggccctctgcaaaaaaaaaaaatatgaattgctgaaggtgaacaactagcagacatctgctgcattgagagggaaacgtcagagctaactagtttgttttatcttggaaagtatgtactgtgccaaggctatacttatctagaaaaatttatggaaagaaacaggaaaatgactctttaaaaattccatttaaagtttaggtccctcagaggaacctacctattacctcaaaatatttgaccatgtatcattttaaccatgatgaacttgaataatactatattgccactcagagacataaatgataattattaaacaaactcctatgcactaaagaagaaacatattaaaataagccatgtggtgaatgctattgtggagaaaaccaacttttatgacacaaactctaactttttaacttgtctgttttataagactaaatttttaaatatcaaattttccaaaaccagatgcatctggaaattatccaacagtgtaagataatttgaaatatctgcaaacagaatttcagaaaatctaaattcattatcatctgaaatgccacctaactggtgtagatgacagaagttggggtgtgaattaactgataatattcccttattccatgtgtatttatagagtaactcctgtgtccaaggcagtagacaggaaccaggtgaagatgatgccacaaagtctctgaagcaggtcctcgtctcagggagataacagtccagggagtctaggtggagaatgacattgaaatatgaactgagcaaagactggaaaggtgaaggtcatcattggccaatgtctttcttctcaattacaaaaacgagtcgtgggattgagtcatgatcatcaccccgtgtgacactgaaaggagtgaagagactatattggatattcagttaattatatgtcccatgcttcatggtctcagagagctttatttatccttagcctggccagatgattttgctctgtataaccattgccataagaagtcaagggtcgaatgttgaagggagtgtaaagagaagtatcacaatgtctttaaattgtccttatgttacaggttcactattattccaaaggaagataattcttagtcattgaaccatgtgagagagtaaaaactctaattacctttgttaccttgaatctaagaggataatgaaactttgatagggacagaaaggacttccccctcttcttttccctggaataatgtctcatcctatgttgtctccctagactctcaaggtacacccaagtgagtgtagggtaaaagaagataattggcatgctaaactaacagtggttaaacaaatttccaccttaccttcactctaagcaaaaggtctatttttaaaattgagtaagcaacaacattttaaaaatacaacatccaactatctcggtacaactatctttttcaattttatatgttgctatcatgttcagcagttcctttacataaaaatagatggtttgtttgggttttctacacaaataacataaataaattttgcaaatatttgtaaaatgccaaatatatcaattacaatatgaaaatccccctcattcttcccaacttttctccccattcctgaagtaacccctgttaagagtttggtctctatcttttcagtttgctttctgtataagtgtatttaaattttgtgcatatatatatacagttttggattattaaaaacataaatgctataatattgtacttactattttataactttttaaaacttaacagtagatcttgggccaggcatagtggctcatgcctgtaatcccaatactttgggaggccgaggtgggcagattacctgaggtccggagttcgagaccagcctgatcaacatggtgaaactccttctctactaaaaatacaaaattagccaggcacatggtggcacatgcctgtaatcccagctactcaggagactgaggcagaagaatcgcttgaaccagggtggcggaggttgcagtgagctgagatcgcgccattgcactctagcctgggcaacaagagcaaactctgtctgaaaaaaatatatgtatacgtctttaatctttgcatgtaatcacatacaggtctattttctgtatagactccttggtgttggtataaatgtagcagatttaatcactcatgaaaatagacattaaattgttttatttttgctctttcaacagtagtgtaattgatattcttatgcatgattcttgtacatgtgtgtgagtgtttctgtatgagacattcctcaaagtagaatagtagatatttacattgtttccagtctttagctatcatgacagcaaggcaggcaacttatcttggttcccggatgttcttgtggaaacaactttctttttcttctttttgcttttctatttaaagattaatggataaggatatagggaatacagttgtagttcctctctgtgtggactctaaatctcagttaccttatcagaactatagttacctaatatttaagttttaaaaagtaaattaaagttaattatataagacaaaagagataaagacattttgctgggatgaaaagcataaaacttaaaaaagtaatcacctaaatatacgtttgtaaaatgatcaataattgagttcaactcaacaaatattttgccatgcaaaaggtactatcttttacattacataagatgcaaaggaagatggaattcctacccttagagaccttataatctaggaagtggaaaacacaaatccataacaaattataataagtcagaatataaattttatttttaaaactgcaaaaaaaaaatcttcagttgaatgttcttgatgagatcctttcttaaatatgagatatccatgaaaaaatccaatactctttgcagctaggtgctaaaatatatcactttcaggcctctcttaagtttgttaatggggcaactttcactctattttacaggtgctgtggagagggtgaagaaaattcgagactatgcttttgtgcacttcagtaaccgagaagatgcagttgaggctatgaaagctttaaatggcaaggtaaggtagaacaaagaaaaaaatatgcagattccatccaagtttggtgcggacattacattcaaaagcacagtcctgcgagtctgtctcactccagccttctctgacatggctgcctcagtccatttaagatgtactaaagagaaatgatggaaaatacaatggaaattggagatgtacctaatatatccaactttagctctattttttttctaatttggacaaggcactgaaactcaatttccatggaaagaggagattgaagtagattatttcaaggtgtttttcacaattccagttttctctcattacttcaaagagattatcatcattttattgcagtattttagaagctgatgaattaaaaacatgtcaagatagctgttattctgccttcatagtttgatcttccacttggaaagcttagagtctctcaccttgccaagagtcggggctggggaagcttctatatcctcactactgagttgtatacagaaaccaatataatatttatgtatcaagccaataaacatttaaaaccttctatagaccacttatcatgctaggatacaaaaagaatcaaagatgtttgacaaacagttaaaagttggttggaggaataagacaactacacaaacatgtgcaaagtacagtcaagtaaatatcataaaaagagatcaacgtggtgagcaagttcgaaggaagaagtgggccataaagaataaaacatcttcatggatatgagaagataatatttcaaagaagccatgaaaattgggtataatttcaaaaatcaaaatatggaaagacaggcagtcttagacagaggcacagaatttgagaagagcaagacaagattggagaacaggaaaatgtggagtaacttggttggaacataatgcacatgggactgttttacctatcggatctggcatcagccagggtcccaaaagaaaacaaatagcaaaggaattaattacaaaagtgtgagttggggctagggaaatcaaaaaaagagtgtcagaatttggggctcacagcattctggagtcaaaggtacaaggggctggagaattaatagaaatcctgaaagcggagagaattgtgtagagcagatctctttgcaagaagtagctgggtgcagtggctcacacctgtaatctcagcactttgggaggctgaggtggaaggattgcttgaacccaggactccgagaccagcctggacaagatggcaagacctcatctctacaaaaaattagccaagcatggtggcatggactactatagtagtcccagctactccagaggctgaggtgggaggatctcttgatcccagaaggctgagactgcagtgatctatgattgtgccattgcatttcagcctggacaacagagtgcaaccccatcattaaggaaaaaaagaaaagagaacagaaagaagtagtgattttggtcaaaggacacagtcagcccaaggtgacctttcaaagagggatccagcagaataaatgccccaaattcactgttgtctctttctccaacctcctaccagcattccccattggctaaatccaatcacaatctagaaggctaaagaggaccccatgtagaatagtctcccagagcagagagcaggctggagctggtaggtggtgaattccatagggagtggatccagatggcaaagggaggatctcttagcctagacccttcagacactgtgaagcctcttgcatattagctcctcaagggcccactcaaactcaaaaatatttgagtgctggagggtgggttggagaatgtattcactctgaaatacaaaaaagacaactgcaaagcaaaaactaatgcatagttaaatacatgtctataaagaaaagcattatttcatcttcattgattatcacatttaatatcataaagtgttattacttggaaaacaatctataagttcgattttctctatatggaaacattcctgagtatacacgatgattgctgggaaggaacagccatcacaaattaaatgccaataattacattggaataattacaatggaaacatattcaaattattcccaatttttgcagctagatatttttatagtaatatttacacatattatttagttaaatatatatttaatattatacttgactataatacagtatggaaaaatctaaagtaagttttcctaggaactatggtttttgagcatatatatagaaatttatatttaatttattagatgaggtgagatcttgaatttttttgtgcagtaattatcacatttacaatttaggaaaattaacctagtaagaattgcctttttaatacaggtgagaaaaacatgtcatagaaactagttataaagctattatagtcacccaaatgggacaaaataaggtcttgaacttaggtaggaatggaatgcaggagacaaccatgtacagaacagtaggggtaaattcaaaggacccaggaactgattggagataggggtgaaaaagaaggagaagtcattataagcttccaatatgggaggttgttatgaaattcaggaagggaagcctattcagagagaaaaataatgaattgagcttcagaatgattattcatttattccaagtctgtgaagtgaaagaaattttactaagcttgggttatactgattaataagatgttgtcctagctcttgaaatatctaaactaaataactaaaatacaatattgagtttaaaatgcttttggatgggacagaggaaggcagaacaaaatttattacaagaaatctgaatttgcagcaaagaatcattccatgcaaaatgtatcttgatatggaagtgcctataagcagagacctatttgagtctctcatcttcctcaagatcagactgaagatataaataatcttatacccaaggtcctcccctattttcagtatgccagttctattttttctaattacatcaacatcaatataagattgttgaatatcagctggattctatcagagtattccatctaacttatttgacactggctcctgctactaaaaagtaaggtaaatacataatactggagacaaaccgacccacaggctgaacaaaatggtcaggatgaccatgaagcacaagctttgtccagcagacttcgataatccaggaaccactgagtcacggttgaaatcagagggtaagggtgcacactcagctgctgtgaatgtgaatttctgtagaaacactttaaataaaagtcttctgtatataaaacagaggatcatcacattcttattcaaaaaaaaaagccacaaatgctttctgacaatactttgttcctttgataataaatacatcaatctatcaagaatttgttgagagccatgtttgtttcttgtgtaggtaagcaatacatagaagttagtagcacatgtagatctacatacattcattatcttcgacatctggagctgttcacaatctcaaaatttgtggcaataatttttgaggacaagttttaagcggtgcatgatctgagaacctcaatactactctcactcccaactactcctgaccactgtcatccctgtttattagttcccaactgttcagcagatcaaaaggtggcttcacaaggaagaacctgtggcaggcaatgcaacagcctcccagattcctcttcctttggtagagccacagtatgcctttcttctcctgtgaaaatttacttgtgcggctgatttaaggagcaaggcagttagtaggctatcagctggatgggcctgatttggctgcccatgctaaatgctaataattgataagatttcgttattggtttcctttcaccttcatcttattgtgctaggtaatggagattttataccatccataacaaaactaaattttattgtagttatataattatatagatatgtaatttaaaacacatatatgacctaataaaaatattacataagtttctgtctcagtgactgtaaccagattataaatttataatgaatatgtcaatggacaccggccaatatgtcactgtcaaacctgttggcatcttatgcatcttaccaacaaaatgggtcataaaaaattcaactggcatacaaacacaaacctataaaatgacaagatacaatcaataaacctacagcaagtagtctaacacttagaaggaatcatgttattagggctcagccacacttggttatctttgtatcatctcatgttgatttttctttgctctctacaacaggtgctggacggttcccccattgaagtcaccctagcaaaaccagtggacaaggacagttatgttaggtacacccgaggcacaggtggaaggggcaccatgctgcaaggagagtatacctactctttgggccaagtttatgatcccaccacaacctaccttggagctcctgtcttctatgcccctcaggcttatgcagcaattcccagtcttcatttcccagccaccaaaggacacctcagcaacagagccattatccgagccccttctattagaggtaactccagtcagatcttgctttagggtaatcccacgatttcaaatgactcgaattgcagtagccttagatacattggatgggtccactgggagaaaagcactgaaaatttttttattaaaaatcaagtattatatcaatagttttcctttgataggagtcttttttcacattacaactggttattgaaaaaacgtggtcagaacattgtgcaaatgagcctagtgcacagattcaaactttacatgggtccataaaatttgcacaaactaaaacctagttctataactgttagcttcttttgtatcacttgtccaccattgtgcagtcatctgtggctcattacaacacttctggaatagactagtatagtccagtggaaaccaattacctcaaatagcaaaaccctttcaaggcatgtgtgtatttgatggtgactcagtggcattgtcttaatatacaaatcacaaattattataaatataatctaacagacacacgagaggttctcatctgacgtcctccaagaaatatgtgttatactttcttaaaaaccaaattattaaggaaattactgtatttctttagtacatggagaactaaataaacaccaaatagaaaaaaccctcagactgctaataaaacaataaacatttcacatttgaatcctgtaagaattctaaatgtttgagtagttttccaagctccatttcttcaaggaaactagaagataaagaaagcctttattttatttccaagaaactagcttatccctgcctttttggctccttcagattagtttttctaggatatggctaatgctaactgaatcccagttctgtcctctaaatacgacatgaatctgggcaatttgataaataaactgtgtatttaggtctattcttgggtttgacttagaagcaaagaggcaaatctagattactgtggaaacaattatcttactatgttttactatttttcagtataactgaattgatcatgctaggagctctagtcttcaacggccttaaaagtttcatactcataattacttgccccaaaattttaatgcaaattaaataatcaggatttcctttaggataacttgtctatgtgacaataaattatatcattgtaacattatgcatttataatgaaaaatcacatcattaaccaatccatacattaaatacaaattgtcaaccagttatgatgtagaaaaaaacaaactccactataatggaaaaaaatgcctatcttataacaagaaaggaatcaatgaatgatattcatggatgaagggtaatcaatttaattttgtatctctatgccaccaacataaaatcaaagtagcaacatggcaaaacaaaaatcaaaaggaatctatgcatgaaactcaatggaaacttaagcctcacattgaccgaacatcagagagaggatggtgaacctaaaagcatgctttctaaatgcttgttttccaataagtcactggaggtgggataattagagatgccagaatttatttctaatatactgatcagcgacagtctctatgcaagactgatttcaaaacaagtggtcgcattatgttggaatgttgtaataaatacattcccagtctttattagtctatattagaaaatatcaagaaaatatcaagaaaaggtaacctaaagacaaaaattgcaaaatttctctcccaacatttttaagttgcttatgtacttatcaacagtattgtacttatcagtggcatggctttcttctgaaggtagtttttcaaaaaagagaaagacatctatcttaaaccttaccttatgttgagtttcattacattagaatcctagagtttcccacccatcttcagggcaataatcaacgtgaattgggctaacttttccattacagtaagattcagggtgattttctggtggtttgattcagtttaatgccttcttacattcctagaaatttacatgaatgtacctgtaggggctgcgggagtgagaggactgggcggccgtggctatttggcatacacaggcctgggtcgaggataccaggccaaaggagacaaaagagaagacaaactctacgacattttacctgggatggagctcacgccaatgaatcctgtcacgttaaaaccccaaggaattaaactcgctccccaggtaggtaatgttattccagaaatagtttctttaagttgtgccaatgagcatcattcacttttaccaaatgatttgacttgatccctactcaagctatgggtttttttttttcttttaatatataaagcattcaatcaatgtcacaccctttgagggaatgctgggcatcaggtgccgttattctattataggtcctttctctttttaaaaaaatcttaaagcttaacaccttgaacaatattccactgaaatgcaaagaatctgagagctattaagaattatcaacccataccctaaatgaaaataaaatgaggcaaagaccccatctcagaataaatgcctattgtatgtgagccaggttggttggcaaatgaagggcattatggcagagccctgaaaagaccatttaggacatcttttcttggaaatactgtatctaagagaaaactccaccatgatatctaggtgaattactaactaggagatgtcgtcagtcataccaaattaactcttcctccatagttatattgcggcagtggcattacagagaggggtttttttgtgttcatttaagagttctaagtttccatacttcaacattaacctgtgttttttattattggaaaattaaaagttatacaagtttcaatacggaaactgactcccagggttaaaaataaaacaaaacagggtctatgctaaccctgagactgttccctgtgggagtgtgataggtgctgatctctctgaaatatattttggctgaataatcagttacagtttgtgtgtcttaaaggaaacttccctattcccatgacaaaggtgggcttatagccccacatttatggaagtagcccttataatttcaatccagaagaaaacttggttttctaaaagctcatctggggtaatttttcaatgggatttctgactttcttttatgcacaaattagcttatctaaggtttctgctagccccacccagggaattaaaaaaaaaagaaagcctaccagagtgctaaacagcaatcatttgtttgttcacaaaagatttagtaaacttctcttatgcaccatgtagtgcactaggtgctggagatgcagaagtgaatataaagaaatccttaccactaaaaattagatggcaatccagtgggaaaaaaactgacacataaacatataaccataatgaaatctggaagtgatatgatgaagtttgttcagaaaatataaaagccagtcacccagtttctcttgggttaaaacccaaaaccaagaacctgattaaagtactggaatccttagaaatcttttaagaaaataaaaaataatagacacaaattctgtataatagtagttctgatagctgtgatagtatgaaaagagatctcttcgtttcacatcaaactcaagttagaaattcatcatgtcttatattaatatgtcagtaataatctctatcaattaataaaagtgtctctgtccccagaaaaataactggggacagccagtgtacccgctgcactctgctattggacaagaccaaagacaactattcttatacaaaagacaaattaataatattgttctctgaccttctacaactatggcttagaaatgaacaccatgccaccaccaaccaatatagatggggcctaaacaaccccaaagtagagttatgaacttccagccagaagcagagacaaatactgccctgcaaagtcagcagtgagagctggtgttccttccacccgggtttgctcacctggcataaacaggcaggcatcatatctaccagcaaaagctccattagtattgcccaagaactgtagagaatgctctgaggatgacaagagatcctacacaacaagagtattaaaaagaaaatgtcaatagagaaaataggaacaatctacaaattaataaactggaaaatatcatgctattgcagctgggttagttagcataaatgaggtaagaaaccccagagacacatcctgaggcactgttctgtcttccagagttattttctcagccttatgccctgctttccacagtctactgaatctgctgactaacgagagcgtcaggacctctggacaaccaaaaattgcttcagaataaagaaactatgaatctttagaaaatattcagtttggagtaattacagaagaaaacaacgtgcaggaggatgctaaaaagcatattgtcattaagcaaaagaaaaaaaaaagccagaaatagtgtcttaccaaacaaataaaaacattacaactgtcaacacccgtctaggaatgctcagtaaataccaatccattaaactttttaattctatttttatgaaattgtgagattaaaaccctatggcaaatgacatgaaataaaaatatattcttaggttatcatctctaaacgattataaacacagagactgagggtaataatgagtaaatctatacaaatctatgcacacagtgttggagcaacagtttttacaaaaacaaatattccaaacaaatcataaaattaagcttttggtaaaaagttacagctaatggactaaactaaaaatatattacagtaaatggtgtgaaagtgattttatgaactaatagacctaacattttaaatggagttcaaattgagttcatcttaaattttatgagtcattaaaccatgtggcattttatatcttctgatattagcttgaactctgaaaattagagtttaaaaatctgaaaacttgggatataatgtgaatgtgtcatttgaggggaaaataactttttttattcttgtagatattagaagagatttgccagaaaaataactggggacagccagtgtaccagctgcattctgccattggacaagaccaaagacaactattcttatacaaaataaccattcctgccctagccagccagaatcctgcaatgtgcgtaacttctccaaattccttaccattttagagcaggatacgcttctaaattaaatgcacttaatcaccatctaaacaagatactgaggtctactacctactcatttgtcattaaatgtaagcattgaaatgtatagttgggatgtgccagccatgaagctggcatataaaggcccccaaatttcatatacacaatgaaggttgtgtagtatcaccaaccattggaaaacagtcagtagtgtaaaatgtaatagaaataaaaactatagcccattcttggctgggcgcggtggctcatgcctataatcccagcactttgggaggctgaggcaggtagatcacttgaggtcaggagtttgagaccagcctggccaacatggtgaaaccccatctctactaaaaatacaaaaattagccgggtgtggtggcaggcgcctgtagtcgtagttattagggaggctgagacaggataatcacttgaacctgggaggtggaggttgctgagatcatgccactgcactccaacctgggcaatagagtgagactccatttcaaacaaacaaagctatagcccattatttcatctgtgcaatgtttaagttgaagcatatgaaattaccatttttatacataataagacaatggcccatcatttaatttgggaccaaatatatgacctctctgttagcggatatcacctcctgaattttaccatgagcctgcatttctatatcttttagaaacaatttgtttcacttgtgctttagaaaaggcatgtagtgaattatttgtatgtcccaggaatgtcataaaccactgatgttctagatcaatttgtaaatattcctacatgcaacttatatttagaagctttaatagcaaacagaatgtaaaacacccactcaaaaaaccaccatccagtcattttacaaaactgatagtgccttaggttttcacctgactcagttgtaccagtttaaaaatgcctaccctgactgacttgctttccagccaccctttcacacctccgaagctgagtgcctatgtggatgaagcaaagacgtacgcagccgaatacaccctgcagaccctgggcatccccactgatggaggcgatgctggcaccatggctactgctgctactgctttcccaggtatgggaaaattgtgccagaaatggcgccctgcagagagtatttgggaaacccttttttggtaccactgaatcatagaaatgtttgtgtaccccacttacggcaatctgacttgcaccacctggggaccaaaaagagtttcccaaacgagtgactcattaaaacacaaggcaagaaaatcaagctgggcaacagagtgggttctggtctcggctcagctttaaactgctgtgtgatcttgaatgagtcagtctctctgagtctggtttgttttggtttttacttttagtaagagagtttgaaactacctgtagatatatgatgtttactggagctcttactgtgtgctaggcactcacctaaatggtcaggaagcattataatatcttagtaacgctcatgataccaccaagagttaggttccatcagcaagtccagtacacaaatgaagagcccatgtatgaaaagattaagtaatttggccaatggcaattagcttttcagcaatttttttctccctctccaatcttcaatctacttttattaatttgattccaaaagagattttttaaagtcaaaaattgtgtgtgtgagagtgagagacagacagagagcctttcaatatgtgaatatacaggactcaacttatttgttctataattttggtctggggcatatgctgattgcttaaagcaaagcctatctgtggatcaacaatgcgttcagaggtcacatgttaaaagatttgtccccatttattatgtacccacaacaaatggtaacaattaaaaatgaatcctttagagactgaaggggaccacgaagaaagttgtttagaaaattttttctcagatgaaaattttatccttttttttttttttttttagtaaaagattatttctattgaagaaatctatcattttgatttgtcattgtgtcagcatgttccacataagtccaaattagagctctaagtatacctagaaacatgacattcttatcgattaattaattcatatactggtcatgcgtaggtgtgagaaattttatattttcctcacacccactgcacctggggctcattctcagcattttgttttgttttgttttctcaaaagttgtgaatatatcctccattctggtcagtgaggctaatattaatgatgtttcacatctgaaagtgacctcaattctggctttatttctctgtaagaacatggagacgctctgtggtgagtccctcggaggttaccaaggtaatagctgacactgcagagggcaaggtccttcctgggcagtgcggggccagcagccggtgcttgctttccacataacagggtgtttacatgtccatgtactcaggacgcctgctagggcttctcagttcttagagaaagttgagcccctgtcttgcagaaagctgcaccccaaagacatctaacaaatgtactattaaactaatgagccactgtcagaagaatttcaaagtttgctttcacgttggaaaggatttgcttttacccaagaatgttttatcttattctcacttctattggcttctgtgaaaagttaaaaatatttggggtatgagtgttgtctggggaatgcctaagaatctgagagcctctcttataactttgctgtggagtcaagcaaagaataataggctccaagggaagaatcggtttgaaacccaaaacaccctggcacttaacaaattgaccaagttgaaaaacaaaattcaaaatccctaaaggtaaaatctgtgccacgaacacatgcatgcatgtgtgcacacgctcacacaattatattactcacttagtttagaaatactgtttccaaaattatgaaagtaactgttaacacatgtctcctatccttggaaaatggaccaaacttctctagtgcattaatcagcattactgaccaaagtgaatgcgccaagaacggattcaatttttgttcaaaataatccaaccagcttcctgccaccactttctgagaagccaccagaaaaaaatcaatgtgtctggggttgggcaggcttctaccgcacataaaccaaatgaggtttaaaacaacaatgaaaaacaggtgtccatttgtacttaagtacacttctgtctttctcttgttatcttttacaatttctggatcatctcagaaatactcaggtttttagactaaaacaatgcagagaaataatgctttagatggtagctaacatgctctcttcttcaaaagtgaatgttcatccactagacacaatgctaacatgaaagcgcatagattttttttttaattcttggatactttggttctctgagggggagaaccagcatcaaggcctctgaatacaccaagcacaggcttgatgaggggcttgggaggtacgaaggtcttctgccattcatagttagatctaaatacggacaggttaggcatttgcttttaaggtattatggagaactacacttaaccattacagtatttctcctactgatctcccagtctctacacatcttctcgggtaaaggcaaatgttttaaatgtttcatttaggcaggtgtggggttgcaggagaaccggtcccacagaactccacctccccacataccatgcccggccagcccctcagtcaccttttctcatttataaatttggaaaccttcaaaaagttataaatcacaagctttaatcaactcagcctcctgttaagtttctctttcttgggccctatcccaagcatctttaattctcttaagtatgaagtgattctgacacaggtggttgatcccacttttgggaacattgtcctaggactttttgaatgtgattgaaaatcactgagtccacatattgtgtccttaagggcactttttaaaacatacatattaacttttagctttaagtaatatgttaaaagttcgtatgtctaaaagaagacaaataagccaaaagggagcaaatgtgaattcctttcatgaagttttctctgcctcttcctccaggatatgctgtccctaatgcaactgcacctgtgtctgctgcccagctcaagcaagcggtaacccttggacaagacttagcagcatatacaacctatgaggtctacccaacttttgcagtgactgctcgaggggatggatatggcgccttctgaagatacttttttaaatttaagaataagacacacaaaactctatt

SEQ ID NO: 3 - DNA, 80232 bp, Mus musculus
  source 1..80232: mol_type = other DNA
gtttgatacgatgattagaatataaccaaagtggcatgctgaattcgccataatcaaggaaccttttccgggtggggaacctcagaaattgctcaggtatggcccatgctttttgtttgtattttctgtttcaattctgggttgtagtgtctggtagattgcatgaaaacaacttgtgctcttaactatctctgagcctaatgcagagaattgtcgtcgtttgggaatgaaaccaaccggaaagctaaacagtgtcttagactgagcggggataaatttcatggtatacttcttgagtgggtatgcttccacaacttgtgggaagtggagtcatccgtgggttcacaagtggagcccatgtgctgcaaacctaacctccagctagctgttggggagagctgtgtttcactgtctgtcctgatctctaggacccgacaacagaaatgtgattaagacatagtttcacatctcccacagccttctgagttctgtgtatcctctaatactttagacaccacctattcaattaacttctgtgtgctactacattataaaaaaaaatcaggataaattcttgctatttcttctgctattagcctgctaaccaaaaataatatcctaagctactttcttttgagtctagcaaggaaagaaaacattatggaaaagtgtgtgttttgagtgtgttaaatatcaaccctgaagtgtctatttattagattgctttcacagactaagcaaccagcacagcacatgtatttttgcaatatcatttttctcgcacctaaaagataactaagagccaaaatacaatggctctaaagtgggaaatagatcagggcattttctgtcctataagaaacctcaactctctgcctgaagagccagtgttggttcactataaataaagcaagccatcaagcagccccatgaaacacatttctgtactagtgcaaggtacggatggaagtctccatgaaaactgagaaatcatttgtgcatcctaggagattaacgttttgtaagccaaatattagtaaattcccactcgagttgtaatttaggagaatcaaattgagttggtctagcttgaaacactaaagtaaaaggcagcagcatgtactgtcagggaatggaatctggagataaaagggaagtcacttacctgtccaaacaatcaggagttccctggctgacacagagaagtagacacacctggtttatttttaagaccatgttttctggaataatagtggtacttctcagcttaagaaagtgcatacagcttgatgctccaatttactatggattaaatataggggaaagataaaatacagttggctacaaacagattgttttaagtgacactcacagaacaccataatggatccaaaatcttgggtagccattttaactagacaagttaacctgtgatgtttcaatatcaacattacctctactgcacttgaggagcatagggagtcacaaacatgcgtctccaacagtcaatcaaatgagctgagtaagacaacagagagcaggccatcttaccatctgggtgctcttcagtgtaacagtaatcattgttaagtgttagattaccaagcacatttctaagcacatgttctaagtttaaattcatgttattcttagaatacccgtatgaggctaatactatcattatactatactactatatatcatactgttattagattgaggctcagacaaattatttaacagacttgatgttaaaggcaagtgacatagccaggttacgcagtcaggcggtctacttgcagagcacatgggtgctctgtaccctgtcactaggattcactctgtgtacatgggtgtttatcattgggactagccaaaagaacaagtaatgcagtttcctaaccttcctttagagagaacttcttcctccaggagcatttgtctatcagcatctctccaaaattttactcgttaggctacaggctaagagtaaaatatttttgcagcccatgcctatttgcaatgcgcagatcaatggaaatctccacatttggattgatctcagccaaaccttccaagaaatcacattctttcacaatcctccacaaagccatgcttagattaaaggctttaaaattaatcccagaaagagtgccatactcttttacagacataatcaggctggaacagttaccccagacctccctggcctgtgaagctgtgcacattacttgagttcctgagatctcagattcctcggatgtgaaatggatgtgtgatgtgcctacaggacattataaatattattaagtagagcactggtgcttaggaaccagttgacctcttctaccatggtcccattaataacatttttttttcagaaaaacttaaacttaccacccactagctttctcaaaacaattccattaaaatagaaaactatataaataaagaaactcattatttacaaaagccaccgcacttctttggaacctgtacacatacacacacacacacacacacacacacacacaaaactacaaacaggataaatttcattttagagactttctcaaagctgctactaaaaattcaacatgtctgtattgacatctatagtttcacactctgacattctgacactgtacatgttaactttattacacagacagatggtttaaaaactatttcagtagctatcaaaaaactctggtatccctttagaatgctgggtaatactttgggtactattgcccaatggctttcattaccacatgtgcctgtacacgactcagaaagttcaaacaagacagtattgatcgacagaggataaacactgctttatcttatccatggacacagtaactaggtttctctcaagtgcaactccaacagtccaaacttcctcaggaaaggcagttggaagagaagcctcactctagaggtgaccatgcatattcaacatcaaaacccatctgtcccttagcttaagactgcaaatctagtcagttatcattaacaaatatttactgccaaatggatcaggaaagatgatttatcattttaagccatcaaccctattccaaacaggctatggcttttaagattatttagaattttatttttcaaatttcgtcatttttcccttccctttcctcctctcgatccttcccaagttcctcccattgctcttactcaaatatatgacctgtatatatgtgtgtgtgtgtgtgtgtgtgtgtgtgtgtgtgtgagtgtgtagggtgggaagatgatagatagatagatagatagatagatagatagatagatagatagatagatgatagacagacagacagacagatagagatagatagatagatagatagatagatagatagatagatagatagatagatagagagaaatagatagatagatagatagatagatagatagatagatagatagatagatagatagatagatattcttaaatatataaatacaaccgactcagaccatatggcattacttgaataaatttagggctgatcattttaatttaatacaaccaaagttttgtgttgaatatttctgatgactccaaataaaagactaaaatatagtcacacactgataattggtttggggattgggcccaggacctctatggtacccaaattctcatatattcaagtctcctatagaaaaaagtgcagtgtgtacacacaatgcttgcacattcttttaaatactcttagttcgctcggggctacttcctgtcccttgcacattgtaaacactattaaagactttcatgctgaattgtttatatcttattcaagataaatgcctgtttatgttcaagacagatgtatttctttcaaataattttaggtttttttaaagaattgtttatgaattttatgtatatcagtacattgtagctgtcttcagacacaacagaagagagcatgttgtgacccgctatgtggttgctgagaattgaactcaagatggggaattgaactcagggcctctggaagaatagtcagtgctctaactgttgagtcagttttctagctccatcctccaaatatttttaatctgtggttggctgaatctatgggtagagaacctacaaattcagatgtccatgtatattcgctattgttttgtcttcactaaactatgctgacatatgcaactgtctattttgacattttgaaatatatctaagcaaagtgttttctttatggctttgtggaggaaattttacgtgtaccagtgttcacaatcatgtgaacatgttccaggcttgcttgttctactggggattgttttctagtgatactgagaggacatagtcataaagttatgataattccctatacttgcagagtgctctgtgtcagtaaataatatgtctaatgagaattaccatacaacaatttaaccagactgactgtctaaataagcaaactcaattacaagtctataaaatcctatttttcaatttggtagtaatgactttacttccttcaataattaggaaataactattagttgggatgctctattgatttaaagaacactaggtctgagtatgggaattccttacccttgtactttgttaccatggaatattctactttataaatgtatttcagggccagaaagatagctcagcaggtaaaagcaatcactatgtaagccggaaccccaagttcaattcccagaatgcataatgaaaagcaagtactaactatccaaatttctcctctggcatttacacatccacactcatacccatacacatgcacacaataatcaacaatcaataacaacaataataacaataataataattttataaattaaatgtatgttctttgacaatttcatacacacacacacacacacacacacacacacacacacacacaaacacacacacataaggttgctttcaacctaacctctctctcttttgaaaaatattagtgctaattctttgaaaattgtatacactgtattttgatcatatttaccttcaattcctctatgacccattcccaccttccataaccctcaccttcatgtcctttttaaaaatgtaatacctaataaccgatcaaatgaaatgtgtgctgtccgtacacatctgggtatgagactgtccacagatggttgacctatcagaagccatacctcctcttttacaattctatttatgcatttatttagatatctttgaggatttcacaagtactgtcttacatcatttatcttccctaccatcctttcaactcttccccatgtccccgcaacttcctattgaagtcatgacttcctgttccataattattatagttagacacacacacacacttacaaatacaagctgctgagcccctttagtgctgctcatatgtgtttagacctgaccactttgaactggatgacctatcagggatttgatccctggagaagactgattctacctttcccattagccattatttgccaatagctctccacctctggatggagccttaggagatttccacaatccacataagcatgtcagttctggccttgtttatatgccatattattgagatttcatgggtgcatcttccatgacatacataagagatcccatccatcaagagattgcctggtcctctgatgagccacattctaaaataataaataaataaataaataaataaataaataaataaataaataaataaaacctgcctcttcgagaagccatcaactgtacacagtttctctgttaaggctgggaggctcatgaagtccttctcactctatgctagatcactgactggattgatcttgtgcagtatgtaggtcttctgtatgttttcttcaggcatctacaactgctgagttcataaattctgcaatctatcatgtcactttttttttattttgacctgtctccaccatcttctcttacaatttttctgcccctcttctagaatggtccccaagtaatgaggagtgagcgtgatatataggtcacaattgtagctgtcactctaaggtcatttattctctgtactgtgaccagatgtgagtttctacataaactattgtccaatcactacaaaacagaactttctgatgaagtctgagagagacctacactaatcaacaagtagaaagatataaatttatagggaagtttgatactatgtcagtttagcagaatagtagtgtcagattcaaccctccagcttatgagttccccaaccatgtgttcttggccaggttttcagtacaaagcgtgagtttcattctgtggggcaggccttaactctggtcagaaagtggctggttacacctataacatttgtgccactatggtaccatgtcttaccatgccagttcaaggattccaagctaaactaagactctgaatgacttttttccctcctaggctgcatagcaccttccagtactatgaaagcattccaactgaaaggaggcttcctagtcagtactgacttgattcctctatgttctgtatacaatgtgcatgatgtcgccagcagaaaagtatcttattcatttcggttgggcaactaagcaatgtcaatatcctctattctttgtgaggtctctgggatacctctgatcaacaactaaaggcaaggtattccacctagcactagactttggcagggagggcaggctgtggtttttgaaaaggcatcatcctcttcatcctctgtgtagagtaacatttaactattttatataactacgtgtatatgtttatatatgttatgtgaaggttataaaaatggattcctttatagctttttcaaacatctctagtgttaactatccttcctccaaactcctcattgtccctatatctttttgtctctttcatccttgtcctgattaatcattttgtcacattgaagtcactttgttttgtttgtgacccaccgagtttgcccactagacagttattatgatgctgagtttggaaataagcattggagactaatgggctcatcaatgggggcacaactgaaaacactgtatcccattctcccagaatccatcagttcagaagtaaggaatagggccctgtaagcttcttccccatccataactgattgtttgcaaagcttgtcttatgaagacccagtgcaaataactgtagttggtgtgacttcatggttacaatggccatgtcatttctatagaatagcattttgcagttcttctccctatattctggcttctatatgcttcccataccttcttccagaataatctctgagccacagagggagtcatataagtgtatgtttagggatgagccttcaacctatcattcttaaaatcttgggcagtcatgagtctcttcattcactgctattcatggaagagagaagtttctctcattagaagcaagaataacatttgtcatgtgcataagtatacatgtgcagagggcagcttggtggtaagtctatttatctaaagatcagtactaaattggcctccaggatctgtgacctcctgaaacagaatagtattgttttcctttttaaccaagtttacaacgccaaccataaattccctcttgtggagcaggcctcaccttacatcctgagagtacttggttagccccataacagtcacaccactaatgtgtatgtgagcatatttttgataggttaatataatagctcattctgtaggtaactgagtaagaccattgatatattttttttctgctagtatgctaagtagcagtttctgacactttcaagctgtacagcagaatggaagtatctagttcagttacaacttgagatgtgtgtgtgtgtgtgtgtgtgtgtgtgtgtgtgtgtgtgtgtgtgtctgtgtgtgtgtgtgtctgtgtgtgtgtgtgtctgtgtgtgtctgtgtgtgtgtgtgtggtgtcatcagaaatagactcttatcatctagttctggtgagcaactaagattaaagatgggaacctgtacccagcaattcttgtaaaagcatcccacactggatggctactgcttctgcaggttttaaagtatttcatgcctaagaatttaaatgagtttaatgtcaaacttcttcatgctcattttagtatgatctcgtttaaagtaaatgagttatgattcaccattccaaggattcaagatggttaaagaaaatttttacctgtttatgtagaatactagtgattcacaggtcttcagctattctagtagttatcaactgacttatcaatcagtcagagactgaatgaaacaataaattaaggatatatcaaggtgactgaaaagccagttccaaagaggcttcagaggaaagcagccctaaaacaaactgaaccattcagttactatggaaggatgcagggggagaaatggaagcatagacaatcataaaatctcctaacagactgttgggagagtgcaagccattacagacagatgcaattgttttctgtttctagcagtgttagtgatcttcactttcgctgtcttattgggcttctgaatagcacttatctactctgctggggctgttagcacgaagcaagcggcttattagatgtactcaaaccctattgcttctggtatttgttctggaagtcagaaatctaaatctgataccatgggggaatgaacaatgttggcagacaaatcttcaaatcttctttcttgaagctgcataacggcacccccacacacaatcccagcaggatatggacatttactcctaaccaactagaacttgcaagctgtaattatgagaatagtggtattaatcataagttaacttcaacattctcccctggctcttccacatgctaggcactttggtgaaccagtttaacccttctgcaaaatagatgctattactatatcatatttccaataaagaaactaaggcttaggaacatgaagaacaagccatgccatacgcatcagcttgcaaattaagagcagtctgatgattctaaacagtattgcagcttggaatgggaaggatctcttgacagagagaaactaaaagaatgtcccaagaatagaggatgtctttttcaaaatagtaatcagaattcaaaactgaagccttctagtgggacattgctttggattaccactcactgtacacatctctaattcagcctggttgtctgccaattctaggtggcgtagatcctccaatgttaataagacctctctcttcattaagtctcgctttcttctctatgatcagattcatagacaaacagaaactcagttaaaggaaactcagctgtgacttatggactcagaagttcaactcacagaagaaatatgtgtgtgtgcaaaggtatagtttacacagtggcaggactagtcatagagttatcactgaacacatgaagcaattgtcataaaggacttggctacctcacgtccaaattcttgtaatgtctggaagataaattatctcctgggaaacgcagccttctccaactagaaagaataactagccctgtgtgtatacaggaagtcctggatgagaccaactttcatttgggactttaaatgaagacccaaatgctgcaaactgtgacttctccagtctgaatagagtatgagtcatttagcagtagagtaaatttcctctcatatgtttcttagagaaacaaattagttgggaggcagactgagttctagcactcaaagcctaaacaagattgagctatgtgtcatggtgattagggaagggaaagttgaatagtgcaggagagagtaaaatgaaggacttgaagcaggtgtctgtgttaacttattaaacaagtctaacttattagtcaagggagcatcacatatactagacactgatcccagcatgtttctcactgtaacaaacacatgttgcatgtgtagtgacctgaactggtgtcaccctgagaatgttattatgtggttctttaagatacctgagtgggaatccctaaaatggggctggcaagagcacttgtgggctggactgatcacctgaatttgacatccacatcccacaaggtagcagagtggagcagacttctgataatcatcctctgactccacacacataccatatttgcacctaaccattaaagcaagcaaagtagaaagatgaatctttacagtagagttcaaatctctcgggctttgttattgacatagttacagccagagaaaccaaaaccacttcacactcaaagctacttataaaacttcaggtgattcttttaaaagatgactattagtcatctaatgttagtgttacaaaaatgcaggtattaaatacaatatcctgtcccaaaatcccctgcaaaagcatagaaacttcatgaagagcataccccagaagagaaaaccaaatgtcaaagatagggtgggggtggatggctaagtggatcatcaagactgcatcgaaagaattggatcagttcagtatccactaagggacaccaagaaaattcttccttctatgtgggactaagtggtgcccttctgctaccaagaattctagcagctcgtcttgttccaacagtcctaaacattgttctaagcaatctccactccacctctagctccacattagttagcagctgaccattcttgtgaaaaacacacaagactgatcctcctgtcacaagtgatacacagatgcctacttccctgttgcatctggatgctggactcacccttgaagaagttggagttattataacaacacacactcagaattgacttaaatcgcatgtgccagcacctcctcatgtaactttatctttttctaagtgcagtgttgcatgcaaagatgactatctaaaatcgaagaagtctctaaagttctccaactgagattaagtgatcaacacttttttacagaggtttttcctaaaaaactgtgtccctgttaactatggtaaactagttgtttcctaaaggcagaaaaagtattctattcttttttaagttacattattattaatttataagatgaaagagcccccctcgctaaaaagatacaggagaaaggtcaagtaagctttgagtaccctttgccaagttccacgatgcatagtcatactctccctaccctggtgaattgctactgttcctctgtagagaaggcatccctaacctgaactcagaaggaagaaacagcttaattaacttaacccaaatttgcaaagtgttaagtgaaagcactccagtcttcaagcatcagtcaatcgagaatgatggcaccataatcatgaataataatcaatattagatgagagactctgggttttaatatcatgcagcatgattgtaaaattatgtaaagcagaacaaattatgtctggaatgatctgagaggttgcatgtgcaaaattgattattattcagttgtagaaataatgaattggaatagttgcaggaaaatggataagaaaagggaacataaacacatggtcttacttgtatgtgaatcgtagcctgtaatatacatagaagcagacagttgggtataatagcatttaaaaaggaggccacaaaggggtaatagtagataaggaaggacagattgcaggcgcaaggacacaccgtgtcatgaaagaagatataaaggtaattgttcctatagtttcaaccgtcagagtagttttcttttatgtatgatggataaacatatacagatataattgatagggaaagtgtcaaagactagacacagctttgtgtcatcggaagagccattctaactgtacagaagtttactgagatgtatagtttagaaataggcaagtattattttgagttgttatgtgatgtttttactcatgagtttatttaataaatatcttcagtaagtacaagcaataataaagtgtgggcgttaaccatgcatgtaattccagcatagggatactgaggcagacggattacaggtttcaggacaacctaggctgcacatagaaccccatctcagagcaaaactaaataaacaaatagagcttttcgaattgtcatctctgtaacaagccacacaatttgacccatacaattaaccacaggataaacttaaaggaagtggaaaactacgtatgttttctgatcctaaaactctcttatctccaatcatccacccagatttcctaaacgaaataagtgcagtttctaccgttaacattttctgaactcttttcttggactaaaaattttttaaaagccactgttccctttgtaatttttaatgttgaaagagcagtagttgtgtctgagtatctccatttcattgtatctgatcttcctgttatgctttctaagggatttttgtttccaggataatcataaaagaaaggagaaatagacagcatgcgttactgtcaccactggtcaccagggcttcatttgtccttgtgcatagacatccacagtcattgccagtctgtgagcacatagctactctcaagacattatggagtgggtatgaacttgttttagccatagactgtgaaaaaggacaacatccaataagaatatgtgtacataggaatcaagaatagtcaggatgtaggctcgctgaagctcctatccagggtattgggctacacagactgttaaaagcctctaaggtatggtaaggtaaggtacttggctctttcctatccttttggactcatgtgaaagtttcctgacttgagctattcattctctcttcatagctgtttccttccctctaaacaccactacagattaccattttgtaagtttatattaatatctttattcaggtattaataaaaatacttcttagttggacttcattgagactaaattcagcccaaatcaattgagccagatctaataatgatctagtggaatgagattattttagaagccaattatccattgaaagtcatgaatatgtcgacaacaaattactagagaaaatgtgggctcattttctcctctgtgatctagtttctaccttccttcaaatcctcttttcaatacatttttatgtgtcaaccttttttgacagtatacattcatttgttctcagttactttgtttgaacacacatggtatttatattagagctccaatgagctattattaactcaattgcctagtgctgttatcaagggacgatttatatcgtttctacagatgaattgaatcacaaacacacacagacacacactcacacatacataccacacacatacatacacacacacacacacacacacacacaccaccaccaccaccaccaccaccaccaccaccaccaccacatcacacactatacacaaacatataccacatacaccttgagaagggataacacctaagaggagctgactccatccaggagtctgttctctcttcaaaggcctctactatctcgtatgtgttatcagccagctctactgaggttaccctcgggtgaataatagtaaggaccagtacacatcatggcacaaggataaggccttaaagcaagcaactaaagtcagagttaagaaatgcactcgggctagacacaatctctgagcagatgattgtggtgatgctgataaaagaagaggaaagggagagtcagtgctgcgaatggcacttgctaacctttaagatcacagttaagtctcatgcctcctgcttcgggatgtggagaacttaatgcttcctcttctattcttcccttgtaggatttagtttcaagctaaatcagagagatttaaagaccttctctttgatagctagaggaagtttcacgcagttaaatggtgaaatatctttgaagcagaaaaaagcctgtctctctaatacctgttttaatgagactattacttaactaaaaatgtttccagtggaccacagactatataattttaaacagatttttaaataaaaatgatgattctacttacctaaagtttgtcctcaaacaaacaaacaaaaacacaaaaacaaatctatatatcaggactgacttgcaaaaatgataatcaatgcaccatctgattatgttaaatttttctcaagaattctggaaacgttttggaattagattgtgccttagtgaaaaaaattattttaacacagtaagaaagttaaataaaaattggaaaatatgtttcctcttcactatgcatgctttaatgccttgacttagaacaaatttgaatgaatggatgaatgaataagtaaatacttatattcattcatacatttgtgagtatgtgtgtataaaacatacaaaatatcatgtctatgagatatctgcttattatttttctccagtaaactctaagtcaatagctccagtggtccccccccccacccatgcagttttttcctgtaaatattttattactgtgcaatttctcagcatttggggtttgttagctcaagttactctaagtatgacctcaaatgattctaaatcagatggtgtgtggattttaagcataagcaagagataaccatagtgactaatggcccaaaacatccttgcaaaagctgtttctttagaaacttgtgcctttcaacattgctctccaaaaataatggaatgctgtcagcaactccttggccatgagaatcatcaaagatactgtagcaagaataagagacagaggcagggtaaggaagagggattacaagtacaactacctgggctggagaagcggctcagtggtgaagaacactgattgttcttccagaagtcctgagctcaattcccaacaaccacacggtgactcacaagcataactgaccagagaggacaagaccccactctcagggccatcatgtttagtgttgtacaaggttctgtaggtttggtgacccagcatcatacaacacaacaacatcaggcatttggtccacagttgacttccttctgtctgactttctatagtctagaagagctaaagagctatggaacaaacttaattctttaagcaaattgaaggtagttcccccaagccacacctacttctttcattagaaaaaagtcaattaggaaaagtttagttattaagggcagagatctgagccttaggacatttagcttcagattgtctgtctgttacttgttacccacggcatcttgggcatgtcaactaacttctctctttctggcttactgctttctccactaatcgtagaaatgctaacacctacttcacagaattcttatggggataagaataagtcatgttttaaatgtgaaacacagcatgttggccttgcttattattcttacttattatttaaatttttttccatataacagccaaaaagcagtaacggcattttaacttggtcacaaaaggttgtcatctgtaagctttgtgatggtggtgacagcatgcacctttaatcccagcatttgagaggcagaggcacacagaactctatgaattccaggacagtctgctcttcagagtgagttccaggactgccagggctacatggatgaacactatctcaaaagaaccaaaataattaattttatgatgatgatgatgatgataatggtgatgttactattgctattattgtggtttacatgtgacaataatattgtaaaaatattattatcacatataaatgacaagtttttaagttatctttattcctttcgacccactctgccctttgtttatctgtccactgatagatgagtctctccattaatgatttaagcatggggggttttttgttgttgttgctgtttgcattttacagatatcattcttatctactatttatctgccaaccatttattgtagtcgctgccagagtgacattacaattatgtgtgcttgtgatttttatgtagttaaatctggcagtcctttccatgtgggattttacacttactttgaaagactccatgccacagagtatgagtttgttcttctgggttctggtagttacataatgcttataaaggttatctctttaatcctcttagaagttggacataggttattaggtgatagtccaaatttatctctttctcaaattgtccctgaccccccccctcccgtgatctgaaataacaccttttggcaaacaccaatctcaaaacaactacaaatgaaggctttagattccgtgacgtggatttttctgttattgggagtgtttaattccttactgttgggtaagctggctcaagatcattccatgtgtttaataaatcctgtggcaaccatcacatgtattttaaccgccagaagaattatgaggttaatttctcaggttttaaaataatagaatctaaatatgacagatgtggaagaacagatttcctgacataatacacttttcacttaaactgttctttagggttttctgctaaactttatcattttcttcacatgatttcgggcacttcttatgtttattatttccctttattggtcagattttctcctggtgtcttgtttgttctcttagcaagcattcattcttaccctcggagtgctctctgactagctgcactacagtgttattgaactgactggcttattctgaataataatgaacagctacactgccctgatattacacctgtgtggtgcctttatcaatacaaggacactgagttctattcctaatgcatggagatcttcattttctttaaactaatatagatatctttttatttagtaccacttgtctgtttattatgaaatccgtgtatgttttcattaatgaagagaattaaatttatagaacccctaatactcaataatctttgtgttttatattgatgctgtctcgtaatcttggtgctgtgctgatagtccatatttaaactcatctgtgatttttttctcttagttttctgatttattatgtattttgtggtttttatcaaagatgtgatatgtccacaaataatttcatagatttacatcattttccattcttcaaagggttttctgtaatgaaacaattattcctttctcttttttatattcattttatttgtttgtttgtttgtttgtttatgtgtatgtgtgatgtgtgtgtaagcacttacacccagcaaagggcgtcagatccccctggagttgtgagcatcctgacatgggtgctaggtactgaatggtgatcttcttgagagagtagttaatattcttaaccactgacccatctctccagccccaatttcttcaataatgtttaaaatatgcctaataaaactatctgtttggaaaaaagcattaagtgtttttcaattcaagtcaacagttattagtttatgtaaaaccttgatttatccttggaacaaatttataaaaataaaatcacactttcctaagtaaatacccaataaaccattgcatctattttttttaattattagaagcaatatacccacttgtaagaagtctctgacttgtgatatggactcttgtctcatttgaagaattcacatctctctgcctgatcataaggcctgcagccgtccagttgattcttctctcctgtcacctctcactcttcgtgtccagtctatagaactttgattagtaattctatttttgtctctggtccctattggtcaactgtgatcatttcctagtttcttctcttgagtagttatgtgcagtggtttctgttttcagtaaatttttttcagattagggtttttgacccaattttataatgaaaattctacttcaccaagtattcacattgtaacaaaatctgaatctttatattgtaaattttagaagccatatatcataagataaaaaaatatattcatttttctaccacccagtttagccagttagccttttataatcaacaaccttatgaatataaatttaacataaatgaaatttgaccacacactctttttacaacttatcttccaattattttcccaaaaagtctaccgcacttgtgactcctttgactggcatatcaaccggtattatattactgttcatgccaatatctccttgtcactaattacttgggcaatgtctaagctttctttcaaactctgtgagagttgaaaaaagaaaaaaaaaacagtattcagggagttaaaatcatggaagaagaacagaagttgaaatgtcgttgtgttctcaaccaatataaaatttactactgaggttttttttttaattataaatacaattgatagtcactaaaacaactttaatttctttatttcaagatgatctggacccaaattaatgcatcatacattaacagatgccttaacaacagaaaggttgttctgattaattaattgactgatatatattttgaagtaataaggtgatacattaagactttttaaaatgctttttagttttttaagttaaaatataattgcatcatttccctccttctctttcctcccttcaacccttgccagtatcacccttcatttcctctccaaatgcatggtgtctatttttttaatccatgtaatcgccacatagacctttggaatgtgtggatatgcatattctttttctatctttcatgatgcaaactcaaatgaactattgatttcatttgtaaacatttttctaagtcccattttgcaactaatttatacttcttttattgagcaatgcaaggtaatatttatttcacatcaccaagagattcttatcacaaccccttcatttctgtgcaatctggaactgaattgcttctcaaaacaacttgtacatgttttacaatagttactgtacccctcatttatatcatgcatgaccaggtcacaataaaacattagagaaaaaattgatatgacttagagagattcgaacagcaagttacaccttcaacaacttcaaatgcacatggggtctggtgtcaccagcgccctgaggttcccacgcaatgagaaggaccttccactcctgatcaagtcgcagtctctttccccatttagttttcttgtccaaccccttccagagcatcaaataagagggtggctagtaattggagcagcagaactacgctttccaaattctaaactgccaaagacaggaaattcgtatagaggaaatcaatatttattggactcttcattaaagttgaggataactgtttcattccactttttttgtttgttttctaaggcatttttattagatattttcttcatttacatttcaaatgctattcccaaagccccctataccctccccccaccctgttccccaacccacctactcctgcttcctggccctggcattcccctgtactggggcatatgatcttcgcaataccaaaggcctctcctcccattgatggccgactaggccatcctctgctacatatgcaactagagacacaactctggggggtactggttagttcatattgttgttcctcctatagggttgcagactcctttagctccttgggtcctttctctagctccttcattaggggccctgtgttccatccaatagatgactgccccacccagggattcatcccatatacaaccatcaaacccagacactattgcttatgccagcaagattttgctgacaggaccctgacatagctgtctcttgtggggctatgccaatgtcgtttcacttttaatcaagatgcacactgacatccacggtctgcatctcttttctgacatctactctctgcacctcctttttttctctccctgtaatttctgcttttactttctaagctctagtgtctttgcttttaaacaggctctcctcccaaatacattgccagtgtttgtctcaggccatacccatttcacatttggcacaaactacaagtagaagacagcccttccatttatcatgcttacaggtcagggagaatgagagtcaggggaacattataacactgtgtgaggcagtacagagtcgtgcaggctttcctgagaacaaagagaggatggcatctaatgctacccagggaggttgtcaaagaaagcatcatggagcaaacagctttcaaccaaatccagatggaatcaatgagacccaacagtgaagttagaagacacaggaagggaaagtgtaagcagtgagcctggagacgtgaagaagcgtgtgagtgctaagagaaagggcatgtgaaggatggggaaagcagacatgggatgtgctactgaggagcatgtgccatactgatgacatgggactttatctaccacatggagctatgtagagctataatagccaacactgtgcaagtattaaatgccagggaccattttgagtatttaataattaccttaatatttaatcctcagaagactatgaaataagatatactgctatccgaattttctatttaaagacattgaggctcaaagagcctggatggaagtagaaggatatatgggaatgctttctttttcttttaacattggggtctccttatgcagcccaggctaaccttaaattcatgattttcctgctgcagcctcccatatagacttgggtaaccgacatgaccatcacacttggctcagagtctatttttatgtagctatattcagtggcatacatatttgagataagagtttcattgctaagaaaaatatagtcctagagactatggagacattggcccgagactgttaaatggcagcgaggcagtgctagtgaagagccgtggcatttttgtatcatatttttcaaatgaatactgtccagggagtttgtcctggttatttttttattgtaatttgacaaaaccttataaccttggaagagggaacctcctttgaagaattgcttccatcagattgtcctgtgggcatgtatatttattgctaattgatatgaggggccagcccaccatgggcagtaccattcataagcaggtagacctatgctgtataaaacaacaagctaaacaggattcagtgagcaagccaggaaactgcattcttccactgtttctgcttgagttcctgtcctgaactccctaaatgatgggctataccctgtaagttaaacaaaatccttttctttcctaaattgcttttggtcatggtgtttatcagagcagctttgaaaataaactacgacaaggcctttctggatttactttcttgcccctgtatctcacagctgtcctccgttatgacttattagcagaaatattaaatcacattgaatcccatgtgctagaatcacagaatacatccgaagtgttttctcattcatcttcattacagccacttgtccacagcctgttttgactacgattcacaaatgaaaagcctgacattcagagtgagctggaaatttaattctttcctgcaatgctgccctgaccctcttccaacaacttctatttttatctctttatctgcactgacaaacagaactgtgccacggacctacctaagtttccatagcagaacctggcaattgaaagtggaggtgcttggggatgccttgccttgcctgcctcctctgatgagtgctacgatccaatgcctaatgatactctgtggcacaacaagccaggagcttttttccaaagctgtatcagtatgttgctctcctaagccccagggtaaatgggggatatcattgctgcacgtgagacagtttcgagattgaggaagacaacggtgaaaggcagaaagagaacagaaaggctcttcccacgttagattctgcctgattcttggaccttggacagctcacctcccccattcccagacctctttccttgcttctaaactacttcagcatcctggcgctcttatccttatggtgttccatctagatctccttcgatggatcttccttatctcttggcttcttgagttggagttatctgcaggtctgccttagatacacaccctctccccagtccttacatgagctcatccaatctcaagactaaacatctgtcataggtcagctccaagtcagtgtctcagctgaaactctcccttgaaattcagacttggatttctgtctacctgtcactcagtttatctgtttggatagctaaaggcactgtgaaaatcagcaaccccataacccaacttctgcttcttcttccacaaatttcctcaactctattcttgctttgcctttttgttttgttttgttttgttttgttttgggttgggatgggttttgtttgagagttctggtgtctaaaccaaggacgtcaaacatgtcactcaagtgccatggcaggagaaaacacacccaatccctgaagtgccttaggctgcaaattgtgagtttcgttttaatgtgttgattgcaatgatttgagatttagatgtcgtaaagaatttctcagaacttcacactgtagtccaatggagcacagaggcctttctgatgtcaaaatgtctttaataacctttactaaatattaacccaaactcatggcaggttttgcaaacagcgaattcatcatgtaactatgaactgttacctttttgatggaaatgatgaagtctagcaaaaaacaaactacagcttagccgacagactgtagactttgccattgaaactattaaggcacatagaaataaccttatcagattccccagtccttcctggtcctccaggataagctaggcgaacaatgggagaacctgagagctttctgattaaacagtaactttaacgacatttaataaatttagcaacatttacataaaagtagctttaggtaaaatgtaactcttgcaaaagtttctttataaatcttcaaaaatggtttttatagttttatttacaagattagttcatagcaatttctataactgtatgcaccaacagataaggattataaattgaatatgggcatcaacttatctatgttttagtatccataggaacagtatgttctccaactacagaagtaaagctacatgacctaacctgaattgcttcataatgtcaaatcaccactatcatgtaaaagacactaatttcctaatcaaattaaaagcaaggctatagcttctggatagatgagatggttcagcacatgttgccaagtctaacttcctcagttcaatccccagaacccaaatgctggaagaagagaactaacttctacaagttatcttttgacctctgtgtgtgtttcatgcatgtatacacacacacacacacacacacacacacacacacactcattcattaaagtacataaaaattaaaaaagaaacttttctgtgaacactaaaatcttgtgagaagtgtgtgtgtgtgtgtgcgcacgcatgtgtgtgcccaaatgaattttaaaatcttttaaccagtcttgtttgactgtggaataaatagacaagtgtctcttgcaaaacacagaatagacagacaaagctcaatctcataatttttacttcctaccatttctctctctccctctccctctccctctccctctccctctccctctccctctccctctccctctccctctccctctccctctccctctccctctccctctccctctccctctccctcctctcctctcctctcctctcctctcctctcctctcctctcttcttctgccttccctctttccgtatgtgtgtgtgtgtgtgtgtgtgtgtgtgtgtgtgtatgtgtatgtgcgcacacatgcacgtgtgtgacatgcacatgtatatgaattgcaagtgcacacctgcaggcctgaggaacacatcaggcaccctactccatcactctctgcctcacttctttgaaccaaggtcttctgctgtataccaccagagccaggttggtggcaaacaaactccagggatgccactatggggctagggttacaagttttgtagggggtgctggagattcaaactcaggtcttctggcttgagcaccaagcactgttacctgctaagccatatcctttcacatcccttccctttttttaattggcatgaactcatctgtttgtatatttagtgatatcttaaagtaactgaactgggaatgagtggactatgttctaattagagaatggatataattcagttttcatttcaaaaggttgaacatctaaaaattatatataattataaacacatgattttaaaaaaacaaactgttgtccagtcttgtttgaatgtagcagttagcagctataaccttaaggattcctttaaagacctttcctaaagatgcccttcacttgaagggcaatgtagacttggctatagtaatttacttaatttatcttaaatttggtttcagtgagcaaccctaattaacctgattttttgctgataatcactctcaatggaatcaaatcacaaatccggggatggattgagcggcacccagaaggaagcggcactccgcgcactggtccagcgcacaggatatagcttggtccaggtaggaacttcttgtgacatctgctgagactcaccactaaatccagagtcctggaaacattaatatccctcgatgcagaaagccagcagccctggtatgactcctgtgtaaatgtctttcaggggaaagatgggaatagcatatttcattcagctctgaaggtaatctggactcttcctacctgtattatccactagaccttaatctgttgcttgaaagtataacggctaaccacagtcatggagacaattacagtttcaaaaggccattttagaatacaaaagcatggttaggatccccagtttttagtgaaggtgaccttagctaatacttcaggagtcagtggagaacaaacttcaggaataatcactttggtttcaagagaggtccctgcagataaagacttcaagatgtggcttccaagaaggtaactttttttttttcctagctattctactttgtctagtctatggtatttaacgacaacttctgcccatgttagacctttgaaaggcaaaaatacaagggccatgaaaaagacctaattattaagaattatttgtatatgactattgccattgggataatgccttttagagaatatatgctgcattaatgagcaggctagtgttagaatcatcaaaactatctcattagaatacattacacccccttttgtaataaaacaatgtatttggacccaacaaccattagttttcacctacatacttgtccaataaactcaaatctccctactgttcagaatacaaatattacatgtgagtagtgacagaatatgccatgtcttggttttattagctctcctagagttggatttttggaaataccctagatttacttatactgatgctgtaaatcataaatgcatattatacatgatacagtgcctcaagtatttatatttttatcccaataaggagtgctagtcaggacttttaagtacttcatgcatttttttattctgtagtgactttttaaagtttcattaagtcaagggagacactgagatatttgtttcactaaaagctgtgttattttagaattagtgaactgtaaatgtttttaatctctaaaattaataaatagtttaaatatttgtgtatcttttcgccacactaaaataatcacagaaaattctaaagaacttagtctacaaaagaaatatattatatttcatgttatttctttttacacatgtcaacaggcatagttggctgtaaaggtggtctttcttttttaaaagagttgggttttttttctttccttttttagccaattctttaaattttgtaaacattttagtcaaattcttctgtaaatattgtagaaaaagaaagtgactttatcagttttttttggtttttttgttttttgtttttttttttttgttgtgttttgttttgttttgttttgttttgttttctgggggtggggctttcctcaagcctgaaacatttattcaggggaagcaacagtgaaaagacattttaacctttttaaaacctaatggtcaattttaagcacataaaggaagtccacctaggatagaagtgtgtacaaacctgtcgagaacagcaaagagtaccttgctatcacagacctaagtcaaagaaagtaaggaaatgattaaataaatagccagagccttcaacaagcaaaactcaagagggagatcctacagcgtagagtttcagtttaactttattctcagtgtggacagaccacgtgcaagatagaatttagggcaggggaagcctaatgatattttaggagcattcaagtgaaaaaaaggtttcttgtgaaaatgctaagatttcagtcaagtataatttcgtagcttcaactttccggttcttgcctaaacacatcagggtggaagagaattcctcttgtatttctcagcttactctttgctctgttaactgcaagttctcacttgaccttgaagcagcaggatggataaagcaaaagagagcagggaggaggtgcttgaataatggtggcacaggggactaagaaactgaagccactgggagcccctgtaagatcgcagagagccccatagcactgctcggggaccctcacctggccacactttctttgaccagacatctttgcactgagctcatttagacactagccacctcgtgagaggatggaagataagccatgggtcactttagagaggtatggaggcattcaaaaactaccttgttggacaaaagtcgctgcactacttaagtaatatttgttagaatgattactggatctgaagttctctaataatatggaggtacaacatcccaaaaactaggtgctctagtatatataatctaaagtaattatatatatatatatatatgtatatatatatatatatatatatatacacatacacacacacacacacacacacacacactttattgtgttttcaaatacatacagaattaaatggctttgcattggcaaatgtgtcctgcattgggctcaaaatgtagaatgtgacacaatcaggaggtctgtagtctcacctgctccttatcgcacataagatcttacacaagtttcctaacttctggggaccttaatttcccctaacgtcagtaaggtccatccccacactgttagccttgatctgtgatctgtatcgtctcagcttctggtggacagctaactggacggagagtagacaaaacctccggctcaatgattcagggatacctccctactcttcctgatgttcacagttcctatgctggaataaaggaatgactaatacaaaatagaagaaagtatcagttttaaaaaaaatctctgagcaaaaatgaagatgttaactaacttctgattggaaggatttgtctttctgtgtctttccagtggtatcaaaatccaaaccacagaggtgaaagagctgggcactcttatcaccagcagggcaaacacagctatcaaaaatagaattttacaagttacttaataacataagctagcctttaatgtggcaactgatgcaacaacgtgtgcttgtacttatgagaattaaaagtgtaaattggaacctcttttttttaatataactggatatccttctgtattttacccaagttctttgggaaaagagattcaatgcaacatccttaaaaccaaaatatttaaatacatattgctcctaaatatttggaaattgtgccacggttttaaaatgcaaatagcccttgccatttgaggttatgccatttgagggttttatttttctgcttttccactcctaattacattccctagctttctgaagacagaaaatactgttttgagagtcaaaggggaaaagtcccaggacatggaaaagtgcccacacctcaacagtcagctttaaaagctgaatgcttgaaataacatgttatgaaactacatatgcattattttaaaactggaaaataaaatagcctctctaattatgtaagcgttttcatgcctgatccttgtctttgttttccagtctttttgcagtgacagagttatttgaagggaaagtatcataaaatataaaatactaccattattgctcaattagaaaaactgggcaattttcacagaagaaaattcactatctcatctgggtgatgggtacctggagttattgggcaagccttgtgtttaatgtttgtttcgtagtgacagagtccaaccctcctcttggaagttaggacaacttccctagaattacaaaccacagactgggaaggcaatgagcaatcagccaagtatagctatcacctgtatctaaactcagtgctggatgcaaacagcattatgtttccaaagataatagtgaatgaaaaagaaaaagaaaagaaaaatagaaaggaggaggacatgaccactcctaaattataaagatttttattgtcgatacaagggagaaagcagggagagggaagagtgtaggctgaacatggccggcagataaaaccagaccatgagaggagggagagcaagagaggaaccactgacctacgaagagaggtgacgccaagagactaagatggcaaaagggtaaaagggctgacatagccaaaatggctggactatatggagaagagctgctgggggctgggggtaggacagcccagcccagtccctgggctggagaaatttaggctgcgggggcatgccagccatactctgtaacagataggaacagagggatgctggaagctggcagccaggtctactttaatatgttaaatagatactggtttagccatttgtcctgggaaacagatgcaaccttctttatttccaccattcatgctaagtcaagcaagcactgagactttacccctccatgtattgatgtattgatagattccacaaagagagttgtagtttcttcctttgtgaagaacattctcattggaaaacaaaatcaagaaagaaagaaagaaagaaagaaagaaagaaagaaagaaagaaagaaagaaagaaaaaaaaagaaaggaaggaaggaaggaaggaaggaaggaaggaaggaaggaaggaagagcgagcagcaacaattaaagtcaggtcctgagcaccttgggtttgatctataagccaggtcacatgacagaatggcaggtgaccttcttcaggcacgatgccactgaagctgagatttagggactggagcaagcaaacctttcaagggtctgagaagacaccagcatccctggcagaggaaccctgtcttaaccaaacttttgtcaaatgcataccacctttgaggaagtaaagagattggtttcaatgattgctacattaaccaggtatgagtctggttactttaacaatctcccatctttagttctcatctttaaactctacatgtaggctgcctgattaaaggggtttcaatggaattatatactcttagtctaatttgcaaacaatactaaacacacacaaatatataccataaagtaaaaagaatgaatggcattatgtcaacataaatctgatttggataccaatcacgcttccctttggaaaggcaacccacactaagatgactagcctccttttagttaaatgtatcaaactgtcaaagggtcagacaaaaagcgaatctggaaagaacaccaacagaagagcaacaattgagatacaagagtgcttagcagccaaagtttagaaggaacaaactgttggccaagaacactgtatccaagtaaaagcagcatttagaaagaggtatcggaagtgtctcaaacaaaagcccacagaacttatcaccaccagacttgtcttagaaaatatgttacagtgaaatcttcaggacaaaaagagtaacatgaagattgctgagctatgctggttccgaatcctctagaagattaatcatgctacgtaagtgtgctagcttgattgctgtaataaacaccatgaccaaaagcaacttggagagggaaggctttatttcatcttccaagtgacaagtccatccttgagagaagccaaggtagaaattcctgcagggcaggaactttaagctaaagccatggaagaacattacctactgctggctctttcttttttataggaccagctcccctggtttagggatagagtcacctacagtgaactgggccctctggcaccaaggaacaatcaggaaaatgccccacaaacacatccacatgccgatctactagaagcaattcctctgttaatgttccctctttccagctgtgtcacaatgacacagaagattgggcatcaccatgaattgcttatatctttagtgtagtgaacaagataagaaattattacaaaatataacaacatgaacccaggtagacctcggagtaagactgtttgccaacatttgcaaggacctggattccacttccacctctgcaagatggtagtagtcctactgatagtgataatagcaataatattagcaaggaaagatgtgttgccaaaggtgtaaattgtaacatcaattattctttaaaaacaacatcctagttattggttaaaaataatttgttgaaactatagaactgttttttaatgggcctcttgctaaacactaagcaaaacctcattacagggatctggagagatggtggctcagaggttaagagtaatggggtttgatttttagcacccaaatagctaacaagtatctgtaatttcagtttcagagaatccagtatccacttctgacctctggatactaagcacacacatattacataggcaatctggtatcaaaagacccatacacatagggtatttgaggagcacacataaaattagtcctattttaaaatcctcattatatctacaattttaaacagaaatggaaatacactaccagagaaaactcaattaaccaagtggaagacagtaaaagaaggaaggaataaacaatttacaaagcaacaaaatggaagtagttagtctttaaaaatcaataattaccttggatataaatggactggatttttcagttaaaaagacagatttgatacacacatatgcacacatgcactgctatgctgcttgcaggaattcatgtcaccttcaggggcgtgtgaactgaaagtgaagcagtaggaagatatttatgcacagagaacacaaagaagttataagaagttgtgcagacaaccgttgcacatatgtgaaataaaaaactttaaagaagacaaaggttatcaaataaagaattcaatttgtcaaaaggcttcaaatggtgggataaaagcagtggacagcatggggactgcagggggaatgcagggggaattgcagggggactcaggggcaactgcaagggggactgcaggagaaatgcagggtacactgcagggggtactgcagggggcactgcaggggaactgcagaggagacaagctgctatcaccaacactggttcagaaagaggcggatagcaaaatcagagcagctttaacttagtcttttccatcattgcacataaatcctaaagacacagagaaaacagacttcacggtgaagcacagcttctggtactttctcttctgttgcgagctctcaccacctcttccttcccaggctgtccaaaacttagatcatgtatgtcagttctaagaacgcagagtgggaagagtcaaattcttggagacatgagcttctacacgcttgtccctccagccactcacaggccaattttattttaaagctcagaagaaccttgcttcttctagacagagatctagtaatcaaatgaaggaacaattctgtgagccccttcatgagtgggtccaacgtcatgtgtttggaaacttcctcatcccaggaaatacatgtgtttctgcaactgttttcaaatagtgatgaattgatcctcaaataccctaataggaacttagcacaagagattctaaacagaagagtcaatatcatcacaagctagcattgtggtgccttccctcagactagaggcaaagaagtgttttatttcatcaatttaaaaattagtgtcaacttcattatcaaaatgtgcagaggcagacaggggctttaacattggactctgttttcaggcaatctcaagagtttcctaagagctatccacttgttgattttggttagcaaatagaaatatgtgaatcttcatctttagactctgaaccctagccagagataaagacaaaacagaacaaataaacaacaacaacaaaaccccacaacatttcaaatacagatcaatggagaaattgatgacttcagctttgaatctttataatgttgacctaaaaggagtgggcaggttactgaggcagtcttacacgaaatgttgtaacagtattaagtaaagttaaccccactcagaattattccattttcaattcattcaggctttctggtaaggaaagttgaagtcatatcttaatccttcatccctggtaatcattcaaacagaaagagcaggacaaatgatttgagctctcagaagataaaggtttcaaccaataatgtgatcatgatttccttgttttgtaaatgtctttaaaactatcttgatgtatactatatccatacacacacaaaaaaaccacacatagatagatagattgataggtaggtagatagatagatagatagatacatagatacatagatacatagatacatagatagatagatagatagatagatagatagatagatagatagataaaagtcattatatagctatgtatatgaactggaaatttgctgatttttcaaactgtaagaatagtacttgtaaggcttccattgattagcacacattcatcctgtaaacatttttacctattccaatacagtatcaaaactccttctgaaaaacatcctgacactcccagaagactactacaattacctagtaaaggctactttttaatagagtaaaagcaagaaagatgagcactgtcttgctaagtccctcagctttcaaagaatcatctttcacaaaagtatgataaaaagaataaaagataccaactaaagccagctgcattagaggagacagcccttggcatgtgtagaaatagggacagacagaagagcaattatccaaatccagtcttccttaccatgtccatgtacacacgagcagtctatgccatttagctagatctttagttgctactctattagttagttttagaagttatacctagccgggtgtggtggcgcacacctttaatcccagcactcgagaggcagaggcaagcggatttctgagtttgaggtcagcctagtctacaaattgagttccaggacagccagggctatacagagaaaccctgtctcgaaaaacacccccccaataaaaaaagaagttatacctaacttgaatcttttaaattttttctattcataaccccctttttgcctagtaagtttttgtgcatccatatatatatatatatccatatatatatatatgggtatataaaataggcatacgaatcaaacattgactaataataagtcaaagtaaatttatctttaaaaaatttcctttgatatatacgtggctttatcactgaccaaaattgaaagcaaacttaatactctgagctggtgcccttgcttcgcatggagaattaaatcttggctacacatttggtagtgaaaggtacagaccatcactaatggttttcagaactgattgatgttttgatcttgtaatcctcaatgctgagaacaactcatcatatacacacgtgatacagattagtagaggctgggacatttcagaaagtgttggaaactatgtcccaaaagcaagccaaaattcatctaatgattattttatgaaactcaagtcagtaattcaaacaaatttttttttaaaaatcaaacatcctaacgtaataaaacccaaaggtatactaatttaatggttacgtaatgagaggtaagggtaagaaaatagtcaattcgcataactaaatcattatgctctgtcaaaccaaattttttttaatttcattttatttttaattcattttttacactccatattccattccccgcccctccccatccaccctccgactgttccacatcccacacctcctccccaccccaccccatctccacgtggatgcccccccccaccccacctgacctctaaactccctggggcctccagtctcttgacaaaccagaattttttttttaatgtaagtaaaacctactatgatttgtgacataggatagttttgaattcgagccctagtgtattagatagcactcactagcactgagtggcccaataggcaatgccatgtgtgcatgcctgcatgttgtatgttacaaccaaggctgcagcaaagtttcaggatgcagtataggtgagcgcccccagaaacatctaggattcatcacacatttacttttggataccttttttttttgcttgttgagtgttaagaaactgactgataccaaaatgttccggactcgatgtttattgacacaaccccccacaactcagcttggagtttaacaagctggggctactagctaaagggcacactgtaaaagcccgaaacacatggatgtattggccaagttcttcaccattcatatctaaagttccagaggcacctagatgttggaacgatgcatgtccaaaaccatgagtgcgcgtgggcagagcacagcgatctcagaactcagcagtgaaacacatgattcgctgcccatgattatgcaaactgctttacaaaatgcgagcaggtctctgaagctagttggcactttcgatcttctcttttatggagaaacaaatatgcatgctcagaatatgctttctgacttctgattttatctatgcttgaatttagtatccttgtggatggtgaacaatgttataaggtatgggttgttttttaaagaaagttgcttttgaaggcttcagacattgagatactgattccctgagaagtttgcagcacgtgctcctcagagataaagaagtatcaagagatgttgttaaaatagtccatttgtttttttaaactgtcttcttctgtatgatactactaataacttaaaatgaaggagggactatgtaccaaagcaattgatcttcaaaagtctttaggcaaatgatattttatgtaactatatcttataccttgcatagattataaaaaataactaataaggatagtagatagatagatagatagatagatagatagatagatagatagacagacagacagatgagagatagatgagaggtgggtagattgatagatgataggtagatagatagatagatagatagatagatagatagatagatagatagatagatgatagaaagagacatagaacatagtgagtcaatttagtgttgctcgtatatacaggtatttgtatttcaaaaggacccacccagatggacctggagagcatcatcctgagtgaggtaacacaatcacaaaggaactcacacaatatgtactcactgataagtggatactagcccaaaacctaggatacccacgatataagatacaatttcctaaacacatgaaactcaagaaaaatgaagactgaagtgtggacactatgcccctccttagaagtgggaacaaaacacccatggaaggagttacagaaacaaagtttggagctgagatgaaaggatggaccatgtagagactgccatatccagggatccaccccataatcagcatccaaacgctgacaccattgcatacactagcaagattttatccaaaggacccagatgtagctgtctcttgtgagactatgccggggcctagcaaacacagaagtggatgctcacagtcagctaatggatggatcacagggctcccaatggaggagctagagaaagtacccaaggagctaaagggatcttcaaccctataggtggaacaacattatgaactaaccagtacccctgagctcttgactctagctgcatatgtatcaaaagatggcctagtcggccatcactggaaagagaggcccattggacacgcagactttgtgtgccccggtacaggggaacgccagggccaaagggggggagtgggtgggtaggggagtgggggtgggtgggtaagggggacttttggtataacattggaaatgtaaatgagctaaatacctaataaaaaatggaaaaaaaaaaaaaaggacccacccaaaactgctgctggaagtttaaattttgaaaaccaggctaaggggaaactcttctgtatgaaactcaaaggcatgtcacagatttataattatgagaaaattaacccaccatcctcaagtgcacaacactgaagtgtgtattaaaattttgtgccagagctcaattttatcactaataaaaccatggctgtcctagttgcgaaggaagctagaaaagcaaatggctgacatgtttaaaaacctcaggaaggtatttttcccaaatatacataaagttccaatgcatggtggacaacgtaaacttttagcattaattctaagacttactgcccagcatgacctattaaacaaagttatctggttgtatgcagggtcaagaagatggtagccataatttcatcaagaaaaacttgaaacatctgaggaggaggaaattccaaacactcttgacataaactgagttactaggcagcctcagcatgcgataaataaaaaaaggaacagggcttgtagctagaatcgagtcatttatttttaattagacagttaaaagttttctacctatgtatgtgggtgaagaaacgaaaacgattttacagacctgattagaaagaaaggtaaaagttcccagcattgaataaacatcctctcacttttaagaagattctaagaaaaaatctctagtctatttccctaaactattaatatgtttgggttgctgaggagaataaggcccagaggacatgagccagaggggaaaagcacggttctagtctgcctttccctattttaaactcctcaactacaaaaatagggcctgaaatatcagcccactgtcctctcagagtaatggacagctattctgaccccgtgactaatgccccaggcctgcttcaaattgctctgaacttctcccgctggtgcattccaagcctgatgggtggttcccagggcaaatgctccccattgcatgaacttcagggcatctctaaaccggtcggactctgcaatccccaagactattattgggcattttcttcatgtgtttatagaaaccacaaaaacacaattaataagcttctgaaccacatgaacaagtggggctctcaaaccgtgccgaacacatccatatttttagtactacagcactcaagacaatagaaatgagatttcaaaagggcaaactgcttactgcttacttattttcaagccaaaggaatcttgtgaaatcaaagactccagatgccactttgccattagggaatatgaattcgatttcccgttctgaggggtaaaaatactggccaaattgatgcttgtattctaataattagctgggtattataaatatctgaaatgcacgagaaacaatacagcgtagacagctatcacctcacccacatagtgatatttggcccagggagggaatggtgaacagcgtcaataatttgcccctctgtgtacagtcagagtaagatatgttctccccagaattttgtgtaattgaatgactggaaattagaaagtgttgagtgaagcaggacactttctgcttagtgacaggacattttcaaatgctgagtttaatgtctatgttggttgaaagtccctagactttccaaccttaaggtcataatatgtaatgattatgtaatatataattacatattttgatacatactttacatatattatgtaattatgtaatatagctaagaattaagctgataaactcaggctgacttcctatgttcaaactctgccttctctatataactgccttatgacctcagcaaattcctctctgaccttttatcttctataaaatgtcactattatggctcctggatcaactactgctctagggccaggatactatgcatgcaggagctgatgtctgggtattagctggaaaaccaaaaaataaaactttggcaatcttcacaaatgtcaaattgatatgtttagagaactcttggatgggctatagatgccacattcttggcaggggtatactttttgttgttgttttactaaaaacataaaaaatcatcatttctctggttttttagtgatatcaagatttggctactatggtaagactatagggtaaggaattacaagtactctataggtgaagctttgaagattttagtaagatgaagcctgcccaatcgcttgaagcagaaatgtaacccaggcatccttttgtcttatcagttaaagcaacatcaaatacaataccaagaatacaaaggtgaacctgatttcagacaccttttgaaatactgcaatccagagaacattagggcagactctgaacaaataatgtctaaagttagcatacactgagagtaaaagtggtattacaagctgattcacttttattctaaacaataaacagtcaccccacaaaacaatatctttaaactaaaacatacgtcttaagagtgtacatagaaaacatttggaaaggcagactgaagtttctaccagtaattactgttgaggacgggtagggtgatacattggggtgtttttctttctattttgtatattatatttgttattcaacaccatgcatcctggcacaaaactgcctaatggattgatggtgagttttgtggtgaatgccaacatttagcccctttaatagtcatgataaaacgtgatggtctagtgttctgactgtgctgatgtgcaatgtggcttatcttcccccagcaaatgaactgcagcatcaagtagcactggattgtgcccaaactttggtctttaaacccctaggaaaacagatcattggaggaaaaagccagtatcattcttctcccaaaagaccctctattaggtcaagagagaaaaatagaagtcagctctatttggactgagaaagttgtggttcctcacataaagcgacaacattaggaaacatatcctactatccttcagaaacatacttaaatccctcagcagctatccccaaaattagccagttcacatctcccagtgttgatcttcatgtttttatatttagagggtctctccattgcctggcttgccctaggaaagaagggaaaagaggaattagtaacagctgagcattggccaagagccaagccttgcctatttgatgtaggttaccctcttgacaaaggccactgctgccactactttgcacatgaggaaactaaggtgctgagagactgtcatttacctaaagtccctaagctagtgaataatgaatgaagtcaaaactatccagtctgacagtgcttgcttctacacacaattctaatgttaaatcagagcccagatgtcaattgtaattgaccacttgataatctgcctttgagacattcatacaacaataggacaaactatctccttaattgttaagcagctaggactcagttaagggttctaccacagtaaaatgaaatgacaaagtgtataaatttcttaaaagacaaaatacagcgccacagactttgactctccagagctatggtgggttctgagtcactcctcagtattagggcgcatactcccacgttgtaattaataaggaatcatggaaatatatctttgaataactactattcttcagtactttacgtgtcagccgtgcttaccttgggcaagcttttgaagcagaaaagtgctgcattgaggcataagcaaatctttttatatcctagataatcaattattgttacttgacaatctcaaatatttcccaaaatagtccttagctaaaatgatttttacaaatgaggaaaatgaactaaagattttgttgttttcttttctccctcatacaccattcctccctgaacacacaggaaaatggacaaaggaagtatggtggccctccaccaggctgggattctacacccccagaaaggggctgcgagattttcattgggaaacttccccgggacctctttgaggatgaactcataccattgtgtgaaaaagtgaggctaaatttaatttcttttttcaggagaaaaatatcattctctttagtttgctctgactggttttcgcctttctttatatctctgtgtatgatgcatgtgtattgtgtgtgtgtgtgtgtgtgtgtgtgtgtgtgtgtgtgtgtgtgtgtgtttatgctcatgtgtgtgagtgtggatgcaaacctcatgcacatgtgcagaggccagaggacagcacccagtatctgccatcactttctcttatgtttgagtcgggggtttcttatcgttcactgctgtacatgcaaggctagctgacttccagatttctcccgtctcggcatcctggctctctgtagaagccctgggattgcagatgtgtgctactgcaactggctttacatacatcctcctgtttgtaaggcaagcactttgcccagtgtgccatctctccagtgcccagtttgatggttctctcctaagtccacacatcaagtatggacagtgttaaaatcttccctgacatcaatgactgtccccttatattagcacctctttgcccagctctttgtcctgttgagctgatgaattgaataaccaccgctgacaactttcaaattcatatctcttcctccaatctcagggctggcagctgaaccactgcaaattcaatgtatctaacaatacaaagcagagtagcattctgccaacaacaagatattgctccaatttgacatcttaccacgactttataaagaacagtatttcatataaaaattaatgtatgcttgtcatggcttaaatatgtatctgtatacagacataaaagtttcttctgcaagtagaattgattatcttattaatgcaatgaacagattaaaaacatgttactcaatgtccttaagagcaaggttactcaatgtccttaaatgattactaaggaatatatttccctacttccaatactttaccaggtgcatattttcagcccaaacacttaatatgaggatttggctcttcccctacatgattaaaaaaaaaaaaatcaattgtcagcatgtgtagtctcagaaccggaacccctggttttatacacagaatcctagctgtcatactaaaactccggaaaaacaggtaaaaactgtgctcatcttacaggaagaagaaacgaatccttctcaatgagtcctttggagttcagtagagaaaggctacacagaaagcagccaagcactgaagtattgtgcaggcttaatggcatctcttcccatatgcctattgtttacatttggaccaccctgcttacaaaagctaggggcaataaactgcttagcctgtattactcaggatagttgggagtatagaggaagagtaagggcaagcagggtagatggagaacaagtatcctttcctctgtggttccaatcctctaccagctgctagcactcctagagtaagggcaagcaggatagctggagaacgagtccccttttctatgaggttctgatcctctatcagttgtattgaggctagcattccttttcatcctagaaactgctcaaataagagtgctccccaggccacctatgaaatacactgtatgcattctgacatcatcgcagtggggaaagtacagccaggaagttaggttagcaaggtcccaaaacgagtaccctcgtttcctgttataaatgtcagtgacagagcatagccaagtaaaggtcgaagcatggagacgttagagaacaactcagaaggcatgggcaaatctaccaggaaactgtgcttccggtgacaaatgcatttttgggtccccaagacattacataaatttccagatacttgtatgacaaaggtcatttgctctgccaaaagtacattctgttagctgaggctgaaatgccatttgtctatattttctttatttaactcggcttttcccatgagttagataattaatctaaagccagcataaaagtatgcaaaccattccccatggatacaaatgccaaacatggcaggaggcagtttggcaagtatggagtgtgtttctgtgtgtgtgtgtttcggtgtgtgtttctgtgtatgtgtgtgtgtgtgtgtgtgtgtgtgtgtgtgtgtgtggtgcatgtgtttacgcacatttgggagaataaatatgtaaataagaatgttctgtttctattcattacagattggtaaaatttatgaaatgagattgatgatggattttaatggtaacaacagaggctatgcatttgtaacattctcaaataagcaggaagccaagaatgcaatcaagcaacttaataattatgaaattcggtaagttcctgatctgcttgacacaagctgatcctaaagcttcacctgcacctgcttcatatcaagatagcatacatgtgtaagctagctaaactaggaataaaatgctacatctttcttcgttaaaaacggtctggttaaacatttatttaatctcattactgaatgagccataaaacccacagagacaaaatttattaattaactaatttcattaatacgttctgaatactcttccagatattagaaatatatcaagatatcctctgttcccatagaacacaaatacaaaagattaaataaataactcgcaaacataaaaccgaggaggggtaagattgtgcggagagaatgctacttgaatttgttggttttaagaaaatactctatgaaaaaggcaaatttgatctgatgtttagatgaaaaattcagtcaagacagtcagaccctaaacaaggttattcaaagaaaagagaaagcagcaaatggcaagtacaaaaatcctaaaattgaaacaaaattgatgtgtgatgtgtttgaaagacaagaagaaaaatccaggagggggaaaagaaaaaaaaatggttaaaggtgggacagttggagaacattggtccctgctggagaatctgatctcatttacataaagacctggaactggaggagaattttaagtggctttatgattttaaagcatggttacagctgctggctacacagccaactacagtagagcaggacccgaaagagggaaactgtgtcaaaggctgaggcacattctagatccagacaaggaatgacagtgactcagtgagtgaccacaagcaaatgggacaaaaaggattaagaactagatatcctgaaggcaggggaggggcgaacaggatgactccatatcacattcagaatttgggcaaataacatgggatttgattccaagcatctgtgaaagaggctattccattgggaaagagagactaggggagagaaggaagcgtgcgaagtgtcctcgttgaggtatttgaccttaaagatgcacatcagacacctcaggcaagatacagtgcaactgtatgagagtagaggcaggaggagggtcacagttatagcattctgcagcattgtaaaccatgagaccacaggagggcatgaccgatgatgtataaccaataatctgtttctataagaccatttttctttaaaaacaaacaaacaagaaaacaccaccagggataaaacctgtaataaatcctttcttaaagagaccctggaccacctgggagggcgaggaacagccatgcaacagctaagtctttgggccagccaagcttcactcaagggccagcaagtagttgcttctttgtgtctcaatttcatttctttatgtatgagtatgtatatgtgcgctgagtgtgcatgtgtgcgcatgtacatatgtacctgcatagtccagaaaaggatccctgatctcctgtgtctggacatagcaggtggttttgagctgccctacatggatgctgggagccaaattgggtcctttgcaagagcagtacgagcttcaactattgacccatctttccagccccaactctcatctataaagtggagattatatccatttcccagatttgattgtaaagataagtcaaagagttttgaaaacttagtgcactatttaggacatagtagccatcagatgataaaacttataacagttcaactccaagattcagtttccatattaatcccttttctgtaatgtggacaaaccttaaaattcctggaatcttaacttggatggccacagacagagtggcctcctctctagtatttccctacttcttatatgaaagtacattgcctcacaatgcatcttcatgtcctgggaaggaattaacagctagctcagttttcttcaactctttagctacatgtagaatgactgggtgtggcaacctttaactgcctaagctggtcagttccaaggctggattctagaacagtttgagagaaagcacaagtaggcctcctctcctctgtggaactaaaaaaaatctgtgtggtatgcagaaaactggatatcccaatcaggagacctgggggcattttcccctttggtaaagcctttgacttcttagaacatcgcttttatccttgtacttgatcatatcacacagagattctgtaaatatcatacaaaaataatatacaccgacaaattcagaaaaggctttgcaaaactgtcatttaacttgctttataaaaatcactttagggaagtggggtcgcgtcagcgagcctgtgtggcgggaaacctggggacgcgactccaaccccaagcacagccaacccaagggctgcccctttttcccccgcgtggtctgctcctctagttcacctaaggataatggtgacagctgaagtactgaacatggggaagaaactctatgagggtaaaacaaaagaagtctatgaattgttagataacccaagcagagtcctcctgcagtccaaggaccagatcacagcagggaatgcagctagaaagaaccacccggaaggcaaagctgccgtcgccaataagatcaccagaggcatatttcagttgttacaggaagctggtataaaaactgctttcaccaagaaatgtggggagactgctgctttcattgcaccccagtgtgaaatgattcccattgaatgggtatgctgaagaatagcaactggatctttcctcaaaaggaaccctggtgtacaggaggggtacaagttctatccatccaaagagatgttcttcaaggatgatgccaataatgacccacagtgttctgaggagcagctcatgcaaaattgtgttttgctggccttgttatagggaagactgacgtgggcatcatgagtcgtgacacacaagctatttttgaaattctggggaagtcctggctgccccagaactgtgcgctgaagatatgaagattgaatttggtgttgatgtaaccacatgtaaccaccaaaaagattgttcttgctgatgtcattggtcatgattcctagaggctatggccatcaggagatcggagccagcagaaagacaaacagtcttaacattgatctcaaagaagtaactccggaaggactccagatggtaaagaaaaactttgagtgggttgcagactgagtggagttacttctcaagtcagacagtcagtgcagggtggtagtgctgatgggctccacctctgaccttggtcactgtgaaaaaaattaagaaggcttgtggaaattttgggattccatgtaacatcctctcataaaggaccagatgaaacgttgagggttgaaacacaatgtgaaggggatggtgtacctgctgcatttgtagcagtggcaggcagaagcaatggtctaggcccagtgatgtctggtaatactgcatatccggttatcagctgtcccccgatcacaccagactggggcgctcaggatgcgtggtcatctcttcgactgcccagtggtcttggctgttcaactatactttccccagaaggatcagctcagtttgctgctcagatatttgggttaaacaatcatttggggtgggccaaaaacttcgagtgagtatattgaacacctggatatccttaaagcaggctgataagaaaatcagacaatgcaatttgtgaaagagcaatttaatttttaggagaaaagttacttagtcctagtttggatttaaagaaaaacatcaggcaaaatcatttcacataagagaaaacaattcagtttactaatgaacaactggtactcagtgtttatgagttggcacattgtcagtggatattactaatattttctagctctacaaagtgatatgtccctgtaaccttagcactccagagcctgaggcagcaggatctctaagttagggccaccctgaactgtgtattgagactctacttgcaggggagggaatctttatagttagtaaagttggcatgacattactaaatttattctcaccctatgtatggtcattgttgggtaaatatgtaattatggaactggagcaatgtgtctgctatatgaaccactatgaagaaccagtatggctctcttgccgacaacctgtatctgtcagtttggttcccagtatctgtgtagattgcctcacaataaataacctttaactgaggggatctgaccccatggcttccttatgtacctgctctcaaatacatttccccacccaacacacaaaattttaaaaagtaaattttttaaaaagaatattgtttctaaatggtaatagcatttttttattaatcagtagataatgcttgatctgagttgatgctttgaaaattaaaatacttgtattgtgtataacaaaaaaaaatcactgtataataatgaaatcacaaatgaaaacatacagggggctggagagataattcaatggttaagagcacctgaagcgcttgcagaagactggactgggttcagttcccatcatcctcatggagagtagtaactgtctgtaactctaatttcaggggctctaaccttatgttctctcaggcactgggcacaggcctggtacacatacacacgggtgggtactcatacatacacataaaattaaataaatatctcttaaataataccttttaaaaataaggtgtaaaattataccatttttaaaagcattcacctagattaaggtagccactccaacaaagtatatattctagttagatggtttctggaatgagaggattcctcgtctctccgtcttctatgaaatacagacattttatcagtaaggacaatcctaaggagggtttggtttgaaatatatgtgaaattgactatgcaggagactgatagctagttgtatctattccacctgagcttactcacagggtcctgccaatggtattgaaaatagagaagatatgaaataaataccaccatgctacagggtaattattcatgaacagtcttctctgtgaatcccactgtcttttctataaaacagatgaaaccacttctctcacacagggtgtcatacaaataacaagaataaggtacttaaatctctggccctcaaaggattgggtgttctgtaagttgactggcaactgtgggggtgactggtggggagggcagagaaaggagggaaatataataatttagaaagataacgtattcaagaacagtcagttctccatttggaagccagaagcaaggtaaagaaacagagaatctttacaaattaggaagtgggataaattcaacatgaactagtcaggaagagaccacagagctgccttcacgacttccattctgctgtggtcaatctgccagagagagagagagaaagaaaaaaaaaaccttccttccagaaaggaacaataacctttactttgctgtcagccgccatttctccagggatggatgaggccattacccagaaaagacctgcagttatcacatcatcttcctccccatcaaatattcactgagcatcctctaaatgctggcttctggagacattaaagtcatgtccttggcttgtgagaaggtccattttgtctccagacaattctaggaaaggatgttgaccagccatacacatttagaaattctagactctctcctgctggcaaatgatatcagaacagtccactaaccatcaaaaagaaaaacaaacaaaaccagtgtgggcatccttcctcttactataggcagctctgcaaagctgaataaacagaggccccagggccagctgcacctctcagaatcaataaggccttgaccaaatcacttaatttcttggaatctcaagttcattatctataaaatagaaacatcacctagtttgctcatcttgcaggcttttttgtatgggacaaataatagaagaaaatctgttacagacaagaaactatttctgttttattttattttatttattttgtgtgtgtcatgtacaccatgagtgcatggtggtcaaaggacaacttgtgggaatttgttctagccttccaccaggtgagtccacaggtagaacatagatcatcacacttacaatcaggcaccattacccactgagccatattgctggctctttgtaattttgggattgggggcagtgcattttttacggtaatcatatcagaaccgcttcttgatttggcagtgacatacatgcatcatgagtagtagcaaaccccagtagcacagggtgagtcggtgaagtgaggagcagggcccagtatagacaaatgccttagtgctgagacatcaggatatggagaaaacgatgtcaaatggtgaacccacatccagtctaaagagaccaggttccaatcagctttccatctagtttcccttcttacaaagttcagaagtcaagactgcgaacatccaggaggccagtggcagaatctgtgtagcacacacaatggtcctggtaccacaaaagaaaagagggaaggggtatgagtctatactgaaacatctttcctctaaagaaaccatattgtatttggaagtgttatacacttgcgacctctaccaccaccctccattacacacagattctcacttatgaaaactgcttaattttcccccaccccgtttaggactggccgtctcttgggagtctgtgccagtgtggacaattgccgattgtttgtgggaggaatccccaaaaccaaaaagagggaagaaatcttatcagagatgaaaaaagtcacagaaggagttgttgatgtcattgtctacccaagtgctgctgataaaaccaaaaaccggggatttgcctttgtggaatatgagagtcaccgagcagccgccatggctaggcggaggctgctgccaggtaaaccatttctgaacagagactcccagctcgcagcagaccccggcatgagcgtccctgttgatcttcctttaagctcttcatcttgtttatttctctcacagttgattcagttccataaaatgcttatagatgtgtattttggaaaagtcacacctctgcacatcaggagaactttgatcgatagcaatcccacagagaatcctccgccaagaaactttttaagattaaagaaacccatcatggatatagctgcatgaaagttcacaggcgagttagaattgggctacgtcacctatcacggtgtacatgcacaccagtgtgtgacacaccatcccaaggatagccaacagacatgctttcaatgtgtttcccagtttgtaataatttccatctaattggagataagaaatatgaatgttcttcctgtacacctctatttagacacttcagacacacatgtgagcaccaatcatcaattcatgagtttaacgacacaccaacactgcctaggtcagactaacacaggcaaatatgcaaagttccttggcttgctgtaaaatagtcactaaaactcaacacagtcgaatgccaaagtgaattcataataatggaagaaactatctaaaccactagttctcaactgtcataatactgagattcttttttaatacaggtcctcatgttatggtgaccccaaccataaaattatttcattactatttcacaactgtacttttgttactgttaccactcattatataaatatctaataggaaggatatctgatgtgtgacccacaggatgacaaccagtgatctagagtgttaagtagctggcactagggtctactcttatgaaaactatgagagatggtgggggaggtggagcaggacaaaactctgacattattttgactttatacagtctttcctgactgtactttttgtctttaatggtggtgttgattgaacagagggtcttgagctacctaggcccacattctgccactcagctatgtccttgctctgtgtacgttccttataagttaccatctaaggcattgggtatcattgaggtatgttcagtgttttggtagactctcttccccctcatcttcttttctttacctcctctgattctcagacagttgtgagccttttgtaggctttcagaattgaagtcaagtcatctagaggggcagctatgctcctaacagctgggtcgtccctccagctcccgttactattttatatggtttagaatagaggtctgatttccacgtgtggattaggataccccactttacccagaaatcttttaaatattatttagcattgcctgacctcaaatctgagctcctccttcctctgcctcatcaggtaccactatgcccagtggagcctagtgctatttattaaagagactacactttctgtttgtatgcttttggcacattctgccaaagaccacaattgatttgtaagaaataagagggtggacatttgcttcccctatataataattacaccaatagatccttcccgacactaataataatttctgattttgttctgtccagttctgtgttccctacaagataaaacttacttccaaacagtgaattaaacaagcaacctatattttccacaacgtgtagaaactgagttgatctctcacccttatcccaccacttcaacctggagcctccagggatatggtggctgagtggcaaaatcccaaggggaaagaagtagtgataggaagttactgttcggtggcaatctttgaagctgtaaaagtgggtgctgccccccctccttccacaagatcatgataagaaagaactgagaggctcatgggaaacacacagcatagtaggaagcacacagtaagttactactgtagagtacatgaacctctgctgacttgggtcacacccactcttttaagacaacaagttacaatttcttatcatcaactgtcttcttagtcttctctgaattgggacttttaaatatacatatacatatacatatacatatacatatacatatacatatacatatacataacatatacatatacatacatatatgttttctccgagatcccacattacctgtactcagactgagaatgtggaaggagcacagttggtctgagccctggatcaaatctgagagaccgaggacaaagaatgtttggccccatccaccctgactcctttgtgtatgtctaagtgatggtgtctggctaatagcaacacattccaactaccgcggatgctgtccaaatgacctcttaatgtttcatggagtcagactctgtgtctgagccctaaatctgctctttctccccttttattatctcaccttaaaaactacctccccccccttgaaaaagctgtcctgagagtcaaatgcacccactgcgtcctcactctttctccatttcaaaccacatcctgcttctctctatacattcaatcattatgataagccactagtcacactgaagactctcctaccagactctcactttatttaaatgtctttaaaaatcaataataatcttttttagtggtcttggtcatttaagaccttaacggtcagttcaatttaaaagaccatcagcatgcgctgagatgatgtcgtgatttctaaataattcatcactcttctgatcattgtcaacaggaagaattcagttgtggggacatcctattgcagtagactgggcagaaccagaagttgaagttgatgaggacacaatgtcttccgtgaaaatcctgtacgtaaggaaccttatgctgtctacctcggaagaaatgattgaaaaggaattcaacagtattaaaccaggtgagacaaatgtgcaattacttcttcaaaatgtgctctaaaaacacttacaccagaggccattacccacaatgttaaggggaatggcttttatttagtcacttctcctaataagttgtatattgttcaggtatttcaccatttttggatccaaaagagacatgtggtcctacagcacttaggaatattacggtttatattcatctgtgtagttatttaatgaatacacatcacctctgccaaactaaattctatgagttcccctccctctagttagctcctctgtccctagaccctcagactaagaacaaagtaagcatttactatgttaatgatgtctggctcttactgaaggtctaacctctaacacacctttccttgggggcaaatcatccttggtagattgaacagggtattgaacaacatgcaaggtccatgccttcccctgccagctatagcaacctgcccaggtgacaaccaaaggcatctcctgacatttcctattcatctgtctgggaatgtcctcagtcacgccagcctgtgtctatcataaaaacatcttcatgcaactgagagtttcttgggcaatttctataggatgctaacaaacatgggccatgatgctgtcctactaagcctcctgacagtgtttacaccctcagattgtaagtggatgtgatgttacatgtgtattacaatggtaataagcatataactttttttaacagataattagcagggctatcatctttttcagttgagagattctcaaacatttctcttccttcaccctcaagaactctgccttccctgacattcagtatcatggtatggttggataccaacagctgtgaatccatagttactacagaaactgggagaaaaaatcctctagaattagagtgggcgtgttcacatcacatgccatttagcatcataatgccctatcctgcaggttcccaggtggtgtctattatttcctataatgcggaaaaaacagagctcagagtagttaaatcgtcacacagctagcagctggcagaccccagatttaacaaccttctgaaccctcacaccagccctcattcctttcacacattcattcatgcagctgctactccatggtagctccagcatgccacgcactgtgggaggaattaacagatgtgagctgtgtgtggaaggaatttatagagaagtcacagctaaattatcctgtgcttggaatgcagagcaaaatccgaagacaagtcactcatggataaatggtagacctcaatgcggttgtaccggactgggaactagtacatatgtgtataggtttcacctgctccaggttgggttttgtttttcccagagtgaataatacttactttcccttccaattaaaatggcacttttgttttgaaagaggttagagtgaggaaattttgaaagactaactcttttctttatagtgagtgattatcagtccagtctaaacatctaagataaatatgcctagactatagtgtcctagaagctgtaagaaaagaatgaatgccaacttgttccttcttgcctcttcttcctttctcttcccccatctcctcccatatcttcagttacaaccacccccagcataatactatattattattaaaacatttccagcaatcctggggcacttcttagaccagtggcacaccacttggggctctataataatgtgtgtgatgcctaaaagattatgaacaactagaagatatatatcctatggagatagagaaaaaaatccacaaatttcttctccagtgctacaatataatgtatcaagatcgtattgtggaaagagaaaaataacatcaatttttatctttttaaagatttgggtttttgagagacctcattattcattgaaaaaatctaactttgtatatcatttgaaccctggctgacctaggaaaatacggcatcaatattctggaaaacaaatgacagtggtcacagaaacctcgggccattgatactttgttctcatgaagtctagggtatctgctgtcgtgctaagtatggtattctgtaaaatctgttcccttaaacagctcgacagtagcagaatcttctgaaggactgctgaaagcaaattgcttaccccatctggttgagtgtacacttttgtattgcatttcttacatccccagatgcagctaatgttactgagtaaagaactaccctttgagcgtgttttcctaatgcctgttaggatcacagatctagttcttctttgccgatgggtaagccatgaataatgatagcaagcactgaggaggaatatcttaaaggaaagcctatggccattgctattctacagaagctagtcaaccctggatataaataactctgaatgtggtggctcttgtttggtctctaagtagggttagaccagggtagtatctagatgggagaaattcatgttgtattattcaaacccctactttctaatttactttctatgctcaaattttcacaaataaaactttccataagcagagtctgaatattgctgctaaaaaagcataaaaatttcaatattagcaagaatttcagaacacccaaatccattatcaatacaaatgctacataattaacatgactgatggggtatatggaaattgattgataatacattgtactctgcgtgtttactgagagactagctcttgtatccttgacactaaggaagaggtaagtgaaaatgatattttgtgcagctaaagcacacacacccccacacacaccttaggaaattatagctcagaagagcagagtggaggggaggggtggaggccgtcctggatgatccttggttctcattgaccaagacaagcgtgggatgaagccttctcacccttccctctaacactgaaggactaaggagattgacttaaaatctcagttaattatatgtcctgtgtttcatgggttcatatgcctttatttcattgtctcagtctggccagataattttgctctgtgtaattattgtcatgagacactaagggcttcttaagatactaaatggagagagaactactgcaatagtttaaaattacccttagatcacatgtctcccgttgttccaaaggaaggtaattcatggtcattgaaccatgtggtacagtatgagctctaaatgctccagtgaccctgaatctagagagcaactgaactctgataggagagggattttcttttcttctgattcttcctgagaacagtgttacgtcatgttgactctgagaactacatgttaaatgaagaaacccagcctttcaaaatcacgccagctgaaagataagcagtgtgctcaggtggtagtgaaacaagattccacttaagcatcaactaaagaaaacatttgtttggaatactgagtgggcaacaatatgcttaaaagataacattaagcatctttgtccaactaattttttaatatgttatccttaatatgtacaacggcctttctacataaaaataggagaggttgctttctggatgtttgtacacaataaactaaacgaatctttataaaagcaggtcaattaaaatgttttcatcgtgggactggaaagcttgcactgttcttgtagaagaccccaattcagttgctagcattcatgtcagctggctcactaatgcctgtaacttgaactccctgggatctatcaccaactcctggcctccacaggcccctgcactcacatgcacataactacacagacacacacagacacacacacacacacacacacacacacacacacctatatttaaactgctttcactttccccaacacttttctctgttcctgaagtaagcatgcttagcagtttgatatccacctttccaggtatctctgaatataggtggacttgagtttgtctgtatttacatgtgggttgagatatttaaaacagatactcacattttatcccattataaattttagaaacagacaatagggtttcgatctttccatgtaagtgcatataatcttactctaatctctttaaagactcaatggagcttgtaaaaaaaaaagcctcaaattgaatcatttctgagaaaatgggtcttgctttactttgttatttctacggtggcctaaccagcatctttatagaaaatccttgggcataggcagaaactcttcagtataagaaacaagttagaaatagtagataagtagttttttccaatctttcaaatactacatgcagaaactattttagatttcagatgcactttcagaagcaaaatttttttttggggggggggaggttcgagacagggtttctctgtactggaactcactttgtagaccaggctggcctcgaactcagaaatccacctgcctctgcctcccaagtgctgggatttttttttttagatttatttattattgtatataagtacactgtagctgtcttcagacaccatgattactactttttttttcatttttattaggtatttagctcatttacatttccaatgctataccaaaagtcccccatacccacccaaccccactcccctacccacccactccccctttttggccctggcgttcccctgtactggggcatataaagtttgcgtgtccaatggacctctctttccagtgatggccgactaggctatcttttgatacatatgcagggggtactggttagttcataatgttgttccacctatagggttgcagaaccctttagctccttgggtactttctctagctcctccattgggagccccgtgatccatccattagctgactgtgagcatccacttctgtgtttgctaggtcctggcatagtctcacaagagacagctacatctgggtcctttccataaaatcttgctagtgtatgcaatggtgtcggcgtttggatgctgattatggggtggatccctggatatggcatgcgccaccacttaaagatgttaattaagtgcatgggacatatgattctagaccttaggtagctgtgtgctgtgtgaattacgaacccacctttgttagaactgtaacttcctcgtatttatataaaagtaaaataaaattaactacacagtaattagcacaccacattggtaacagctcccctaattccagatttcctttatagacaataaaatagggtattctcagcagagtgagtgtggcgtttctaatgggagtgagttaagtctttcccacgggcttctacagtgcccagaatcacactccattgacaccatgatcacttaccagtaaatgacttcatgaaaagcctaggaagatagtactgagctgtgaaggaagtgtcgcagataccacatcacattctcgtgacactggatattcatgtgacctgattttccttctcctcccagcagacctggatgaatgttccaccatagggcacaggatcaggcactccatggagccacactaacacataatagggggtgggagggaagtcagagcagctaatactcctggctgaaatctgaccaatgggaatagactgggaaattgtgacttctctcatgcccatggatcattgcacagtattgtttcttctccacaacagagactgggggaagacttcagcagccaggggatatacccgcccctcctctccaaggctgcatgagtagttccctgttcatccccttgctctagctgcaattgcactttctcacttctataccttggttgaacctccaagatacagcagtattttgaagtaaatgagttaaataaatatatgtgtataagatgaccaatatcaagctagtttagtaacacattaccttgtatagaatattatcttttatatttcttaccatttatatttttatatctccttactaagatattattctttcctttaaatttcctataatctagggttagaaaacttagttcataactataataaatcaggttatcagcattttatgacaagtaaaacttatattgactatacagaattctaaacctagacatgcaaaggaatctcacctttggaagctagccacatcactgtctcaagtttgctaatagtgtggctgccatcacatcaacaggtgctgtggaaagagtgaagaagatccgagactacgcttttgtgcacttcagtaaccgagaagatgcagttgaagccatgaaggctttgaatggcaaggtaacataagaaagaagaaaatgcacacagattccatccatacttggtataggcatgctctaccaaagagcaacctctggagtccatgttagcccagctctctttccccaccactgacccagaagctgcagtcctttggggatgtatggtagagaaatggcaggaaaagatacagagaacaaggaagtggctaggtagctaattctaacacttacttctttttatgacttgaacaagtcactgacctcaatttttatgtgaagaagggattcaagtttttccagatgttcccctagtccttgctagtgttctattgctgtgaagaagcaccatgaccaagacaactcttataaaaggagccatttcattggggtcttgtttatagtttcagaggtaagcccattatcatcgtgacagaaaacatgccacggcaggcaggagagtagctgacagctctacatcctggtctgcaggcatcaggcagagagagacacttggcctggtttgggcttttgaagcctcaaagccccagaaacacacctattccaacaaggccatatcccctaatccttccaaacaggtcatcagctgggaactaagaatacaaacacatgagcctgtggtgcacattctcattcaaagcaccaaatcccccaagttccaattatttctaattattgtactttataatgcagtgatttaaggaacattagccagaaaaaaaggtcaagatagctgtcattctgatctcggtatttgatcttcccatcagggaggcttagaattcctctccttgccaggagtgaaggctaggagaaagtcaacttctaaattcctactgtttatttgtctacagaaacttatatagtatttggatcaatcaaataaacactaaaacccatctatgggccactgatctgtaatgtccagaatgcaaaattaatcaaagcaaagatgcttatcaaggggttgagagtctggagaactaagttaggcccacaaacacctctgcaaagtagtcatctcgatgttattaaaagacatttaaaagtaagaagttcaaatgaaggagttaagaaaaccatcatgtattagaagattctgtgcaaaagaatgtatgaaaattggatatactttcaaagggtaagttatggcaaggcgagtctgtcctaatggcagaatttgaaatattaagaaaacatctgaggacagcaaattgggtcagagcacaaggcccaggagactgctctgcccattaagtcttctatcagttcagattccagtagaggttgggtagaaagaaattcactccacaaatgtgagaaagggggattcaaaaatagagtgtcagagactagcactcatagatttctggcagtaaatgcacaatagaaagagcaggtaagaaatgcctcagcaagttcatctgaacaatgcttttaataccctccacccttgccccattggtcccatgaaagcatctcttctaaacagcagttaccaaatggttgtcattgcctgcgccagcaatccttggtctctcaggcagtcctcaacttgatcctcctgcaggagatgcttttgaggttgcacggcccccagggttttcacagaggtggccaccactgttcctcctacaggagctgaagagttagtagaagcccaaaatgagaccaagctgtagagagcaagccccttggaaggaacactgatctagaatcatactaccctgaggctgctgggtcttctccaaaaggtcttgggttaaatcatatagtgggtccaagaagacatctcagggaggaaaccaggaaaacacatcctaagttcactgtaggctcatccctatcctcatagccaaggttcctgttagccaaactcaatgacaaatcagagggcccaggagcccgaagaggaacttatatagaataatcaccagaacagactgcagactggacactggtgggaactggatgtgaagaacaagggaaaggcttcccagcagagattccttcagacctgggggagcatagggcatgtgcaatcttagggacctgcaacactcttgtagggaattaagcggtgactctgaagtatatatatatttaaacaaaactgtaaagtaaaatcaattgatatttaaatgtaccttatgaccaaaacgttatttcttcattgattactatattagcatctttagaatgatactacttcaaaaacaatgtttaaggtagctttccctcaataaacatgcctgaattcgcatggtatctactgagaagaaccagcaaccataaacccagtgctaaataatacactggaataattccagttgaaaagtgtttaagttataccaatttttaaggctttgttttggcttaaagtttaagaaaagatagtccattttggatcccgggattccggcaaccagatgacccacgctaccgtctcgccagcaaaacgcacgcaggccactcggatccttctgcagtacagctttaatatatctcgagagatagatgatcagcttcaggagaggagacccagagagcagaaaacccttcccttttatttatttttttttcctttgaactgttttattgagataattataatgacaaaaagtcataacttacacagacctatagtaatggttgtgacttaaaagtaatttaaaaataaattgttcatcaaagtttcatattaacatttcttttttttttcattttttattaggtatttagctcatttacatttccaatgctatacccaaagtcccccatacccacccaaccccactcccctacccacccactccccctttttggccctggcgttcccctgtactggggcatataaagtttgcgtgtccaatggacctctctttccagtgatggccgactaggccatcttttgatacatatgcagctagagtcaagagctccagggtactggttagttcataatgttgttccacctatagggttgcagatccctttagctccttgggttctttctctagctcctccattgggagccctgtgatccatccattagctgactgtgagcatccacttctgtgtttgctaggccctggcatagtctcacaagagacagctacatctgggtcctttcgataaaatcttgctagggtatgcaatggtgtcagcgtttggatgctgattatggggtggatccctggatatggcagttcccttttataggctgcggaaaaaaacggttggagacgtgcaccactgggattggctgcccactatcagtcagttaacgccacgagagaggcagggctcaggtaatggaacgataccctgacgcctgcgcacattttactacgagcaacggatgtcagcgccatcttgtaacggcgactgtgagggcggctcctcacagtggggaagggatgaagatgaactagtccaattctttccagacttacagacagaaacaatggcattcattataatagtctctatgccaagttcaagtggctgtattacgttgggatgttttaataagcacatacagaatattatgactctaaaaaaaaattaataaacatgtaactaaaaataatttaaattacttttttacttatcaatccccttatttatactgctttcttccaaagtagctggaggcagtggtaggagcatgggatagctagtcacactgtatccagtcaggaagcacaattattatttgtttattacatagacacttctataacaatgtttacaaatatgtagctaaatatgtgcttaatattactagtaaatactattaattgctatttttaagcataatataaaaattatgcattagtttttctaagctgagtattatacctatggttatttctatttattgggaaaagttagactttggggatcttttgtaagatagtgatcatattacaatttaaggagaccagtgaaatgccacacggtggctagcaagatcactcctttcctttaaaaaaaaaagatttattaattaactaactaattaatttgggtacttaagatagagaggggtatctctttgtaggctagactttctgggaactcactatatagatcaggctggcctcaaactcacagagatcctcctgtctacctccctagtgctgttattaaaggtgtatgccacctgtctgactattgcaataattcttaagggcattttttgattttttaattagatattttatttacttacatttcaaatgttattccctttccaagtttcccctccacaacccctctatcccctcccccaccccttaattctatgagggtgctcacccacctacccacccactcccacctcactgccctggcattctcctacactacaacatcgagccttcacaggaccaagggtctctcctcccattgataaggccatcctctgccacatatacaacttgagtcataggtccctccatgtgtactctttgtttgatggtttagttcctgggagatcttggggggtgggggtgagctctggttggtttatattgttgttcttcctatgagttgcaaaccctttcagctccttcagtttaaggccatattttaagtggtatctgacttgagagttctcagtatccctattacccatatccctgacatccctatttagaaacttccaacttcacagtagggcaaacatttgcaacagtctattaggttcctcttacttgagtagaatctaactaggctcctctccctccccaggaaaaacctattaggtagctgccccaatgagccttttgttgagcctggcaagacttgctcaccagagtgttaataaatgataagatatctttactggtttctatcacctgcattttatcatgctgcataacctgtatttcacccataaagatccccagctgtttatacttacataactatgtgattttaaacacaagtatcacacttaatagaaaatattgcattattttatgtctcagtgactgtctctgtattataaattcagtagtcaaaatggcattgttaaacctgttgccattctgtgcaactatcaacaagactactcggagacttgtcaactgatacagaagcatgggcttggtaagggacaaaaccattaagaattgcaagtcaccagatggtagaccaagacttgtcggatgacttctcagcttggcttttcctcgctctctacaacaggtgctggatggttccccaatagaagtgaccttggccaagccagtggacaaggacagttatgttaggtacacccggggcaccggtggcaggaacaccatgctgcaaggagaatacacctaccctctgagccatgtttatgaccctaccacaacctaccttggagctcctgtcttctataccccccaagcctatgcagccattccaagtcttcatttcccagctactaaaggacatctcagcaacagggctctcatcaggaccccttctgtcagaggtaacactagctagttcttgtctttccataactcctaaatttcaaatgactaataaactatagtttagatgaactacaaactatatagtccagtagccttaaagagattgggtggaatcactgggagaagaaaaaacaaagagtagaaaactgtttttaaaaatcgaatatatcagctctcttgtcttatagaatctattttttcacattacaactggttaatggaaatacttgttagaacatgatatgaatgagcccaatatataaattagatccttgagggatccacaaagtatgcacagaatacaacctagctctataaccattgctccttcctccttattagaccaactagactgattacagaactttcaagggagagcagtattgcccgatgagaacctcctccaatgaggcattctcatatgaactccttcgcagatatgtgtgtcatacctttgttattccatgtgtttgtctgcatgttggtcacaagggcttactgtatattccaggctgctcaagaactcccagcacctgcctctgcctcctgactgctggagtggcagatgtgtaacccatctatgtgcaatcactttgaaaagaggcaagcaattaaagaaattaatatttctttagtataacggaaaccaaatacattgaaataggaaaaaaaactcaatctcactgccagactgtgttttatgtttaaatcctacacagattcactgtttgagcaggtttctgagctcagaaaattcagtaaatgataaaagtaatttgtctctgcctcgtggtatcttttattgtagtttttcttggttatagccaacattatatggatcccaatcctgtattccacatatgatagcaatctagaaaatagaaacaaatgatgtgtttaaatacactcttgggtgtaacttagaataggcccatccagattactatggaaacaattctcttactatgttacattatttttcatgcgagaaaaatgaattgaacatcttaggacctcttcagcggccttaaaagttcttttctcacaatacaattacccctaccaaaattgttatacaaaagtgacaatgaagattttatttgaaatactttctatatattataacaaattatgtcatgagacattatgcatatatatgtaatgcatcattaattaatccatatatcttacatattatcaaccagttgtgatgtagaaaaaaagcaaactccattccaaggggaaataatacatagcattaacaaaaacataatcaatgaatgatatttatgggcaaagagtgatccacttaatggtttattcctttatgtcaccaataggaaatcactgtagaaatatggataaacaaaaatcaaaaaggaatctttgtatgaaactcaacaaaattaagcctcacatcaacagtgcataagagagtggtgaacttaaggaaaagatgctgcctaaatgtttgtctgatgacccaccatggatgtgataatcaaattcacttctgccttactggcagagtggcgtttactctaatagtctctatcccagactagctataattcaaatagtcatattatgttgggatattttaataagagcatactgaaccttagtgattctgccaaaaattttaacaaaactagttaaaaaaaattccttctgacaaaatttgaagcggctgtgtacttatcagtctccttgaaggaattacttccttccgaaggagatttaagaaagcaagagtctatggtgagccttacctgaggttgagtttcattacattagaatcttaggttatctgtccgtcctccgggcaataatcaatgtgacttgggctaacttttccattacagtaagattcagggtgattttctggtggtttgattcagtttaatgccttcttacattcctagaaatttacatgaatgtccctgtaggggctgcgggggtgagaggactgggcggccgtgggtatttggcatacacaggcctgggtcgaggataccacgtcaaaggagacaagagagaagacaaactctatgatcttctgcctgggatggagctcaccccgatgaatactgtctctttaaaaccacaaggaattaaacttgctcctcaggtaggtaagatcaatccagaggttgttaagttgggccagggaccattgttccgatgtttagagttggtcttaatttgggatactgttctgtggcaatcagcttagcatacttcgagtggctgctgagaccagctgccattctggcactaaatggaacttcatcaagacccagcaaaatgaaaaacaaacgaaatcctgaaaaggaatgtcaaccaaaatcttaaatgaaaataaaacaaagcacagacccccagttcagaataaatgtctactaaagcatgttggtcaacaactgcagagaagaggtcccagagccggatccctctgtgtactgtttaggactattcttggaaacatggcattaacagaacaacaccaatagacatgccaaagtggatgggtaaaagtcctgtagaccccaacctccaaaaaaaaaacaactatagcaactgagtaactgtagccaagcagagagcaggaccctttcctagagtagagcacaccaattggttgtctagtaccaaatggaaaatggtcagccctgaaatatacatacaagtaacatatatatatatatatgtgtgtgtgtgtgtgtgtatacacacatatatattcacatacatatatatatatacatacatatatatgtacatatatatgtatgtatatatataggcatgcaataacaattaatgaaaaaagagacaacgaatttgaaggcaagtagggagggcatgtgggaggctttggagaaagggaggggaagggagaaatgctgtaatgttctctctctgtgtctctctctctctgtctctctctctctctccctctcctttttattaaaattgtggaagacatttgaccatggaacctaggtaaatcctaagctgtaaaatgccatcaataatataaagccttctgtactggggcagcgttatgatggtaattgtgtcatgatggtatctactattgtgtttctatgtttctgtgtgactgtctttttttttaattggtagaaactgaaagttatcattattattccaatagggaaactacatactatgtttgataaattaaaacaaagcagactcaatccctcttaagcccaggttctcctattactgtgcatcacctgctctccctccgaagccactttggccaaatagctatagttatagtctgtggattgtaagagaaacatccttgctctcaggaggaaggtagaactctgatttaagaaaacaagtagcctcagagttgcaaccaagagccaaacaagcatggtatttggaagcttgaccagagaatgaaagcagaggcattatcaatgatttcttttatgtaactttaagtaatttttcttaaagtatattcaggcaatcagaaaaataaataaataaataaataaataaataaataaataaataaataaacagaggaatgtctatccccatggcaaacaggaatcattagtttcaaaaataacacttattgagcttttctggtgtagcatggttgatagacactagagatgcaaaaatgaatataacaaagatccttgcaatcataaatcaagtagctacctagtgggataatactgacatatgaacagacgattacaaggaaatcaggtaagggtcgtgatgaaaatattcgttctgagccaaactctctgcttacacattaaagctagatgagagggtggccataatgagagatgtttgactaaggtgtctctaaattttttcaaatagagttgaaaagttgaagaccaggccaccccagccaatgtggctggagactaatcccccgttagaggtaggaatcgccaaccagtagacagggatcaagaagtgccctgcccaagaggaagctgcagagaccatgcctctcagtgtatttgttcaactggcataaacaggcagacagcctatctaccagtcaaaagctaactgatatggctcgggaaccttagagcatgcttataaacgatgggagatgctactaagtgctttttaataatggaaaaataacttcagtcttcaaattaataaactggaaaatgtcatgggagacagcagctgtattaatgtgagaacaatgagctaggaggctcaagagaactgggcttggggctgggggaggcactgttcctttatattttatatgcagtttcttctgacacagatactaaatctactatgtgaatggagggtcaggacctcagcatccccaaaactttagggtaaagtaaacatgaatcttttgggggaaatctctgttcagggtgattacaaaagtaaagagtaaataagagcagcggtcgccatcttggtccgggacccgccgaacttaggaaattagtctgaacaggtgagagggtgcgccagagaacctgacagcttctggaacaggcagaagcacagaggcgctgaggcagcaccctttgtgggccggggacagccagccaccgtccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccgcccggctggggaggcggcctaagccacagcagcagcggtcgccatcttggtccgagacccgccgaacttaggaaattagtctgaacaggtgagagggtgcgccagagaacctgacagcttctggaacaggcagaagcacagaggcgctgaggcagcaccctgtgtgggccggggacagccggccaccttccggaccggaggacaggtgcccacccggctggggaggcggcctaagccacagcagcagcggtcgccatcttggtcccgggactccaaggaacttaggaatttagtctgcttaggtgagagtctgtaccacctgggaactgccaaagcaacacagtgtctgagaaaggtcctgttttgggccttcttcttcggccaggaggaggcccaaatacaagatatctgcgcaccttccctgtaagagagcttgccagcagagagtgctctgagcactgaaactcagaggagagaatctgtctcccaggtctgctgatagacggtaacagaatcaccagaagaacaatctctaaacagagtcaactataactactaactccagagattaccagatggcaaaaggtaaacggaggaatcttactaacaggaaccaagaccactcaccatcaccagaacccagcacacccacttcgcccagtccagggaaccccaacacacctgagaacctagacctagatttaaaagcatatctcatgatgatggtagaggacatcaagaaggactttaataaatcacttaaagaaatacaggagaacactgctaaagagttacaagtccttaaagaaaaacaggaaaacacaatcaaacaggtagaagtccttacagaaaaagaggaaaaaacatacaaacaggtgatggaaatgaacaaaaccatactagacctaaaaagggaagtagacacaataaagaaaactcaaagcgaggcaacactagagatagaaaccctaggaaagaaatctggaaccatagatttgagcatcagcaacagaatacaagagatggaagagagaatctcaggtgcagaagattccatagagaacatcggcacaacaatcaaagaaaatggaaaatgcaaaaagatcctaactcaaaatatccaggaaatccaggacacaataagaagaccaaacgtacggataataggagtggatgagaatgaagattttcaactcaaaggtccagcaaacatcttcaacaaaattattgaagaaaacttcccaaatctaaagaatgagatgcatatgaacatacaagaagcctacagaactccaaatagactggaccagaaaagaaattcctcccgacacataataatcagaacatcaaatgcactaaataaagatagaatactaaaagcagtaagggaaaaaggtcaagtaacatataaaggcaagcctatcagaattacaccagatttttcaccagagactatgaaagccagaagagcctggacagatgttatacagacactaagagaacacaaactgcagcccaggctactatacccagccaaactctcaattatcatagagggagaaaccaaagtattccacgacaaaaccaaattcacgcattatctctccacgaatccagcccttcaaaggataataacagaaaaaaaccaatacaagaacgggaacaacgccctagaaaaaacaagaaggtaatccctcaacaaacctaaaagaagacagccacaagaacagaatgccacctttaacaactaaaataacaggaagcaacaattacttttccttaatatctcttaacatcaatggtctcaactcgccaataaaaagacatagactaacaaactggctacacaaacaagacccaacattttgctgcttacaggaaactcatctcagagaaaaagatagacactacctcagaatgaaaggctggaaaacaattttccaagcaaatggtatgaagaaacaagcaggagtagccatcctaatatctgataagattgacttccaacccaaagtcatcaaaaaagacaaggagggacacttcattctcatcaaaggtaaaatcctccaagaggaactctcaattctgaatatctacgctccaaatacaagagcagccacattcactaaagaaactttagtaaagctcaaagcacacattgcgcctcacacaataatagtgggagacttcaacacaccactttcaccaatggacagatcatggaaacagaaactaaacagggacacactgaaactaacagaagtgatgaaacaaatggatctgacagatatctacagaacattttatcctaaaacaaaaggttataccttcttctcagcacctcatggtaccttctccaaaattgaccacataataggtcacaaatcaggcctcaacagattcaaaaatattgaaattgtcccatgtatcctaacagatcaccatgcactaaggctgatcttcaataacaaaataaataacagaaagccaacattcacatggaaactgaacaacactcttctcaatgataccttggtcaaggaaggaataaagaaagaaattaaagactttttagagtttaatgaaaatgaagccacaacgtacccaaacctttgggacacaatgaaagcatttctaagagggaaactcatagctctgagtgcctccatgaagaaacgggagagagcacatactagcagcttgacaacacatctaaaagctctagaaaaaaaggaagcaaattcacccaagaggagtagacggcaggaaataatcaaactcaggggtgaaatcaaccaagtggaaacaagaagaactattcaaagaattaaccaaacgaggagttggttctttgagaaaatcaacaagatagataaacccttagctacactcactaaagggcacagggacaaaatcctaattaacaaaatcagaaatgaaaagggagacataacaacagatcctgaagaaatccaaaacaccatcagatccttctacaaaaggctatactcaacaaaactggaaaacctggacgaaatggacaaatttctggacagataccaggtaccaaagttgaatcaggatcaagttgaccttctaaacagtcccatatcccctaaagaaatagaagcagttattaatagtctcccagccaaaaaaagcccaggaccagacgggtttagtgcagagttctatcagaccttcaaagaagatctaactccagttctgcacaaactttttcacaagatagaagtagaaggtattctacccaactcattttatgaagccactattactctgatacctaaaccacagaaagatccaacaaagatagagaacttcagaccaatttctcttatgaacatcgatgcaaaaatccttaataaaattctcgctaaccgaatccaagaacacattaaagcaatcatccatcctgaccaagtaggttttattccagggatgcagggatggtttaatatacgaaaatccatcaatgtaatccattatataaacaaactcaaagacaaaaaccacatgatcatctcgttagatgcagaaaaagcatttgacaagatccaacacccattcatgataaaagttctggaaagatcaggaattcaaggccaatacctaaacatgataaaagcaatctacagcaaaccagtagccaacatcaaagtaaatggagagaagctggaagcaatcccactaaaatcagggactagacaaggctgcccactttctccctaccttttcaacatagtacttgaagtattagccagagcaattcgacaacaaaaggagatcaaggggatacaaattggaaaagaggaagtcaaaatatcactttttgcagatgatatgatagtatatataagtgaccctaaaaattccaacagagaactcctaaacctgataaacagcttcggtgaagtagctggatataaaattaactcaaacaagtcaatggcctttctctacacaaagaataaacaggctgagaaagaaattagggaaacaacacccttctcaatagccacaaataatataaaatatctcggcgtgattctaacgaaggaagtgaaagatctgtatgataaaaacttcaagtccctgaagaaagaaattaaagaagatctcagaagatggaaagatctcccatgctcatggattggcaggaccaacattgtaaaaatggctatcttgccaaaagcaatctacagattcaatgcaatccccattaaaattccaactcaattcttcaacgaattagaaggagcaatttacaaattcatctggaataacaaaaaacctaggatagcaaaaactcttctcaaggataaaagaacctctggtggaatcaccatgcctgacctaaagctttactacagagcaattgtgataaaaactgcatggtactggtatagagacagacaagtggaccaatggaatagaattgaagacccagaaatgaacccacacacctatggtcacttgatcttcgacaagggagccaaaaccatccagtggaagaaagacagcattttcaacaattggtgctggcacaactggttgttatcatgtagaagaatgcaaatcgatccatacttatctccttgtactaaggtcaaatctaagtggatcaaggaacttcacataaaaccagagacactgaaacttatagaggagaaagtggggaaaagccttgaagatatgggcacaggggaaaaattcctgaacagaacagcaatggcttgtgctgtaagatcgagaattgacaaatgggacctaatgaaactccaaagtttctgcaaggcaaaagacactgtctataagacaaaaagaccaccaacagactgggaaagaatctttacctatcctaaatcagataggggactaatatccaacatatataaagaactcaagaaggtggacctcagaaaatcaaataacccccttaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtgagaatggctaagatcaaaaattcaggtgacagcagatgctggcgaggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagaagccccaactggtaagaaggacacatgctccactatgttcatagcagccttatttataatagccagaaactggaaagaacccagatgcccctcaacagaggaatggatacagaaaatgtggtacatctacacaatggagtactactcagctattaaaaagaatgaatttatgaaattcctagccaaatggatggacctggagagcatcatcctgagtgaggtaacacaatcacaaaggaactcacacaatatgtactcactgataagtggatattagcccaaaacctaggatacccacgatataagatacaatttcctaaacacatgaaactcaagaaaaatgaagactgaagtgtggacactatgcccctccttagaagtgggaacaaaacacccatggaaggagttacagaaacaaagtttggagctgagatgaaaggatggaccatgtagagactgccatatccagggatccaccccataatcagcatccaaacgctgacaccattgcatatactagcaagattttatcgaaaggacccagatgtagctgtctcttgtgagactatgccggggcctagcaaacacagaagtggatgctcacagtcagctaatggatggatcacagggctcccaatggaggagctagagaaagtacccaaggagctaaagggatcttcaaccctataggtggaacaacattatgaactaaccagtacccctgagctcttgactctagctgcatatgtatcaaaagatggcctagtcggccatcactggaaagagaggcccattggacacgcagactttgtgtgccccggtacaggggaacgccagggccaaagggggggagtgggtgggtaggggagtgggggtgggtgggtaagggggacttttggtatagcattggaaatgtaaatgagctaaatacctaataaaaaatggaaaaaaaaaaaaaaaaaaaaaaaaagagtaaataagcagacaggtaacaaaaacaggttctcgtaaaaatagctaaaagtggcacattgccaactcaataaaacaagtcaccgctgtgaatacggactaatgttaatctgtaaatactgccccaaaggatggaaaggtggatcagtggttaaaaacatttgatgctcttgaaaggatgcagtcagatccctaacagccatatacagtagctgacaactgtaactccagttccagattgatgccctctgtgggcctctgagggcacatgcatgtgatgcagatacacccaggcaaaacacttgcttgtatgcataaaataaaacaaatgaaataggtgagaaaacaaatgctaacccataagacctcctgatttttttttcaatgtgaagttaaaaaaaaaatttatgaatggcaagaagtaaagatatgttctcaagttaactgccttaaccataacaataaccataaccataataatcctaaccctaaccctaaccctaaccctaaccctaacctaaccctaactctaacccttgaaccctaaccctaaccctaacctaaccctaaccctaacccttgaaccctaaccctaaccctaaccctaaccctaaccctaaccctatgaacaagggcttaaatggagataagagagaaatccacccacatagtgctacaaccacaacttcctaaaaactggaataaatacacacacaaaaagtaaagttaagctcttggtaatacatgtgcattcatataatgtgtgcatgtatacacacacacacacacacacacacacacacacacaaacacacacacacaaacacacacacacacacacacacacacacacacacacacacacatatatatatatatatatatatatatatatatatatatatatggactaaactaaaactactagagtaaatggtgtgagaaaatgattttatgaactataataaatgttcacagtttaaatggagtccaggttaagttcaacttaaattttatgagtcattacacctcatgacatttttctctcttctattagttgaatctgaagttcagagctcgaagcctaaaatctagagaagcaatgcaaatgtgacatttgagggagaaaccgttgtttttctcttgtagatattagaagaaatctgtcagaaaaataactggggacagccagtgtaccaactgcattctgccattggacaagatcaaagacagttattcctctacaaagtaactatcccagcgctggccagccagaatcctgcaatgtgggtaccttctccaagctcctagtaactgaaattttagataaaattagcctgggtattaactgtgtttgatcatcactccaataacataaccagcccactcccttgtaattaaataaaacagcaggcactcgctcggctggtatttatgcaccagctgttagaccggtacctaaaagccacaaagtctcaaacatcaggaaggctctctaggataactgactaatggaaaggagaggtgattagagcctgtaagtggttccttcctgcagtgtttaggttgatataggtgacctgtgtaaagttgccacttcaccgtataaccctctgtcccaccttttaaatttcatatcaaagacccgatcattaaaagatatcaccttccagattctaccaccaggctgcatttatatatatttaggaaccatcttttaccttagaagagtcatgcagcaaaaattacatgtgttcccaggaatatcatctgctaccgaggtgctggatcaatgcacaaatatgcctgcgtaggcttatatgtagaaactttttacaacaaacaggaaacacacacacacacacacacacacacacacacacacacacacacacaaaatcccatcatccaatcatctttaacctgagtacttaaatttctgaataattaacattgctgactgctttccagccaccctttcataccaccaaagctaagcgcctatgtggatgaagcgaagaggtatgctgcagagcacactctacagacactaggcatccccacagaaggaggggacgctgggactacagcacccacggccacttctgccactgtgttcccaggtactggggagcagggatcagtcacgccagaatcagcaggctgcagaatgtctggggggaaggggagagcttctttgtagaaccctcaactacatgcagacagaaaagagagggggaaacagaaccaaagcagcctaagctggttggcatctgtctgcctcttctttgaatggccgcgggatcctgaatgagtcagcacccctgagactgttgacttgctgagtctgaattggtgtgaattggcttttactattattaagtgctttggtactactattagctggtatttttctgagctctagctatgaactgggccctcactaaagtgctcagcaagctacatattccccccaacgacccttatggtgcccagaggtcagggtcagctgctttgctccattacactctacctcatcttttttaaatgaaggaaggatatgtgtgtgtgtgtgtgtgtgtgtgtgtgtgtgtgtgtgtgtgtgtgtgtgtgtgtttacatgagtgcaacttcctgaagaggccacaggtgttagattcactgaagctggagttgtaggcagttgggcagccaacatgagtggatcctgtgtgcaaacagtatgtattcaaccacaagccatctatctctccatcccctttatctcatcctttgagacagaggctatcactgaacctggtgttaccagaaagactggaatttctttccaggattcttattgtttaatacaaaaattttaaaacagattaaaataagcttgaggacagttaagttatagggatagatagacagacaaatagacagacagatgatggatggaagatagatagatagatagatagatagatagatagatagatagatagatagatagatagatagatagatagataggcaggcaggcaggcaggcaggcaggcagataacaaataggtaagcaagctgtaatctcagcagctgctcgaaaggcaattgaaaaggaaggaaagtaagccaagctgtttgccctggaaatccgcaaaaagccacgtggcaaaaggaattgggtgggactgcttcagaaaaagtaagaaagccacagtaccagggaaagcatgcttagcttatatccaaacaacagagaaagaaaaggtatcatttccattggttaaataagatagcttttacagttctccgtgtatccatagagagaccctgtttcaaaacctacaaagagcaaacccaaaaatggacaactaaaaccaatgcttttctacacaactaagcccaggtgctggatccccttggagcacattttcaacatacatactcacttgttataagcaatatgttaattctatagtactgctaaaagacaacaatgtgccaaagagggagggaatctgaatacttttcataaaattgtctctgcctcttcctccaggatatgctgtccccagtgccaccgctcctgtgtctacagcccagctcaagcaagcagtgacacttggacaagacttagcagcatatacaacctatgaggtctaccctacttttgcactgaccacccgaggcgatgcatatggaaccttctaacgatccctgtcctctaagaataagcctttatagaaggatctgtagaagatacgaacctctagccagatccctcatgaccacaagtcataccttcccccaaagcaatgcctttcctgagaccacaagatttattcgtcgtattactcatgaaatgtgaatattattcttcaacaaaatcaaatgttaagcagaaacctgaagatccacggacagcaaaagttgttcttccaaggatcctgagtgtctcaggaagaggataacaccctgacaaattagcatatgctcttagtacagagagctgggcagtccgtagtaaggcacagccagtaaaatgcccatcttagtcactggctggcagctgcttcttcattcacagaacttagacaccagaggaaagcaggaagctctgtaagaggccttcagcattgcatacagtgtggagtatgatcccaggctgagctcagcaaaactgtctgaaacattatagattccttctcacttttggtctggacattccacactgttcgcctgtcataggtcagcatcctcctgtccctgcaacctctggggactttctagaaatcagtacaatgtatttcagatatgcttgagacaaatccaagccctgtatttattcaactctttaagccactgttttccttcagaatcctttccctgctcaaggattcccaaatcacagaaaccatgccagtaccaagttctagagtgaaagatgactttttttggggggggggggtgtcaggaaattctggcttgtccatactcatttgctttcatctcagactcattttgctgatcccaaaaactacaaagtaaatcagatctacataaaaatcactatgatattttcataatcagaaacttgaccaaaaagtcatttcaaagagaaaagtgtttggcttttgtctcgctgggtagcccaggctcaccttgatattgatcttgtgatcctcctgcctctgctccccccatgctggtgttgtttacacacaacaccatactcatctccaagaagttgcattaaaattgaggttacctttgagttctctacccagttcatattaatctaccaaaaatcactgtccatcccatcttttgattgtctttcaatatgtgcagctggctttactatgactgagtcccttgatagtgaaactattccaggaaagccttacacccatcatggttgcttagagaccatcccttttagcattcagtaaaagtgttgctttccaaaatgtcccaatttagaccatctgatagtgactgggggagggaggctatatacatgtaaaaaggatgtaatgttctgatacaaattagatataactccagccttattagggattttaggagatacggcacttgaattcccattaaaacaaaacaaaaacaaaaaagcttttggcttttgtgaaagtctgtagaactcaagagacaataaagacaaaagtacagaaaata

SEQ ID NO: 4 - DNA, 9221 bp, Homo sapiens
  source 1..9221: mol_type = other DNA
tttgatatgacgattagagcataacccgagtgacacgttgaattcgccataatcaaggaaaccttttccgggtggggatctctgaaattactcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 5 - DNA, 9517 bp, Homo sapiens
  source 1..9517: mol_type = other DNA
tttgatatgacgattagagcataacccgagtgacacgttgaattcgccataatcaaggaaaccttttccgggtggggatctctgaaattactcagataacagtgctgtgccaaaaacctgtggattttctctacaaaaattatcatgactgttatctttttgatggaaaagatgaagttcagcgaagaacaaatctacaacttggccagaagactgtagactttgccttcaaagagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagtttt

SEQ ID NO: 6 - DNA, 9350 bp, Homo sapiens
  source 1..9350: mol_type = other DNA
tttgatatgacgattagagcataacccgagtgacacgttgaattcgccataatcaaggaaaccttttccgggtggggatctctgaaattactcagataacagtgctgtgccaaaaacctgtggattttctctacaaaaattatcatgactgttatctttttgatggaaaagatgaagttcagcgaagaacaaatctacaacttggccagaagactgtagactttgccttcaaagagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagtttt

SEQ ID NO: 7 - DNA, 9400 bp, Homo sapiens
  source 1..9400: mol_type = other DNA
tttgatatgacgattagagcataacccgagtgacacgttgaattcgccataatcaaggaaaccttttccgggtggggatctctgaaattactcagataacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagtttt

SEQ ID NO: 8 - DNA, 9221 bp, Homo sapiens
  source 1..9221: mol_type = other DNA
ataatcaaggaaaccttttccgggtggggatctctgaaattactcagataacagtgctgtgccaaaaacctgtggattttctctacaaaaattattgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaacgttattctgaaatgaaatcaaatgttaaagagccttttgtttaacagaaagctggagagccactgccccaaaagttgttcttccagtcttcctgagtgtttggggagtatgtttacaaatcctgccagggtgaagagagatcttgtagctggcaagatttgggagggatggagcagacctgggcagtcagtaataatcaagggaagccaagtaaactccagagtcttacctctggctcacagctgcttttgtgatacacaaagtcacaaagaggaaaagggaagggaggaggttgcttttcaaaatgcattcaccatggctcactgtgtggggcatgattgcaggcctgagctcatgaaagactgtccaagattttgtggtttcaacctctcttcagcctgaaaattccatccagtcgatttgtcacagatcaggatcttcctgtcccctgcagcccatggggacttcctagaaatagatatgatatgttgtagatgtggttaaaacaaatccaggccccattgtttattcagctcattagtccacacctatttttccctagaactcctaccttttccaataatcatgccagcatgaaatgaccttttctactttaaaattttttgacttgttcatattgcttgattttcatcatctcagactcatttttctcctacctaatatcataaaaaagtaaatcagatatacagaatatcatttatgatacttcttcctttcagcaaattgactgaaaagtcattttaaagagagaagtgcagatttgctctcatttctttgatattgttgcagtatccaccccaccatctagcaatcagagatgcttatattaacttgggattagctttgcattatctaacccatttattttaaatctgccaggaaatcctctaactttccttcctttttgtttcagtaagtatcaggcagcttcaccatacctgagtccttttgtcttgaagctgccacagaaaaatcttacagcaatcattgctgattagaaactgtttcagacaatcagcatgggtgttatttaccaaattccccccagagtcctaggcctcttctccagaaatatctgatgatgaagtgaggggagggcaacggtgctacaaaacacggaacagaggtaaagagaaggcactactttcttgccatacttgtaaatgattgctttgttcaaacataaataatcttaagtccaacaccaaatacctgttactcctacatcaatctcattagtggtttaagacacagtactagaattttcattttttaaaatcccttggcccttaaaaaaatttacagaacacaaaggaaaacataaacacaaagacatggaaaattttgtcaactccttaatggaattctgtgatcaaaaagcaggccagattctaatcaaaatcaggtaaattttaatcacaatcagaagtacttgtaacatttcagttgtcctaactccaatgagataacaaagcctccaaggctacagctgaaactctgaaaggccctgtgctttctactttacatttagcgtctaatatttcctaggacagtagttcccaaagtaggctgtacattagaatctcctggagagctttttaaatgctaatgccaataacatatctcataaaatttacactagaatttccttggatgggtgcctggcatcagtattttttaatgatccctaggtgatttcaatgtgcacctaggtttgggaattgctatattcttgtatttatactctatctctttatttcttttcctttttatttcacttttagtaaatagaaaaatgcaattaaaaaatcatctaagttaagacaaatagccaaatcatatttatcccataaatagtatgaatttaaatgtaacttacgtaggcttcaaactaactctaaaaatgagaaagtaaaacaatcttgtcctaaaacaattcttagatccaaatttacactcccctacagatctgcttttattctcaaagacagtgattacattgacataacaaatgtccattggggcaggcaaatttctactgagtctaaaaagcattccaaattctaggagcaaaagttctgattttccttaaaatctttgcaaataaacagcaaagggtattctggatgtcttgtcaatgcaaattaggtttgatttagagaagttttaaatttaatcgcaatgaaaagattagttaatacatgaatctttttatgagaaaaaatcattattctggataaagtcataattgtttgtttacaatattaagagcagacacctaaacatacaataataccaaagtattgagaaaattaccactggaacaccatctcaagttatcacaccatttacctctttagagccagttttgtataagtaggtagaagtggttttggtttttttgctaaaattttactttgttttagtaatatctagggaatactttgaattccattaaaataccttcatagaaagtgttaatgtgtctaccatgccatatgtttatttttactttccccaagttaggtagatagagcaagtcctgctaccataagtttaaaattattaagacaacgggcttttttaaattgctggtagaaccaaaggctaattttgcataggtttatatttttagacttaagaaattagcacctatataataccttcataccttctgcatcactatcaatttttctgtaatttcttatttgaatgcttcctaattctttacagaaaaaattaaaatttgtcacccaaccaggtgataataacaaaacaataatttaggaacatccagtaaatttgaattagttctctaaattttggtaattaatagcttattgataattaaaatcagcctaatgttgcttaagtaggaggaaattgatcagcaatgggattgggtatctattttctttcctataaaagtgctgcctcttctcaaattgctttatttctctcccactgcattagtatatctagaacagcaccttcaactgcaacaaaatataattctcctagatgactaggatatcctccaatctggtatcatgccaaacaacactatgtttggggaacagcacccgaaatgctgctaaaaagctacctcatgtcaagaaaatatggatttcaaaaaatatctggcacagtggcccatgcctataaccccagtactttgggaggccaaggcaggaggattgctggaggccaggagttcaagaccagcctgggcaacatggagagactccgcctctacaaaaaaaaaaaaaacaaatttaatttaattggccagttgtggtggcgtgggcctgtggtcccagctacttgggaagctaaggcaggaggattgctggaggcctggagggcaaggctgcagtgagccatgttcacatcgctgcactccagcctgggtgacagagcaagaccctgtctccaaaaaagcaaaaataaatatctaacaaaaaggaacctgtttccaaaccacagtttcttaactttttcatgatggaatagaaattaaactataagtatatactgataatagctgtgcaggaaattggtttttattttacttaaaagtattttttagaaaaaatgttattctgattgcatagattagcaaaatattgcatcaatataatgttccatttttattaatttttgttatattctaaatgaccgttttataattctgtcttggacaactgggccacagttagaatccacatagacagggaggttcttaggtgtgccatttgtagaataaagggcaatcaagttaatattcggggtaaattttcattgcattctaacctatgaagtttgataaagtatattattattgaacaactctaacactaagatttcctaaaagtaacttacaacaaaaaatgaactatgtgctcaatattcacataaagtcatgattattctcttcttgcattcatataaaagttcccagggggaagaaaatatatctaaaaattagactagaaacttggcactttttatgtattgtagatacactttcatatattgtaaatacatctgttaaagataaaactaagtttctaacatagcactacaaggaaactttggataaatatttttaaaaagaaaatgttaataggataacttaagattttgtatatacttattggaagacaggaagaaactaggcagtaaatattcctacctgacgtgcaaccattgtcaatcaggttttgttaaactgtcaaatttgattttgaaattttagtttgcccaaataatatcttgaaaatgctctgaattttactagaacatattcatagaaagtgtaatgtatctaccaggctctatgtttgtgtttgccaacattagaaaaacaaagtaagttgcgctataatgagtgaaactaaggaaaatgagatttttctgactcccattccctctcttaaaaagtaaaatatttaagctttttttattaaaggaaaaatgggtacagaatactctattgccctccaaatgtccaacatttttaaagccatgttgtccaacaatgtttatacagaaaatagaatgcataggtttaatatagatagcaatgtatatgtaggctcatttgtgggagattgcaaactttatgtgcccccagttctccttggttttgtaggaaagctaaaagaaaaccttgtactctcactaataatgaaattcatagccaatttagaaagggaaaataacctgaaaaccaaaatacctcattcatctgaagatgcctggaataaaatatggaattcttctgtacctacctcccccttacagacttttctcctattttgagagagaaattcaagaagaagaatacagcttgttctctttccacaaatgatactttgtctaagggaataggagataccaagaaacagtatctttaaaaaagtaagctgcccacaggccttcttggagaatgaagaatatcagccccattcagggtttaccaatgtctctctgaggtgacaatacattctggttttccaggacagtcctggcttaggtctgctgtccttctataaatattaacaatgatccctcttatttccaaacgtgtcccagtttagacaacaaattatacagtgtctgcccccaacacacacacacacaccccaccatctttatgggttgttgttgttttcttgttgtttttaattttgtagaattggctttgtcttctccaaagcagataacagcttaaactaaagagcctagctacttctttctgactgagcagggtcatagtaaacagagaagaatggatcacttcctcctctgaaatctccatcaatccactttctgtccaactatctctctacccagcctatttttggtcctagttttcctctcataacccctgggctgtgagtcctgagttctgccttcatatggcagagcaagctagtcattattatgccaggttaggtggtggggcaagacaaaagatagctcttggggacacttgcttggtctgtagagatccacagggcactagtagggtattagagcatttattggtttctttcttccttgagttgagttacttctatcccaaaacagacaggtcttacgttaaccttcaccttaaaaaggatattgccttttttccttctgcttcacaaatttgctccaacactaattcccttgcatttgatttcatcattatgattgttcatcaaccattcttcacgaaaaatcctcacatcacgtcatataaaggttttattatttttaatggaaaataacattgagagattattttcctttttcataaaatttccctttttccctccctctattctcttcactaacattggaaattaaacaagttttacaggctctattgttattaaccctcaaatctattcttcagagaatatatgatcagcctgtgtttactatgcacctactcttataccagaaactgtgaatacaaatacatatatttcctacttctctctttaattggggcacacagtcccatggtccattctgcatttttcaaaattgaatataattgcaacacctgataggttttgccacttagaattctgaaaataaatcaactctgttctaaaccttaagtttccactattattgccaaactctggactgatacaataagccttttaaaaggtgaatttaacacttcacctattatggttatttaagtaaaatgagttttcaaatatttcccccaggagaaatataaaggactatgcaaaaatagaaggagaaaaaggttagttagaggtccagaaggagaattaaaacatggaattgataaattcacctactgatttatgatgttaatgagcaggtacctatacattttatttacttgaaaacattttggccattaactatttctgttttctaaatacatttgtgtttataaaattttaaaatctatatcacaagacttagacatgcctttctttgcaataaagtatatatacctttttacaaatgtaaattttattttgaaacttggcattttctttataattcctaagatatctttgggcacacagactacagataaatctatttaaatcaattcatcattatcactggtttaaatatttaatgtaaccattggaatttttattcttattccttcagagaattctccttttcttctatgtttctgactgcaatacagcccgatattgttattccctatatcttaatgaaaagcaagctgtcagcaaaatgcatttatagtttccagatgtttaacaatctctttcttcttttcttttttgttattatttttatttatgtgaagttagttagctgaggtaccaaaaaagagacaaagacagtattaaaaagtgtgcataaagcacttatttttgtactttagtacaatgttttatagaactttgtgatctatctaaatatattaagtaaaattacaccattcacttgttgggaaaataatctttggtttggaagatattaacataatgggcatcttagaatcataaatcacatgaaatgagagacaatgcaatattgtataattcctggatgatgcaattgttttaattgaattttcaagtgccattataaagttttaaaaattatcaa

SEQ ID NO: 9 - DNA, 2211 bp, Homo sapiens
  source 1..2211: mol_type = other DNA
tttgatatgacgattagagcataacccgagtgacacgttgaattcgccataatcaaggaaaccttttccgggtggggatctctgaaattactcagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaatttagaggttcctatagatggaagcagtgtgtctgggcacatgcccagagccagaagcgagcatgagcacagcaattcctggcctcaaaaaaggaaataatgctttgcagtccatcattttgcaaactttgctggaaaaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaa

SEQ ID NO: 10 - DNA, 1997 bp, Homo sapiens
  source 1..1997: mol_type = other DNA
tgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccaggaagaattcagttatggggacatggtattgcagtagactgggcagagccagaagtagaagttgatgaagatacaatgtcttcagtgaaaatcctatatgtaagaaatcttatgctgtctacctctgaagagatgattgaaaaggaattcaacaatatcaaaccaggtgctgtggagagggtgaagaaaattcgagactatgcttttgtgcacttcagtaaccgagaagatgcagttgaggctatgaaagctttaaatggcaaggtgctggatggttcccccattgaagtcaccctagcaaaaccagtggacaaggacagttatgttaggtatacccgaggcacaggtggaaggggcaccatgctgcaaggagagtatacctactctttgggccaagtttatgatcccaccacaacctaccttggagctcctgtcttctatgccccccagacctatgcagcaattcccagtcttcatttcccagccaccaaaggacatctcagcaacagagccattatccgagccccttctgttagagaaatttacatgaatgtacctgtaggggctgcgggagtgagaggactgggcggccgtggctatttggcatacacaggcctgggtcgaggataccaggtcaaaggagacaaaagagaagacaaactctatgacattttacctgggatggagctcaccccaatgaatcctgtcacattaaaaccccaaggaattaaactcgctccccagatattagaagagatttgtcagaaaaataactggggacagccagtgtaccagctgcactctgctattggacaagaccaaagacagctattcttgtacaaaataactattcctgctctagccagccagaatcctgcaatccaccctttcacacctccaaagctgagtgcctttgtggatgaagcaaagacgtatgcagccgaatacaccctgcagaccctgggcatccccactgatggaggcgatggcaccatggctactgctgctgctgctgctactgctttcccaggatatgctgtccctaatgcaactgcacccgtgtctgcagcccagctcaagcaagcggtaacccttggacaagacttagcagcatatacaacctatgaggtctacccaacttttgcagtgactgcccgaggggatggatatggcaccttctgaagatgcttttttaaatttaagaataagacacacaaaactctattaaaaaaaaaaaagaaataaacctctaactcggtccccaatgatcataaataatatgtttcctaaagaaatgcctttccagagactgtatagcttataccaattatagaatcatgaagtaagaa

SEQ ID NO: 11 - DNA, 885 bp, Homo sapiens
  source 1..885: mol_type = other DNA
tttgatatgacgattagagcataacccgagtgacacgttgaattcgccataatcaaggaaaccttttccgggtggggatctctgaaattactcagataacagtgctgtgccaaaaacctgtggattttctctacaaaaattatcatgactgttatctttttgatggaaaagatgaagttcagcgaagaacaaatctacaacttggccagaagactgtagactttgccttcaaagagtgagcaaccctaattaacctgattttttgctgataatcactctcaatggaatcaaatcacaaatccggggatggattgagcggcactcagaaggaagcagccctccgcgcactggtccagcgcacaggatatagcttggtccaggaaaatggacaaagaaaatatggtggccctccacctggttgggatgctgcaccccctgaaaggggctgtgaaatttttattggaaaacttccccgagacctttttgaggatgagcttataccattatgtgaaaaaatcggtaaaatttatgaaatgagaatgatgatggattttaatggcaacaatagaggatatgcatttgtaacattttcaaataaagtggaagccaagaatgcaatcaagcaacttaataattatgaaattagaaatgggcgcctcttaggggtttgtgccagtgtggacaactgccgattatttgttgggggcatcccaaaaaccaaaaagagagaagaaatcttatcggagatgaaaaaggttactgaaggtgttgtcgatgtcatcgtctacccaagcgctgcagataaaaccaaaaaccgaggctttgccttcgtggagtatgagagtcatcgagcagctgccatggcgaggaggaaactgctaccag

SEQ ID NO: 12 - DNA, 19 bp, synthetic construct
  source 1..19: mol_type = other DNA
gtggacaactgccgattat

SEQ ID NO: 13 - DNA, 19 bp, synthetic construct
  source 1..19: mol_type = other DNA
ctgaaggtgttgtcgatgt

SEQ ID NO: 14 - DNA, 19 bp, synthetic construct
  source 1..19: mol_type = other DNA
caacagagccattatccga

SEQ ID NO: 15 - DNA, 19 bp, synthetic construct
  source 1..19: mol_type = other DNA
agacgtatgcagccgaata

SEQ ID NO: 16 - DNA, 19 bp, synthetic construct
  misc_feature 1..19: note = Synthetic oligonucleotide
  source 1..19: mol_type = other DNA
tggtttacatgtcgactaa

SEQ ID NO: 17 - DNA, 19 bp, synthetic construct
  misc_feature 1..19: note = Synthetic oligonucleotide
  source 1..19: mol_type = other DNA
gcacttcgcttcacctctg

SEQ ID NO: 18 - DNA, 19 bp, synthetic construct
  misc_feature 1..19: note = primer
  source 1..19: mol_type = other DNA
ccgtgtgcacttcgcttca

SEQ ID NO: 19 - DNA, 19 bp, synthetic construct
  misc_feature 1..19: note = primer
  source 1..19: mol_type = other DNA
gcacagcttggaggcttga

SEQ ID NO: 20 - DNA, 23 bp, synthetic construct
  misc_feature 1..23: note = primer
  source 1..23: mol_type = other DNA
catggagaccaccgtgaacgccc

(19) United States
(12) Patent Application Publication
(10) Pub. No.: US 2023/0257748 A1
(43) Pub. Date: Aug. 17, 2023

(54) USE OF A1CF INHIBITORS FOR TREATING HEPATITIS B VIRUS INFECTION

(71) Applicant: Hoffmann-La Roche Inc., Little Falls, NJ (US)

(72) Inventors: Dennis JUL HANSEN, Farum (DK); Souphalone LUANGSAY, Basel (CH); Alan James MUELLER-BRECKENRIDGE, Munchenstein (CH); Lykke PEDERSEN, Kobenhavn NV (DK); Johanna Marie POSE VICENTE, Birsfelden (CH)

(73) Assignee: Hoffmann-La Roche Inc., Little Falls, NJ (US)

(21) Appl. No.: 18/171,130

(22) Filed: Feb. 17, 2023

Related U.S. Application Data
(63) Continuation of application No. PCT/EP2021/072985, filed on Aug. 19, 2021.

(30) Foreign Application Priority Data
Aug. 21, 2020 (EP) 20192212.7

(51) Int. Cl. C12N 15/113; A61K 47/54; A61K 48/00; A61K 47/26; A61P 31/20

(52) U.S. Cl. CPC C12N 15/113; A61K 47/549; A61K 48/0033; A61K 47/26; A61P 31/20

(57) ABSTRACT

The present invention relates to an A1CF inhibitor for use in treatment of an HBV infection, in particular a chronic HBV infection. The invention in particular relates to the use of A1CF inhibitors for destabilizing cccDNA, such as HBV cccDNA. The invention also relates to nucleic acid molecules which are complementary to A1CF and capable of reducing the level of an A1CF mRNA. Also comprised in the present invention is a pharmaceutical composition and its use in the treatment of a HBV infection.

### FIELD OF INVENTION

The present invention relates to A1CF inhibitors for use in treating a hepatitis B virus (HBV) infection, in particular a chronic HBV infection. The invention in particular relates to the use of A1CF inhibitors for destabilizing cccDNA, such as HBV cccDNA. The invention also relates to nucleic acid molecules, such as oligonucleotides including siRNA, shRNA and antisense oligonucleotides, that are complementary to A1CF, and capable of reducing the expression of A1CF. Also comprised in the present invention is a pharmaceutical composition and its use in the treatment of a HBV infection.

### BACKGROUND

Hepatitis B is an infectious disease caused by the hepatitis B virus (HBV), a small hepatotropic virus that replicates through reverse transcription. Chronic HBV infection is a key factor for severe liver diseases such as liver cirrhosis and hepatocellular carcinoma. Current treatments for chronic HBV infection are based on administration of pegylated type 1 interferons or nucleos(t)ide analogues, such as lamivudine, adefovir, entecavir, tenofovir disoproxil, and tenofovir alafenamide, which target the viral polymerase, a multifunctional reverse transcriptase. Treatment success is usually measured as loss of hepatitis B surface antigen (HBsAg). However, a complete HBsAg clearance is rarely achieved since Hepatitis B virus DNA persists in the body after infection. HBV persistence is mediated by an episomal form of the HBV genome which is stably maintained in the nucleus. This episomal form is called “covalently closed circular DNA” (cccDNA). The cccDNA serves as a template for all HBV transcripts, including pregenomic RNA (pgRNA), a viral replicative intermediate. The presence of a few copies of cccDNA might be sufficient to reinitiate a full-blown HBV infection. Current treatments for HBV do not target cccDNA. A cure of chronic HBV infection, however, would require the elimination of cccDNA (reviewed by Nassal, Gut. 2015 December; 64(12):1972-84. doi: 10.1136/gutjnl-2015-309809).

A1CF (APOBEC1 complementation factor) is a component of the apolipoprotein B mRNA editing enzyme complex which is responsible for the posttranscriptional editing of a CAA codon for Gln to a UAA codon for stop in apolipoprotein B mRNA. The introduction of a stop codon into apolipoprotein B mRNA alters lipid metabolism in the gastrointestinal tract. The editing enzyme complex comprises a minimal core composed of the cytidine deaminase APOBEC-1 (Apolipoprotein B mRNA editing enzyme 1) and a complementation factor encoded by the A1CF gene. The A1CF protein has three non-identical RNA recognition motifs and belongs to the hnRNP R family of RNA-binding proteins. It binds to apolipoprotein B mRNA and is probably responsible for docking the catalytic subunit, APOBEC1, to the mRNA to allow it to deaminate its target cytosine (see Chester et al., EMBO J. 2003 Aug. 1; 22(15):3971-82).

Many reports on the apolipoprotein B mRNA editing enzyme complex are focused on the cytidine deaminase APOBEC1, rather than on the APOBEC1 complementation factor. It has been shown that APOBEC1 does not only edit apolipoprotein B mRNA, but also viral genomes including HBV.

In a mouse model for HBV replication, Renard et al. showed that mouse APOBEC1 edited HBV in vivo (Renard et al., J Mol Biol. 2010 Jul. 16; 400(3):323-34. doi: 10.1016/j.jmb.2010.05.029). In contrast, rat APOBEC1 did not inhibit HBV DNA production (Rösler et al., Hepatology. 2005 August; 42(2):301-9).

Gonzalez et al. showed that human APOBEC1 edits HBV DNA. In cells co-transfected with HBV and human APOBEC1, several G to A hypermutations were identified in the HBV genome. Further, the presence of human APOBEC1 impacted replication of HBV DNA. Specifically, it was shown that an increased expression of APOBEC1 resulted in a decreased amount of HBV DNA (Gonzalez et al., Retrovirology. 2009 Oct. 21; 6:96. doi: 10.1186/1742-4690-6-96).

To our knowledge A1CF has never been identified as a cccDNA dependency factor in the context of cccDNA stability and maintenance, nor have molecules inhibiting A1CF ever been suggested as cccDNA destabilizers for the treatment of HBV infection.

Furthermore, to our knowledge the only disclosure of oligonucleotides potentially related to the regulation of A1CF expression are suggested in WO 2016/142948. However, WO 2016/142948 relates to the alteration of splicing of a number of listed targets including A1CF, to produce alternative splice variants. The oligonucleotides are however decoy oligonucleotides encoding splicing-factor binding sites and does therefore not bind to the targets as such. WO 2016/142948 also mentions a list of treatments including cancer, inflammation, immunological disorders, neurodegeneration, Alzheimer disease, Parkinson, viral infections (HIV, HSV, HBV). There are however no specific examples of oligonucleotides targeting A1CF nor their use in HBV.

### OBJECTIVE OF THE INVENTION

The present invention shows that there is an association between the inhibition of A1CF and reduction of of the amount of cccDNA in an HBV infected cell, which is relevant in the treatment of HBV infected individuals. An objective of the present invention is to identify A1CF inhibitors which reduce the amount of cccDNA in an HBV infected cell. Such A1CF inhibitors can be used in the treatment of HBV infection.

The present invention further identifies novel nucleic acid molecules, which are capable of inhibiting the expression of A1CF in vitro and in vivo.

### SUMMARY OF INVENTION

The present invention relates to oligonucleotides targeting a nucleic acid capable of modulating the expression of A1CF and to treat or prevent diseases related to the functioning of the A1CF.

Accordingly, in a first aspect the invention provides an A1CF inhibitor for use in the treatment and/or prevention of Hepatitis B virus (HBV) infection. In particular, an A1CF inhibitor capable of reducing the amount of HBV cccDNA and/or HBV pre-genomic RNA (pgRNA) is useful. Such an inhibitor is advantageously a nucleic acid molecule of 12 to 60 nucleotides in length, which is capable of reducing A1CF mRNA.

In a further aspect, the invention relates to a nucleic acid molecule of 12-60 nucleotides, such as of 12-30 nucleotides, comprising a contiguous nucleotides sequence of at least 10 nucleotides, in particular of 16 to 20 nucleotides, which is at least 90% complementary, such as fully complementary to a mammalian A1CF, e.g. a human A1CF, a mouse A1CF or a cynomolgus monkey A1CF. Such a nucleic acid molecule is capable of inhibiting the expression of A1CF in a cell expressing A1CF. The inhibition of A1CF allows fora reduction of the amount of cccDNA present in the cell. The nucleic acid molecule can be selected from a single stranded antisense oligonucleotide, a double stranded siRNA molecule or a shRNA nucleic acid molecule (in particular a chemically produced shRNA molecules).

A further aspect of the present invention relates to single stranded antisense oligonucleotides or siRNA's that inhibit the expression and/or activity of A1CF. In particular, modified antisense oligonucleotides or modified siRNAs comprising one or more 2′ sugar modified nucleoside(s) and one or more phosphorothioate linkage(s), which reduce A1CF mRNA are advantageous.

In a further aspect, the invention provides pharmaceutical compositions comprising the A1CF inhibitor of the present invention, such as the antisense oligonucleotide or siRNA of the invention and a pharmaceutically acceptable excipient.

In a further aspect, the invention provides methods for in vivo or in vitro modulation of A1CF expression in a target cell which is expressing A1CF, by administering an A1CF inhibitor of the present invention, such as an antisense oligonucleotide or composition of the invention in an effective amount to said cell. In some embodiments, the A1CF expression is reduced by at least 50%, or at least 60%, or at least 70%, or at least 80%, in the target cell compared to the level without any treatment or treated with a control. In some embodiments, the target cell is infected with HBV and the cccDNA in an HBV infected cell is reduced by at least 50%, or at least 60%, or at least 70%, in the HBV infected target cell compared to the level without any treatment or treated with a control. In some embodiments, the target cell is infected with HBV and the pgRNA in an HBV infected cell is reduced by at least 50%, or at least 60%, in the HBV infected target cell compared to the level without any treatment or treated with a control.

In a further aspect, the invention provides methods for treating or preventing a disease, disorder or dysfunction associated with in vivo activity of A1CF comprising administering a therapeutically or prophylactically effective amount of the an A1CF inhibitor of the present invention, such as the antisense oligonucleotide or siRNA of the invention to a subject suffering from or susceptible to the disease, disorder or dysfunction.

Further aspects of the invention are conjugates of nucleic acid molecules of the invention and pharmaceutical compositions comprising the molecules of the invention. In particular, conjugates targeting the liver are of interest, such as GalNAc clusters.

### BRIEF DESCRIPTION OF FIGURES

FIG. 1A-L: Illustrates exemplary antisense oligonucleotide conjugates, wherein the oligonucleotide is represented by the term “Oligonucleotide” and the asialoglycoprotein receptor targeting conjugate moieties are trivalent N-acetylgalactosamine moieties. Compounds in FIG. 1A-D comprise a di-lysine brancher molecule, a PEG3 spacer and three terminal GalNAc carbohydrate moieties. In the compounds in FIG. 1A (FIG. 1A-1 and FIG. 1A-2 show two different diastereoisomers of the same compound) and FIG. 1B (FIG. 1B-1 and FIG. 1B-2 show two different diastereoisomers of the same compound) the oligonucleotide is attached directly to the asialoglycoprotein receptor targeting conjugate moiety without a linker. In the compounds in FIG. 10 (FIG. 1C-1 and FIG. 1C-2 show two different diastereoisomers of the same compound) and FIG. 1D (FIG. 1D-1 and FIG. 1D-2 show two different diastereoisomers of the same compound) the oligonucleotide is attached to the asialoglycoprotein receptor targeting conjugate moiety via a C6 linker. The compounds in FIG. 1E-K comprise a commercially available trebler brancher molecule and spacers of varying length and structure and three terminal GalNAc carbohydrate moieties. The compound in FIG. 1L is composed of monomeric GalNAc phosphoramidites added to the oligonucleotide while still on the solid support as part of the synthesis, wherein X=S or O, and independently Y=S or O, and n=1-3 (see WO 2017/178656). FIG. 1B and FIG. 1D are also termed GalNAc2 or GN2 herein, without and with C6 linker respectively.

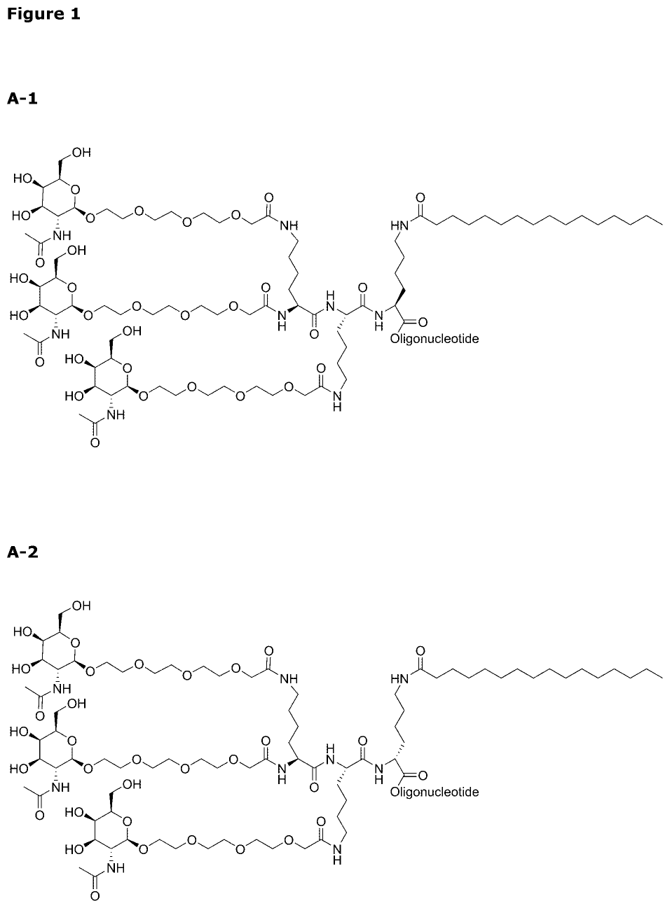

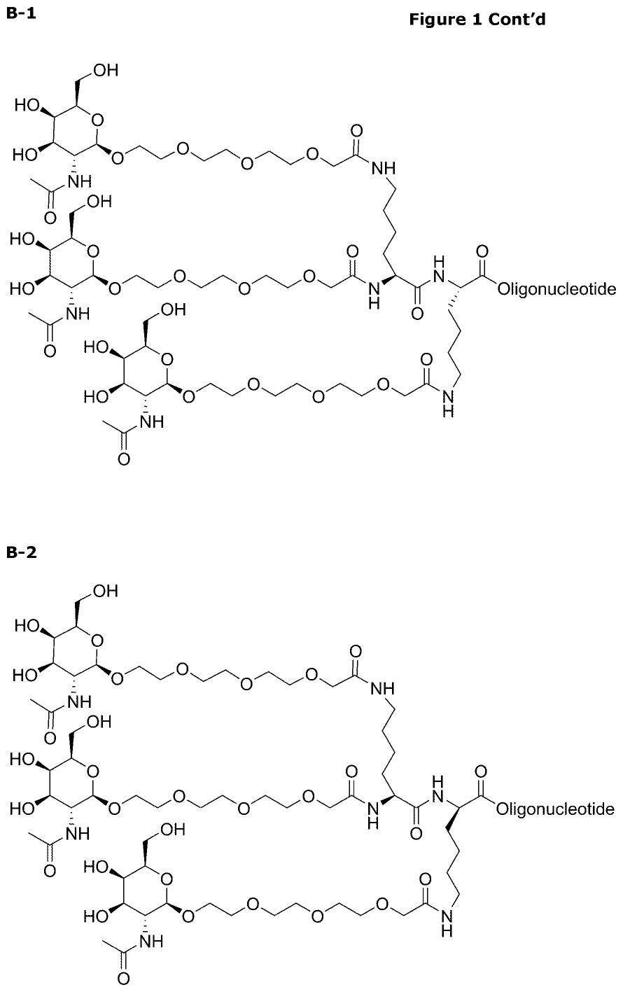

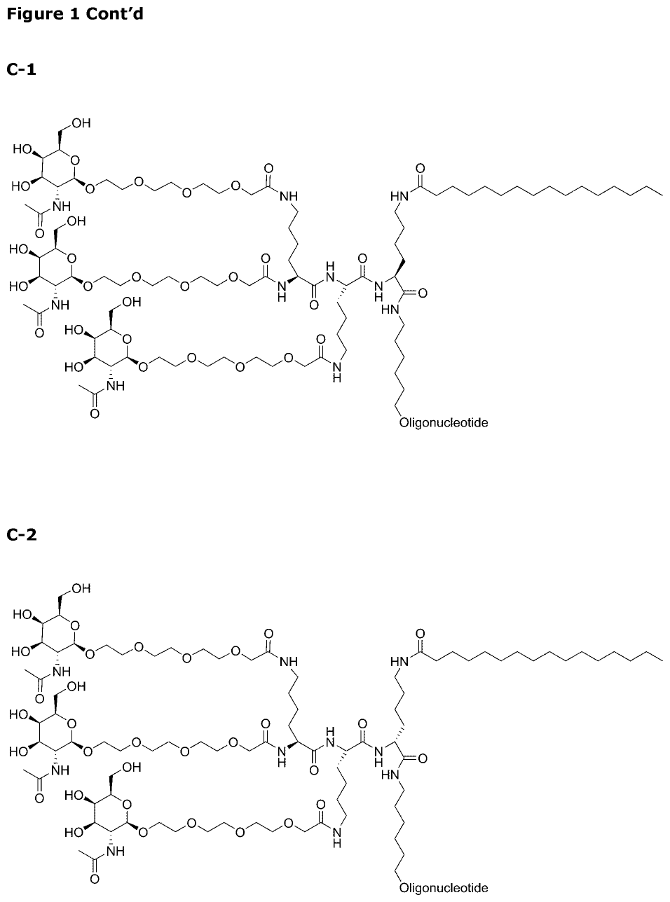

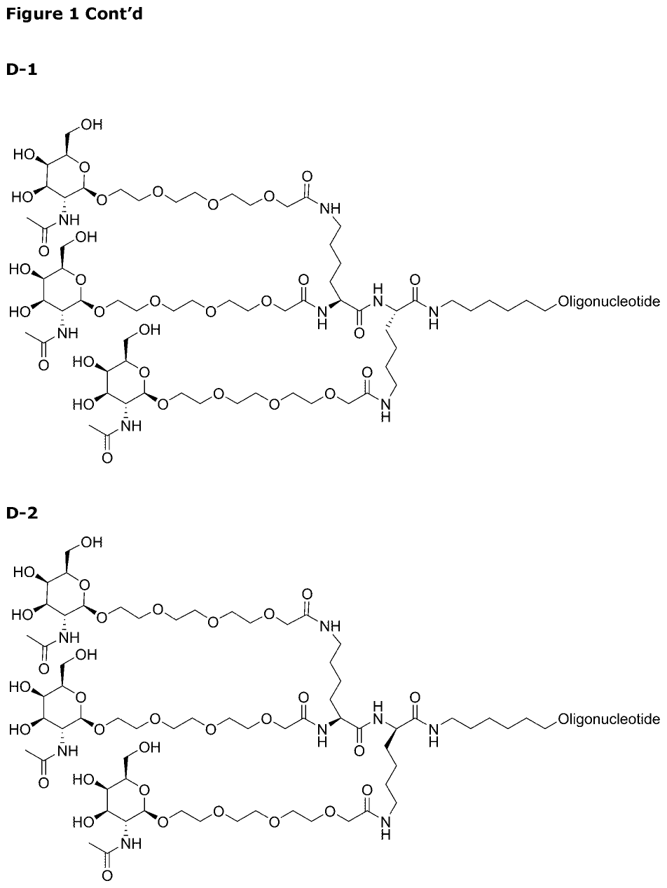

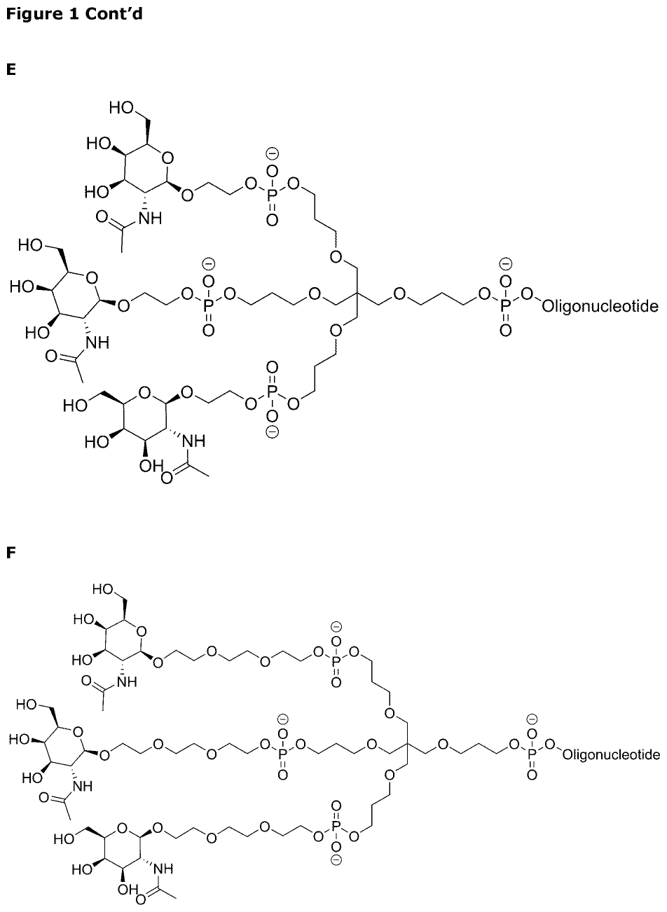

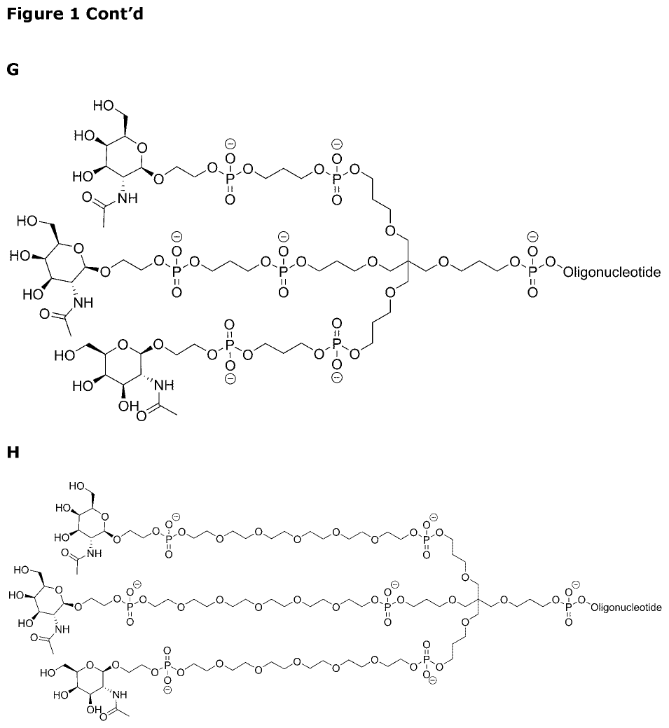

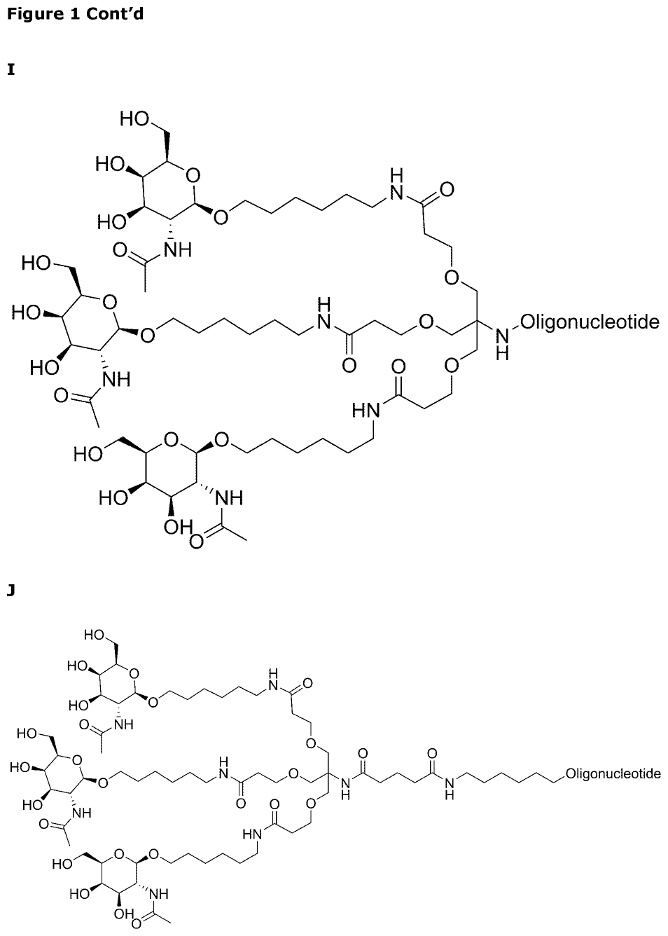

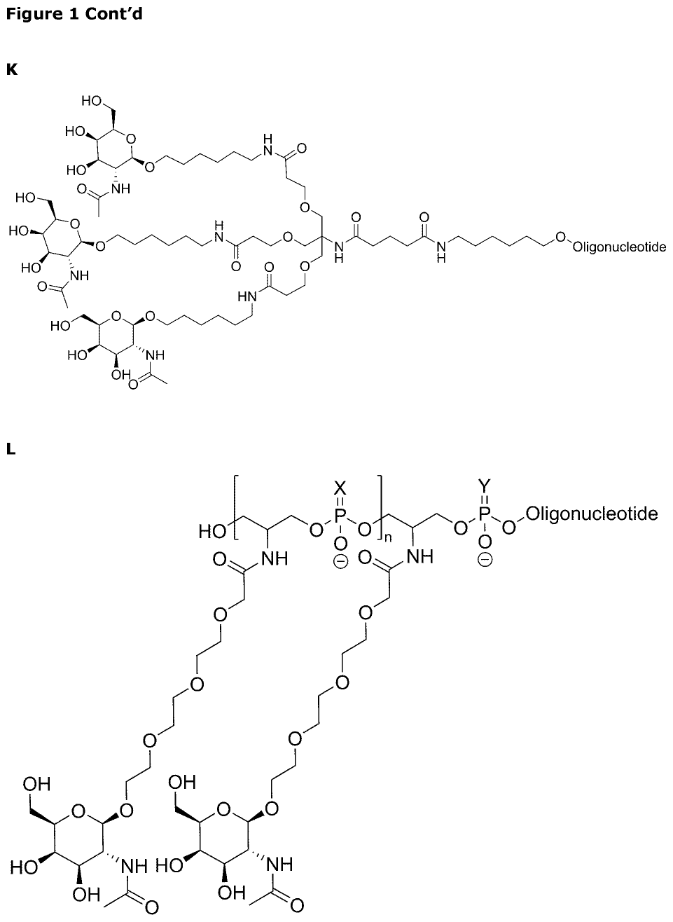

The two different diastereoisomers shown in each of FIG. 1A-D are the result of the conjugation reaction. A pool of a specific antisense oligonucleotide conjugate can therefore contain only one of the two different diastereoisomers, or a pool of a specific antisense oligonucleotide conjugate can contain a mixture of the two different diastereoisomers.

### DEFINITIONS

HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Chronic hepatitis B virus (CHB) infection is a global disease burden affecting 248 million individuals worldwide. Approximately 686,000 deaths annually are attributed to HBV-related end-stage liver diseases and hepatocellular carcinoma (HCC) (GBD 2013; Schweitzer et al., Lancet. 2015 Oct. 17; 386(10003):1546-55). WHO projected that without expanded intervention, the number of people living with CHB infection will remain at the current high levels for the next 40-50 years, with a cumulative 20 million deaths occurring between 2015 and 2030 (WHO 2016). CHB infection is not a homogenous disease with singular clinical presentation. Infected individuals have progressed through several phases of CHB-associated liver disease in their life; these phases of disease are also the basis for treatment with standard of care (SOC). Current guidelines recommend treating only selected CHB-infected individuals based on three criteria—serum ALT level, HBV DNA level, and severity of liver disease (EASL, 2017). This recommendation was due to the fact that SOC i.e. nucleos(t)ide analogs (NAs) and pegylated interferon-alpha (PEG-IFN), are not curative and must be administered for long periods of time thereby increasing their safety risks. NAs effectively suppress HBV DNA replication; however, they have very limited/no effect on other viral markers. Two hallmarks of HBV infection, hepatitis B surface antigen (HBsAg) and covalently closed circular DNA (cccDNA), are the main targets of novel drugs aiming for HBV cure. In the plasma of CHB individuals, HBsAg subviral (empty) particles outnumber HBV virions by a factor of 103 to 105 (Ganem & Prince, N Engl J Med. 2004 Mar. 11; 350(11):1118-29); its excess is believed to contribute to immunopathogenesis of the disease, including inability of individuals to develop neutralizing anti-HBs antibody, the serological marker observed following resolution of acute HBV infection.

In some embodiments, the term “HBV infection” refers to “chronic HBV infection”.

Further, the term encompasses infection with any HBV genotype.

In some embodiments, the patient to be treated is infected with HBV genotype A.

In some embodiments, the patient to be treated is infected with HBV genotype B.

In some embodiments, the patient to be treated is infected with HBV genotype C.

In some embodiments, the patient to be treated is infected with HBV genotype D.

In some embodiments, the patient to be treated is infected with HBV genotype E.

In some embodiments, the patient to be treated is infected with HBV genotype F.

In some embodiments, the patient to be treated is infected with HBV genotype G.

In some embodiments, the patient to be treated is infected with HBV genotype H.

In some embodiments, the patient to be treated is infected with HBV genotype I.

In some embodiments, the patient to be treated is infected with HBV genotype J.

cccDNA (Covalently Closed Circular DNA)

cccDNA is the viral genetic template of HBV that resides in the nucleus of infected hepatocytes, where it gives rise to all HBV RNA transcripts needed for productive infection and is responsible for viral persistence during natural course of chronic HBV infection (Locarnini & Zoulim, Antivir Ther. 2010; 15 Suppl 3:3-14. doi: 10.3851/IMP1619). Acting as a viral reservoir, cccDNA is the source of viral rebound after cessation of treatment, necessitating long term, often lifetime treatment. PEG-IFN can only be administered to a small subset of CHB due to its various side effects.

Consequently, novel therapies that can deliver a complete cure, defined by degradation or elimination of HBV cccDNA, to the majority of CHB patients are highly needed.

### Compound

Herein, the term “compound” means any molecule capable of inhibition A1CF expression or activity. Particular compounds of the invention are nucleic acid molecules, such as RNAi molecules or antisense oligonucleotides according to the invention or any conjugate comprising such a nucleic acid molecule. For example, herein the compound may be a nucleic acid molecule targeting A1CF, in particular an antisense oligonucleotide or a siRNA.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers.

The oligonucleotides referred to in the description and claims are generally therapeutic oligonucleotides below 70 nucleotides in length. The oligonucleotide may be or comprise a single stranded antisense oligonucleotide, or may be another nucleic acid molecule, such as a CRISPR RNA, a siRNA, shRNA, an aptamer, or a ribozyme. Therapeutic oligonucleotide molecules are commonly made in the laboratory by solid-phase chemical synthesis followed by purification and isolation. shRNA's are however often delivered to cells using lentiviral vectors from which they are then transcribed to produce the single stranded RNA that will form a stem loop (hairpin) RNA structure that is capable of interacting with the RNA interference machinery (including the RNA-induced silencing complex (RISC)). In an embodiment of the present invention the shRNA is chemically produced shRNA molecules (not relying on cell based expression from plasmids or viruses). When referring to a sequence of the oligonucleotide, reference is made to the sequence or order of nucleobase moieties, or modifications thereof, of the covalently linked nucleotides or nucleosides. Generally, the oligonucleotide of the invention is man-made, and is chemically synthesized, and is typically purified or isolated. Although in some embodiments the oligonucleotide of the invention is a shRNA transcribed from a vector upon entry into the target cell. The oligonucleotide of the invention may comprise one or more modified nucleosides or nucleotides.

In some embodiments, the oligonucleotide of the invention comprises or consists of 10 to 70 nucleotides in length, such as from 12 to 60, such as from 13 to 50, such as from 14 to 40, such as from 15 to 30, such as from 16 to 25, such as from 16 to 22, such as from 16 to 20 contiguous nucleotides in length. Accordingly, the oligonucleotide of the present invention, in some embodiments, may have a length of 12 to 25 nucleotides. Alternatively, the oligonucleotide of the present invention, in some embodiments, may have a length of 15 to 22 nucleotides.

In some embodiments, the oligonucleotide or contiguous nucleotide sequence thereof comprises or consists of 24 or less nucleotides, such as 22, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 12 to 25 nucleotides, both 12 and 25 nucleotides are included.

In some embodiments, the contiguous nucleotide sequence comprises or consists of 12, 13, 14, 15, 16, 17, 18, 19, 20, 21 or 22 contiguous nucleotides in length The olignucleotide(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s).

In one embodiment, of the invention oligonucleotide is selected from a RNAi agent, such as a siRNA or shRNA. In another embodiment, the oligonucleotide is a single stranded antisense oligonucleotide, such as a high affinity modified antisense oligonucleotide interacting with RNase H.

In some embodiments the oligonucleotide of the invention may comprise one or more modified nucleosides or nucleotides, such as 2′ sugar modified nucleosides.

In some embodiments the oligonucleotide comprises phosphorothioate internucleoside linkages.

In some embodiments the oligonucleotide may be conjugated to non-nucleosidic moieties (conjugate moieties).

A library of oligonucleotides is to be understood as a collection of variant oligonucleotides. The purpose of the library of oligonucleotides can vary. In some embodiments, the library of oligonucleotides is composed of oligonucleotides with overlapping nucleobase sequence targeting one or more mammalian A1CF target nucleic acids with the purpose of identifying the most potent sequence within the library of oligonucleotides. In some embodiments, the library of oligonucleotides is a library of oligonucleotide design variants (child nucleic acid molecules) of a parent or ancestral oligonucleotide, wherein the oligonucleotide design variants retaining the core nucleobase sequence of the parent nucleic acid molecule.

### Antisense Oligonucleotides

The term “antisense oligonucleotide” or “ASO” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide of the invention does not contain RNA nucleosides, since this will decrease nuclease resistance.

Advantageously, the oligonucleotide of the invention comprises one or more modified nucleosides or nucleotides, such as 2′ sugar modified nucleosides. Furthermore, it is advantageous that the nucleosides which are not modified are DNA nucleosides.

### RNAi Molecules

Herein, the term “RNA interference (RNAi) molecule” refers to short double-stranded oligonucleotide containing RNA nucleosides and which mediates targeted cleavage of an RNA transcript via the RNA-induced silencing complex (RISC), where they interact with the catalytic RISC component argonaute. The RNAi molecule modulates, e g., inhibits, the expression of the target nucleic acid in a cell, e.g. a cell within a subject. such as a mammalian subject. RNAi molecules includes single stranded RNAi molecules (Lima at al 2012 Cell 150: 883) and double stranded siRNAs, as well as short hairpin RNAs (shRNAs). In some embodiments of the invention, the oligonucleotide of the invention or contiguous nucleotide sequence thereof is a RNAi agent, such as a siRNA.

siRNA

The term “small interfering ribonucleic acid” or “siRNA” refers to a small interfering ribonucleic acid RNAi molecule. It is a class of double-stranded RNA molecules, also known in the art as short interfering RNA or silencing RNA. siRNAs typically comprise a sense strand (also referred to as a passenger strand) and an antisense strand (also referred to as the guide strand), wherein each strand are of 17 to 30 nucleotides in length, typically 19 to 25 nucleosides in length, wherein the antisense strand is complementary, such as at least 95% complementary, such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and antisense strand 3′ ends may form a 3′ overhang of e.g. 1, 2 or 3 nucleosides to resemble the product produced by Dicer, which forms the RISC substrate in vivo. Effective extended forms of Dicer substrates have been described in U.S. Pat. Nos. 8,349,809 and 8,513,207, hereby incorporated by reference. In some embodiments, both the sense strand and antisense strand have a 2 nt 3′ overhang. The duplex region may therefore be, for example 17 to 25 nucleotides in length, such as 21 to 23 nucleotide in length.

Once inside a cell the antisense strand is incorporated into the RISC complex which mediate target degradation or target inhibition of the target nucleic acid. siRNAs typically comprise modified nucleosides in addition to RNA nucleosides. In one embodiment, the siRNA molecule may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. In particular, 2′fluoro, 2′-O-methyl or 2′-O-methoxyethyl may be incorporated into siRNAs.

In some embodiments, all of the nucleotides of an siRNA sense (passenger) strand may be modified with 2′ sugar modified nucleosides such as LNA (see WO2004/083430, WO2007/085485 for example). In some embodiments, the passenger stand of the siRNA may be discontinuous (see WO2007/107162 for example). The incorporation of thermally destabilizing nucleotides occurring at a seed region of the antisense strand of siRNAs have been reported as useful in reducing off-target activity of siRNAs (see WO2018/098328 for example). Suitably the siRNA comprises a 5′ phosphate group or a 5′-phosphate mimic at the 5′ end of the antisense strand. In some embodiments, the 5′ end of the antisense strand is a RNA nucleoside.

In one embodiment, the siRNA molecule further comprises at least one phosphorothioate or methylphosphonate internucleoside linkage. The phosphorothioaie or methylphosphonate internucleoside linkage may be at the 3′-terminus one or both strand (e.g., the antisense strand; or the sense strand); or the phosphorothioate or methylphosphonate internucleoside linkage may be at the 5′-terminus of one or both strands (e.g., the antisense strand; or the sense strand); or the phosphorothioate or methylphosphonate internucleoside linkage may be at the both the 5′- and 3′-terminus of one or both strands (e.g., the antisense strand; or the sense strand). In some embodiments, the remaining internucleoside linkages are phosphodiester linkages. In some embodiments, siRNA molecules comprise one or more phosphorothioate internucleoside linkages. In siRNA molecules phosphorothioate internucleoside linkages may reduce or the nuclease cleavage in RICS, it is therefore advantageous that not all internucleoside linkages in the antisense strand are modified.

The siRNA molecule may further comprise a ligand. In some embodiments, the ligand is conjugated to the 3′ end of the sense strand.

For biological distribution, siRNAs may be conjugated to a targeting ligand, and/or be formulated into lipid nanoparticles.

Other aspects of the invention relate to pharmaceutical compositions comprising these dsRNA, such as siRNA molecules suitable for therapeutic use, and methods of inhibiting the expression of the target gene by administering the dsRNA molecules such as siRNAs of the invention, e.g., for the treatment of various disease conditions as disclosed herein.

shRNA

The term “short hairpin RNA” or “shRNA” refers to molecules that are generally between 40 and 70 nucleotides in length, such as between 45 and 65 nucleotides in length, such as 50 and 60 nucleotides in length and form a stem loop (hairpin) RNA structure which interacts with the endonuclease known as Dicer which is believed to processes dsRNA into 19-23 base pair short interfering RNAs with characteristic two base 3′ overhangs which are then incorporated into an RNA-induced silencing complex (RISC). Upon binding to the appropriate target mRNA, one or more endonucleases within the RISC cleave the target to induce silencing. shRNA oligonucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA.

In some embodiments, shRNA molecule comprises one or more phosphorothioate internucleoside linkages. In RNAi molecules phosphorothioate internucleoside linkages may reduce or the nuclease cleavage in RICS it is therefore advantageous that not al internucleoside linkages in the stem loop of the shRNA molecule are modified. Phosphorothioate internucleoside linkages can advantageously be placed in the 3′ and/or 5′ end of the stem loop of the shRNA molecule, in particular in the part of the molecule that is not complementary to the target nucleic acid. The region of the shRNA molecule that is complementary to the target nucleic acid may however also be modified in the first 2 to 3 internucleoside linkages in the part that is predicted to become the 3′ and/or 5′ terminal following cleavage by Dicer.

### Contiguous Nucleotide Sequence

The term “contiguous nucleotide sequence” refers to the region of the nucleic acid molecule which is complementary to the target nucleic acid. The term is used interchangeably herein with the term “contiguous nucleobase sequence” and the term “oligonucleotide motif sequence”. In some embodiments, all the nucleotides of the oligonucleotide constitute the contiguous nucleotide sequence. In some embodiments, the contiguous nucleotide sequence is included in the guide strand of an siRNA molecule. In some embodiments, the contiguous nucleotide sequence is the part of an shRNA molecule which is 100% complementary to the target nucleic acid. In some embodiments, the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group for targeting) to the contiguous nucleotide sequence. The nucleotide linker region may or may not be complementary to the target nucleic acid. In some embodiments, the nucleobase sequence of the antisense oligonucleotide is the contiguous nucleotide sequence. In some embodiments, the contiguous nucleotide sequence is 100% complementary to the target nucleic acid.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, one or more of the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise one or more modified internucleoside linkages, such as a one or more phosphorothioate internucleoside linkages, or one or more phosphorodithioate internucleoside linkages.

With the oligonucleotide of the invention it is advantageous to use phosphorothioate internucleoside linkages.

Phosphorothioate internucleoside linkages are particularly useful due to nuclease resistance, beneficial pharmacokinetics and ease of manufacture. In some embodiments, at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleoside linkages, which according to EP 2 742 135 may for example be tolerated in an otherwise DNA phosphorothioate gap region.

### Nucleobase

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides comprising modified nucleosides and DNA nucleosides. The antisense oligonucleotide of the invention is advantageously a chimeric oligonucleotide.

### Complementarity

The term “complementarity” or “complementary” describes the capacity for Watson-Crick base-pairing of nucleosides/nucleotides. Watson-Crick base pairs are guanine (G)-cytosine (C) and adenine (A)—thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RT ln(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA, 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10 to 30 nucleotides in length. In some embodiments, the degree or strength of hybridization is measured by the standard state Gibbs free energy ΔG°. The oligonucleotides may hybridize to a target nucleic acid with estimated ΔG° values below −10 kcal, such as below −15 kcal, such as below −20 kcal and such as below −25 kcal for oligonucleotides that are 8 to 30 nucleotides in length. In some embodiments, the oligonucleotides hybridize to a target nucleic acid with an estimated ΔG° value in the range of of −10 to −60 kcal, such as −12 to −40, such as from −15 to −30 kcal or −16 to −27 kcal such as −18 to −25 kcal.

### Target Nucleic Acid

According to the present invention, the target nucleic acid is a nucleic acid which encodes mammalian A1CF and may for example be a gene, a RNA, a mRNA, and pre-mRNA, a mature mRNA or a cDNA sequence. The target may therefore be referred to as A1CF target nucleic acid.

Suitably, the target nucleic acid encodes an A1CF protein, in particular mammalian A1CF, such as the human A1CF gene encoding pre-mRNA or mRNA sequences provided herein as SEQ ID NO: 1, 4, 5, 6, 7, 8, 9, 10, or 11.

The therapeutic oligonucleotides of the invention may for example target exon regions of a mammalian A1CF (in particular siRNA and shRNA, but also antisense oligonucleotides), or may for example target any intron region in the A1CF pre-mRNA (in particular antisense oligonucleotides). The human A1CF gene encodes 10 transcript, eight of which are protein coding and therefore potential nucleic acid targets.

Table 1 lists predicted exon and intron regions of SEQ ID NO: 1, i.e. of the human A1CF pre-mRNA sequence.

TABLE 1
Exon and intron regions in the human A1CF pre-mRNA.

| Exonic regions in the | | | Intronic regions in the | | |
|---|---|---|---|---|---|
| human A1CF premRNA | | | human A1CF premRNA | | |
| (SEQ ID NO: 1) | | | (SEQ ID NO: 1) | | |
| ID | start | end | ID | start | end |
| E1 | 1 | 95 | I1 | 96 | 21595 |
| E2 | 21596 | 21643 | I2 | 21644 | 22694 |
| E3 | 22695 | 22787 | I3 | 22788 | 25690 |
| E4 | 25691 | 25834 | I4 | 25835 | 34868 |
| E5 | 34869 | 35011 | I5 | 35012 | 41553 |
| E6 | 41554 | 41688 | I6 | 41689 | 43683 |
| E7 | 43684 | 43814 | I7 | 43815 | 49363 |
| E8 | 49364 | 49602 | I8 | 49603 | 57380 |
| E9 | 57381 | 57545 | I9 | 57546 | 65026 |
| E10 | 65027 | 65124 | I10 | 65125 | 69396 |
| E11 | 69397 | 69670 | I11 | 69671 | 71613 |
| E12 | 71614 | 71819 | I12 | 71820 | 74499 |
| E13 | 74500 | 74636 | I13 | 74637 | 75633 |
| E14 | 75634 | 75782 | I14 | 75783 | 78795 |
| E15 | 78796 | 86255 | | | |

Suitably, the target nucleic acid encodes an A1CF protein, in particular mammalian A1CF, such as human A1CF (See for example Table 2 and Table 3) which provides an overview on the genomic sequences of human, cyno monkey and mouse A1CF (Table 2) and on pre-mRNA sequences for human, monkey and mouse A1CF and for the mature mRNAs for human A1CF (Table 3).

In some embodiments, the target nucleic acid is selected from the group consisting of SEQ ID NO: 1, 2, 3, 4, 6, 7, 8, 10, and 11, or naturally occurring variants thereof (e.g. sequences encoding a mammalian A1CF).

TABLE 2
Genome and assembly information for A1CF across species.

| | | | Genomic coordinates | | | ensembl |
|---|---|---|---|---|---|---|
| Species | Chr. | Strand | Start | End | Assembly | gene_id |
| Human | 10 | Rv | 50799409 | 50885675 | GRCh38.p12 | ENSG00000148584 |
| Cyno monkey | 9 | Fwd | 85376801 | 85454053 | Macaca_fascicularis_5.0 | ENSMFAG00000035948 |
| Mouse | 19 | Fwd | 31868764 | 31948995 | GRCm38.p5 | ENSMUSG00000052595 |

Fwd = forward strand. Rv = reverse strand. The genome coordinates provide the pre-mRNA sequence (genomic sequence).

If employing the nucleic acid molecule of the invention in research or diagnostics the target nucleic acid may be a cDNA or a synthetic nucleic acid derived from DNA or RNA.

For in vivo or in vitro application, the therapeutic nucleic acid molecule of the invention is typically capable of inhibiting the expression of the A1CF target nucleic acid in a cell which is expressing the A1CF target nucleic acid. In some embodiments, said cell comprises HBV cccDNA. The contiguous sequence of nucleobases of the nucleic acid molecule of the invention is typically complementary to a conserved region of the A1CF target nucleic acid, as measured across the length of the nucleic acid molecule, optionally with the exception of one or two mismatches, and optionally excluding nucleotide based linker regions which may link the oligonucleotide to an optional functional group such as a conjugate, or other non-complementary terminal nucleotides. The target nucleic acid is a messenger RNA, such as a pre-mRNA which encodes mammalian A1CF protein, such as human A1CF, e.g. the human A1CF pre-mRNA sequence, such as that disclosed as SEQ ID NO: 1, the monkey A1CF pre-mRNA sequence, such as that disclosed as SEQ ID NO: 2, or the mouse A1CF pre-mRNA sequence, such as that disclosed as SEQ ID NO: 3, or a mature A1CF mRNA, such as that a human mature mRNA disclosed as SEQ ID NO: 4, 6, 7, 8, 10, or 11. SEQ ID NOs: 1-13 are DNA sequences—it will be understood that target RNA sequences have uracil (U) bases in place of the thymidine bases (T).

Further information on exemplary target nucleic acids is provided in Tables 2 and 3.

TABLE 3
Overview on target nucleic acids.

| Target Nucleic Acid, Species, Reference | Sequence ID |
|---|---|
| A1CF Homo sapiens pre-mRNA | SEQ ID NO: 1 |
| A1CF Macaca fascicularis pre-mRNA | SEQ ID NO: 2 |
| A1CF Mus musculus pre-mRNA | SEQ ID NO: 3 |
| A1CF Homo sapiens mature mRNA, | SEQ ID NO: 4 |
| variant 1 (ENST00000374001) | |
| A1CF Homo sapiens mature mRNA, | SEQ ID NO: 5 |
| variant 2 (ENST00000395489) | |
| A1CF Homo sapiens mature mRNA, | SEQ ID NO: 6 |
| variant 3 (ENST00000282641) | |
| A1CF Homo sapiens mature mRNA, | SEQ ID NO: 7 |
| variant 4 (ENST00000395495) | |
| A1CF Homo sapiens mature mRNA, | SEQ ID NO: 8 |
| variant 5 (ENST00000373997) | |
| A1CF Homo sapiens mature mRNA, | SEQ ID NO: 9 |
| variant 6 (ENST00000373995) | |
| A1CF Homo sapiens mature mRNA, | SEQ ID NO: 10 |
| variant 8 (ENST00000373993) | |
| A1CF Homo sapiens mature mRNA, | SEQ ID NO: 11 |
| variant 9 (ENST00000414883) | |

In some embodiments, the target nucleic acid is SEQ ID NO: 1.

In some embodiments, the target nucleic acid is SEQ ID NO: 2.

In some embodiments, the target nucleic acid is SEQ ID NO: 3.

In some embodiments, the target nucleic acid is SEQ ID NO: 4.

In some embodiments, the target nucleic acid is SEQ ID NO: 5.

In some embodiments, the target nucleic acid is SEQ ID NO: 6.

In some embodiments, the target nucleic acid is SEQ ID NO: 7.

In some embodiments, the target nucleic acid is SEQ ID NO: 8.

In some embodiments, the target nucleic acid is SEQ ID NO: 9.

In some embodiments, the target nucleic acid is SEQ ID NO: 10.

In some embodiments, the target nucleic acid is SEQ ID NO: 11.

### Target Sequence

The term “target sequence” as used herein refers to a sequence of nucleotides present in the target nucleic acid which comprises the nucleobase sequence which is complementary to the oligonucleotide or nucleic acid molecule of the invention. In some embodiments, the target sequence consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments, the target sequence is longer than the complementary sequence of a nucleic acid molecule of the invention, and may, for example represent a preferred region of the target nucleic acid which may be targeted by several nucleic acid molecules of the invention.

In some embodiments, the target sequence is a sequence selected from the group consisting of a human A1CF mRNA exon, such as an A1CF human mRNA exon selected from the group consisting of e1, e2, e3, e4, e5, e6, e7, e8, e9, e10, e11, e12, 13, e14, and e15, (see for example Table 1 above).

Accordingly, the invention provides for an oligonucleotide, wherein said oligonucleotide comprises a contiguous sequence which is at least 90% complementary, such as fully complementary to an exon region of SEQ ID NO: 1, selected from the group consisting of e1-e15 (see Table 1).

In some embodiments, the target sequence is a sequence selected from the group consisting of a human A1CFmRNA intron, such as an A1CF human mRNA intron selected from the group consisting of i1, i2, i3, i4, i5, i6, i7, i8, i9, i10, i11, i12, i13, and i14 (see for example Table 1 above).

Accordingly, the invention provides for an oligonucleotide, wherein said oligonucleotide comprises a contiguous sequence which is at least 90% complementary, such as fully complementary to an intron region of SEQ ID NO: 1, selected from the group consisting of i1-i14 (see Table 1).

In some embodiments, the target sequence is selected from the group consisting of SEQ ID NO: 12, 13, 14 and 15. In some embodiments, the contiguous nucleotide sequence as referred to herein is at least 90% complementary, such as at least 95% complementary to a target sequence selected from the group consisting of SEQ ID NO: 12, 13, 14 and 15. In some embodiments, the contiguous nucleotide sequence is fully complementary to a target sequence selected from the group consisting of SEQ ID NO: 12, 13, 14 and 15.

The oligonucleotide of the invention comprises a contiguous nucleotide sequence which is complementary to or hybridizes to a region on the target nucleic acid, such as a target sequence described herein.

The target nucleic acid sequence to which the therapeutic oligonucleotide is complementary or hybridizes to generally comprises a stretch of contiguous nucleobases of at least 10 nucleotides. The contiguous nucleotide sequence is between 12 to 70 nucleotides, such as 12 to 50, such as 13 to 30, such as 14 to 25, such as 15 to 20, such as 16 to 18 contiguous nucleotides.

In some embodiments, the oligonucleotide of the present invention targets a region shown in Table 4 or 5.

TABLE 4
Exemplary target regions

| Target | start SEQ | end SEQ |
|---|---|---|
| region | ID NO: 1 | ID NO: 1 |
| 1A | 1 | 31 |
| 2A | 33 | 112 |
| 3A | 114 | 137 |
| 4A | 147 | 171 |
| 5A | 196 | 211 |
| 6A | 215 | 238 |
| 7A | 251 | 267 |
| 8A | 269 | 290 |
| 9A | 301 | 388 |
| 10A | 390 | 425 |
| 11A | 428 | 448 |
| 12A | 455 | 470 |
| 13A | 498 | 514 |
| 14A | 516 | 540 |
| 15A | 560 | 594 |
| 16A | 612 | 647 |
| 17A | 671 | 709 |
| 18A | 719 | 777 |
| 19A | 802 | 818 |
| 20A | 833 | 873 |
| 21A | 888 | 929 |
| 22A | 944 | 959 |
| 23A | 963 | 978 |
| 24A | 980 | 998 |
| 25A | 1023 | 1057 |
| 26A | 1103 | 1132 |
| 27A | 1146 | 1171 |
| 28A | 1175 | 1201 |
| 29A | 1203 | 1303 |
| 30A | 1305 | 1330 |
| 31A | 1333 | 1351 |
| 32A | 1353 | 1435 |
| 33A | 1450 | 1473 |
| 34A | 1481 | 1512 |
| 35A | 1515 | 1538 |
| 36A | 1540 | 1559 |
| 37A | 1562 | 1588 |
| 38A | 1591 | 1613 |
| 39A | 1615 | 1649 |
| 40A | 1634 | 1648 |
| 41A | 1678 | 1723 |
| 42A | 1725 | 1741 |
| 43A | 1742 | 1788 |
| 44A | 1803 | 1848 |
| 45A | 1856 | 1871 |
| 46A | 1873 | 1887 |
| 47A | 1896 | 1941 |
| 48A | 1943 | 1961 |
| 49A | 1970 | 2010 |
| 50A | 2012 | 2034 |
| 51A | 2038 | 2053 |
| 52A | 2072 | 2098 |
| 53A | 2137 | 2164 |
| 54A | 2160 | 2179 |
| 55A | 2181 | 2211 |
| 56A | 2213 | 2230 |
| 57A | 2232 | 2264 |
| 58A | 2266 | 2308 |
| 59A | 2310 | 2357 |
| 60A | 2374 | 2392 |
| 61A | 2394 | 2413 |
| 62A | 2419 | 2437 |
| 63A | 2439 | 2454 |
| 64A | 2471 | 2524 |
| 65A | 2526 | 2554 |
| 66A | 2556 | 2576 |
| 67A | 2581 | 2597 |
| 68A | 2599 | 2620 |
| 69A | 2646 | 2737 |
| 70A | 2739 | 2754 |
| 71A | 2766 | 2786 |
| 72A | 2788 | 2822 |
| 73A | 2823 | 2882 |
| 74A | 2884 | 2951 |
| 75A | 2953 | 3051 |
| 76A | 3053 | 3067 |
| 77A | 3070 | 3118 |
| 78A | 3121 | 3166 |
| 79A | 3179 | 3196 |
| 80A | 3200 | 3215 |
| 81A | 3257 | 3278 |
| 82A | 3279 | 3300 |
| 83A | 3334 | 3366 |
| 84A | 3354 | 3368 |
| 85A | 3391 | 3412 |
| 86A | 3392 | 3410 |
| 87A | 3400 | 3420 |
| 88A | 3414 | 3430 |
| 89A | 3435 | 3452 |
| 90A | 3435 | 3450 |
| 91A | 3465 | 3480 |
| 92A | 3473 | 3508 |
| 93A | 3473 | 3502 |
| 94A | 3477 | 3500 |
| 95A | 3477 | 3499 |
| 96A | 3487 | 3501 |
| 97A | 3520 | 3535 |
| 98A | 3520 | 3538 |
| 99A | 3540 | 3591 |
| 100A | 3593 | 3620 |
| 101A | 3655 | 3711 |
| 102A | 3717 | 3735 |
| 103A | 3737 | 3761 |
| 104A | 3773 | 3802 |
| 105A | 3804 | 3829 |
| 106A | 3838 | 3860 |
| 107A | 3862 | 3878 |
| 108A | 3880 | 3899 |
| 109A | 3926 | 3965 |
| 110A | 3973 | 3987 |
| 111A | 4001 | 4021 |
| 112A | 4023 | 4039 |
| 113A | 4051 | 4086 |
| 114A | 4094 | 4108 |
| 115A | 4121 | 4154 |
| 116A | 4191 | 4242 |
| 117A | 4244 | 4294 |
| 118A | 4397 | 4416 |
| 119A | 4442 | 4475 |
| 120A | 4442 | 4459 |
| 121A | 4505 | 4529 |
| 122A | 4514 | 4529 |
| 123A | 4515 | 4529 |
| 124A | 4542 | 4556 |
| 125A | 4544 | 4561 |
| 126A | 4563 | 4577 |
| 127A | 4585 | 4599 |
| 128A | 4603 | 4623 |
| 129A | 4627 | 4643 |
| 130A | 4687 | 4706 |
| 131A | 4721 | 4746 |
| 132A | 4756 | 4770 |
| 133A | 4772 | 4812 |
| 134A | 4814 | 4845 |
| 135A | 4847 | 4886 |
| 136A | 4896 | 4934 |
| 137A | 4950 | 4978 |
| 138A | 4980 | 5003 |
| 139A | 5006 | 5022 |
| 140A | 5057 | 5079 |
| 141A | 5109 | 5154 |
| 142A | 5177 | 5192 |
| 143A | 5194 | 5216 |
| 144A | 5235 | 5249 |
| 145A | 5264 | 5315 |
| 146A | 5317 | 5353 |
| 147A | 5369 | 5387 |
| 148A | 5389 | 5409 |
| 149A | 5411 | 5425 |
| 150A | 5434 | 5491 |
| 151A | 5505 | 5523 |
| 152A | 5525 | 5551 |
| 153A | 5565 | 5581 |
| 154A | 5628 | 5644 |
| 155A | 5674 | 5689 |
| 156A | 5730 | 5750 |
| 157A | 5755 | 5778 |
| 158A | 5780 | 5810 |
| 159A | 5812 | 5864 |
| 160A | 5869 | 5889 |
| 161A | 5891 | 5915 |
| 162A | 5952 | 5971 |
| 163A | 5986 | 6020 |
| 164A | 6128 | 6164 |
| 165A | 6166 | 6187 |
| 166A | 6192 | 6215 |
| 167A | 6240 | 6257 |
| 168A | 6259 | 6283 |
| 169A | 6331 | 6392 |
| 170A | 6394 | 6423 |
| 171A | 6434 | 6470 |
| 172A | 6472 | 6497 |
| 173A | 6499 | 6533 |
| 174A | 6535 | 6556 |
| 175A | 6558 | 6593 |
| 176A | 6612 | 6631 |
| 177A | 6631 | 6647 |
| 178A | 6649 | 6671 |
| 179A | 6673 | 6719 |
| 180A | 6730 | 6746 |
| 181A | 6804 | 6833 |
| 182A | 6835 | 6853 |
| 183A | 6895 | 6915 |
| 184A | 6917 | 6931 |
| 185A | 6933 | 7001 |
| 186A | 7017 | 7042 |
| 187A | 7059 | 7074 |
| 188A | 7076 | 7096 |
| 189A | 7098 | 7115 |
| 190A | 7129 | 7151 |
| 191A | 7153 | 7181 |
| 192A | 7197 | 7260 |
| 193A | 7262 | 7280 |
| 194A | 7296 | 7421 |
| 195A | 7463 | 7483 |
| 196A | 7489 | 7509 |
| 197A | 7525 | 7539 |
| 198A | 7533 | 7548 |
| 199A | 7550 | 7567 |
| 200A | 7569 | 7590 |
| 201A | 7607 | 7629 |
| 202A | 7624 | 7638 |
| 203A | 7640 | 7660 |
| 204A | 7648 | 7674 |
| 205A | 7663 | 7682 |
| 206A | 7680 | 7707 |
| 207A | 7696 | 7717 |
| 208A | 7719 | 7737 |
| 209A | 7739 | 7778 |
| 210A | 7778 | 7795 |
| 211A | 7780 | 7795 |
| 212A | 7845 | 7898 |
| 213A | 7900 | 7916 |
| 214A | 7918 | 7938 |
| 215A | 7940 | 7960 |
| 216A | 7971 | 7990 |
| 217A | 8003 | 8046 |
| 218A | 8058 | 8075 |
| 219A | 8086 | 8173 |
| 220A | 8201 | 8218 |
| 221A | 8220 | 8252 |
| 222A | 8278 | 8314 |
| 223A | 8322 | 8340 |
| 224A | 8357 | 8372 |
| 225A | 8389 | 8414 |
| 226A | 8416 | 8432 |
| 227A | 8446 | 8472 |
| 228A | 8504 | 8526 |
| 229A | 8528 | 8543 |
| 230A | 8570 | 8587 |
| 231A | 8603 | 8637 |
| 232A | 8665 | 8687 |
| 233A | 8689 | 8722 |
| 234A | 8773 | 8793 |
| 235A | 8795 | 8814 |
| 236A | 8836 | 8851 |
| 237A | 8854 | 8906 |
| 238A | 8921 | 8993 |
| 239A | 9019 | 9047 |
| 240A | 9053 | 9101 |
| 241A | 9103 | 9121 |
| 242A | 9123 | 9159 |
| 243A | 9171 | 9185 |
| 244A | 9187 | 9211 |
| 245A | 9213 | 9229 |
| 246A | 9231 | 9249 |
| 247A | 9251 | 9276 |
| 248A | 9282 | 9326 |
| 249A | 9374 | 9390 |
| 250A | 9407 | 9426 |
| 251A | 9460 | 9476 |
| 252A | 9507 | 9525 |
| 253A | 9535 | 9590 |
| 254A | 9607 | 9628 |
| 255A | 9636 | 9683 |
| 256A | 9685 | 9703 |
| 257A | 9705 | 9733 |
| 258A | 9735 | 9818 |
| 259A | 9820 | 9837 |
| 260A | 9839 | 9896 |
| 261A | 9898 | 9915 |
| 262A | 9917 | 9939 |
| 263A | 9960 | 10000 |
| 264A | 10002 | 10020 |
| 265A | 10031 | 10066 |
| 266A | 10082 | 10166 |
| 267A | 10208 | 10228 |
| 268A | 10230 | 10257 |
| 269A | 10259 | 10278 |
| 270A | 10289 | 10321 |
| 271A | 10325 | 10340 |
| 272A | 10355 | 10369 |
| 273A | 10374 | 10396 |
| 274A | 10406 | 10421 |
| 275A | 10459 | 10510 |
| 276A | 10512 | 10573 |
| 277A | 10592 | 10610 |
| 278A | 10612 | 10635 |
| 279A | 10658 | 10714 |
| 280A | 10716 | 10764 |
| 281A | 10770 | 10818 |
| 282A | 10820 | 10838 |
| 283A | 10858 | 10873 |
| 284A | 10905 | 10928 |
| 285A | 10930 | 10949 |
| 286A | 10959 | 11037 |
| 287A | 11045 | 11077 |
| 288A | 11084 | 11107 |
| 289A | 11109 | 11125 |
| 290A | 11135 | 11190 |
| 291A | 11207 | 11256 |
| 292A | 11269 | 11314 |
| 293A | 11316 | 11334 |
| 294A | 11336 | 11366 |
| 295A | 11388 | 11445 |
| 296A | 11472 | 11496 |
| 297A | 11507 | 11542 |
| 298A | 11567 | 11598 |
| 299A | 11613 | 11648 |
| 300A | 11664 | 11685 |
| 301A | 11687 | 11740 |
| 302A | 11748 | 11802 |
| 303A | 11810 | 11840 |
| 304A | 11842 | 11861 |
| 305A | 11863 | 11878 |
| 306A | 11885 | 11914 |
| 307A | 11922 | 11940 |
| 308A | 11944 | 11975 |
| 309A | 11978 | 12009 |
| 310A | 12011 | 12029 |
| 311A | 12032 | 12053 |
| 312A | 12070 | 12101 |
| 313A | 12107 | 12126 |
| 314A | 12132 | 12162 |
| 315A | 12165 | 12180 |
| 316A | 12240 | 12256 |
| 317A | 12270 | 12292 |
| 318A | 12309 | 12346 |
| 319A | 12348 | 12367 |
| 320A | 12381 | 12403 |
| 321A | 12412 | 12428 |
| 322A | 12442 | 12456 |
| 323A | 12446 | 12467 |
| 324A | 12492 | 12512 |
| 325A | 12501 | 12517 |
| 326A | 12532 | 12560 |
| 327A | 12548 | 12563 |
| 328A | 12549 | 12563 |
| 329A | 12557 | 12571 |
| 330A | 12575 | 12593 |
| 331A | 12594 | 12611 |
| 332A | 12599 | 12635 |
| 333A | 12619 | 12633 |
| 334A | 12639 | 12657 |
| 335A | 12640 | 12656 |
| 336A | 12645 | 12701 |
| 337A | 12645 | 12659 |
| 338A | 12668 | 12683 |
| 339A | 12702 | 12721 |
| 340A | 12703 | 12721 |
| 341A | 12704 | 12722 |
| 342A | 12705 | 12723 |
| 343A | 12706 | 12724 |
| 344A | 12707 | 12725 |
| 345A | 12708 | 12726 |
| 346A | 12709 | 12727 |
| 347A | 12710 | 12728 |
| 348A | 12711 | 12729 |
| 349A | 12711 | 12730 |
| 350A | 12715 | 12732 |
| 351A | 12735 | 12795 |
| 352A | 12815 | 12835 |
| 353A | 12857 | 12873 |
| 354A | 12875 | 12900 |
| 355A | 12902 | 12937 |
| 356A | 12972 | 13033 |
| 357A | 13035 | 13056 |
| 358A | 13090 | 13123 |
| 359A | 13173 | 13219 |
| 360A | 13245 | 13275 |
| 361A | 13301 | 13316 |
| 362A | 13318 | 13342 |
| 363A | 13344 | 13400 |
| 364A | 13402 | 13433 |
| 365A | 13455 | 13547 |
| 366A | 13566 | 13580 |
| 367A | 13582 | 13607 |
| 368A | 13614 | 13628 |
| 369A | 13622 | 13667 |
| 370A | 13669 | 13694 |
| 371A | 13716 | 13757 |
| 372A | 13759 | 13804 |
| 373A | 13806 | 13840 |
| 374A | 13863 | 13897 |
| 375A | 13899 | 13917 |
| 376A | 13919 | 13934 |
| 377A | 13936 | 14008 |
| 378A | 14010 | 14049 |
| 379A | 14086 | 14100 |
| 380A | 14103 | 14118 |
| 381A | 14124 | 14163 |
| 382A | 14174 | 14258 |
| 383A | 14288 | 14319 |
| 384A | 14367 | 14412 |
| 385A | 14422 | 14447 |
| 386A | 14463 | 14480 |
| 387A | 14483 | 14546 |
| 388A | 14548 | 14574 |
| 389A | 14626 | 14641 |
| 390A | 14643 | 14668 |
| 391A | 14673 | 14691 |
| 392A | 14747 | 14767 |
| 393A | 14783 | 14803 |
| 394A | 14820 | 14841 |
| 395A | 14849 | 14871 |
| 396A | 14862 | 14877 |
| 397A | 14899 | 14927 |
| 398A | 14956 | 14974 |
| 399A | 14982 | 15007 |
| 400A | 15017 | 15055 |
| 401A | 15057 | 15087 |
| 402A | 15089 | 15104 |
| 403A | 15104 | 15138 |
| 404A | 15141 | 15180 |
| 405A | 15196 | 15239 |
| 406A | 15241 | 15265 |
| 407A | 15273 | 15291 |
| 408A | 15293 | 15318 |
| 409A | 15325 | 15363 |
| 410A | 15365 | 15385 |
| 411A | 15392 | 15424 |
| 412A | 15426 | 15460 |
| 413A | 15462 | 15476 |
| 414A | 15478 | 15501 |
| 415A | 15521 | 15570 |
| 416A | 15573 | 15587 |
| 417A | 15598 | 15631 |
| 418A | 15649 | 15664 |
| 419A | 15665 | 15690 |
| 420A | 15721 | 15753 |
| 421A | 15755 | 15782 |
| 422A | 15784 | 15838 |
| 423A | 15840 | 15857 |
| 424A | 15859 | 15880 |
| 425A | 15885 | 15928 |
| 426A | 15930 | 15949 |
| 427A | 15977 | 16020 |
| 428A | 16022 | 16039 |
| 429A | 16041 | 16120 |
| 430A | 16131 | 16145 |
| 431A | 16162 | 16199 |
| 432A | 16210 | 16234 |
| 433A | 16240 | 16283 |
| 434A | 16299 | 16345 |
| 435A | 16371 | 16391 |
| 436A | 16393 | 16408 |
| 437A | 16435 | 16481 |
| 438A | 16483 | 16520 |
| 439A | 16522 | 16540 |
| 440A | 16535 | 16552 |
| 441A | 16554 | 16574 |
| 442A | 16581 | 16645 |
| 443A | 16647 | 16672 |
| 444A | 16701 | 16744 |
| 445A | 16746 | 16761 |
| 446A | 16763 | 16793 |
| 447A | 16795 | 16818 |
| 448A | 16820 | 16853 |
| 449A | 16856 | 16874 |
| 450A | 16884 | 16914 |
| 451A | 16916 | 16946 |
| 452A | 16948 | 16968 |
| 453A | 16970 | 17043 |
| 454A | 17046 | 17077 |
| 455A | 17095 | 17116 |
| 456A | 17119 | 17157 |
| 457A | 17171 | 17189 |
| 458A | 17213 | 17233 |
| 459A | 17272 | 17320 |
| 460A | 17322 | 17338 |
| 461A | 17340 | 17356 |
| 462A | 17358 | 17385 |
| 463A | 17389 | 17446 |
| 464A | 17448 | 17483 |
| 465A | 17485 | 17506 |
| 466A | 17584 | 17604 |
| 467A | 17606 | 17638 |
| 468A | 17659 | 17676 |
| 469A | 17687 | 17741 |
| 470A | 17743 | 17758 |
| 471A | 17760 | 17777 |
| 472A | 17779 | 17794 |
| 473A | 17807 | 17826 |
| 474A | 17836 | 17862 |
| 475A | 17880 | 17910 |
| 476A | 17914 | 17930 |
| 477A | 17932 | 17956 |
| 478A | 17958 | 17976 |
| 479A | 17978 | 18005 |
| 480A | 18009 | 18052 |
| 481A | 18054 | 18075 |
| 482A | 18102 | 18128 |
| 483A | 18150 | 18201 |
| 484A | 18203 | 18240 |
| 485A | 18242 | 18279 |
| 486A | 18331 | 18354 |
| 487A | 18351 | 18365 |
| 488A | 18374 | 18406 |
| 489A | 18404 | 18419 |
| 490A | 18408 | 18439 |
| 491A | 18441 | 18471 |
| 492A | 18473 | 18524 |
| 493A | 18526 | 18566 |
| 494A | 18568 | 18617 |
| 495A | 18624 | 18640 |
| 496A | 18642 | 18658 |
| 497A | 18648 | 18697 |
| 498A | 18699 | 18763 |
| 499A | 18723 | 18739 |
| 500A | 18777 | 18792 |
| 501A | 18808 | 18825 |
| 502A | 18827 | 18850 |
| 503A | 18858 | 18920 |
| 504A | 18923 | 18974 |
| 505A | 18976 | 18993 |
| 506A | 18995 | 19058 |
| 507A | 19060 | 19087 |
| 508A | 19089 | 19180 |
| 509A | 19254 | 19273 |
| 510A | 19292 | 19308 |
| 511A | 19326 | 19350 |
| 512A | 19352 | 19395 |
| 513A | 19410 | 19488 |
| 514A | 19507 | 19530 |
| 515A | 19559 | 19585 |
| 516A | 19587 | 19607 |
| 517A | 19614 | 19632 |
| 518A | 19634 | 19678 |
| 519A | 19688 | 19739 |
| 520A | 19741 | 19783 |
| 521A | 19807 | 19821 |
| 522A | 19859 | 19876 |
| 523A | 19878 | 19908 |
| 524A | 19910 | 19949 |
| 525A | 19951 | 19969 |
| 526A | 19972 | 19997 |
| 527A | 20022 | 20044 |
| 528A | 20046 | 20069 |
| 529A | 20088 | 20106 |
| 530A | 20108 | 20140 |
| 531A | 20142 | 20169 |
| 532A | 20174 | 20216 |
| 533A | 20242 | 20259 |
| 534A | 20271 | 20304 |
| 535A | 20427 | 20448 |
| 536A | 20436 | 20458 |
| 537A | 20446 | 20460 |
| 538A | 20481 | 20499 |
| 539A | 20512 | 20531 |
| 540A | 20540 | 20555 |
| 541A | 20555 | 20569 |
| 542A | 20557 | 20572 |
| 543A | 20600 | 20619 |
| 544A | 20631 | 20645 |
| 545A | 20638 | 20655 |
| 546A | 20647 | 20661 |
| 547A | 20683 | 20716 |
| 548A | 20719 | 20738 |
| 549A | 20747 | 20766 |
| 550A | 20780 | 20796 |
| 551A | 20784 | 20805 |
| 552A | 20837 | 20868 |
| 553A | 20839 | 20856 |
| 554A | 20870 | 20895 |
| 555A | 20883 | 20900 |
| 556A | 20912 | 20932 |
| 557A | 20930 | 20966 |
| 558A | 20936 | 20954 |
| 559A | 20970 | 20987 |
| 560A | 20986 | 21000 |
| 561A | 20990 | 21022 |
| 562A | 20998 | 21014 |
| 563A | 21006 | 21020 |
| 564A | 21008 | 21022 |
| 565A | 21010 | 21030 |
| 566A | 21018 | 21040 |
| 567A | 21018 | 21035 |
| 568A | 21065 | 21105 |
| 569A | 21107 | 21135 |
| 570A | 21139 | 21154 |
| 571A | 21161 | 21224 |
| 572A | 21233 | 21258 |
| 573A | 21266 | 21293 |
| 574A | 21295 | 21328 |
| 575A | 21330 | 21357 |
| 576A | 21373 | 21393 |
| 577A | 21395 | 21434 |
| 578A | 21436 | 21454 |
| 579A | 21456 | 21522 |
| 580A | 21529 | 21580 |
| 581A | 21582 | 21612 |
| 582A | 21652 | 21666 |
| 583A | 21667 | 21685 |
| 584A | 21671 | 21685 |
| 585A | 21720 | 21738 |
| 586A | 21740 | 21754 |
| 587A | 21763 | 21800 |
| 588A | 21811 | 21839 |
| 589A | 21837 | 21877 |
| 590A | 21902 | 21942 |
| 591A | 21944 | 21960 |
| 592A | 21975 | 22012 |
| 593A | 22014 | 22032 |
| 594A | 22049 | 22089 |
| 595A | 22110 | 22143 |
| 596A | 22145 | 22159 |
| 597A | 22161 | 22188 |
| 598A | 22190 | 22210 |
| 599A | 22229 | 22260 |
| 600A | 22275 | 22334 |
| 601A | 22360 | 22406 |
| 602A | 22408 | 22422 |
| 603A | 22424 | 22440 |
| 604A | 22442 | 22472 |
| 605A | 22491 | 22531 |
| 606A | 22559 | 22579 |
| 607A | 22584 | 22677 |
| 608A | 22695 | 22734 |
| 609A | 22736 | 22765 |
| 610A | 22767 | 22787 |
| 611A | 22812 | 22826 |
| 612A | 22849 | 22864 |
| 613A | 22866 | 22886 |
| 614A | 22933 | 22998 |
| 615A | 23014 | 23046 |
| 616A | 23082 | 23101 |
| 617A | 23114 | 23146 |
| 618A | 23168 | 23190 |
| 619A | 23192 | 23225 |
| 620A | 23286 | 23309 |
| 621A | 23314 | 23422 |
| 622A | 23419 | 23440 |
| 623A | 23424 | 23438 |
| 624A | 23428 | 23464 |
| 625A | 23475 | 23499 |
| 626A | 23505 | 23522 |
| 627A | 23505 | 23526 |
| 628A | 23505 | 23520 |
| 629A | 23541 | 23561 |
| 630A | 23549 | 23572 |
| 631A | 23549 | 23571 |
| 632A | 23575 | 23592 |
| 633A | 23624 | 23648 |
| 634A | 23650 | 23672 |
| 635A | 23678 | 23699 |
| 636A | 23685 | 23699 |
| 637A | 23779 | 23794 |
| 638A | 23921 | 23939 |
| 639A | 23966 | 23997 |
| 640A | 23993 | 24027 |
| 641A | 23994 | 24008 |
| 642A | 24029 | 24046 |
| 643A | 24053 | 24068 |
| 644A | 24095 | 24109 |
| 645A | 24103 | 24163 |
| 646A | 24103 | 24133 |
| 647A | 24198 | 24218 |
| 648A | 24201 | 24218 |
| 649A | 24223 | 24240 |
| 650A | 24258 | 24272 |
| 651A | 24274 | 24294 |
| 652A | 24356 | 24370 |
| 653A | 24364 | 24394 |
| 654A | 24423 | 24438 |
| 655A | 24454 | 24474 |
| 656A | 24454 | 24495 |
| 657A | 24457 | 24474 |
| 658A | 24497 | 24530 |
| 659A | 24532 | 24555 |
| 660A | 24562 | 24576 |
| 661A | 24578 | 24604 |
| 662A | 24617 | 24662 |
| 663A | 24665 | 24698 |
| 664A | 24697 | 24725 |
| 665A | 24727 | 24746 |
| 666A | 24777 | 24815 |
| 667A | 24818 | 24835 |
| 668A | 24843 | 24880 |
| 669A | 24904 | 24924 |
| 670A | 24926 | 24944 |
| 671A | 24954 | 24970 |
| 672A | 24995 | 25014 |
| 673A | 25044 | 25059 |
| 674A | 25061 | 25080 |
| 675A | 25082 | 25098 |
| 676A | 25108 | 25129 |
| 677A | 25131 | 25193 |
| 678A | 25226 | 25251 |
| 679A | 25276 | 25296 |
| 680A | 25368 | 25383 |
| 681A | 25397 | 25412 |
| 682A | 25414 | 25436 |
| 683A | 25438 | 25483 |
| 684A | 25475 | 25499 |
| 685A | 25501 | 25529 |
| 686A | 25547 | 25561 |
| 687A | 25564 | 25590 |
| 688A | 25598 | 25627 |
| 689A | 25629 | 25776 |
| 690A | 25778 | 25844 |
| 691A | 25842 | 25873 |
| 692A | 25875 | 25894 |
| 693A | 25898 | 25942 |
| 694A | 25944 | 26000 |
| 695A | 26002 | 26027 |
| 696A | 26037 | 26053 |
| 697A | 26055 | 26086 |
| 698A | 26088 | 26116 |
| 699A | 26131 | 26186 |
| 700A | 26190 | 26211 |
| 701A | 26225 | 26246 |
| 702A | 26249 | 26265 |
| 703A | 26276 | 26321 |
| 704A | 26334 | 26355 |
| 705A | 26357 | 26386 |
| 706A | 26398 | 26415 |
| 707A | 26426 | 26455 |
| 708A | 26469 | 26510 |
| 709A | 26544 | 26580 |
| 710A | 26582 | 26602 |
| 711A | 26612 | 26655 |
| 712A | 26671 | 26691 |
| 713A | 26711 | 26725 |
| 714A | 26736 | 26754 |
| 715A | 26760 | 26780 |
| 716A | 26788 | 26831 |
| 717A | 26833 | 26850 |
| 718A | 26860 | 26898 |
| 719A | 26920 | 26936 |
| 720A | 26938 | 26965 |
| 721A | 26984 | 27050 |
| 722A | 27065 | 27107 |
| 723A | 27118 | 27177 |
| 724A | 27187 | 27226 |
| 725A | 27228 | 27243 |
| 726A | 27271 | 27311 |
| 727A | 27330 | 27364 |
| 728A | 27382 | 27407 |
| 729A | 27430 | 27450 |
| 730A | 27446 | 27477 |
| 731A | 27479 | 27517 |
| 732A | 27519 | 27534 |
| 733A | 27549 | 27582 |
| 734A | 27584 | 27634 |
| 735A | 27636 | 27663 |
| 736A | 27682 | 27705 |
| 737A | 27723 | 27745 |
| 738A | 27771 | 27834 |
| 739A | 27899 | 27921 |
| 740A | 27923 | 27980 |
| 741A | 27994 | 28080 |
| 742A | 28097 | 28120 |
| 743A | 28122 | 28139 |
| 744A | 28142 | 28156 |
| 745A | 28176 | 28214 |
| 746A | 28217 | 28232 |
| 747A | 28234 | 28270 |
| 748A | 28272 | 28299 |
| 749A | 28309 | 28341 |
| 750A | 28343 | 28370 |
| 751A | 28372 | 28395 |
| 752A | 28409 | 28423 |
| 753A | 28412 | 28436 |
| 754A | 28457 | 28482 |
| 755A | 28495 | 28513 |
| 756A | 28530 | 28550 |
| 757A | 28552 | 28574 |
| 758A | 28585 | 28600 |
| 759A | 28626 | 28681 |
| 760A | 28693 | 28739 |
| 761A | 28741 | 28763 |
| 762A | 28774 | 28817 |
| 763A | 28819 | 28833 |
| 764A | 28882 | 28900 |
| 765A | 28888 | 28914 |
| 766A | 28931 | 28949 |
| 767A | 28964 | 29011 |
| 768A | 29037 | 29084 |
| 769A | 29086 | 29101 |
| 770A | 29097 | 29134 |
| 771A | 29150 | 29202 |
| 772A | 29206 | 29251 |
| 773A | 29253 | 29285 |
| 774A | 29296 | 29316 |
| 775A | 29318 | 29337 |
| 776A | 29346 | 29404 |
| 777A | 29406 | 29424 |
| 778A | 29455 | 29494 |
| 779A | 29499 | 29553 |
| 780A | 29555 | 29579 |
| 781A | 29586 | 29636 |
| 782A | 29638 | 29696 |
| 783A | 29703 | 29758 |
| 784A | 29754 | 29812 |
| 785A | 29814 | 29843 |
| 786A | 29850 | 29915 |
| 787A | 29917 | 30096 |
| 788A | 30094 | 30108 |
| 789A | 30114 | 30130 |
| 790A | 30132 | 30153 |
| 791A | 30234 | 30275 |
| 792A | 30305 | 30321 |
| 793A | 30338 | 30352 |
| 794A | 30359 | 30392 |
| 795A | 30425 | 30446 |
| 796A | 30463 | 30478 |
| 797A | 30480 | 30520 |
| 798A | 30526 | 30555 |
| 799A | 30567 | 30583 |
| 800A | 30585 | 30602 |
| 801A | 30604 | 30634 |
| 802A | 30636 | 30652 |
| 803A | 30648 | 30664 |
| 804A | 30654 | 30677 |
| 805A | 30690 | 30741 |
| 806A | 30743 | 30759 |
| 807A | 30826 | 30852 |
| 808A | 30878 | 30897 |
| 809A | 30885 | 30899 |
| 810A | 30887 | 30907 |
| 811A | 30909 | 30952 |
| 812A | 30973 | 31031 |
| 813A | 31037 | 31052 |
| 814A | 31044 | 31073 |
| 815A | 31086 | 31109 |
| 816A | 31111 | 31125 |
| 817A | 31127 | 31161 |
| 818A | 31196 | 31242 |
| 819A | 31266 | 31294 |
| 820A | 31296 | 31320 |
| 821A | 31322 | 31350 |
| 822A | 31349 | 31376 |
| 823A | 31378 | 31408 |
| 824A | 31463 | 31529 |
| 825A | 31531 | 31548 |
| 826A | 31585 | 31601 |
| 827A | 31594 | 31621 |
| 828A | 31626 | 31642 |
| 829A | 31644 | 31727 |
| 830A | 31729 | 31752 |
| 831A | 31777 | 31834 |
| 832A | 31846 | 31862 |
| 833A | 31892 | 31925 |
| 834A | 31908 | 31924 |
| 835A | 31913 | 31939 |
| 836A | 31913 | 31928 |
| 837A | 31998 | 32017 |
| 838A | 32022 | 32055 |
| 839A | 32068 | 32089 |
| 840A | 32111 | 32134 |
| 841A | 32136 | 32180 |
| 842A | 32182 | 32226 |
| 843A | 32228 | 32247 |
| 844A | 32261 | 32289 |
| 845A | 32332 | 32354 |
| 846A | 32371 | 32385 |
| 847A | 32404 | 32442 |
| 848A | 32461 | 32477 |
| 849A | 32485 | 32504 |
| 850A | 32520 | 32548 |
| 851A | 32569 | 32597 |
| 852A | 32607 | 32621 |
| 853A | 32638 | 32659 |
| 854A | 32661 | 32693 |
| 855A | 32700 | 32715 |
| 856A | 32717 | 32755 |
| 857A | 32757 | 32789 |
| 858A | 32791 | 32813 |
| 859A | 32815 | 32831 |
| 860A | 32826 | 32864 |
| 861A | 32870 | 32894 |
| 862A | 32904 | 33042 |
| 863A | 33047 | 33098 |
| 864A | 33110 | 33134 |
| 865A | 33136 | 33158 |
| 866A | 33169 | 33203 |
| 867A | 33205 | 33234 |
| 868A | 33241 | 33266 |
| 869A | 33275 | 33302 |
| 870A | 33312 | 33343 |
| 871A | 33356 | 33416 |
| 872A | 33418 | 33433 |
| 873A | 33453 | 33482 |
| 874A | 33498 | 33521 |
| 875A | 33523 | 33537 |
| 876A | 33539 | 33556 |
| 877A | 33556 | 33570 |
| 878A | 33615 | 33629 |
| 879A | 33639 | 33656 |
| 880A | 33658 | 33672 |
| 881A | 33674 | 33693 |
| 882A | 33697 | 33714 |
| 883A | 33716 | 33731 |
| 884A | 33730 | 33780 |
| 885A | 33791 | 33829 |
| 886A | 33831 | 33845 |
| 887A | 33852 | 33870 |
| 888A | 33882 | 33948 |
| 889A | 33959 | 34003 |
| 890A | 34020 | 34039 |
| 891A | 34050 | 34066 |
| 892A | 34088 | 34121 |
| 893A | 34135 | 34175 |
| 894A | 34170 | 34185 |
| 895A | 34194 | 34216 |
| 896A | 34221 | 34250 |
| 897A | 34252 | 34272 |
| 898A | 34274 | 34299 |
| 899A | 34313 | 34375 |
| 900A | 34391 | 34406 |
| 901A | 34411 | 34455 |
| 902A | 34479 | 34495 |
| 903A | 34497 | 34531 |
| 904A | 34533 | 34555 |
| 905A | 34560 | 34592 |
| 906A | 34607 | 34632 |
| 907A | 34648 | 34695 |
| 908A | 34697 | 34717 |
| 909A | 34752 | 34766 |
| 910A | 34775 | 34802 |
| 911A | 34818 | 34843 |
| 912A | 34866 | 34911 |
| 913A | 34913 | 34928 |
| 914A | 34930 | 34944 |
| 915A | 34958 | 34987 |
| 916A | 34992 | 35025 |
| 917A | 35073 | 35117 |
| 918A | 35100 | 35116 |
| 919A | 35125 | 35147 |
| 920A | 35137 | 35153 |
| 921A | 35168 | 35233 |
| 922A | 35259 | 35289 |
| 923A | 35291 | 35309 |
| 924A | 35305 | 35319 |
| 925A | 35311 | 35334 |
| 926A | 35339 | 35354 |
| 927A | 35372 | 35412 |
| 928A | 35414 | 35440 |
| 929A | 35452 | 35489 |
| 930A | 35505 | 35576 |
| 931A | 35579 | 35662 |
| 932A | 35690 | 35705 |
| 933A | 35707 | 35722 |
| 934A | 35711 | 35725 |
| 935A | 35732 | 35747 |
| 936A | 35802 | 35818 |
| 937A | 35823 | 35843 |
| 938A | 35826 | 35842 |
| 939A | 35864 | 35883 |
| 940A | 35865 | 35883 |
| 941A | 35866 | 35884 |
| 942A | 35867 | 35885 |
| 943A | 35868 | 35886 |
| 944A | 35869 | 35887 |
| 945A | 35870 | 35888 |
| 946A | 35871 | 35889 |
| 947A | 35871 | 35890 |
| 948A | 35878 | 35892 |
| 949A | 35900 | 35921 |
| 950A | 35952 | 35967 |
| 951A | 35969 | 35987 |
| 952A | 35989 | 36039 |
| 953A | 36056 | 36070 |
| 954A | 36095 | 36123 |
| 955A | 36185 | 36205 |
| 956A | 36239 | 36255 |
| 957A | 36268 | 36287 |
| 958A | 36294 | 36364 |
| 959A | 36366 | 36384 |
| 960A | 36386 | 36445 |
| 961A | 36457 | 36471 |
| 962A | 36525 | 36542 |
| 963A | 36570 | 36587 |
| 964A | 36589 | 36659 |
| 965A | 36661 | 36675 |
| 966A | 36690 | 36718 |
| 967A | 36735 | 36767 |
| 968A | 36782 | 36802 |
| 969A | 36804 | 36832 |
| 970A | 36869 | 36890 |
| 971A | 36903 | 36923 |
| 972A | 36927 | 36982 |
| 973A | 36995 | 37010 |
| 974A | 37012 | 37029 |
| 975A | 37031 | 37074 |
| 976A | 37076 | 37136 |
| 977A | 37172 | 37237 |
| 978A | 37247 | 37280 |
| 979A | 37292 | 37316 |
| 980A | 37315 | 37337 |
| 981A | 37339 | 37384 |
| 982A | 37398 | 37413 |
| 983A | 37415 | 37443 |
| 984A | 37487 | 37535 |
| 985A | 37553 | 37595 |
| 986A | 37614 | 37644 |
| 987A | 37676 | 37692 |
| 988A | 37727 | 37761 |
| 989A | 37763 | 37782 |
| 990A | 37784 | 37809 |
| 991A | 37837 | 37854 |
| 992A | 37879 | 37906 |
| 993A | 37908 | 37947 |
| 994A | 37935 | 37950 |
| 995A | 37949 | 37988 |
| 996A | 38003 | 38073 |
| 997A | 38075 | 38107 |
| 998A | 38128 | 38160 |
| 999A | 38162 | 38184 |
| 1000A | 38193 | 38215 |
| 1001A | 38221 | 38264 |
| 1002A | 38266 | 38296 |
| 1003A | 38295 | 38311 |
| 1004A | 38313 | 38327 |
| 1005A | 38329 | 38343 |
| 1006A | 38342 | 38414 |
| 1007A | 38429 | 38446 |
| 1008A | 38448 | 38477 |
| 1009A | 38479 | 38566 |
| 1010A | 38576 | 38615 |
| 1011A | 38628 | 38649 |
| 1012A | 38651 | 38706 |
| 1013A | 38708 | 38759 |
| 1014A | 38761 | 38786 |
| 1015A | 38821 | 38852 |
| 1016A | 38886 | 38900 |
| 1017A | 38902 | 38926 |
| 1018A | 38985 | 39003 |
| 1019A | 39045 | 39062 |
| 1020A | 39082 | 39098 |
| 1021A | 39124 | 39156 |
| 1022A | 39173 | 39191 |
| 1023A | 39267 | 39304 |
| 1024A | 39306 | 39343 |
| 1025A | 39346 | 39371 |
| 1026A | 39377 | 39391 |
| 1027A | 39447 | 39463 |
| 1028A | 39465 | 39484 |
| 1029A | 39515 | 39539 |
| 1030A | 39558 | 39595 |
| 1031A | 39605 | 39628 |
| 1032A | 39668 | 39685 |
| 1033A | 39687 | 39710 |
| 1034A | 39707 | 39740 |
| 1035A | 39794 | 39814 |
| 1036A | 39802 | 39834 |
| 1037A | 39841 | 39861 |
| 1038A | 39868 | 39892 |
| 1039A | 39868 | 39884 |
| 1040A | 39870 | 39884 |
| 1041A | 39874 | 39892 |
| 1042A | 39880 | 39901 |
| 1043A | 39889 | 39908 |
| 1044A | 39896 | 39916 |
| 1045A | 39904 | 39921 |
| 1046A | 39917 | 39932 |
| 1047A | 39957 | 39986 |
| 1048A | 39961 | 39984 |
| 1049A | 39961 | 39983 |
| 1050A | 39971 | 39985 |
| 1051A | 40010 | 40035 |
| 1052A | 40053 | 40067 |
| 1053A | 40092 | 40136 |
| 1054A | 40157 | 40175 |
| 1055A | 40177 | 40201 |
| 1056A | 40212 | 40230 |
| 1057A | 40263 | 40281 |
| 1058A | 40347 | 40382 |
| 1059A | 40389 | 40406 |
| 1060A | 40432 | 40451 |
| 1061A | 40453 | 40480 |
| 1062A | 40487 | 40516 |
| 1063A | 40568 | 40587 |
| 1064A | 40655 | 40670 |
| 1065A | 40699 | 40723 |
| 1066A | 40723 | 40744 |
| 1067A | 40746 | 40772 |
| 1068A | 40774 | 40804 |
| 1069A | 40838 | 40853 |
| 1070A | 40855 | 40891 |
| 1071A | 40894 | 40915 |
| 1072A | 40935 | 40961 |
| 1073A | 40964 | 40978 |
| 1074A | 40999 | 41032 |
| 1075A | 41085 | 41111 |
| 1076A | 41114 | 41129 |
| 1077A | 41133 | 41172 |
| 1078A | 41175 | 41207 |
| 1079A | 41209 | 41234 |
| 1080A | 41247 | 41274 |
| 1081A | 41285 | 41310 |
| 1082A | 41290 | 41305 |
| 1083A | 41316 | 41381 |
| 1084A | 41383 | 41405 |
| 1085A | 41407 | 41437 |
| 1086A | 41439 | 41459 |
| 1087A | 41474 | 41490 |
| 1088A | 41527 | 41591 |
| 1089A | 41593 | 41615 |
| 1090A | 41617 | 41698 |
| 1091A | 41700 | 41722 |
| 1092A | 41724 | 41740 |
| 1093A | 41742 | 41772 |
| 1094A | 41819 | 41837 |
| 1095A | 41842 | 41900 |
| 1096A | 41916 | 41961 |
| 1097A | 41963 | 41979 |
| 1098A | 42008 | 42030 |
| 1099A | 42058 | 42079 |
| 1100A | 42108 | 42128 |
| 1101A | 42147 | 42176 |
| 1102A | 42210 | 42225 |
| 1103A | 42218 | 42234 |
| 1104A | 42239 | 42256 |
| 1105A | 42258 | 42281 |
| 1106A | 42317 | 42332 |
| 1107A | 42345 | 42381 |
| 1108A | 42391 | 42430 |
| 1109A | 42434 | 42448 |
| 1110A | 42455 | 42478 |
| 1111A | 42495 | 42538 |
| 1112A | 42551 | 42587 |
| 1113A | 42586 | 42623 |
| 1114A | 42667 | 42688 |
| 1115A | 42690 | 42708 |
| 1116A | 42710 | 42725 |
| 1117A | 42727 | 42765 |
| 1118A | 42767 | 42786 |
| 1119A | 42788 | 42802 |
| 1120A | 42804 | 42820 |
| 1121A | 42822 | 42836 |
| 1122A | 42839 | 42864 |
| 1123A | 42879 | 42915 |
| 1124A | 42917 | 42950 |
| 1125A | 42966 | 42995 |
| 1126A | 42997 | 43029 |
| 1127A | 43059 | 43132 |
| 1128A | 43094 | 43108 |
| 1129A | 43137 | 43161 |
| 1130A | 43166 | 43203 |
| 1131A | 43174 | 43193 |
| 1132A | 43205 | 43221 |
| 1133A | 43211 | 43227 |
| 1134A | 43213 | 43227 |
| 1135A | 43215 | 43230 |
| 1136A | 43215 | 43233 |
| 1137A | 43223 | 43280 |
| 1138A | 43282 | 43297 |
| 1139A | 43291 | 43310 |
| 1140A | 43299 | 43337 |
| 1141A | 43312 | 43326 |
| 1142A | 43339 | 43394 |
| 1143A | 43415 | 43453 |
| 1144A | 43455 | 43474 |
| 1145A | 43485 | 43582 |
| 1146A | 43584 | 43601 |
| 1147A | 43603 | 43647 |
| 1148A | 43663 | 43757 |
| 1149A | 43769 | 43822 |
| 1150A | 43824 | 43849 |
| 1151A | 43865 | 43896 |
| 1152A | 43905 | 43930 |
| 1153A | 43942 | 43976 |
| 1154A | 43978 | 43998 |
| 1155A | 43999 | 44066 |
| 1156A | 44076 | 44117 |
| 1157A | 44123 | 44160 |
| 1158A | 44167 | 44210 |
| 1159A | 44212 | 44230 |
| 1160A | 44232 | 44258 |
| 1161A | 44260 | 44329 |
| 1162A | 44344 | 44375 |
| 1163A | 44377 | 44396 |
| 1164A | 44418 | 44446 |
| 1165A | 44476 | 44490 |
| 1166A | 44494 | 44545 |
| 1167A | 44602 | 44641 |
| 1168A | 44648 | 44682 |
| 1169A | 44685 | 44716 |
| 1170A | 44756 | 44771 |
| 1171A | 44774 | 44793 |
| 1172A | 44800 | 44815 |
| 1173A | 44817 | 44846 |
| 1174A | 44875 | 44940 |
| 1175A | 44942 | 45041 |
| 1176A | 44993 | 45007 |
| 1177A | 45043 | 45060 |
| 1178A | 45066 | 45092 |
| 1179A | 45094 | 45134 |
| 1180A | 45177 | 45193 |
| 1181A | 45209 | 45235 |
| 1182A | 45237 | 45302 |
| 1183A | 45277 | 45292 |
| 1184A | 45304 | 45325 |
| 1185A | 45326 | 45368 |
| 1186A | 45387 | 45402 |
| 1187A | 45411 | 45441 |
| 1188A | 45473 | 45501 |
| 1189A | 45503 | 45519 |
| 1190A | 45521 | 45557 |
| 1191A | 45563 | 45584 |
| 1192A | 45586 | 45614 |
| 1193A | 45650 | 45681 |
| 1194A | 45698 | 45722 |
| 1195A | 45707 | 45721 |
| 1196A | 45710 | 45726 |
| 1197A | 45715 | 45732 |
| 1198A | 45734 | 45783 |
| 1199A | 45746 | 45771 |
| 1200A | 45774 | 45795 |
| 1201A | 45793 | 45808 |
| 1202A | 45796 | 45815 |
| 1203A | 45813 | 45833 |
| 1204A | 45848 | 45875 |
| 1205A | 45877 | 45891 |
| 1206A | 45893 | 45920 |
| 1207A | 45935 | 45955 |
| 1208A | 45965 | 45990 |
| 1209A | 46020 | 46040 |
| 1210A | 46044 | 46064 |
| 1211A | 46072 | 46122 |
| 1212A | 46124 | 46151 |
| 1213A | 46153 | 46184 |
| 1214A | 46197 | 46222 |
| 1215A | 46224 | 46256 |
| 1216A | 46287 | 46304 |
| 1217A | 46306 | 46336 |
| 1218A | 46388 | 46402 |
| 1219A | 46404 | 46424 |
| 1220A | 46426 | 46469 |
| 1221A | 46484 | 46504 |
| 1222A | 46520 | 46559 |
| 1223A | 46572 | 46623 |
| 1224A | 46631 | 46667 |
| 1225A | 46669 | 46721 |
| 1226A | 46723 | 46751 |
| 1227A | 46753 | 46767 |
| 1228A | 46787 | 46822 |
| 1229A | 46825 | 46849 |
| 1230A | 46864 | 46881 |
| 1231A | 46883 | 46899 |
| 1232A | 46901 | 46930 |
| 1233A | 46957 | 46991 |
| 1234A | 47005 | 47020 |
| 1235A | 47036 | 47088 |
| 1236A | 47090 | 47122 |
| 1237A | 47149 | 47179 |
| 1238A | 47155 | 47172 |
| 1239A | 47209 | 47260 |
| 1240A | 47262 | 47286 |
| 1241A | 47296 | 47320 |
| 1242A | 47351 | 47383 |
| 1243A | 47385 | 47404 |
| 1244A | 47446 | 47506 |
| 1245A | 47563 | 47578 |
| 1246A | 47580 | 47620 |
| 1247A | 47635 | 47649 |
| 1248A | 47651 | 47687 |
| 1249A | 47691 | 47723 |
| 1250A | 47725 | 47751 |
| 1251A | 47753 | 47771 |
| 1252A | 47773 | 47796 |
| 1253A | 47827 | 47843 |
| 1254A | 47828 | 47843 |
| 1255A | 47845 | 47868 |
| 1256A | 47884 | 47914 |
| 1257A | 47939 | 47979 |
| 1258A | 47994 | 48018 |
| 1259A | 48020 | 48035 |
| 1260A | 48050 | 48066 |
| 1261A | 48080 | 48130 |
| 1262A | 48132 | 48199 |
| 1263A | 48179 | 48193 |
| 1264A | 48207 | 48281 |
| 1265A | 48283 | 48302 |
| 1266A | 48336 | 48372 |
| 1267A | 48374 | 48394 |
| 1268A | 48405 | 48423 |
| 1269A | 48427 | 48452 |
| 1270A | 48464 | 48493 |
| 1271A | 48511 | 48529 |
| 1272A | 48540 | 48567 |
| 1273A | 48569 | 48586 |
| 1274A | 48588 | 48616 |
| 1275A | 48625 | 48653 |
| 1276A | 48655 | 48703 |
| 1277A | 48744 | 48768 |
| 1278A | 48770 | 48786 |
| 1279A | 49133 | 49149 |
| 1280A | 49133 | 49159 |
| 1281A | 49173 | 49192 |
| 1282A | 49194 | 49233 |
| 1283A | 49235 | 49258 |
| 1284A | 49260 | 49287 |
| 1285A | 49298 | 49546 |
| 1286A | 49548 | 49570 |
| 1287A | 49584 | 49611 |
| 1288A | 49613 | 49630 |
| 1289A | 49640 | 49656 |
| 1290A | 49658 | 49686 |
| 1291A | 49710 | 49735 |
| 1292A | 49714 | 49728 |
| 1293A | 49737 | 49769 |
| 1294A | 49768 | 49791 |
| 1295A | 49793 | 49807 |
| 1296A | 49809 | 49845 |
| 1297A | 49859 | 49884 |
| 1298A | 49886 | 49926 |
| 1299A | 49928 | 49963 |
| 1300A | 49965 | 49991 |
| 1301A | 49993 | 50081 |
| 1302A | 50098 | 50143 |
| 1303A | 50156 | 50175 |
| 1304A | 50177 | 50205 |
| 1305A | 50242 | 50282 |
| 1306A | 50293 | 50308 |
| 1307A | 50312 | 50340 |
| 1308A | 50342 | 50382 |
| 1309A | 50384 | 50407 |
| 1310A | 50409 | 50451 |
| 1311A | 50453 | 50471 |
| 1312A | 50478 | 50502 |
| 1313A | 50504 | 50524 |
| 1314A | 50552 | 50566 |
| 1315A | 50577 | 50622 |
| 1316A | 50648 | 50671 |
| 1317A | 50673 | 50697 |
| 1318A | 50699 | 50714 |
| 1319A | 50719 | 50767 |
| 1320A | 50769 | 50803 |
| 1321A | 50805 | 50820 |
| 1322A | 50822 | 50840 |
| 1323A | 50842 | 50911 |
| 1324A | 50913 | 50975 |
| 1325A | 50991 | 51007 |
| 1326A | 50995 | 51070 |
| 1327A | 51089 | 51105 |
| 1328A | 51110 | 51196 |
| 1329A | 51200 | 51235 |
| 1330A | 51244 | 51281 |
| 1331A | 51315 | 51331 |
| 1332A | 51333 | 51376 |
| 1333A | 51378 | 51422 |
| 1334A | 51465 | 51505 |
| 1335A | 51517 | 51534 |
| 1336A | 51550 | 51567 |
| 1337A | 51663 | 51699 |
| 1338A | 51752 | 51809 |
| 1339A | 51811 | 51894 |
| 1340A | 51901 | 51945 |
| 1341A | 51954 | 51978 |
| 1342A | 51994 | 52014 |
| 1343A | 52016 | 52050 |
| 1344A | 52046 | 52078 |
| 1345A | 52080 | 52111 |
| 1346A | 52119 | 52148 |
| 1347A | 52153 | 52188 |
| 1348A | 52223 | 52255 |
| 1349A | 52268 | 52300 |
| 1350A | 52302 | 52326 |
| 1351A | 52329 | 52361 |
| 1352A | 52363 | 52406 |
| 1353A | 52408 | 52437 |
| 1354A | 52439 | 52462 |
| 1355A | 52469 | 52531 |
| 1356A | 52535 | 52639 |
| 1357A | 52641 | 52662 |
| 1358A | 52664 | 52681 |
| 1359A | 52683 | 52718 |
| 1360A | 52721 | 52756 |
| 1361A | 52758 | 52796 |
| 1362A | 52812 | 52831 |
| 1363A | 52842 | 52861 |
| 1364A | 52865 | 52887 |
| 1365A | 52889 | 52904 |
| 1366A | 52913 | 52933 |
| 1367A | 52935 | 52950 |
| 1368A | 52952 | 52998 |
| 1369A | 53000 | 53022 |
| 1370A | 53024 | 53054 |
| 1371A | 53065 | 53087 |
| 1372A | 53089 | 53107 |
| 1373A | 53161 | 53176 |
| 1374A | 53183 | 53230 |
| 1375A | 53232 | 53260 |
| 1376A | 53262 | 53306 |
| 1377A | 53330 | 53358 |
| 1378A | 53365 | 53398 |
| 1379A | 53400 | 53437 |
| 1380A | 53452 | 53486 |
| 1381A | 53503 | 53551 |
| 1382A | 53511 | 53526 |
| 1383A | 53553 | 53571 |
| 1384A | 53573 | 53602 |
| 1385A | 53610 | 53625 |
| 1386A | 53627 | 53662 |
| 1387A | 53656 | 53676 |
| 1388A | 53680 | 53694 |
| 1389A | 53696 | 53752 |
| 1390A | 53761 | 53775 |
| 1391A | 53777 | 53834 |
| 1392A | 53856 | 53879 |
| 1393A | 53882 | 53924 |
| 1394A | 53936 | 54023 |
| 1395A | 54020 | 54040 |
| 1396A | 54028 | 54054 |
| 1397A | 54034 | 54054 |
| 1398A | 54042 | 54087 |
| 1399A | 54057 | 54078 |
| 1400A | 54062 | 54076 |
| 1401A | 54099 | 54117 |
| 1402A | 54105 | 54126 |
| 1403A | 54105 | 54136 |
| 1404A | 54177 | 54198 |
| 1405A | 54178 | 54198 |
| 1406A | 54186 | 54208 |
| 1407A | 54196 | 54210 |
| 1408A | 54209 | 54223 |
| 1409A | 54237 | 54254 |
| 1410A | 54256 | 54283 |
| 1411A | 54287 | 54314 |
| 1412A | 54316 | 54340 |
| 1413A | 54336 | 54363 |
| 1414A | 54366 | 54392 |
| 1415A | 54399 | 54441 |
| 1416A | 54443 | 54464 |
| 1417A | 54478 | 54496 |
| 1418A | 54507 | 54534 |
| 1419A | 54554 | 54569 |
| 1420A | 54585 | 54604 |
| 1421A | 54615 | 54642 |
| 1422A | 54644 | 54668 |
| 1423A | 54670 | 54688 |
| 1424A | 54730 | 54751 |
| 1425A | 54758 | 54788 |
| 1426A | 54794 | 54809 |
| 1427A | 54824 | 54878 |
| 1428A | 54880 | 54945 |
| 1429A | 54966 | 54984 |
| 1430A | 55001 | 55035 |
| 1431A | 55037 | 55084 |
| 1432A | 55086 | 55110 |
| 1433A | 55112 | 55143 |
| 1434A | 55156 | 55183 |
| 1435A | 55185 | 55232 |
| 1436A | 55234 | 55263 |
| 1437A | 55277 | 55299 |
| 1438A | 55305 | 55345 |
| 1439A | 55359 | 55384 |
| 1440A | 55393 | 55453 |
| 1441A | 55465 | 55479 |
| 1442A | 55484 | 55505 |
| 1443A | 55514 | 55547 |
| 1444A | 55549 | 55592 |
| 1445A | 55608 | 55633 |
| 1446A | 55671 | 55690 |
| 1447A | 55701 | 55725 |
| 1448A | 55727 | 55744 |
| 1449A | 55798 | 55816 |
| 1450A | 55818 | 55839 |
| 1451A | 55851 | 55878 |
| 1452A | 55931 | 55951 |
| 1453A | 55953 | 55975 |
| 1454A | 55997 | 56020 |
| 1455A | 56049 | 56081 |
| 1456A | 56090 | 56138 |
| 1457A | 56150 | 56183 |
| 1458A | 56185 | 56202 |
| 1459A | 56212 | 56252 |
| 1460A | 56274 | 56290 |
| 1461A | 56317 | 56343 |
| 1462A | 56345 | 56373 |
| 1463A | 56375 | 56404 |
| 1464A | 56416 | 56437 |
| 1465A | 56447 | 56472 |
| 466A | 56474 | 56489 |
| 1467A | 56491 | 56543 |
| 1468A | 56552 | 56575 |
| 1469A | 56596 | 56620 |
| 1470A | 56637 | 56682 |
| 1471A | 56706 | 56747 |
| 1472A | 56755 | 56772 |
| 1473A | 56775 | 56817 |
| 1474A | 56819 | 56848 |
| 1475A | 56872 | 56887 |
| 1476A | 56899 | 56922 |
| 1477A | 56963 | 57001 |
| 1478A | 57046 | 57074 |
| 1479A | 57076 | 57090 |
| 1480A | 57092 | 57106 |
| 1481A | 57108 | 57141 |
| 1482A | 57185 | 57206 |
| 1483A | 57221 | 57242 |
| 1484A | 57253 | 57294 |
| 1485A | 57296 | 57311 |
| 1486A | 57322 | 57342 |
| 1487A | 57345 | 57362 |
| 1488A | 57364 | 57403 |
| 1489A | 57406 | 57586 |
| 1490A | 59248 | 59300 |
| 1491A | 59302 | 59320 |
| 1492A | 59343 | 59388 |
| 1493A | 59399 | 59420 |
| 1494A | 59427 | 59452 |
| 1495A | 59454 | 59491 |
| 1496A | 59487 | 59511 |
| 1497A | 59537 | 59575 |
| 1498A | 59580 | 59594 |
| 1499A | 59600 | 59663 |
| 1500A | 59665 | 59686 |
| 1501A | 59688 | 59710 |
| 1502A | 59716 | 59747 |
| 1503A | 59755 | 59788 |
| 1504A | 59790 | 59808 |
| 1505A | 59815 | 59830 |
| 1506A | 59832 | 59857 |
| 1507A | 59855 | 59875 |
| 1508A | 59887 | 59908 |
| 1509A | 59925 | 59949 |
| 1510A | 59957 | 59979 |
| 1511A | 59992 | 60007 |
| 1512A | 60009 | 60048 |
| 1513A | 60061 | 60102 |
| 1514A | 60134 | 60150 |
| 1515A | 60152 | 60182 |
| 1516A | 60184 | 60208 |
| 1517A | 60226 | 60244 |
| 1518A | 60314 | 60351 |
| 1519A | 60353 | 60400 |
| 1520A | 60423 | 60438 |
| 1521A | 60454 | 60473 |
| 1522A | 60475 | 60501 |
| 1523A | 60503 | 60526 |
| 1524A | 60528 | 60571 |
| 1525A | 60580 | 60607 |
| 1526A | 60617 | 60634 |
| 1527A | 60638 | 60664 |
| 1528A | 60668 | 60713 |
| 1529A | 60731 | 60761 |
| 1530A | 60763 | 60795 |
| 1531A | 60797 | 60832 |
| 1532A | 60847 | 60867 |
| 1533A | 60881 | 60897 |
| 1534A | 60899 | 60919 |
| 1535A | 60935 | 60966 |
| 1536A | 60979 | 61025 |
| 1537A | 61051 | 61068 |
| 1538A | 61070 | 61098 |
| 1539A | 61110 | 61127 |
| 1540A | 61152 | 61200 |
| 1541A | 61205 | 61241 |
| 1542A | 61243 | 61308 |
| 1543A | 61307 | 61323 |
| 1544A | 61325 | 61369 |
| 1545A | 61376 | 61390 |
| 1546A | 61392 | 61406 |
| 1547A | 61408 | 61423 |
| 1548A | 61427 | 61441 |
| 1549A | 61480 | 61522 |
| 1550A | 61537 | 61580 |
| 1551A | 61596 | 61615 |
| 1552A | 61607 | 61623 |
| 1553A | 61631 | 61659 |
| 1554A | 61671 | 61688 |
| 1555A | 61686 | 61738 |
| 1556A | 61764 | 61805 |
| 1557A | 61807 | 61829 |
| 1558A | 61838 | 61863 |
| 1559A | 62301 | 62315 |
| 1560A | 62357 | 62396 |
| 1561A | 62398 | 62419 |
| 1562A | 62429 | 62452 |
| 1563A | 62454 | 62482 |
| 1564A | 62492 | 62532 |
| 1565A | 62540 | 62560 |
| 1566A | 62564 | 62608 |
| 1567A | 62631 | 62645 |
| 1568A | 62647 | 62695 |
| 1569A | 62697 | 62717 |
| 1570A | 62719 | 62739 |
| 1571A | 62753 | 62768 |
| 1572A | 62768 | 62784 |
| 1573A | 62795 | 62824 |
| 1574A | 62827 | 62863 |
| 1575A | 62903 | 62920 |
| 1576A | 62922 | 62937 |
| 1577A | 62939 | 62976 |
| 1578A | 62978 | 63026 |
| 1579A | 63028 | 63070 |
| 1580A | 63087 | 63131 |
| 1581A | 63141 | 63175 |
| 1582A | 63181 | 63223 |
| 1583A | 63225 | 63242 |
| 1584A | 63244 | 63278 |
| 1585A | 63280 | 63306 |
| 1586A | 63310 | 63340 |
| 1587A | 63344 | 63371 |
| 1588A | 63373 | 63439 |
| 1589A | 63444 | 63458 |
| 1590A | 63460 | 63494 |
| 1591A | 63510 | 63527 |
| 1592A | 63529 | 63544 |
| 1593A | 63548 | 63576 |
| 1594A | 63588 | 63607 |
| 1595A | 63609 | 63624 |
| 1596A | 63642 | 63680 |
| 1597A | 63682 | 63713 |
| 1598A | 63759 | 63778 |
| 1599A | 63788 | 63822 |
| 1600A | 63849 | 63863 |
| 1601A | 63851 | 63869 |
| 1602A | 63894 | 63911 |
| 1603A | 63913 | 63941 |
| 1604A | 63920 | 63937 |
| 1605A | 63933 | 63947 |
| 1606A | 63935 | 63989 |
| 1607A | 63964 | 63981 |
| 1608A | 63992 | 64011 |
| 1609A | 63999 | 64015 |
| 1610A | 64004 | 64030 |
| 1611A | 64022 | 64050 |
| 1612A | 64065 | 64079 |
| 1613A | 64079 | 64113 |
| 1614A | 64093 | 64113 |
| 1615A | 64101 | 64118 |
| 1616A | 64161 | 64179 |
| 1617A | 64194 | 64208 |
| 1618A | 64214 | 64285 |
| 1619A | 64300 | 64327 |
| 1620A | 64338 | 64373 |
| 1621A | 64375 | 64413 |
| 1622A | 64433 | 64460 |
| 1623A | 64480 | 64494 |
| 1624A | 64501 | 64523 |
| 1625A | 64525 | 64540 |
| 1626A | 64556 | 64573 |
| 1627A | 64603 | 64635 |
| 1628A | 64645 | 64665 |
| 1629A | 64667 | 64701 |
| 1630A | 64729 | 64751 |
| 1631A | 64792 | 64808 |
| 1632A | 64810 | 64858 |
| 1633A | 64860 | 64875 |
| 1634A | 64882 | 64906 |
| 1635A | 64939 | 64956 |
| 1636A | 64958 | 64978 |
| 1637A | 64992 | 65008 |
| 1638A | 65010 | 65135 |
| 1639A | 65137 | 65157 |
| 1640A | 65159 | 65173 |
| 1641A | 65187 | 65203 |
| 1642A | 65208 | 65291 |
| 1643A | 65293 | 65322 |
| 1644A | 65362 | 65387 |
| 1645A | 65389 | 65405 |
| 1646A | 65407 | 65462 |
| 1647A | 65464 | 65575 |
| 1648A | 65577 | 65615 |
| 1649A | 65626 | 65640 |
| 1650A | 65642 | 65671 |
| 1651A | 65673 | 65700 |
| 1652A | 65702 | 65723 |
| 1653A | 65725 | 65764 |
| 1654A | 65781 | 65797 |
| 1655A | 65806 | 65823 |
| 1656A | 65825 | 65839 |
| 1657A | 65841 | 65883 |
| 1658A | 65896 | 65939 |
| 1659A | 65947 | 66010 |
| 1660A | 66012 | 66026 |
| 1661A | 66038 | 66058 |
| 1662A | 66062 | 66109 |
| 1663A | 66120 | 66136 |
| 1664A | 66131 | 66214 |
| 1665A | 66216 | 66253 |
| 1666A | 66255 | 66271 |
| 1667A | 66273 | 66306 |
| 1668A | 66315 | 66331 |
| 1669A | 66335 | 66384 |
| 1670A | 66409 | 66426 |
| 1671A | 66410 | 66425 |
| 1672A | 66430 | 66459 |
| 1673A | 66470 | 66492 |
| 1674A | 66494 | 66524 |
| 1675A | 66499 | 66513 |
| 1676A | 66501 | 66515 |
| 1677A | 66537 | 66552 |
| 1678A | 66566 | 66606 |
| 1679A | 66619 | 66638 |
| 1680A | 66640 | 66657 |
| 1681A | 66656 | 66674 |
| 1682A | 66677 | 66693 |
| 1683A | 66695 | 66734 |
| 1684A | 66740 | 66788 |
| 1685A | 66790 | 66828 |
| 1686A | 66830 | 66845 |
| 1687A | 66847 | 66870 |
| 1688A | 66872 | 66892 |
| 1689A | 66923 | 66948 |
| 1690A | 66960 | 66974 |
| 1691A | 66973 | 67017 |
| 1692A | 67037 | 67061 |
| 1693A | 67072 | 67106 |
| 1694A | 67108 | 67150 |
| 1695A | 67159 | 67174 |
| 1696A | 67176 | 67191 |
| 1697A | 67189 | 67218 |
| 1698A | 67226 | 67268 |
| 1699A | 67247 | 67263 |
| 1700A | 67298 | 67322 |
| 1701A | 67325 | 67342 |
| 1702A | 67348 | 67362 |
| 1703A | 67399 | 67429 |
| 1704A | 67446 | 67464 |
| 1705A | 67466 | 67505 |
| 1706A | 67507 | 67524 |
| 1707A | 67526 | 67552 |
| 1708A | 67600 | 67617 |
| 1709A | 67618 | 67634 |
| 1710A | 67636 | 67660 |
| 1711A | 67677 | 67708 |
| 1712A | 67710 | 67744 |
| 1713A | 67759 | 67788 |
| 1714A | 67837 | 67859 |
| 1715A | 67861 | 67884 |
| 1716A | 67886 | 67905 |
| 1717A | 67929 | 67951 |
| 1718A | 67956 | 67993 |
| 1719A | 67995 | 68014 |
| 1720A | 68018 | 68032 |
| 1721A | 68034 | 68048 |
| 1722A | 68050 | 68074 |
| 1723A | 68081 | 68134 |
| 1724A | 68144 | 68215 |
| 1725A | 68232 | 68288 |
| 1726A | 68290 | 68313 |
| 1727A | 68315 | 68358 |
| 1728A | 68360 | 68381 |
| 1729A | 68397 | 68414 |
| 1730A | 68416 | 68434 |
| 1731A | 68436 | 68453 |
| 1732A | 68455 | 68504 |
| 1733A | 68536 | 68562 |
| 1734A | 68564 | 68579 |
| 1735A | 68591 | 68606 |
| 1736A | 68655 | 68706 |
| 1737A | 68708 | 68727 |
| 1738A | 68729 | 68833 |
| 1739A | 68909 | 68957 |
| 1740A | 68959 | 68992 |
| 1741A | 68994 | 69026 |
| 1742A | 69038 | 69058 |
| 1743A | 69054 | 69068 |
| 1744A | 69060 | 69106 |
| 1745A | 69108 | 69135 |
| 1746A | 69137 | 69166 |
| 1747A | 69168 | 69207 |
| 1748A | 69209 | 69224 |
| 1749A | 69236 | 69252 |
| 1750A | 69253 | 69297 |
| 1751A | 69299 | 69318 |
| 1752A | 69320 | 69355 |
| 1753A | 69376 | 69404 |
| 1754A | 69406 | 69464 |
| 1755A | 69466 | 69578 |
| 1756A | 69586 | 69629 |
| 1757A | 69631 | 69663 |
| 1758A | 69677 | 69696 |
| 1759A | 69708 | 69722 |
| 1760A | 69724 | 69760 |
| 1761A | 69762 | 69779 |
| 1762A | 69784 | 69798 |
| 1763A | 69800 | 69822 |
| 1764A | 69833 | 69866 |
| 1765A | 69869 | 69912 |
| 1766A | 69921 | 69957 |
| 1767A | 69974 | 69989 |
| 1768A | 69996 | 70128 |
| 1769A | 70131 | 70166 |
| 1770A | 70203 | 70217 |
| 1771A | 70297 | 70320 |
| 1772A | 70322 | 70361 |
| 1773A | 70363 | 70388 |
| 1774A | 70393 | 70417 |
| 1775A | 70419 | 70438 |
| 1776A | 70447 | 70465 |
| 1777A | 70493 | 70509 |
| 1778A | 70517 | 70541 |
| 1779A | 70561 | 70590 |
| 1780A | 70598 | 70640 |
| 1781A | 70669 | 70688 |
| 1782A | 70690 | 70730 |
| 1783A | 70767 | 70782 |
| 1784A | 70784 | 70800 |
| 1785A | 70802 | 70816 |
| 1786A | 70832 | 70881 |
| 1787A | 70885 | 70906 |
| 1788A | 70910 | 70984 |
| 1789A | 70986 | 71002 |
| 1790A | 71004 | 71018 |
| 1791A | 71020 | 71071 |
| 1792A | 71073 | 71103 |
| 1793A | 71105 | 71135 |
| 1794A | 71178 | 71193 |
| 1795A | 71209 | 71238 |
| 1796A | 71240 | 71265 |
| 1797A | 71267 | 71297 |
| 1798A | 71316 | 71337 |
| 1799A | 71407 | 71438 |
| 1800A | 71449 | 71495 |
| 1801A | 71497 | 71605 |
| 1802A | 71607 | 71709 |
| 1803A | 71711 | 71740 |
| 1804A | 71742 | 71767 |
| 1805A | 71769 | 71785 |
| 1806A | 71787 | 71882 |
| 1807A | 71884 | 71926 |
| 1808A | 71920 | 71945 |
| 1809A | 71970 | 71984 |
| 1810A | 71993 | 72021 |
| 1811A | 72039 | 72109 |
| 1812A | 72111 | 72167 |
| 1813A | 72204 | 72267 |
| 1814A | 72295 | 72333 |
| 1815A | 72336 | 72355 |
| 1816A | 72377 | 72391 |
| 1817A | 72393 | 72411 |
| 1818A | 72413 | 72436 |
| 1819A | 72470 | 72492 |
| 1820A | 72494 | 72556 |
| 1821A | 72558 | 72576 |
| 1822A | 72577 | 72591 |
| 1823A | 72598 | 72640 |
| 1824A | 72642 | 72687 |
| 1825A | 72687 | 72707 |
| 1826A | 72735 | 72793 |
| 1827A | 72797 | 72811 |
| 1828A | 72816 | 72837 |
| 1829A | 72843 | 72879 |
| 1830A | 72881 | 72896 |
| 1831A | 72928 | 72958 |
| 1832A | 72974 | 73027 |
| 1833A | 73046 | 73061 |
| 1834A | 73066 | 73087 |
| 1835A | 73099 | 73122 |
| 1836A | 73126 | 73141 |
| 1837A | 73160 | 73182 |
| 1838A | 73193 | 73221 |
| 1839A | 73241 | 73263 |
| 1840A | 73277 | 73309 |
| 1841A | 73311 | 73330 |
| 1842A | 73334 | 73350 |
| 1843A | 73367 | 73390 |
| 1844A | 73403 | 73438 |
| 1845A | 73444 | 73466 |
| 1846A | 73477 | 73497 |
| 1847A | 73503 | 73537 |
| 1848A | 73539 | 73577 |
| 1849A | 73596 | 73673 |
| 1850A | 73675 | 73691 |
| 1851A | 73708 | 73724 |
| 1852A | 73726 | 73774 |
| 1853A | 73776 | 73800 |
| 1854A | 73802 | 73866 |
| 1855A | 73871 | 73910 |
| 1856A | 73935 | 73969 |
| 1857A | 73971 | 73985 |
| 1858A | 74013 | 74064 |
| 1859A | 74076 | 74097 |
| 1860A | 74114 | 74129 |
| 1861A | 74136 | 74165 |
| 1862A | 74167 | 74186 |
| 1863A | 74188 | 74254 |
| 1864A | 74271 | 74372 |
| 1865A | 74374 | 74388 |
| 1866A | 74401 | 74432 |
| 1867A | 74449 | 74474 |
| 1868A | 74476 | 74516 |
| 1869A | 74518 | 74555 |
| 1870A | 74550 | 74582 |
| 1871A | 74614 | 74680 |
| 1872A | 74704 | 74752 |
| 1873A | 74774 | 74797 |
| 1874A | 74802 | 74856 |
| 1875A | 74867 | 74923 |
| 1876A | 74903 | 74917 |
| 1877A | 74937 | 74951 |
| 1878A | 74953 | 74975 |
| 1879A | 74958 | 74972 |
| 1880A | 74969 | 75003 |
| 1881A | 74974 | 74989 |
| 1882A | 74991 | 75005 |
| 1883A | 75023 | 75039 |
| 1884A | 75024 | 75039 |
| 1885A | 75051 | 75066 |
| 1886A | 75080 | 75098 |
| 1887A | 75095 | 75109 |
| 1888A | 75135 | 75200 |
| 1889A | 75189 | 75203 |
| 1890A | 75223 | 75238 |
| 1891A | 75245 | 75282 |
| 1892A | 75293 | 75310 |
| 1893A | 75312 | 75335 |
| 1894A | 75337 | 75355 |
| 1895A | 75364 | 75394 |
| 1896A | 75411 | 75432 |
| 1897A | 75434 | 75467 |
| 1898A | 75481 | 75512 |
| 1899A | 75514 | 75530 |
| 1900A | 75532 | 75547 |
| 1901A | 75572 | 75651 |
| 1902A | 75667 | 75687 |
| 1903A | 75689 | 75740 |
| 1904A | 75739 | 75760 |
| 1905A | 75762 | 75849 |
| 1906A | 75859 | 75876 |
| 1907A | 75885 | 75900 |
| 1908A | 75907 | 75929 |
| 1909A | 75931 | 75949 |
| 1910A | 75951 | 75973 |
| 1911A | 75975 | 76073 |
| 1912A | 76075 | 76092 |
| 1913A | 76094 | 76110 |
| 1914A | 76118 | 76141 |
| 1915A | 76143 | 76158 |
| 1916A | 76160 | 76176 |
| 1917A | 76179 | 76211 |
| 1918A | 76224 | 76240 |
| 1919A | 76247 | 76267 |
| 1920A | 76269 | 76290 |
| 1921A | 76292 | 76306 |
| 1922A | 76308 | 76335 |
| 1923A | 76343 | 76364 |
| 1924A | 76366 | 76390 |
| 1925A | 76392 | 76409 |
| 1926A | 76428 | 76486 |
| 1927A | 76488 | 76502 |
| 1928A | 76519 | 76539 |
| 1929A | 76541 | 76564 |
| 1930A | 76575 | 76589 |
| 1931A | 76606 | 76620 |
| 1932A | 76640 | 76654 |
| 1933A | 76645 | 76663 |
| 1934A | 76653 | 76678 |
| 1935A | 76727 | 76756 |
| 1936A | 76782 | 76799 |
| 1937A | 76805 | 76819 |
| 1938A | 76831 | 76858 |
| 1939A | 76907 | 76927 |
| 1940A | 76942 | 76985 |
| 1941A | 76987 | 77008 |
| 1942A | 77010 | 77045 |
| 1943A | 77056 | 77085 |
| 1944A | 77101 | 77139 |
| 1945A | 77158 | 77172 |
| 1946A | 77174 | 77197 |
| 1947A | 77199 | 77221 |
| 1948A | 77223 | 77262 |
| 1949A | 77267 | 77281 |
| 1950A | 77283 | 77332 |
| 1951A | 77349 | 77363 |
| 1952A | 77383 | 77465 |
| 1953A | 77478 | 77516 |
| 1954A | 77518 | 77553 |
| 1955A | 77555 | 77579 |
| 1956A | 77594 | 77628 |
| 1957A | 77631 | 77684 |
| 1958A | 77686 | 77715 |
| 1959A | 77733 | 77794 |
| 1960A | 77796 | 77835 |
| 1961A | 77854 | 77875 |
| 1962A | 77883 | 77899 |
| 1963A | 77920 | 77940 |
| 1964A | 77942 | 77963 |
| 1965A | 77978 | 77998 |
| 1966A | 78009 | 78033 |
| 1967A | 78035 | 78075 |
| 1968A | 78092 | 78111 |
| 1969A | 78113 | 78142 |
| 1970A | 78144 | 78176 |
| 1971A | 78189 | 78203 |
| 1972A | 78215 | 78250 |
| 1973A | 78267 | 78298 |
| 1974A | 78313 | 78360 |
| 1975A | 78386 | 78408 |
| 1976A | 78410 | 78449 |
| 1977A | 78467 | 78491 |
| 1978A | 78494 | 78517 |
| 1979A | 78519 | 78552 |
| 1980A | 78554 | 78578 |
| 1981A | 78589 | 78608 |
| 1982A | 78620 | 78657 |
| 1983A | 78659 | 78674 |
| 1984A | 78676 | 78696 |
| 1985A | 78719 | 78769 |
| 1986A | 78771 | 78823 |
| 1987A | 78834 | 78919 |
| 1988A | 78921 | 78938 |
| 1989A | 78953 | 78991 |
| 1990A | 78992 | 79006 |
| 1991A | 78992 | 79007 |
| 1992A | 81650 | 81664 |
| 1993A | 82345 | 82366 |
| 1994A | 82358 | 82372 |
| 1995A | 82390 | 82406 |
| 1996A | 82531 | 82547 |
| 1997A | 82535 | 82550 |
| 1998A | 83245 | 83259 |
| 1999A | 83709 | 83723 |
| 2000A | 83901 | 83918 |
| 2001A | 85858 | 85873 |

In some embodiments, the target sequence is selected from the group consisting of target regions 1A to 2001A as shown in Table 4 above.

TABLE 5
Exemplary target regions

| Target | start SEQ | end SEQ |
|---|---|---|
| region | ID NO: 1 | ID NO: 1 |
| 1C | 39 | 60 |
| 2C | 60 | 78 |
| 3C | 2314 | 2327 |
| 4C | 2911 | 2951 |
| 5C | 3211 | 3224 |
| 6C | 4669 | 4682 |
| 7C | 4670 | 4683 |
| 8C | 5059 | 5073 |
| 9C | 5789 | 5802 |
| 10C | 6577 | 6591 |
| 11C | 7773 | 7786 |
| 12C | 8088 | 8101 |
| 13C | 8773 | 8786 |
| 14C | 11161 | 11175 |
| 15C | 11431 | 11444 |
| 16C | 12446 | 12459 |
| 17C | 12703 | 12721 |
| 18C | 12703 | 12717 |
| 19C | 12704 | 12722 |
| 20C | 12704 | 12718 |
| 21C | 12705 | 12723 |
| 22C | 12705 | 12719 |
| 23C | 12706 | 12724 |
| 24C | 2706 | 12720 |
| 25C | 12707 | 12725 |
| 26C | 12707 | 12721 |
| 27C | 12708 | 12726 |
| 28C | 12708 | 12722 |
| 29C | 12709 | 12727 |
| 30C | 12709 | 12723 |
| 31C | 12710 | 12728 |
| 32C | 12710 | 12724 |
| 33C | 12711 | 12729 |
| 34C | 12711 | 12725 |
| 35C | 12711 | 12730 |
| 36C | 12712 | 12726 |
| 37C | 12713 | 12727 |
| 38C | 12714 | 12728 |
| 39C | 12715 | 12730 |
| 40C | 12715 | 12729 |
| 41C | 12717 | 12730 |
| 42C | 12718 | 12732 |
| 43C | 13178 | 13191 |
| 44C | 15649 | 15662 |
| 45C | 15822 | 15835 |
| 46C | 15890 | 15903 |
| 47C | 16495 | 16508 |
| 48C | 19257 | 19270 |
| 49C | 19891 | 19907 |
| 50C | 22624 | 22642 |
| 51C | 22707 | 22720 |
| 52C | 25655 | 25776 |
| 53C | 25793 | 25844 |
| 54C | 25850 | 25869 |
| 55C | 27809 | 27825 |
| 56C | 28623 | 28636 |
| 57C | 29713 | 29728 |
| 58C | 29982 | 30004 |
| 59C | 30008 | 30021 |
| 60C | 30880 | 30897 |
| 61C | 30882 | 30897 |
| 62C | 30885 | 30899 |
| 63C | 30887 | 30902 |
| 64C | 30986 | 30999 |
| 65C | 31588 | 31601 |
| 66C | 31911 | 31925 |
| 67C | 31913 | 31926 |
| 68C | 32075 | 32088 |
| 69C | 32266 | 32279 |
| 70C | 33213 | 33232 |
| 71C | 35865 | 35883 |
| 72C | 35865 | 35879 |
| 73C | 35866 | 35884 |
| 74C | 35866 | 35880 |
| 75C | 35867 | 35885 |
| 76C | 35867 | 35881 |
| 77C | 35868 | 35886 |
| 78C | 35868 | 35882 |
| 79C | 35869 | 35887 |
| 80C | 35869 | 35883 |
| 81C | 35870 | 35888 |
| 82C | 35870 | 35884 |
| 83C | 35871 | 35889 |
| 84C | 35871 | 35885 |
| 85C | 35871 | 35890 |
| 86C | 35872 | 35886 |
| 87C | 35873 | 35887 |
| 88C | 35874 | 35888 |
| 89C | 35875 | 35890 |
| 90C | 35875 | 35889 |
| 91C | 35877 | 35890 |
| 92C | 35878 | 35891 |
| 93C | 38221 | 38234 |
| 94C | 38388 | 38402 |
| 95C | 38596 | 38615 |
| 96C | 38667 | 38686 |
| 97C | 39710 | 39723 |
| 98C | 41289 | 41303 |
| 99C | 41290 | 41303 |
| 100C | 41294 | 41310 |
| 101C | 41548 | 41562 |
| 102C | 41551 | 41567 |
| 103C | 41572 | 41588 |
| 104C | 41680 | 41693 |
| 105C | 42012 | 42025 |
| 106C | 42319 | 42332 |
| 107C | 43518 | 43532 |
| 108C | 43585 | 43601 |
| 109C | 43586 | 43599 |
| 110C | 43687 | 43707 |
| 111C | 43709 | 43727 |
| 112C | 43741 | 43757 |
| 113C | 43770 | 43812 |
| 114C | 44217 | 44230 |
| 115C | 45386 | 45399 |
| 116C | 45387 | 45400 |
| 117C | 46795 | 46812 |
| 118C | 49386 | 49402 |
| 119C | 49431 | 49444 |
| 120C | 49446 | 49459 |
| 121C | 49518 | 49534 |
| 122C | 49737 | 49751 |
| 123C | 49777 | 49790 |
| 124C | 50578 | 50592 |
| 125C | 52491 | 52504 |
| 126C | 57296 | 57311 |
| 127C | 57374 | 57393 |
| 128C | 57406 | 57426 |
| 129C | 57488 | 57504 |
| 130C | 57512 | 57533 |
| 131C | 59439 | 59452 |
| 132C | 59460 | 59474 |
| 133C | 60638 | 60653 |
| 134C | 60681 | 60700 |
| 135C | 60881 | 60895 |
| 136C | 61260 | 61280 |
| 137C | 62960 | 62976 |
| 138C | 64306 | 64320 |
| 139C | 65023 | 65036 |
| 140C | 65062 | 65099 |
| 141C | 65498 | 65511 |
| 142C | 65850 | 65863 |
| 143C | 66276 | 66289 |
| 144C | 67447 | 67460 |
| 145C | 67508 | 67521 |
| 146C | 67861 | 67874 |
| 147C | 69112 | 69126 |
| 148C | 69383 | 69404 |
| 149C | 69436 | 69464 |
| 150C | 69489 | 69506 |
| 151C | 69541 | 69573 |
| 152C | 69601 | 69617 |
| 153C | 69833 | 69854 |
| 154C | 70939 | 70955 |
| 155C | 71029 | 71043 |
| 156C | 71465 | 71488 |
| 157C | 71531 | 71605 |
| 158C | 71607 | 71629 |
| 159C | 71631 | 71647 |
| 160C | 71649 | 71671 |
| 161C | 71673 | 71707 |
| 162C | 71724 | 71740 |
| 163C | 71751 | 71767 |
| 164C | 71796 | 71809 |
| 165C | 72008 | 72021 |
| 166C | 72777 | 72790 |
| 167C | 73605 | 73625 |
| 168C | 74278 | 74291 |
| 169C | 74295 | 74309 |
| 170C | 74350 | 74370 |
| 171C | 74492 | 74510 |
| 172C | 74518 | 74549 |
| 173C | 74617 | 74639 |
| 174C | 75624 | 75644 |
| 175C | 78777 | 78808 |
| 176C | 78834 | 78850 |
| 177C | 78858 | 78901 |
| 178C | 78992 | 79005 |

In some embodiments, the target sequence is selected from the group consisting of target regions 10 to 178C as shown in Table 5 above.

### Target Cell

The term a “target cell” as used herein refers to a cell which is expressing the target nucleic acid. For the therapeutic use of the present invention it is advantageous if the target cell is infected with HBV. In some embodiments, the target cell may be in vivo or in vitro. In some embodiments, the target cell is a mammalian cell such as a rodent cell, such as a mouse cell or a rat cell, or a woodchuck cell or a primate cell such as a monkey cell (e.g. a cynomolgus monkey cell) or a human cell.

In preferred embodiments, the target cell expresses A1CF mRNA, such as the A1CF pre-mRNA or A1CF mature mRNA. The poly A tail of A1CF mRNA is typically disregarded for antisense oligonucleotide targeting.

Further, the target cell may be a hepatocyte. In one embodiment, the target cell is HBV infected primary human hepatocytes, either derived from HBV infected individuals or from a HBV infected mouse with a humanized liver (PhoenixBio, PXB-mouse).

In accordance with the present invention, the target cell may be infected with HBV. Further, the target cell may comprise HBV cccDNA. Thus, the target cell preferably comprises A1CF mRNA, such as the A1CF pre-mRNA or A1CF mature mRNA, and HBV cccDNA. In one embodiment, the target cell is a human cell. In one embodiment, the human cell is a hepatocyte.

### Naturally Occurring Variant

The term “naturally occurring variant” refers to variants of A1CF gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian A1CF target nucleic acid, such as a target nucleic acid of SEQ ID NO: 1 and/or SEQ ID NO: 2. In some embodiments, the naturally occurring variants have at least 99% homology to the human A1CF target nucleic acid of SEQ ID NO: 1. In some embodiments, the naturally occurring variants are known polymorphisms.

### Inhibition of Expression

The term “inhibition of expression” as used herein is to be understood as an overall term for an A1CF (APOBEC1 complementation factor) inhibitors ability to inhibit amount or the activity of A1CF in a target cell. Inhibition of expression or activity may be determined by measuring the level of A1CF pre-mRNA or A1CF mRNA, or by measuring the level of A1CF protein or activity in a cell. Inhibition of expression may be determined in vitro or in vivo. Inhibition is determined by reference to a control. It is generally understood that the control is an individual or target cell treated with a saline composition.

The term “inhibition” or “inhibit” may also be referred to as down-regulate, reduce, suppress, lessen, lower, decrease the expression or activity of A1CF.

The inhibition of expression of A1CF may occur e.g. by degradation of pre-mRNA or mRNA e.g. using RNase H recruiting oligonucleotides, such as gapmers, or nucleic acid molecules that function via the RNA interference pathway, such as siRNA or shRNA. Alternatively, the inhibitor of the present invention may bind to A1CF polypeptide and inhibit the activity of A1CF or prevent its binding to other molecules.

In some embodiments, the inhibition of expression of the A1CF target nucleic acid or the activity of A1CF protein results in a decreased amount of HBV cccDNA in the target cell. Preferably, the amount of HBV cccDNA is decreased as compared to a control. In some embodiments, the decrease in amount of HBV cccDNA is at least 20%, at least 30%, as compared to a control. In some embodiments, the amount of cccDNA in an HBV infected cell is reduced by at least 50%, such as 60%, such as 70%, when compared to a control.

In some embodiments, the inhibition of expression of the A1CF target nucleic acid or the activity of A1CF protein results in a decreased amount of HBV pgRNA in the target cell. Preferably, the amount of HBV pgRNA is decreased as compared to a control. In some embodiments, the decrease in amount of HBV pgRNA is at least 20%, at least 30%, as compared to a control. In some embodiments, the amount of pgRNA in an HBV infected cell is reduced by at least 50%, such as 60%, when compared to a control.

### Sugar Modifications

The oligonucleotide of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (Tₘ). A high affinity modified nucleoside of the present invention preferably result in an increase in melting temperature in the range of +0.5 to +12° C., more preferably in the range of +1.5 to +10° C. and most preferably in the range of +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### 2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

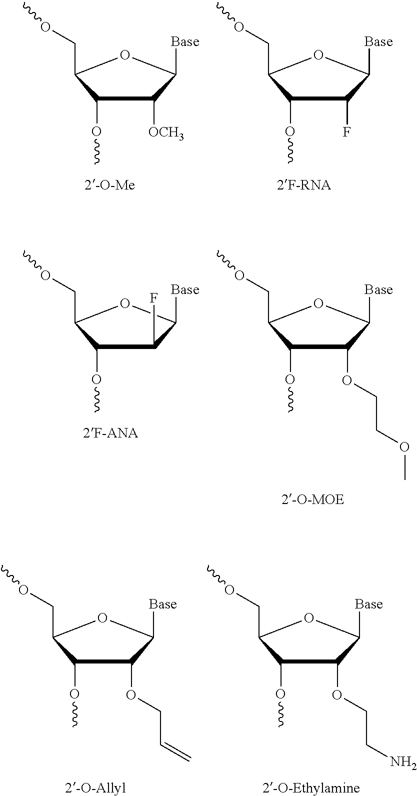

In relation to the present invention 2′ substituted sugar modified nucleosides does not include 2′ bridged nucleosides like LNA.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667.

Particular examples of LNA nucleosides of the invention are presented in Scheme 1 (wherein B is as defined above).

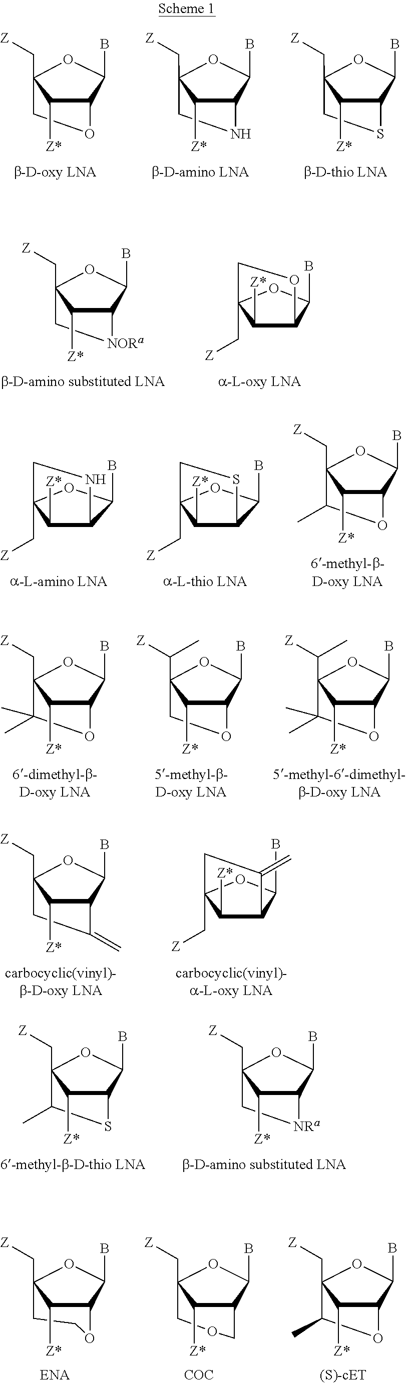

Particular LNA nucleosides are beta-D-oxy-LNA, 6′-methyl-beta-D-oxy LNA such as (S)-6′-methyl-beta-D-oxy-LNA (ScET) and ENA.

### RNase H Activity and Recruitment

The RNase H activity of an antisense oligonucleotide refers to its ability to recruit RNase H when in a duplex with a complementary RNA molecule. WO01/23613 provides in vitro methods for determining RNase H activity, which may be used to determine the ability to recruit RNase H. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO 01/23613 (hereby incorporated by reference). For use in determining RNase H activity, recombinant human RNase H1 is available from Creative Biomart® (Recombinant Human RNase H1 fused with His tag expressed in E. coli).

### Gapmer

The antisense oligonucleotide of the invention, or contiguous nucleotide sequence thereof, may be a gapmer, also termed gapmer oligonucleotide or gapmer designs. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, such as from 15 to 20, such as 16 to 18 nucleosides.

By way of example, the gapmer oligonucleotide of the present invention can be represented by the following formulae:

F₁₋₈-G₅₋₁₈-F′₁₋₈, such as

F₁₋₈-G₇₋₁₈-F′₂₋₈

with the proviso that the overall length of the gapmer regions F-G-F′ is at least 12, such as at least 14 nucleotides in length.

In an aspect of the invention, the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 18 nucleosides which are capable of recruiting RNase H. In some embodiments, the G region consists of DNA nucleosides.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′-substituted sugar modified nucleotide (mixed wing design). In some embodiments, the 2′-substituted sugar modified nucleotide is independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides. In some embodiments, all the modified nucleosides of region F and F′ are beta-D-oxy LNA nucleosides, wherein region F or F′, or F and F′ may optionally comprise DNA nucleosides. In such embodiments, the flanking region F or F′, or both F and F′ comprise at least three nucleosides, wherein the 5′ and 3′ most nucleosides of the F and/or F′ region are LNA nucleosides.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments, the LNA gapmer is of formula: [LNA]₁₋₅-[region G]₆₋₁₈-[LNA]₁₋₅, wherein region G is as defined in the Gapmer region G definition.

### MOE Gapmers

A MOE gapmers is a gapmer wherein regions F and F′ consist of MOE nucleosides. In some embodiments, the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₈, such as [MOE]₅-[Region G]₁₀-[MOE]₅ wherein region G is as defined in the Gapmer definition. MOE gapmers with a 5-10-5 design (MOE-DNA-MOE) have been widely used in the art.

### Region D′ or D″ in an Oligonucleotide

The oligonucleotide of the invention may in some embodiments comprise or consist of the contiguous nucleotide sequence of the oligonucleotide which is complementary to the target nucleic acid, such as a gapmer region F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D″ region may serve as a nuclease susceptible biocleavable linker (see definition of linkers). In some embodiments, the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment, the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₈-F′₂₋₈

D′-F-G-F′, in particular D′₁₋₃-F₁₋₈-G₅₋₁₈-F′₂₋₈

F-G-F′-D″, in particular F₁₋₈-G₅₋₁₈-F′₂₋₈-D″₁₋₃

D′-F-G-F′-D″, in particular D′₁₋₃-F₁₋₈-G₅₋₁₈-F′₂₋₈-D″₁₋₃

In some embodiments, the internucleoside linkage positioned between region D′ and region F is a phosphodiester linkage. In some embodiments, the internucleoside linkage positioned between region F′ and region D″ is a phosphodiester linkage.

### Conjugate

The term “conjugate” as used herein refers to an oligonucleotide which is covalently linked to a non-nucleotide moiety (conjugate moiety or region C or third region). The conjugate moiety may be covalently linked to the antisense oligonucleotide, optionally via a linker group, such as region D′ or D″.

Oligonucleotide conjugates and their synthesis have been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In some embodiments, the non-nucleotide moiety (conjugate moiety) is selected from the group consisting of carbohydrates (e.g. galactose or N-acetylgalactosamine (GalNAc)), cell surface receptor ligands, drug substances, hormones, lipophilic substances, polymers, proteins (e.g. antibodies), peptides, toxins (e.g. bacterial toxins), vitamins, viral proteins (e.g. capsids) or combinations thereof.

Exemplary conjugate moieties are those capable of binding to the asialoglycoprotein receptor (ASGPR). In particular, tri-valent N-acetylgalactosamine conjugate moieties are suitable for binding to the ASGPR, see for example WO 2014/076196, WO 2014/207232 and WO 2014/179620 (hereby incorporated by reference). Such conjugates serve to enhance uptake of the oligonucleotide to the liver.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (region C), to a first region, e.g. an oligonucleotide or contiguous nucleotide sequence complementary to the target nucleic acid (region A).

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment, the biocleavable linker is susceptible to S1 nuclease cleavage. In a preferred embodiment the nuclease susceptible linker comprises between 1 and 5 nucleosides, such as 1, 2, 3, 4 or 5 nucleosides, more preferably between 2 and 4 nucleosides and most preferably 2 or 3 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments, the linker (region Y) is an amino alkyl, such as a C2-C36 amino alkyl group, including, for example C6 to C12 amino alkyl groups. some embodiments the linker (region Y) is a C6 amino alkyl group.

### Treatment

The term “treatment” as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Prophylactic can be understood as preventing an HBV infection from turning into a chronic HBV infection or the prevention of severe liver diseases such as liver cirrhosis and hepatocellular carcinoma caused by a chronic HBV infection.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Preferably, the subject is a mammal. More preferably the subject is human.

As described elsewhere herein, the patient to be treated may suffers from HBV infection, such as chronic HBV infection. In some embodiments, the patient suffering from HBV infection may suffer from hepatocellular carcinoma (HCC). In some embodiments, the patient suffering from HBV infection does not suffer from hepatocellular carcinoma.

### DETAILED DESCRIPTION OF THE INVENTION

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that A1CF is associated with cccDNA stability. This knowledge allows for the opportunity to destabilize cccDNA in HBV infected subjects which in turn opens the opportunity for a complete cure of chronically infected HBV patients.

One aspect of the present invention is an A1CF inhibitor for use in the treatment and/or prevention of Hepatitis B virus (HBV) infection, in particular a chronic HBV infection.

The A1CF inhibitor can for example be a small molecule that specifically binds to A1CF protein, wherein said inhibitor prevents or reduces binding of A1CF protein to cccDNA.

An embodiment of the invention is an A1CF inhibitor which is capable of reducing the amount of cccDNA and/or pgRNA in an infected cell, such as an HBV infected cell.

In a further embodiment, the A1CF inhibitor is capable of reducing HBsAg and/or HBeAg in vivo in an HBV infected individual.

### A1CF Inhibitors for Use in Treatment of HBV

Without being bound by theory, it is believed that A1CF is involved in the stabilization of the cccDNA in the cell nucleus, either via direct or indirect binding to the cccDNA, and by preventing the binding/association of A1CF with cccDNA, the cccDNA is destabilized and becomes prone to degradation. One embodiment of the invention is therefore an A1CF inhibitor which interacts with the A1CF protein, and prevents or reduces its binding/association to cccDNA.

In some embodiments of the present invention, the inhibitor is an antibody, antibody fragment or a small molecule compound. In some embodiments, the inhibitor may be an antibody, antibody fragment or a small molecule that specifically binds to the A1CF protein, such as the A1CF protein encoded by SEQ ID NO: 1, 4, 5, 6, 7, 8, 9, 10 or 11.

### Nucleic Acid Molecules of the Invention

Therapeutic nucleic acid molecules are potentially excellent A1CF inhibitors since they can target the A1CF transcript and promote its degradation either via the RNA interference pathway or via RNase H cleavage. Alternatively, oligonucleotides such as aptamers can also act as inhibitors of A1CF protein interactions.

One aspect of the present invention is an A1CF targeting nucleic acid molecule for use in treatment and/or prevention of Hepatitis B virus (HBV) infection. Such a nucleic acid molecule can be selected from the group consisting of a single stranded antisense oligonucleotide, an siRNA, and a shRNA.

The present section describes novel nucleic acid molecules suitable for use in treatment and/or prevention of Hepatitis B virus (HBV) infection.

The nucleic acid molecules of the present invention are capable of inhibiting expression of A1CF mRNA and/or protein in vitro and in vivo. The inhibition is achieved by hybridizing an oligonucleotide to a target nucleic acid encoding A1CF. The target nucleic acid may be a mammalian A1CF sequence. In some embodiments, the target nucleic acid may be a human A1CF pre-mRNA sequence such as the sequence of SEQ ID NO: 1 or a human mature A1CF mRNA sequence selected from SEQ ID NO: 4 to 11. In some embodiments, the target nucleic acid may be a cynomolgus monkey A1CF sequence such as the sequence of SEQ ID NO: 2.

In some embodiments, the nucleic acid molecule of the invention is capable of modulating the expression of the target by inhibiting or down-regulating it. Preferably, such modulation produces an inhibition of expression of at least 20% compared to the normal expression level of the target, more preferably at least 30%, at least 40%, or at least 50%, inhibition compared to the normal expression level of the target. In some embodiments, the nucleic acid molecule of the invention may be capable of inhibiting expression levels of A1CF mRNA by at least 50% or 60% in vitro by transfecting 25 nM nucleic acid molecule into PXB-PHH cells, this range of target reduction is advantageous in terms of selecting nucleic acid molecules with good correlation to the cccDNA reduction. Suitably, the examples provide assays which may be used to measure A1CF mRNA inhibition (e.g. example 1 and the “Materials and Methods” section). A1CF inhibition is triggered by the hybridization between a contiguous nucleotide sequence of the oligonucleotide, such as the guide strand of a siRNA or gapmer region of an antisense oligonucleotide, and the target nucleic acid. In some embodiments, the nucleic acid molecule of the invention comprises mismatches between the oligonucleotide and the target nucleic acid. Despite mismatches hybridization to the target nucleic acid may still be sufficient to show a desired inhibition of A1CF expression. Reduced binding affinity resulting from mismatches may advantageously be compensated by increased number of nucleotides in the oligonucleotide complementary to the target nucleic acid and/or an increased number of modified nucleosides capable of increasing the binding affinity to the target, such as 2′ sugar modified nucleosides, including LNA, present within the oligonucleotide sequence.

An aspect of the present invention relates to a nucleic acid molecule of 12 to 60 nucleotides in length, which comprises a contiguous nucleotide sequence of at least 12 nucleotides in length, such as at least 12 to 30 nucleotides in length, which is at least 95% complementary, such as fully complementary, to a mammalian A1CF target nucleic acid, in particular a human A1CF nucleic acid. These nucleic acid molecules are capable of inhibiting the expression of A1CF mRNA and/or protein.

An aspect of the invention relates to a nucleic acid molecule of 12 to 30 nucleotides in length, comprising a contiguous nucleotide sequence of at least 12 nucleotides, such as 12 to 30 nucleotides in length which is at least 90% complementary, such as fully complementary, to a mammalian A1CF target sequence.

A further aspect of the present invention relates to a nucleic acid molecule according to the invention comprising a contiguous nucleotide sequence of 14 to 22 nucleotides in length with at least 90% complementary, such as fully complementary, to the target sequence of SEQ ID NO: 1.

In some embodiments, the nucleic acid molecule comprises a contiguous sequence of 12 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the oligonucleotide, or contiguous nucleotide sequence thereof is fully complementary (100% complementary) to a region of the target sequence, or in some embodiments may comprise one or two mismatches between the oligonucleotide and the target sequence.

In some embodiments, the oligonucleotide sequence is 100% complementary to a region of the target sequence of SEQ ID NO: 1 and/or SEQ ID NO: 4, 5, 6, 7, 8, 9, 10 and/or 11.

In some embodiments, the nucleic acid molecule or the contiguous nucleotide sequence of the invention is at least 90% or 95% complementary, such as fully (or 100%) complementary, to the target nucleic acid of SEQ ID NO: 1 and/or 2.

In some embodiments, the oligonucleotide or the contiguous nucleotide sequence of the invention is at least 90% or 95% complementary, such as fully (or 100%) complementary, to the target nucleic acid of SEQ ID NO: 2 and/or SEQ ID NO: 4, 5, 6, 7, 8, 9 10 and/or 11.

In some embodiments, the oligonucleotide or the contiguous nucleotide sequence of the invention is at least 90% or 95% complementary, such as fully (or 100%) complementary, to the target nucleic acid of SEQ ID NO: 1 and/or SEQ ID NO: 2 and/or SEQ ID NO: 3.

In some embodiments, the contiguous sequence of the nucleic acid molecule of the present invention is least 90% complementary, such as fully complementary to a region of SEQ ID NO: 1, selected from the group consisting of target regions 1A to 2001A as shown in Table 4.

In some embodiments, the contiguous sequence of the nucleic acid molecule of the present invention is least 90% complementary, such as fully complementary to a region of SEQ ID NO: 1, selected from the group consisting of target regions 10 to 178C as shown in Table 5.

In some embodiments, the nucleic acid molecule of the invention comprises or consists of 12 to 60 nucleotides in length, such as from 13 to 50, such as from 14 to 35, such as 15 to 30, such as from 16 to 22 contiguous nucleotides in length. In a preferred embodiment, the nucleic acid molecule comprises or consists of 15, 16, 17, 18, 19, 20, 21 or 22 nucleotides in length.

In some embodiments, the contiguous nucleotide sequence of the nucleic acid molecule which is complementary to the target nucleic acids comprises or consists of 12 to 30, such as from 13 to 25, such as from 15 to 23, such as from 16 to 22, contiguous nucleotides in length.

In some embodiments, the oligonucleotide is selected from the group consisting of an antisense oligonucleotide, an siRNA and a shRNA.

In some embodiments, the contiguous nucleotide sequence of the siRNA or shRNA which is complementary to the target sequence comprises or consists of 18 to 28, such as from 19 to 26, such as from 20 to 24, such as from 21 to 23, contiguous nucleotides in length.

In some embodiments, the contiguous nucleotide sequence of the antisense oligonucleotide which is complementary to the target nucleic acids comprises or consists of 12 to 22, such as from 14 to 20, such as from 16 to 20, such as from 15 to 18, such as from 16 to 18, such as from 16, 17, 18, 19 or 20 contiguous nucleotides in length.

In some embodiments, the oligonucleotide or contiguous nucleotide sequence comprises or consists of a sequence selected from the group consisting of sequences listed in Table 6 (Materials and Methods section).

It is understood that the contiguous oligonucleotide sequence (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The nucleic acid molecule of the invention may be designed with modified nucleosides and RNA nucleosides (in particular for siRNA and shRNA molecules) or DNA nucleosides (in particular for single stranded antisense oligonucleotides).

In advantageous embodiments, the nucleic acid molecule or contiguous nucleotide sequence comprises one or more sugar modified nucleosides, such as 2′ sugar modified nucleosides, such as comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

It is advantageous if one or more of the modified nucleoside(s) is a locked nucleic acid (LNA).

In some embodiments the contiguous nucleotide sequence comprises LNA nucleosides.

In some embodiments the contiguous nucleotide sequence comprises LNA nucleosides and DNA nucleosides.

In some embodiments the contiguous nucleotide sequence comprises 2′-O-methoxyethyl (2′MOE) nucleosides.

In some embodiments the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′sugar modified nucleoside.

In a further embodiment the nucleic acid molecule comprises at least one modified internucleoside linkage. Suitable internucleoside modifications are described in the “Definitions” section under “Modified internucleoside linkage”.

Advantageously, the oligonucleotide comprises at least one modified internucleoside linkage, such as phosphorothioate or phosphorodithioate.

In some embodiments, at least one internucleoside linkage in the contiguous nucleotide sequence is a phosphodiester internucleoside linkages.

It is advantageous if at least 2 to 3 internucleoside linkages at the 5′ or 3′ end of the oligonucleotide are phosphorothioate internucleoside linkages.

For single stranded antisense oligonucleotides it is advantageous if at least 75%, such as all, the internucleoside linkages within the contiguous nucleotide sequence are phosphorothioate internucleoside linkages. In some embodiments all the internucleotide linkages in the contiguous sequence of the single stranded antisense oligonucleotide are phosphorothioate linkages.

In an advantageous embodiment of the invention the antisense oligonucleotide of the invention is capable of recruiting RNase H, such as RNase H1. An advantageous structural design is a gapmer design as described in the “Definitions” section under for example “Gapmer”, “LNA Gapmer” and “MOE gapmer”. In the present invention it is advantageous if the antisense oligonucleotide of the invention is a gapmer with an F-G-F′ design.

In all instances the F-G-F′ design may further include region D′ and/or D″ as described in the “Definitions” section under “Region D′ or D” in an oligonucleotide”.

In a further aspect, of the invention the nucleic acid molecules, such as the antisense oligonucleotide, siRNA or shRNA, of the invention can be targeted directly to the liver by covalently attaching them to a conjugate moiety capable of binding to the asialoglycoprotein receptor (ASGPr), such as divalent or trivalent GalNAc cluster.

### Conjugates

Since HBV infection primarily affects the hepatocytes in the liver it is advantageous to conjugate the A1CF inhibitor to a conjugate moiety that will increase the delivery of the inhibitor to the liver compared to the unconjugated inhibitor. In one embodiment, liver targeting moieties are selected from moieties comprising cholesterol or other lipids or conjugate moieties capable of binding to the asialoglycoprotein receptor (ASGPR).

In some embodiments, the invention provides a conjugate comprising a nucleic acid molecule of the invention covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. JB. C. 1996, 271, 6686) or are readily determined using methods typical in the art.

In one embodiment, the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously, the asialoglycoprotein receptor targeting moiety is N-acetylgalactosamine (GalNAc).

To generate the ASGPR conjugate moiety the ASPGR targeting moieties (preferably GalNAc) can be attached to a conjugate scaffold. Generally, the ASPGR targeting moieties can be at the same end of the scaffold. In one embodiment, the conjugate moiety consists of two to four terminal GalNAc moieties linked to a spacer which links each GalNAc moiety to a brancher molecule that can be conjugated to the antisense oligonucleotide.

In a further embodiment, the conjugate moiety is mono-valent, di-valent, tri-valent or tetra-valent with respect to asialoglycoprotein receptor targeting moieties. Advantageously, the asialoglycoprotein receptor targeting moiety comprises N-acetylgalactosamine (GalNAc) moieties.

GalNAc conjugate moieties can include, for example, those described in WO 2014/179620 and WO 2016/055601 and PCT/EP2017/059080 (hereby incorporated by reference), as well as small peptides with GalNAc moieties attached such as Tyr-Glu-Glu-(aminohexyl GalNAc)3 (YEE(ahGalNAc)3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GalNAc conjugate moiety, may be attached to the 3′- or 5′-end of the oligonucleotide using methods known in the art. In one embodiment, the ASGPR conjugate moiety is linked to the 5′-end of the oligonucleotide.

In one embodiment, the conjugate moiety is a tri-valent N-acetylgalactosamine (GalNAc), such as those shown in FIG. 1. In one embodiment, the conjugate moiety is the tri-valent N-acetylgalactosamine (GalNAc) of FIG. 1A-1 or FIG. 1A-2, or a mixture of both. In one embodiment, the conjugate moiety is the tri-valent N-acetylgalactosamine (GalNAc) of FIG. 1B-1 or FIG. 1B-2, or a mixture of both. In one embodiment, the conjugate moiety is the tri-valent N-acetylgalactosamine (GalNAc) of FIG. 1C-1 or FIG. 1C-2, or a mixture of both. In one embodiment, the conjugate moiety is the tri-valent N-acetylgalactosamine (GalNAc) of FIG. 1D-1 or FIG. 1D-2, or a mixture of both.

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand) to covalently attach the conjugate moiety to the oligonucleotide. In a further aspect, a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention.

In a further aspect, the invention provides a pharmaceutically acceptable salt of the nucleic acid molecules or a conjugate thereof, such as a pharmaceutically acceptable sodium salt, ammonium salt or potassium salt.

### Pharmaceutical Composition

In a further aspect, the invention provides pharmaceutical compositions comprising any of the compounds of the invention, in particular the aforementioned nucleic acid molecules and/or nucleic acid molecule conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments, the nucleic acid molecule is used in the pharmaceutically acceptable diluent at a concentration of 50 to 300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

In some embodiments, the nucleic acid molecule or the nucleic acid molecule conjugates of the invention, or pharmaceutically acceptable salt thereof is in a solid form, such as a powder, such as a lyophilized powder.

Compounds, nucleic acid molecules or nucleic acid molecule conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the nucleic acid molecule or nucleic acid molecule conjugate of the invention is a prodrug. In particular with respect to nucleic acid molecule conjugates the conjugate moiety is cleaved off the nucleic acid molecule once the prodrug is delivered to the site of action, e.g. the target cell.

### Administration

The compounds, nucleic acid molecules or nucleic acid molecule conjugates or pharmaceutical compositions of the present invention may be administered topically or enterally or parenterally (such as, intravenous, subcutaneous, or intra-muscular).

In a preferred embodiment, the oligonucleotide or pharmaceutical compositions of the present invention are administered by a parenteral route including intravenous, intraarterial, subcutaneous, intraperitoneal or intramuscular injection or infusion. In one embodiment, the active nucleic acid molecule or nucleic acid molecule conjugate is administered intravenously. In another embodiment, the active nucleic acid molecule or nucleic acid molecule conjugate is administered subcutaneously.

In some embodiments, the nucleic acid molecule, nucleic acid molecule conjugate or pharmaceutical composition of the invention is administered at a dose of 0.1-15 mg/kg, such as from 0.2-10 mg/kg, such as from 0.25-5 mg/kg. The administration can be once a week, every second week, every third week or even once a month.

The invention also provides for the use of the nucleic acid molecule or nucleic acid molecule conjugate of the invention as described for the manufacture of a medicament wherein the medicament is in a dosage form for subcutaneous administration.

### Combination Therapies

In some embodiments the inhibitor of the present invention such as the nucleic acid molecule, nucleic acid molecule conjugate or pharmaceutical composition of the invention is for use in a combination treatment with another therapeutic agent. The therapeutic agent can for example be the standard of care for the diseases or disorders described above.

By way of example, the nucleic acid molecule or the nucleic acid molecule conjugate of the present invention may be used in combination with other actives, such as oligonucleotide-based antivirals—such as sequence specific oligonucleotide-based antivirals—acting either through antisense (including other LNA oligomers), siRNAs (such as ARC520), aptamers, morpholinos or any other antiviral, nucleotide sequence-dependent mode of action.

By way of further example, the nucleic acid molecule or the nucleic acid molecule conjugate of the present invention may be used in combination with other actives, such as immune stimulatory antiviral compounds, such as interferon (e.g. pegylated interferon alpha), TLR7 agonists (e.g. GS-9620), or therapeutic vaccines.

By way of further example, the nucleic acid molecule or the nucleic acid molecule conjugate of the present invention may be used in combination with other actives, such as small molecules, with antiviral activity. These other actives could be, for example, nucleoside/nucleotide inhibitors (e.g. entecavir or tenofovir disoproxil fumarate), encapsidation inhibitors, entry inhibitors (e.g. Myrcludex B).

In certain embodiments, the additional therapeutic agent may be an HBV agent, a Hepatitis C virus (HCV) agent, a chemotherapeutic agent, an antibiotic, an analgesic, a nonsteroidal anti-inflammatory (NSAID) agent, an antifungal agent, an antiparasitic agent, an anti-nausea agent, an anti-diarrheal agent, or an immunosuppressant agent.

In particular, related embodiments, the additional HBV agent may be interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In other particular related embodiments, the additional HCV agent may be interferon alpha-2b, interferon alpha-2a, and interferon alphacon-1 (pegylated and unpegylated); ribavirin; pegasys; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

### Applications

The nucleic acid molecules of the invention may be utilized as research reagents for, for example, diagnostics, therapeutics and prophylaxis.

In research, such nucleic acid molecules may be used to specifically modulate the synthesis of A1CF protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby preventing protein formation or by degrading or inhibiting a modulator of the gene or mRNA producing the protein.

If employing the nucleic acid molecules of the invention in research or diagnostics the target nucleic acid may be a cDNA or a synthetic nucleic acid derived from DNA or RNA.

Also encompassed by the present invention is an in vivo or in vitro method for modulating A1CF expression in a target cell which is expressing A1CF, said method comprising administering a nucleic acid molecule, conjugate compound or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in the liver. The target cell may be a hepatocyte.

One aspect of the present invention is related the nucleic acid molecules, conjugate compounds or pharmaceutical compositions of the invention for use as a medicament.

In an aspect of the invention, the A1CF inhibitor, such as a nucleic acid molecule, conjugate compound or pharmaceutical composition of the invention is capable of reducing the cccDNA level in HBV infected cells and thereby inhibiting HBV infection. In particular, the antisense oligonucleotide is capable of affecting one or more of the following parameters i) reducing cccDNA and/or ii) reducing pgRNA and/or iii) reducing HBV DNA and/or iv) reducing HBV viral antigens in an infected cell.

For example, a nucleic acid molecule that inhibits HBV infection may reduce i) the cccDNA levels in an infected cell by at least 40% such as 50%, 60% or 70% reduction compared to controls; or ii) the level of pgRNA by at least 40% such as 50%, 60% or 70% reduction compared to controls. The controls may be untreated cells or animals, or cells or animals treated with an appropriate control.

Inhibition of HBV infection may be measured in vitro using HBV infected primary human hepatocytes or in vivo using humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of intracellular cccDNA or HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

Due to the reduction of A1CF levels the nucleic acid molecules, conjugate compounds or pharmaceutical compositions of the present invention can be used to inhibit development of or in the treatment of HBV infection. In particular, through the destabilization and reduction of the cccDNA, the nucleic acid molecules, conjugate compounds or pharmaceutical compositions of the present invention more efficiently inhibits development of or treats a chronic HBV infection as compared to a compound that only reduces secretion of HBsAg.

Accordingly, one aspect of the present invention is related to use of an A1CF inhibitor, such as the nucleic acid molecule, conjugate compounds or pharmaceutical compositions of the invention to reduce cccDNA and/or pgRNA in an HBV infected individual.

A further aspect of the invention relates to the use of an A1CF inhibitor, such as the nucleic acid molecules, conjugate compounds or pharmaceutical compositions of the invention to inhibit development of or treat a chronic HBV infection.

A further aspect of the invention relates to the use of A1CF inhibitor, such as the nucleic acid molecules, conjugate compounds or pharmaceutical compositions of the invention to reduce the infectiousness of a HBV infected person. In a particular aspect of the invention, the A1CF inhibitor, such as the nucleic acid molecules, conjugate compounds or pharmaceutical compositions of the invention inhibits development of a chronic HBV infection.

The subject to be treated with the A1CF inhibitor, such as the nucleic acid molecules, conjugate compounds or pharmaceutical compositions of the invention (or which prophylactically receives nucleic acid molecules, conjugate compounds or pharmaceutical compositions of the present invention) is preferably a human, more preferably a human patient who is HBsAg positive and/or HBeAg positive, even more preferably a human patient that is HBsAg positive and HBeAg positive.

Accordingly, the present invention relates to a method of treating a HBV infection, wherein the method comprises administering an effective amount of A1CF inhibitor, such as the nucleic acid molecules, conjugate compounds or pharmaceutical compositions of the invention. The present invention further relates to a method of preventing liver cirrhosis and hepatocellular carcinoma caused by a chronic HBV infection. In one embodiment, the A1CF inhibitors of the present invention is not intended for the treatment of hepatocellular carcinoma, only its prevention.

The invention also provides for the use of a A1CF inhibitor, such as nucleic acid molecule, a conjugate compound or a pharmaceutical composition of the invention for the manufacture of a medicament, in particular a medicament for use in the treatment of HBV infection or chronic HBV infection or reduction of the infectiousness of a HBV infected person. In preferred embodiments, the medicament is manufactured in a dosage form for subcutaneous administration.

The invention also provides for the use of a nucleic acid molecule, a conjugate compound, the pharmaceutical composition of the invention for the manufacture of a medicament wherein the medicament is in a dosage form for intravenous administration.

The A1CF inhibitor, such as the nucleic acid molecule, conjugate or the pharmaceutical composition of the invention may be used in a combination therapy. For example, the nucleic acid molecule, conjugate or the pharmaceutical composition of the invention may be combined with other anti-HBV agents such as interferon alpha-2b, interferon alpha-2a, and interferon alphacon-1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697, WO 2014/179629 and WO2017/216390), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Embodiments of the Invention

The following embodiments of the present invention may be used in combination with any other embodiments described herein. The definitions and explanations provided herein above, in particular in the sections “SUMMARY OF INVENTION”, “DEFINITIONS” and DETAILED DESCRIPTION OF THE INVENTION″ apply mutatis mutandis to the following.
1. An A1CF inhibitor for use in the in the treatment and/or prevention of Hepatitis B virus (HBV) infection.
2. The A1CF inhibitor for the use of embodiment 1, wherein the A1CF inhibitor is administered in an effective amount.
3. The A1CF inhibitor for the use of embodiment 1 or 2, wherein the HBV infection is a chronic infection.
4. The A1CF inhibitor for the use of embodiments 1 to 3, wherein the A1CF inhibitor is capable of reducing the amount of cccDNA and/or pgRNA in an infected cell.
5. The A1CF inhibitor for the use of any one of embodiments 1 to 4, wherein the A1CF inhibitor prevents or reduces the association of A1CF protein to cccDNA.
6. A1CF inhibitor for the use of embodiment 5, wherein said inhibitor is a small molecule that specifically binds to A1CF protein, wherein said inhibitor prevents or reduces association of A1CF protein to cccDNA.
7. A1CF inhibitor for the use of embodiment 6, wherein the A1CF protein is encoded by SEQ ID NO: 4, 5, 6, 7, 8, 9, 10 or 11.
8. The A1CF inhibitor for the use of any one of embodiments 1 to 7, wherein said inhibitor is a nucleic acid molecule of 12-60 nucleotides in length comprising or consisting of a contiguous nucleotide sequence of at least 12 nucleotides in length which is at least 90% complementary to a mammalian A1CF target nucleic acid.
9. The A1CF inhibitor for the use of embodiment 8, which is capable of reducing the level of the mammalian A1CF target nucleic acid.
10. The A1CF inhibitor for the use of embodiment 8 or 9, wherein the mammalian A1CF target nucleic acid is RNA.
11. The A1CF inhibitor for the use of embodiment 10, wherein the RNA is pre-mRNA.
12. The A1CF inhibitor for the use of any one of embodiments 8 to 11, wherein the nucleic acid molecule is selected from the group consisting of antisense oligonucleotide, siRNA and shRNA.
13. The A1CF inhibitor for the use of embodiment 12, wherein the nucleic acid molecule is a single stranded antisense oligonucleotide or a double stranded siRNA.
14. The A1CF inhibitor for the use of any one of embodiments 8 to 13, wherein the mammalian A1CF target nucleic acid is selected from the group consisting of SEQ ID NO: 1, 4, 5, 6, 7, 8, 9, 10 and 11.
15. The A1CF inhibitor for the use of any one of embodiments 8 to 13, wherein the contiguous nucleotide sequence of the nucleic acid molecule is at least 98% complementary, such as fully complementary, to the target nucleic acid of SEQ ID NO: 1 and SEQ ID NO: 2.
16. The A1CF inhibitor for the use of any one of embodiments 8 to 13, wherein the contiguous nucleotide sequence of the nucleic acid molecule is at least 98% complementary, such as fully complementary, to the target nucleic acid of SEQ ID NO: 1 and SEQ ID NO: 2 and SEQ ID NO: 3.
17. The A1CF inhibitor for the use of any one of embodiments 1 to 16, wherein the amount of cccDNA in an HBV infected cell is reduced by at least 50%, such as 60%, when compared to a control.
18. The A1CF inhibitor for the use of any one of embodiments 1 to 16, wherein the amount of pgRNA in an HBV infected cell is reduced by at least 50%, such as 60%, when compared to a control.
19. The A1CF inhibitor for the use of any one of embodiments 8 to 18, wherein the amount of mammalian A1CF target nucleic acid is reduced by at least 50%, such as 60% when compared to a control.
20. A nucleic acid molecule of 12 to 60 nucleotides in length which comprises or consists of a contiguous nucleotide sequence of 12 to 30 nucleotides in length wherein the contiguous nucleotide sequence is at least 90% complementary, such as 95%, such as 98%, such as fully complementary, to a mammalian A1CF target nucleic acid.
21. The nucleic acid molecule of embodiment 20, wherein the nucleic acid molecule is chemically produced.
22. The nucleic acid molecule of embodiment 20 or 21, wherein the mammalian A1CF target nucleic acid is selected from the group consisting of SEQ ID NO: 1, 4, 5, 6, 7, 8, 9, 10 and 11.
23. The nucleic acid molecule of embodiment 20 or 21, wherein the contiguous nucleotide sequence is at least 98% complementary, such as fully complementary to the target nucleic acid of SEQ ID NO: 1 and SEQ ID NO: 2.
24. The nucleic acid molecule of embodiment 20 or 21, wherein the contiguous nucleotide sequence is fully complementary to the target nucleic acid of SEQ ID NO: 1 and SEQ ID NO: 2 and SEQ ID NO: 3.
25. The nucleic acid molecule of any one of embodiments 20 to 23, wherein the nucleic acid molecule is 12 to 30 nucleotides in length.
26. The nucleic acid molecule of any one of embodiments 20 to 25, wherein the nucleic acid molecule is a RNAi molecule, such as a double stranded siRNA or shRNA.
27. The nucleic acid molecule of any one of embodiments 20 to 25, wherein the nucleic acid molecule is a single stranded antisense oligonucleotide.
28. The nucleic acid molecule of any one of embodiments 20 to 27, wherein the contiguous nucleotide sequence is fully complementary to a target nucleic acid sequence selected from Table 4 or Table 5.
29. The nucleic acid molecule of any one of embodiments 20 to 28, which is capable of hybridizing to a target nucleic acid of SEQ ID NO: 1 and SEQ ID NO: 2 with a ΔG° below −15 kcal.
30. The nucleic acid molecule of any one of embodiments 20 to 29, wherein the contiguous nucleotide sequence comprises or consists of at least 14 contiguous nucleotides, particularly 15, 16, 17, 18, 19, 20, 21, or 22 contiguous nucleotides.
31. The nucleic acid molecule of any one of embodiments 20 to 29, wherein the contiguous nucleotide sequence comprises or consists of from 14 to 22 nucleotides.
32. The nucleic acid molecule of embodiment 31, wherein the contiguous nucleotide sequence comprises or consists of 16 to 20 nucleotides.
33. The nucleic acid molecule of any one of embodiments 20 to 32, wherein the nucleic acid molecule comprises or consists of 14 to 25 nucleotides in length.
34. The nucleic acid molecule of embodiment 33, wherein the nucleic acid molecule comprises or consists of at least one oligonucleotide strand of 16 to 22 nucleotides in length.
35. The nucleic acid molecule of any one of embodiment 20 to 34, wherein the contiguous nucleotide sequence is fully complementary to a target sequence selected from the group consisting of SEQ ID NO: 12, 13, 14, and 15.
36. The nucleic acid molecule of any one of embodiments 20 to 35, wherein the contiguous nucleotide sequence has zero to three mismatches compared to the mammalian A1CF target nucleic acid it is complementary to.
37. The nucleic acid molecule of embodiment 36, wherein the contiguous nucleotide sequence has one mismatch compared to the mammalian A1CF target nucleic acid.
38. The nucleic acid molecule of embodiment 36, wherein the contiguous nucleotide sequence has two mismatches compared to the mammalian A1CF target nucleic acid.
39. The nucleic acid molecule of embodiment 36, wherein the contiguous nucleotide sequence is fully complementary to the mammalian A1CF target nucleic acid.
40. The nucleic acid molecule of any one of embodiments 20 to 39, comprising one or more modified nucleosides.
41. The nucleic acid molecule of embodiment 40, wherein the one or more modified nucleosides are high-affinity modified nucleosides.
42. The nucleic acid molecule of embodiment 40 or 41, wherein the one or more modified nucleosides are 2′ sugar modified nucleosides.
43. The nucleic acid molecule of embodiment 42, wherein the one or more 2′ sugar modified nucleosides are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.
44. The nucleic acid molecule of any one of embodiments 40 to 43, wherein the one or more modified nucleosides are LNA nucleosides.
45. The nucleic acid molecule of embodiment 44, wherein the modified LNA nucleosides are selected from the group consisting of oxy-LNA, amino-LNA, thio-LNA, cET, and ENA.
46. The nucleic acid molecule of embodiment 44 or 45, wherein the modified LNA nucleosides are oxy-LNA with the following 2′-4′ bridge —O—CH₂—.
47. The nucleic acid molecule of embodiment 46, wherein the oxy-LNA is beta-D-oxy-LNA.
48. The nucleic acid molecule of embodiment 44 or 45, wherein the modified LNA nucleosides are cET with the following 2′-4′ bridge —O—CH(CH₃)—.
49. The nucleic acid molecule of embodiment 48, wherein the cET is (S)cET, i.e. 6′(S)methyl-beta-D-oxy-LNA.
50. The nucleic acid molecule of embodiment 44 or 45, wherein the LNA is ENA, with the following 2′-4′ bridge —O—CH₂—CH₂—.
51. The nucleic acid molecule of any one of embodiments 20 to 50, wherein the nucleic acid molecule comprises at least one modified internucleoside linkage.
52. The nucleic acid molecule of embodiment 51, wherein the at least one modified internucleoside linkage is a phosphorothioate internucleoside linkage.
53. The nucleic acid molecule of any one of embodiments 20 to 52, wherein the nucleic acid molecule is an antisense oligonucleotide capable of recruiting RNase H.
54. The nucleic acid molecule of embodiment 53, wherein the antisense oligonucleotide or the contiguous nucleotide sequence is a gapmer.
55. The nucleic acid molecule of embodiment 54, wherein the antisense oligonucleotide or contiguous nucleotide sequence thereof consists of or comprises a gapmer of formula 5′-F-G-F′-3′, where region F and F′ independently comprise or consist of 1-4 2′ sugar modified nucleosides and G is a region between 6 and 18 nucleosides which are capable of recruiting RNase H.
56. The nucleic acid molecule of embodiment 55, wherein the 1-4 2′ sugar modified nucleosides are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
57. The nucleic acid molecule of embodiment 55 or 56, wherein one or more of the 1-4 2′ sugar modified nucleosides in region F and F′ are LNA nucleosides.
58. The nucleic acid molecule of embodiment 57, wherein all the 2′ sugar modified nucleosides in region F and F′ are LNA nucleosides.
59. The nucleic acid molecule of any one of embodiments 56 to 58, wherein the LNA nucleosides are selected from beta-D-oxy-LNA, alpha-L-oxy-LNA, beta-D-amino-LNA, alpha-L-amino-LNA, beta-D-thio-LNA, alpha-L-thio-LNA, (S)cET, (R)cET beta-D-ENA and alpha-L-ENA.
60. The nucleic acid molecule of any one of embodiments 56 to 59, wherein region F and F′ consist of identical LNA nucleosides.
61. The nucleic acid molecule of any one of embodiments 56 to 60, wherein all the 2′ sugar modified nucleosides in region F and F′ are oxy-LNA nucleosides.
62. The nucleic acid molecule of any one of embodiments 55 to 61, wherein the nucleosides in region G are DNA nucleosides.
63. The nucleic acid molecule of embodiment 62, wherein region G consists of at least 75% DNA nucleosides.
64. The nucleic acid molecule of embodiment 63, where all the nucleosides in region G are DNA nucleosides.
65. A conjugate compound comprising a nucleic acid molecule according to any one of embodiments 20 to 64, and at least one conjugate moiety covalently attached to said nucleic acid molecule.
66. The conjugate compound of embodiment 65, wherein the nucleic acid molecule is a double stranded siRNA and the conjugate moiety is covalently attached to the sense strand of the siRNA.
67. The conjugate compound of embodiment 65 or 66, wherein the conjugate moiety is selected from carbohydrates, cell surface receptor ligands, drug substances, hormones, lipophilic substances, polymers, proteins, peptides, toxins, vitamins, viral proteins or combinations thereof.
68. The conjugate compound of any one of embodiments 65 to 67, wherein the conjugate moiety is capable of binding to the asialoglycoprotein receptor.
69. The conjugate compound of embodiment 68, wherein the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine.
70. The conjugate compound of embodiment 69, wherein the asialoglycoprotein receptor targeting moiety is N-acetylgalactosamine (GalNAc).
71. The conjugate compound of embodiment 69 or 70, wherein the conjugate moiety is mono-valent, di-valent, tri-valent or tetra-valent with respect to asialoglycoprotein receptor targeting moieties.
72. The conjugate compound of embodiment 71, wherein the conjugate moiety consists of two to four terminal GalNAc moieties and a spacer linking each GalNAc moiety to a brancher molecule that can be conjugated to the antisense compound.
73. The conjugate compound of embodiment 72, wherein the spacer is a PEG spacer.
74. The conjugate compound of any one of embodiments 68 to 73, wherein the conjugate moiety is a GalNAc moiety, such as a tri-valent N-acetylgalactosamine (GalNAc) moiety.
75. The conjugate compound of any one of embodiments 68 to 74, wherein the conjugate moiety is selected from one of the trivalent GalNAc moieties in FIG. 1.
76. The conjugate compound of embodiment 75, wherein the conjugate moiety is the trivalent GalNAc moiety of FIG. 1B-1 or FIG. 1B-2, or a mixture of both.
77. The conjugate compound of embodiment 75, wherein the conjugate moiety is the trivalent GalNAc moiety of FIG. 1D-1 or FIG. 1D-2, or a mixture of both.
78. The conjugate compound of any one of embodiments 65 to 77, comprising a linker which is positioned between the nucleic acid molecule and the conjugate moiety.
79. The conjugate compound of embodiment 78, wherein the linker is a physiologically labile linker.
80. The conjugate compound of embodiment 79, wherein the physiologically labile linker is nuclease susceptible linker.
81. The conjugate compound of any one of embodiments 79 or 80, wherein the physiologically labile linker is composed of 2 to 5 consecutive phosphodiester linkages.
82. The conjugate compound of any one of embodiments 65-81, which display improved cellular distribution between liver vs. kidney or improved cellular uptake into the liver of the conjugate compound as compared to an unconjugated nucleic acid.
83. A pharmaceutical composition comprising a nucleic acid molecule of any one of embodiments 20 to 64, a conjugate compound of any one of embodiments 65 to 82 or acceptable salts thereof and a pharmaceutically acceptable diluent, carrier, salt and/or adjuvant.
84. A method for identifying a compound that prevents, ameliorates and/or inhibits a hepatitis B virus (HBV) infection, comprising:
   a. contacting a test compound with
      i. an A1CF polypeptide; or
      ii. a cell expressing A1CF;
   b. measuring the expression and/or activity of A1CF in the presence or absence of said test compound; and
   c. identifying a compound that reduces the expression and/or activity A1CF and reduces cccDNA.
85. An in vivo or in vitro method for modulating A1CF expression in a target cell which is expressing A1CF, said method comprising administering the nucleic acid molecule of any one of embodiments 20 to 64, a conjugate compound any one of embodiments 65 to 82 or the pharmaceutical composition of embodiment 83 in an effective amount to said cell.
86. The method of embodiments 85, wherein the A1CF expression is reduced by at least 50%, or at least 60% in the target cell compared to the level without any treatment or treated with a control.
87. The method of embodiments 85, wherein the target cell is infected with HBV and the cccDNA in an HBV infected cell is reduced by at least 50%, or at least 60% in the HBV infected target cell compared to the level without any treatment or treated with a control.
88. A method for treating or preventing a disease, such as HBV infection, comprising administering a therapeutically or prophylactically effective amount of the nucleic acid molecule any one of embodiments 20 to 64, a conjugate compound of any one of embodiments 65 to 82, or the pharmaceutical composition of embodiment 83 to a subject suffering from or susceptible to the disease.
89. The nucleic acid molecule of any one of embodiments 20 to 64, or the conjugate compound of any one of embodiments 65 to 82, or the pharmaceutical composition of embodiment 83, for use as a medicament for treatment or prevention of a disease, such as HBV infection, in a subject.
90. Use of the nucleic acid molecule of any one of embodiments 20 to 64, or the conjugate compound of any one of embodiments 65 to 82 for the preparation of a medicament for treatment or prevention of a disease, such as HBV infection, in a subject.
91. The method, the nucleic acid molecule, the conjugate compound, or the use of any one of embodiments 88 to 90, wherein the subject is a mammal.
92. The method, the nucleic acid molecule, the conjugate compound, or the use of embodiment 91, wherein the mammal is human.

The invention will now be illustrated by the following examples which have no limiting character.

### EXAMPLES

### Materials and Methods

siRNA Sequences and Compounds

TABLE 6
Human A1CF sequences targeted by the individual components
of the siRNA pool

| SEQ ID | | Position on | |
|---|---|---|---|
| NO: | A1CF target sequence | SEQ ID NO: 1 | Exon |
| 12 | GUGGACAACUGCCGAUUAU | 49395-49413 | 8 |
| 13 | CUGAAGGUGUUGUCGAUGU | 49477-49495 | 8 |
| 14 | CAACAGAGCCAUUAUCCGA | 69636-69654 | 11 |
| 15 | AGACGUAUGCAGCCGAAUA | 75681-75699 | 14 |

The pool of siRNA (ON-TARGETplus SMART pool siRNA Cat. No. LU-013576-02-0005, Dharmacon) contains four individual siRNA molecules targeting the sequences listed in the above table.

TABLE 7
Control compounds

| | | | | SEQ |
|---|---|---|---|---|
| | | | Sequence | ID |
| Name | Supplier | Order number | 5′ to 3′ sense strand | NO |
| Non-targeting | Dharmacon | #D-001810-01- | UGGUUUACAUGUCGACUAA | 16 |
| negative control | | 05 | | |
| siRNA#1 | | | | |
| Hbx positive | GA life | Custom made | GCACUUCGCUUCACCUCUG | 17 |
| control | science | | | |

### Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC:

The crude compounds are purified by preparative RP-HPLC on a Phenomenex Jupiter® C18 10 μm 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography

### Tₘ Assay:

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Na-phosphate, pH 7.0). The solution is heated to 95° C. for 3 min and then allowed to anneal in room temperature for 30 min. The duplex melting temperatures (Tₘ) are measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

Clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml insulin, 50 nM Dexamethazone, 5 ng/ml EGF, 0.1 mM Asc-2P, 2% DMSO and 10% FBS (Ishida et al., 2015). Cells were cultured at 37° C. incubator in a humidified atmosphere with 5% CO₂. Culture medium was replaced 24 h post-plating and every 2 days until harvest.

### HBV Infected PHH Cells

Fresh primary human hepatocytes (PHH) were provided by PhoenixBio, Higashi-Hiroshima City, Japan (PXB-cells also described in Ishida et al 2015 Am J Pathol. 185(5):1275-85) in 70,000 cells/well in 96-well plate format.

Upon arrival the PHH were infected with an MOI of 2GE using HepG2 2.2.15-derived HBV (batch Z12) by incubating the PHH cells with HBV in 4% (v/v) PEG in PHH medium for 16 hours. The cells were then washed three times with PBS and cultured a humidified atmosphere with 5% CO₂ in fresh PHH medium consisting of DMEM (GIBCO, Cat #21885) supplemented with 10% (v/v) heat-inactivated fetal bovine serum (GI BCO, Cat #10082), 2% (v/v) DMSO, 1% (v/v) Penicillin/Streptomycin (GIBCO, Cat #15140-148), 20 mM HEPES (GIBCO, Cat #15630-080), 44 mM NaHCO₃(Wako, Cat #195-14515), 15 μg/ml L-proline (MP-Biomedicals, Cat #0219472825), 0.25 μg/ml Insulin (Sigma, Cat #11882), 50 nM Dexamethasone (Sigma, Cat #D8893), 5 ng/ml EGF (Sigma, Cat #E9644), and 0.1 mM L-Ascorbic acid 2-phosphate (Wako, Cat #013-12061). Cells were cultured at 37° C. incubator in a humidified atmosphere with 5% CO₂. Culture medium was replaced 24 hours post-plating and three times a week until harvest.

siRNA Transfection

Four days post-infection the cells were transfected with the A1CF siRNA pool (see Table 6) in triplicates. No drug controls (NDC), negative control siRNA and HBx siRNA were included as controls (see Table 7).

Per well a transfection mixture was prepared with 2 μl of either negative control siRNA (stock concentration 1 μM), A1CF siRNA pool (stock concentration 1 μM), HBx control siRNA (stock concentration 0.12 μM) or H₂O (NDC) with 18.2 μl OptiMEM® (Thermo Fisher Scientific Reduced Serum media) and 0.6 μl Lipofectamine® RNAiMAX Transfection Reagent (Thermofisher Scientific catalog No. 13778). The transfection mixture was mixed and incubated at room temperature 5 minutes prior to transfection. Prior to transfection, the medium was removed from the PHH cells and replaced by 100 μl/well William's E Medium+GlutaMAX™ (Gibco, #32551) supplemented with HepaRG supplement without P/S (Biopredic International, #ADD711C). 20 μl of transfection mix was added to each well yielding a final concentration of 16 nM for the negative control siRNA or A1CF siRNA pool, or 1.92 nM for the HBx control siRNA and the plates gently rocked before placing into the incubator. The medium was replaced with PHH medium after 6 hours. The siRNA treatment was repeated on day 6 post-infection as described above. On day 8 post-infection the supernatants were harvested and stored at −20° C. HBsAg and HBeAg can be determined from the supernatants if desired.

Measurement of HBV antigen expressionHBV antigen expression and secretion can be measured in the collected supernatants if desired. The HBV propagation parameters, HBsAg and HBeAg levels, are measured using CLIA ELISA Kits (Autobio Diagnostic #CL0310-2, #CL0312-2), according to the manufacturer's protocol. Briefly, 25 μL of supernatant per well is transferred to the respective antibody coated microtiter plate and 25 μL of enzyme conjugate reagent is added. The plate is incubated for 60 min on a shaker at room temperature before the wells are washed five times with washing buffer using an automatic washer. 25 μL of substrate A and B were added to each well. The plates are incubated on a shaker for 10 min at room temperature before luminescence is measured using an EnVision® luminescence reader (Perkin Elmer).

### Cell Viability Measurements

The cell viability was measured on the supernatant free cells by the Cell Counting Kit −8 (CCK8 from Sigma Aldrich, #96992). For the measurement the CCK8 reagent was diluted 1:10 in normal culture medium and 100 μl/well added to the cells. After 1 h incubation in the incubator 80 μl of the supernatants were transferred to a clear flat bottom 96 well plate and read the absorbance at 450 nm. Absorbance values were normalized to the NDC which was set to 100% to calculate the relative cell viabilities.

Cell viability measurements are used to confirm that any reduction in the viral parameters is not the cause of cell death, the closer the value is to 100% the lower the toxicity.

qRT-PCR for cccDNA and HBV DNA Quantification

Following cell viability determination the cells were washed with PBS once and then lysed with 50 μl/well lysis solution from the TaqMan® Gene Expression Cells-to-CT™ Kit (Thermo Fisher Scientific, #AM1729) and stored at −80° C.

Prior to the cccDNA qPCR analysis, a fraction of the cell lysate was digested with T5 enzyme (15 U/4 μL cell lysate; New England Biolabs, #M0363L). Digestion was done at 37° C. for 30 min.

For the quantification of cccDNA for each reaction 2 μl T5-digested cell lysate, 0.5 μl 20×cccDNA_DANDRI Taqman primer/probe (Life Technologies, custom #AI1RW7N, FAM-dye listed in the Table below), 5 μl TaqMan® Fast Advanced Master Mix (Applied Biosystems, #4444557) and 2.5 μl DEPC-treated water were used. Technical triplicates were run for each sample.

| Primer name | Sequence | SEQ ID |
|---|---|---|
| CCCDNA_DANDRI_F | CCGTGTGCACTTCGCTTCA | 18 |
| CCCDNA_DANDRI_R | GCACAGCTTGGAGGCTTGA | 19 |
| CCCDNA_DANDRI_M | 5′-[6FAM]CATGGAGACCACCGTGAACGCCC[BHQ1]-3′ | 20 |

For quantification of intra-cellular HBV DNA and the normalization control, human hemoglobin beta (HBB), for each reaction 2 μl undigested cell lysate, 0.5 μl 20×HBV Taqman primer/probe (Life Technologies, #Pa03453406_s1, FAM-dye), 0.5 μl 20×HBB Taqman® primer/probe (Life Technologies, #Hs00758889_s1, VIC-dye), 5 μl TaqMan® Fast Advanced Master Mix (Applied Biosystems, #4444557) and 2 μl DEPC-treated water were used. Technical triplicates were run for each sample.

The qRT-PCR was run on the QuantStudio™ K12 Flex with standard settings for the fast heating block (95° C. for 20 seconds, then 40 cycles with 95° C. for 1 second and 60° C. for 20 seconds).

Any outliers were removed from the data set by excluding values with more than 0.9 difference to the median Ct of all 9 biological & technical replicates for each sample. Fold changes for cccDNA and total HBV DNA were determined from the Ct values via the 2^{-ddCT} method, and normalized to the HBB as housekeeping gene. The expression levels are presented as % of the average no drug control samples (i.e. the lower the value the larger the inhibition/reduction).

Real-Time PCR for Measuring A1CF mRNA Expression

For quantification of A1CF RNA levels and the normalization control, GUS B, the TaqMan® RNA-to-Ct™ 1-Step Kit (Life Technologies, #4392656) was used. For each reaction 2 μl undigested cell lysate, 0.5 μl 20×A1CF Taqman primer/probe (Life Technologies, #Hs00205840_m1, FAM-dye), 0.5 μl 20×GUS B Taqman primer/probe (Life Technologies, #Hs00939627_m1, VIC-dye), 5 μl 2×TaqMan® RT-PCR Mix, 0.25 μl 40×TaqMan® RT Enzyme Mix and 1.75 μl DEPC-treated water were used. Technical triplicates were run for each sample and minus RT controls included to evaluate potential amplification due to DNA present.

The qRT-PCR was run on the QuantStudio™ K12 Flex with 48 C for 15 min, 95° C. for 10 min, then 40 cycles with 95° C. for 15 seconds and 60 C for 60 seconds.

The A1CF mRNA expression levels were analyzed using the comparative cycle threshold 2-ΔΔCt method normalized to the reference gene GUS B and to non-transfected cells. Primers used for GUS B RNA and target mRNA quantification are listed in Table 8. The expression levels are presented as % of the average no drug control samples (i.e. the lower the value the larger the inhibition/reduction).

TABLE 8
GUS B and A1CF mRNA qPCR primers
(Thermo Fisher Scientific)

| | |
|---|---|
| A1CF primers | Hs00205840_m1 |
| Housekeeping gene primers | Hs00939627_m1 |

### Example 1: Measurement of the Reduction of A1CF mRNA, HBV Intracellular DNA and cccDNA in HBV Infected PHH Cells Resulting from siRNA Treatment

In the following experiment, the effect of A1CF knock-down on the HBV parameters, HBV DNA and cccDNA, was tested.

HBV infected PHH cells were treated with the pool of siRNAs from Dharmacon (LU-013576-02-0005, see Table 6) as described in the Materials and Methods section “siRNA transfection”.

Following the 4 days-treatment, A1CF mRNA, cccDNA and intracellular HBV DNA were measured by qPCR as described in the Materials and Methods section “Real-time PCR for measuring A1CF mRNA Expression” and “qRT-PCR for cccDNA and HBV DNA quantification”. The results are shown in Table 9 as % of the average no drug control samples (i.e. the lower the value the larger the inhibition/reduction).

TABLE 9
Effect on HBV parameters following
knockdown of A1CF with pool of siRNA.
Values are given as average of
biological and technical triplicates.

| | | | HBV | | | |
|---|---|---|---|---|---|---|
| | A1CF | | intracellular | | | |
| | mRNA* | | DNA | | cccDNA | |
| Treatment | Mean | SD | Mean | SD | Mean | SD |
| A1CF siRNA | 39 | 6 | 40 | 15 | 24 | 3 |
| HBx positive | ND | ND | 56 | 30 | 94 | 60 |
| control | | | | | | |
| siRNA negative | ND | ND | 94 | 37 | 109 | 63 |
| control | | | | | | |

ND = not determined

From this it can be seen that the A1CF siRNA pool is capable of reducing A1CF mRNA, cccDNA as well as HBV DNA quite efficiently. The positive control reduced intracellular HBV DNA as expected but had no effect on cccDNA.

1. An A1CF (APOBEC1 complementation factor) inhibitor for use in the treatment of Hepatitis B virus (HBV) infection.

2. The A1CF inhibitor for use according to claim 1, wherein the HBV infection is a chronic infection.

3. The A1CF inhibitor for use according to claim 1 or 2, wherein the A1CF inhibitor is capable of reducing the amount of cccDNA (covalently closed circular DNA) in an HBV infected cell.

4. The A1CF inhibitor for use according to any one of claims 1 to 3, wherein said inhibitor is a nucleic acid molecule of 12 to 60 nucleotides in length comprising a contiguous nucleotide sequence of at least 12 nucleotides in length which is at least 95% complementary, such as fully complementary, to a mammalian A1CF target sequence, in particular a human A1CF target sequence, and is capable of reducing the expression of A1CF mRNA in a cell which expresses the A1CF mRNA.

5. The A1CF inhibitor for use according to any one of claims 1 to 4, wherein said inhibitor is selected from the group consisting of a single stranded antisense oligonucleotide, an siRNA and a shRNA.

6. The A1CF inhibitor for use according to any one of claims 1 to 5, wherein the mammalian A1CF target sequence is selected from the group consisting of SEQ ID NOs: 1, 4, 5, 6, 7, 8, 9, 10, and 11.

7. The A1CF inhibitor for use according to any one of claims 4 to 6, wherein the contiguous nucleotide sequence is at least 98% complementary, such as fully complementary, to the target sequence of SEQ ID NO: 1 and SEQ ID NO: 2.

8. The A1CF inhibitor for use according to any one of claims 3 to 7, wherein the amount of cccDNA in the HBV infected cell is reduced by at least 60%.

9. The A1CF inhibitor for use according to any one of claims 4 to 7, wherein the A1CF mRNA is reduced by at least 60%.

10. A nucleic acid molecule of 12 to 30 nucleotides in length comprising a contiguous nucleotides sequence of at least 12 nucleotides which is 90% complementary, such as fully complementary, to a mammalian A1CF target sequence, in particular a human A1CF target sequence, wherein the nucleic acid molecule is capable of inhibiting the expression of A1CF mRNA.

11. The nucleic acid molecule according to claim 10, wherein the contiguous nucleotide sequence is fully complementary to a sequence selected from the group consisting of SEQ ID NOs: 1, 4, 5, 6, 7, 8, 9, 10, and 11.

12. The nucleic acid molecule according to claim 10 or 11, wherein the nucleic acid molecule comprises a contiguous nucleotide sequence of 12 to 25, such as 16 to 20 nucleotides in length.

13. The nucleic acid molecule of any one of claims 10 to 12, wherein the nucleic acid molecule is a RNAi molecule, such as a double stranded siRNA or a shRNA.

14. The nucleic acid molecule of any one of claims 10 to 12, wherein the nucleic acid molecule is a single stranded antisense oligonucleotide.

15. The nucleic acid molecule according to 14, wherein the single stranded antisense oligonucleotide is capable of recruiting RNase H.

16. The nucleic acid molecule according to any one of claims 10 to 15, wherein the nucleic acid molecule comprises one or more 2′ sugar modified nucleosides.

17. The nucleic acid molecule according to claim 16, wherein the one or more 2′ sugar modified nucleosides are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

18. The nucleic acid molecule according to any one of claim 16 or 17, wherein the one or more 2′ sugar modified nucleosides are LNA nucleosides.

19. The nucleic acid molecule according to any one of claims 10 to 18, where the contiguous nucleotide sequence comprises at least one phosphorothioate internucleoside linkage.

20. The nucleic acid molecule according to claim 19, wherein all the internucleoside linkages within the contiguous nucleotide sequence are phosphorothioate internucleoside linkages.

21. The nucleic acid molecule according to any one of claims 10 to 20, wherein the nucleic acid molecule, or contiguous nucleotide sequence thereof, comprises a gapmer of formula 5′-F-G-F′-3′, wherein regions F and F′ independently comprise 1-4 2′ sugar modified nucleosides and G is a region between 6 and 18 nucleosides which are capable of recruiting RNase H, such as a region comprising between 6 and 18 DNA nucleosides.

22. A conjugate compound comprising a nucleic acid molecule according to any one of claims 10 to 21 and at least one conjugate moiety covalently attached to said nucleic acid molecule.

23. The conjugate compound of claim 22, wherein the conjugate moiety is or comprises a GalNAc moiety, such as a trivalent GalNAc moiety, for example a GalNAc moiety selected from one or more of the trivalent GalNAc moieties in FIG. 1.

24. The conjugate compound of claim 22 or 23, wherein the conjugate compound comprises a physiologically labile linker composed of 2 to 5 linked nucleosides comprising at least two consecutive phosphodiester linkages, wherein the physiologically labile linker is covalently bound at the 5′ or 3′ terminal of the nucleic acid molecule.

25. A pharmaceutically acceptable salt of a nucleic acid molecule according to any one of claims 10 to 21, or a conjugate compound according to any one of claims 22 to 24.

26. A pharmaceutical composition comprising a nucleic acid molecule according to any one of claims 10 to 21, a conjugate compound according to any one of claims 22 to 24, or a pharmaceutically acceptable salt according to claim 25 and a pharmaceutically acceptable excipient.

27. An in vivo or in vitro method for inhibiting A1CF expression in a target cell which is expressing A1CF, said method comprising administering a nucleic acid molecule according to any one of claims 10 to 21, a conjugate compound according to any one of claims 22 to 24, a pharmaceutically acceptable salt according to claim 25, or a pharmaceutical composition according to claim 26 in an effective amount to said cell.

28. A method for treating a disease comprising administering a therapeutically or prophylactically effective amount of a nucleic acid molecule according to any one of claims 10 to 21, a conjugate compound according to any one of claims 22 to 24, a pharmaceutically acceptable salt according to claim 25, or a pharmaceutical composition according to claim 26, to a subject suffering from or susceptible to a disease.

29. A method according to claim 28, wherein the disease is Hepatitis B Virus (HBV) infection, such as a chronic HBV infection.

30. A nucleic acid molecule according any one of claims 10 to 21, a conjugate compound according to any one of claims 22 to 24, a pharmaceutically acceptable salt according to claim 25, or a pharmaceutical composition according to claim 26 for use in medicine.

31. A nucleic acid molecule according any one of claims 10 to 21, a conjugate compound according to any one of claims 22 to 24, a pharmaceutically acceptable salt according to claim 25, or a pharmaceutical composition according to claim 26, for use in the treatment of Hepatitis B Virus (HBV) infection, such as a chronic HBV infection.

32. Use of a nucleic acid molecule according any one of claims 10 to 21, a conjugate compound according to any one of claims 22 to 24, a pharmaceutically acceptable salt according to claim 25, or a pharmaceutical composition according to claim 26, for the preparation of a medicament for the treatment of Hepatitis B Virus (HBV) infection, such as a chronic HBV infection.
